(19) United States
(12) Patent Application Publication
(10) Pub. No.: US 2001/0023070 A1
(43) Pub. Date: Sep. 20, 2001

(54) Interleukins-21 and 22

(76) Inventors: Reinhard Ebner, Gaithersburg, MD (US); Steven M. Ruben, Olney, MD (US)

Correspondence Address: HUMAN GENOME SCIENCES INC, 9410 KEY WEST AVENUE, ROCKVILLE, MD, 20850

(21) Appl. No.: 09/731,816

(22) Filed: Dec. 8, 2000

Related U.S. Application Data
(63) Continuation-in-part of application No. 09/320,713, filed on May 27, 1999.
(63) Continuation-in-part of application No. PCT/US99/11644, filed on May 27, 1999.
(60) Provisional application No. 60/087,340, filed on May 29, 1998.
(60) Provisional application No. 60/131,965, filed on Apr. 30, 1999.
(60) Provisional application No. 60/169,837, filed on Dec. 9, 1999.

(51) Int. Cl. C12P 21/02; C12Q 1/68; G01N 33/53; C07H 21/04; C12N 5/06

(52) U.S. Cl. 435/69.5; 435/6; 435/325; 536/23.5; 530/351; 435/7.1

(57) ABSTRACT

The present invention relates to novel human proteins designated Interleukin-21 (IL-21) and Interleukin-22 (IL-22), and isolated polynucleotides encoding these proteins. Also provided are vectors, host cells, antibodies, and recombinant methods for producing these human proteins. The invention further relates to diagnostic and therapeutic methods useful for diagnosing, treating, and/or preventing disorders related to these novel human proteins.

This application claims benefit under 35 U.S.C. § 119(e) of the filing date of copending U.S. Provisional application Ser. No. 60/169,837, filed on Dec. 9, 1999; under 35 U.S.C. § 120 of the filing date of copending U.S. application Ser. No. 09/320,713, filed May 27, 1999, which, in turn, claims benefit under 35 U.S.C. § 119(e) of the filing date of U.S. Provisional applications Ser. Nos. 60/087,340, filed on May 29, 1998, 60/099,805, filed on Sep. 10, 1998, and 60/131,965, filed on Apr. 30, 1999; and under 35 U.S. C. § 120 of the filing date of copending International Application Serial No. US99/11644, filed May 27, 1999; each of which is hereby incorporated by reference in its entirety.

### FIELD OF THE INVENTION

The present invention relates to two novel human genes, each of which encodes a polypeptide which is a member of the Interleukin family. More specifically, the present invention relates to a polynucleotide encoding a novel human polypeptide named Interleukin-21, or “IL-21”. The present invention also relates to a polynucleotide encoding a novel human polypeptide named Interleukin-22, or “IL-22”. This invention also relates to IL-21 and IL-22 polypeptides, as well as vectors, host cells, antibodies directed to IL-21 and IL-22 polypeptides, and recombinant methods for producing the same. Also provided are diagnostic methods for detecting disorders related to the immune system, and therapeutic methods for treating, preventing, and/or diagnosing such disorders. The invention further relates to screening methods for identifying agonists and antagonists of IL-21 and IL-22 activity.

### BACKGROUND OF THE INVENTION

Cytokines typically exert their respective biochemical and physiological effects by binding to specific receptor molecules. Receptor binding then stimulates specific signal transduction pathways (Kishimoto, T., et al., Cell 76:253-262 (1994)). The specific interactions of cytokines with their receptors are often the primary regulators of a wide variety of cellular processes including activation, proliferation, and differentiation (Arai, K. K. -I, et al., Ann. Rev. Biochem. 59:783-836 (1990); Paul, W. E. and Seder, R. A., Cell 76:241-251 (1994)).

Human interleukin (IL)-17, a closely related homolog of the molecules of the present invention, was only recently identified. IL-17 is a 155 amino acid polypeptide which was molecularly cloned from a CD4+ T-cell cDNA library (Yao, Z., et al., J. Immunol. 155:5483-5486 (1995)). The IL-17 polypeptide contains an N-terminal signal peptide and contains approximately 72% identity at the amino acid level with a T-cell trophic herpesvirus saimiri (HVS) gene designated HVS13. High levels of IL-17 are secreted from CD4-positive primary peripheral blood leukocytes (PBL) upon stimulation (Yao, Z., et al., Immunity 3:811-821 (1995)). Treatment of fibroblasts with IL-17, HVS13, or another murine homologue, designated CTLA8, activate signal transduction pathways and result in the stimulation of the NF-kappaB transcription factor family, the secretion of IL-6, and the costimulation of T-cell proliferation (Yao, Z., et al., Immunity 3:811-821 (1995)).

An HVS13-Fc fusion protein was used to isolate a murine IL-17 receptor molecule which does not appear to belong to any of the previously described cytokine receptor families (Yao, Z., et al., Immunity 3:811-821 (1995)). The murine IL-17 receptor (mIL-17R) is predicted to encode a type I transmembrane protein of 864 amino acids with an apparent molecular mass of 97.8 kDa. mIL-17R is predicted to possess an N-terminal signal peptide with a cleavage site between alanine-31 and serine-32. The molecule also contains a 291 amino acid extracellular domain, a 21 amino acid transmembrane domain, and a 521 amino acid cytoplasmic tail. A soluble recombinant IL-17R molecule consisting of 323 amino acids of the extracellular domain of IL-17R fused to the Fc portion of human immunoglobulin IgGI was able to significantly inhibit IL-17-induced IL-6 production by murine NIH-3T3 cells (supra).

Interestingly, the expression of the IL-17 gene is highly restricted. It is typically observed primarily in activated T-lymphocyte memory cells (Broxmeyer, H. J. Exp. Med. 183:2411-2415 (1996); Fossiez, F., et al., J. Exp. Med. 183:2593-2603 (1996)). Conversely, the IL-17 receptor appears to be expressed in a large number of cells and tissues (Rouvier, E., et al., J. Immunol. 150:5445-5456 (1993); Yao, Z., et al., J. Immunol. 155:5483-5486 (1995)). It remains to be seen, however, if IL-17 itself can play an autocrine role in the expression of IL-17. IL-17 has been implicated as a causative agent in the expression of IL-6, IL-8, G-CSF, Prostaglandin E (PGE₂), and intracellular adhesion molecule (ICAM)-1 (Fossiez, F., supra; Yao, Z., et al., Immunity 3:811-821 (1995)). Each of these molecules possesses highly relevant and potentially therapeutically valuable properties. For instance, IL-6 is involved in the regulation of hematopoietic stem and progenitor cell growth and expansion (Ikebuchi, K., et al., Proc. Natl. Acad. Sci. USA 84:9035-9039 (1987); Gentile, P. and Broxmeyer, H. E. Ann. N.Y. Acad. Sci. USA 628:74-83 (1991)). IL-8 exhibits a myelosuppressive activity for stem cells and immature subsets of myeloid progenitors (Broxmeyer, H. E., et al., Ann. Hematol. 71:235-246 (1995); Daly, T. J., et al., J. Biol. Chem. 270:23282-23292 (1995)). G-CSF acts both early and late to activate and stimulate hematopoiesis in general, and more specifically on neutrophil hematopoiesis, while PGE₂ enhances erythropoiesis, suppresses lymphopoiesis and myelopoiesis in general, and strongly suppresses monocytopoiesis (Broxmeyer, H. E. Amer. J Ped. Hematol./Oncol. 14:22-30 (1992); Broxmeyer, H. E. and Williams, D. E. CRC Crit. Rev. Oncol./Hematol. 8:173-226 (1988)).

Thus, there is a need for polypeptides that function as immunoregulatory molecules and, thereby, modulate the transfer of an extracellular signal ultimately to the nucleus of the cell, since disturbances of such regulation may be involved in disorders relating to cellular activation, hemostasis, angiogenesis, tumor metastasis, cellular migration and ovulation, as well as neurogenesis. Therefore, there is a need for identification and characterization of such human polypeptides which can play a role in detecting, preventing, ameliorating or correcting such disorders.

### SUMMARY OF THE INVENTION

The present invention relates to novel polynucleotides and the encoded polypeptides of IL-21 and IL-22. Moreover, the present invention relates to vectors, host cells, antibodies, and recombinant methods for producing the polypeptides and polynucleotides. Also provided are diagnostic methods for detecting disorders related to the polypeptides, and therapeutic methods for treating and/or preventing such disorders. The invention further relates to screening methods for identifying binding partners of IL-21 and IL-22.

### BRIEF DESCRIPTION OF THE DRAWINGS

FIG. 1 shows the partial nucleotide sequence (SEQ ID NO: 1) and the deduced amino acid sequence (SEQ ID NO: 2) of IL-21. The locations of conserved Domains I-IV (see below) are underlined and labeled as such.

FIGS. 2A and 2B show the nucleotide sequence (SEQ ID NO: 3) and the deduced amino acid sequence (SEQ ID NO: 4) of IL-22. The locations of conserved Domains I-IV (see below) are underlined and labeled as such. The locations of two potential N-linked glycosylation sites are identified by a bolded asparagine symbol (N) accompanied by a bolded pound sign (#) located above the initial nucleotide of the codon encoding the corresponding asparagine.

FIGS. 3A, 3B, and 3C show the regions of identity between the amino acid sequences of: (1) human Interleukin-17 (designated IL-17.aa in the figure; GenBank Accession No. U32659; SEQ ID NO: 5); (2) mouse Interleukin-17 (designated mIL-17.aa in the figure; GenBank Accession No. U43088; SEQ ID NO: 6); (3) viral Interleukin-17 (designated vIL-17.aa in the figure; GenBank Accession No. X64346; SEQ ID NO: 7); (4) IL-20 (designated IL20.aa in the figure and disclosed in copending U.S. Provisional application Ser. No. 60/060,140; filed Sep. 26, 1997; SEQ ID NO: 8); (5) a partial-length IL-21 protein (SEQ ID NO: 2); (6) the full-length IL-21 protein (designated IL-21FL.aa in the figure); (7) a partial-length IL-22 protein (designated IL-22.aa in the figure), and (8) an IL-22 protein (designated IL22ext.aa in the figure), as determined by aligning the sequences using the MegAlign component of the computer program DNA*Star (DNASTAR, Inc., 1228 S. Park St., Madison, Wis. 53715 USA) using the default parameters.

FIG. 4 shows an analysis of the partial IL-21 amino acid sequence (SEQ ID NO: 2). Alpha, beta, turn and coil regions; hydrophilicity and hydrophobicity; amphipathic regions; flexible regions; antigenic index and surface probability are shown. In the “Antigenic Index” or “Jameson-Wolf” graph, the positive peaks indicate locations of the highly antigenic regions of the IL-21 protein, that is, regions from which epitope-bearing peptides of the invention can be determined. Polypeptides and polynucleotides encoding polypeptides comprising the domains defined by these graphs are contemplated by the present invention.

FIG. 5 shows an analysis of the IL-22 amino acid sequence. Alpha, beta, turn and coil regions; hydrophilicity and hydrophobicity; amphipathic regions; flexible regions; antigenic index and surface probability are shown. In the “Antigenic Index” or “Jameson-Wolf” graph, the positive peaks indicate locations of the highly antigenic regions of the IL-22 protein, that is, regions from which epitope-bearing peptides of the invention can be determined. Polypeptides and polynucleotides encoding polypeptides comprising the domains defined by these graphs are contemplated by the present invention.

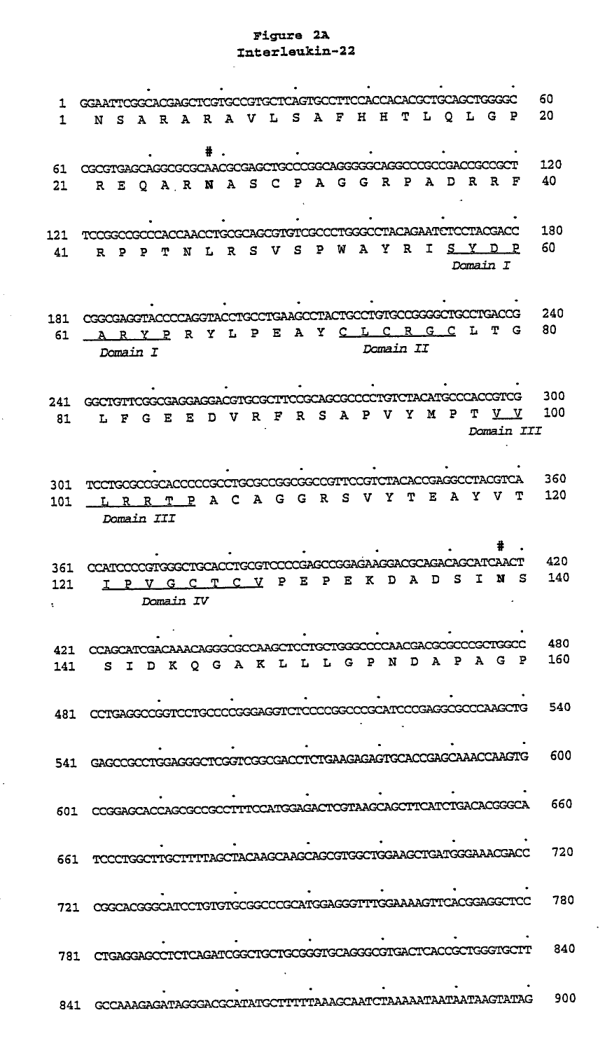

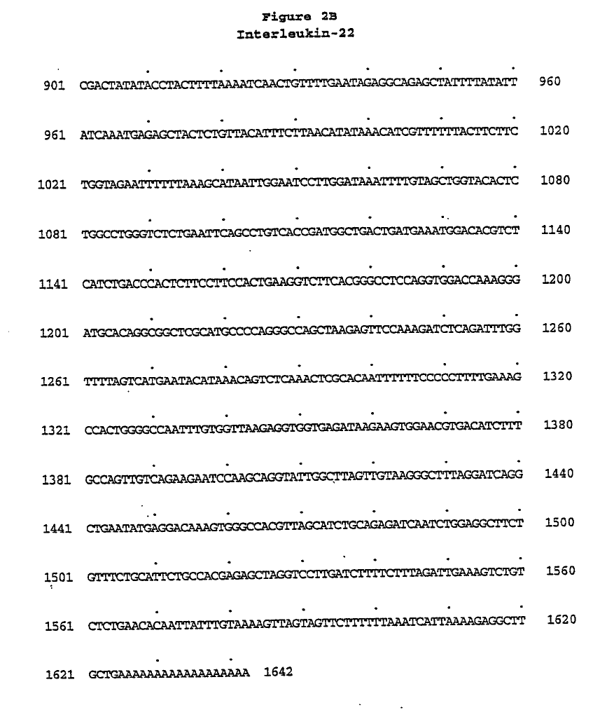

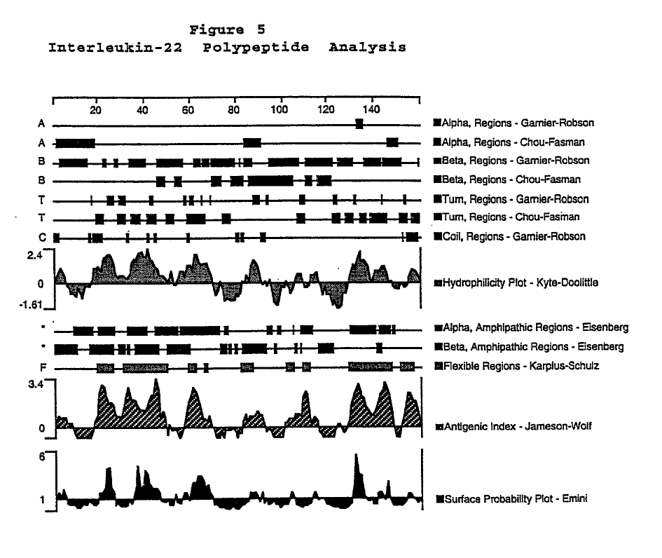

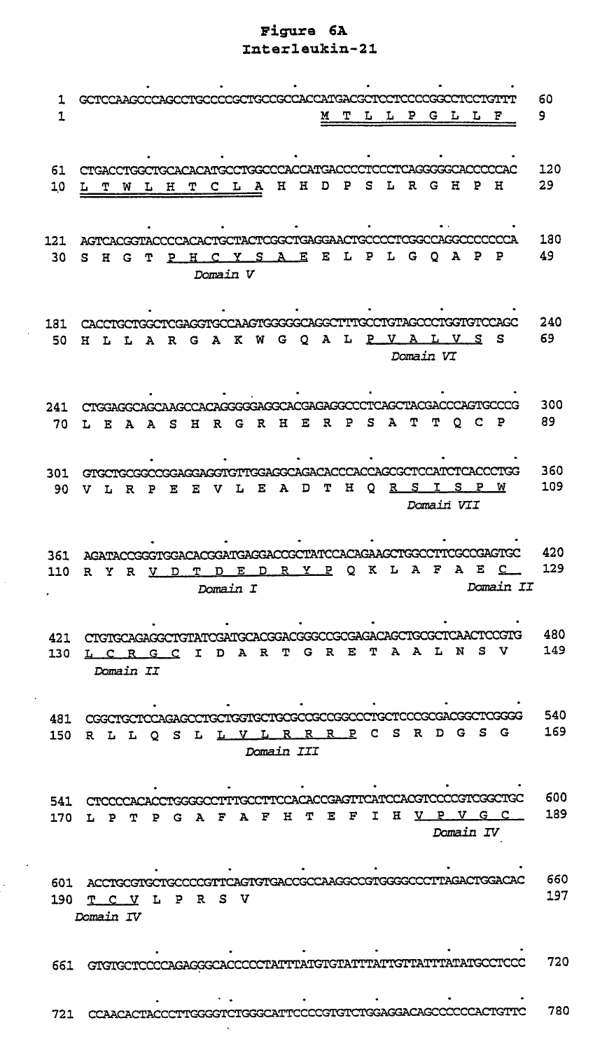

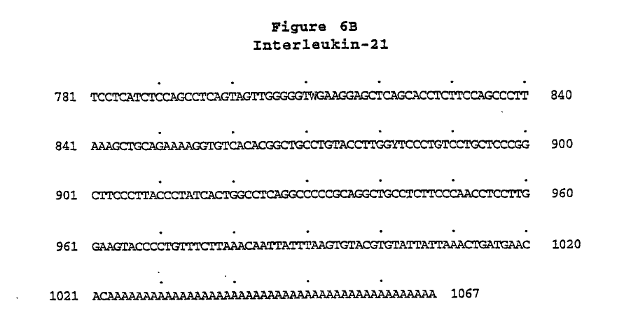

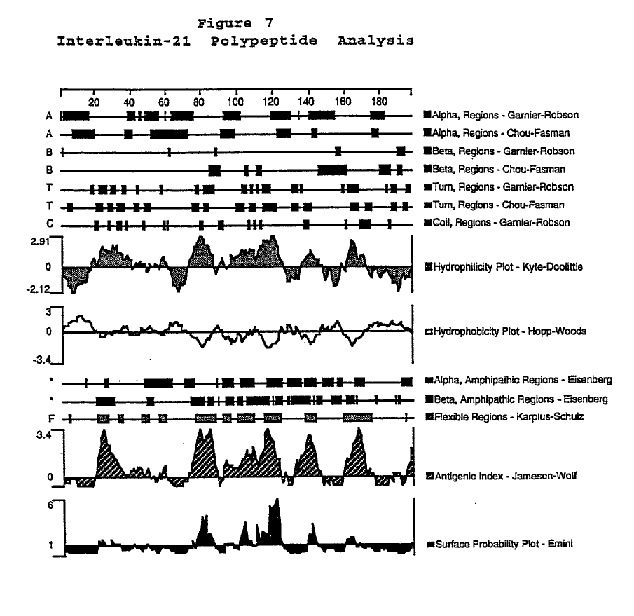

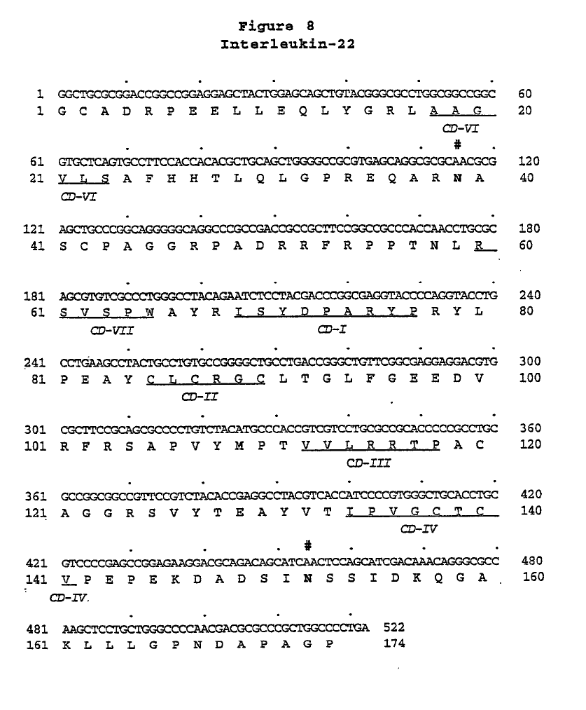

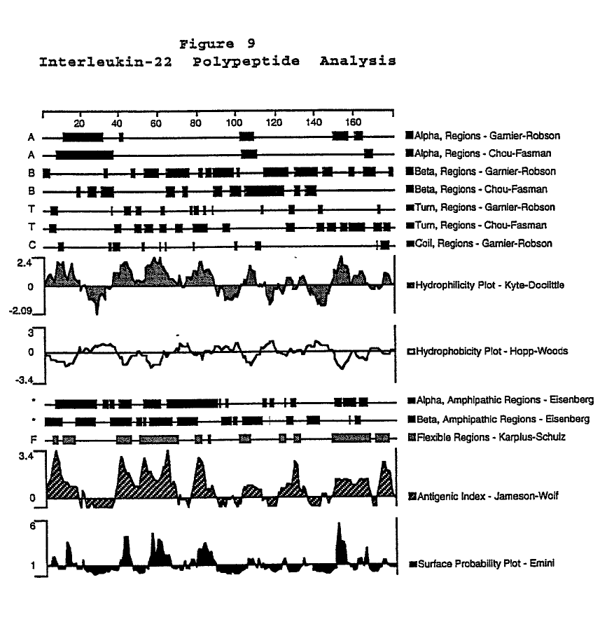

The data presented in FIG. 5 are also represented in tabular form in Table II. The columns are labeled with the headings “Res”, “Position”, and Roman Numerals I-XIII. The column headings refer to the following features of the amino acid sequence presented in FIG. 5 and Table II: “Res”: amino acid residue of SEQ ID NO: 4 or FIGS. 2A and 2B; “Position”: position of the corresponding residue within SEQ ID NO: 4 or FIGS. 2A and 2B; I: Alpha, Regions - Gamier-Robson; II: Alpha, Regions - Chou-Fasman; III: Beta, Regions - Garnier-Robson; IV: Beta, Regions - Chou-Fasman; V: Turn, Regions - Garnier-Robson; VI: Turn, Regions - Chou-Fasman; VII: Coil, Regions - Garnier-Robson; VIII: Hydrophilicity Plot - Kyte-Doolittle; IX: Alpha, Amphipathic Regions - Eisenberg; X: Beta, Amphipathic Regions - Eisenberg; XI: Flexible Regions - Karplus-Schulz; XII: Antigenic Index - Jameson-Wolf; and XIII: Surface Probability Plot - Emini.

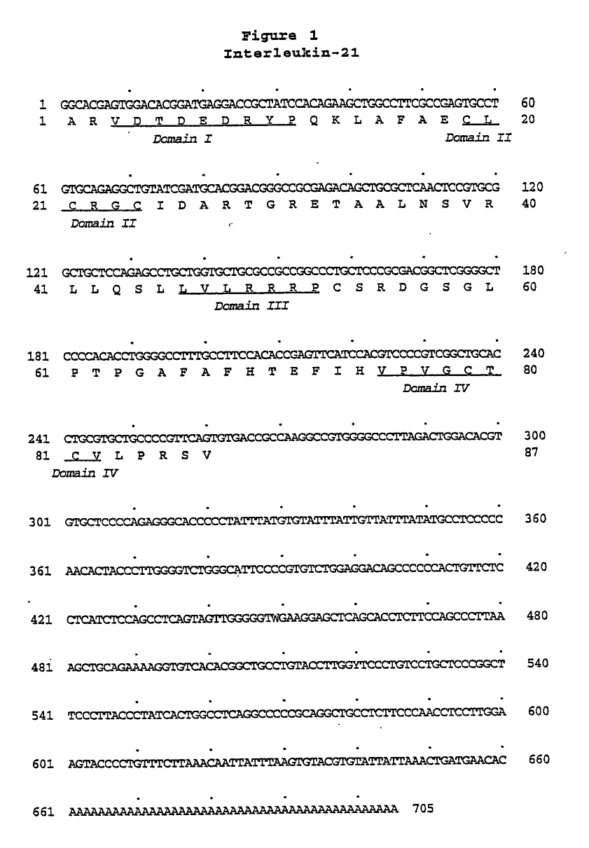

FIGS. 6A and 6B show the nucleotide sequence (SEQ ID NO: 28) and the deduced amino acid sequence (SEQ ID NO: 29) of the full-length IL-21. The locations of conserved Domains I-IV (identical to those shown in FIG. 1) and of conserved Domains V-VII are underlined and labeled as such. A predicted signal peptide from methionine-1 to alanine-18 is double underlined.

FIG. 7 shows an analysis of a full-length IL-21 amino acid sequence. Alpha, beta, turn and coil regions; hydrophilicity and hydrophobicity; amphipathic regions; flexible regions; antigenic index and surface probability are shown. In the “Antigenic Index” or “Jameson-Wolf” graph, the positive peaks indicate locations of the highly antigenic regions of a full-length IL-21 protein, that is, regions from which epitope-bearing peptides of the invention can be determined. Polypeptides and polynucleotides encoding polypeptides comprising the domains defined by these graphs are contemplated by the present invention.

The data presented in FIG. 7 are also represented in tabular form in Table I. The columns are labeled with the headings “Res”, “Position”, and Roman Numerals I-XIV. The column headings refer to the following features of the amino acid sequence presented in FIG. 7 and Table I: “Res”: amino acid residue of SEQ ID NO: 29 or FIGS. 6A and 6B; “Position”: position of the corresponding residue within SEQ ID NO: 29 or FIGS. 6A and 6B; I: Alpha, Regions - Garnier-Robson; II: Alpha, Regions - Chou-Fasman; III: Beta, Regions - Garnier-Robson; TV: Beta, Regions - Chou-Fasman; V: Turn, Regions - Garnier-Robson; VI: Turn, Regions - Chou-Fasman; VII: Coil, Regions - Gamier-Robson; VIII: Hydrophilicity Plot - Kyte-Doolittle; IX: Hydrophobicity Plot - Hopp-Woods; X: Alpha, Amphipathic Regions - Eisenberg; XI: Beta, Amphipathic Regions - Eisenberg; XII: Flexible Regions - Karplus-Schulz; XIII: Antigenic Index - Jameson-Wolf; and XIV: Surface Probability Plot - Emini.

FIG. 8 shows the nucleotide sequence (SEQ ID NO: 31) and the deduced amino acid sequence (SEQ ID NO: 32) of an IL-22. The locations of conserved Domains I-IV and VI-VII are underlined and labeled as such. The locations of two potential N-linked glycosylation sites are identified by a bolded asparagine symbol (N) accompanied by a bolded pound sign (#) located above the initial nucleotide of the codon encoding the corresponding asparagine. The two potential N-linked glycosylation sites are located at Asn-39 (N-39, A-40, S-41) and Asn-152 (N-152, S-153, S-154) of SEQ ID NO: 32.

FIG. 9 shows an analysis of the IL-22 amino acid sequence provided in FIG. 8 and SEQ ID NO: 32. Alpha, beta, turn and coil regions; hydrophilicity and hydrophobicity; amphipathic regions; flexible regions; antigenic index and surface probability are shown. In the “Antigenic Index” or “Jameson-Wolf” graph, the positive peaks indicate locations of the highly antigenic regions of the IL-22 protein, that is, regions from which epitope-bearing peptides of the invention can be determined. Polypeptides and polynucleotides encoding polypeptides comprising the domains defined by these graphs are contemplated by the present invention.

The data presented in FIG. 9 are also represented in tabular form in Table III. The columns are labeled with the headings “Res”, “Position”, and Roman Numerals I-XIV. The column headings refer to the following features of the amino acid sequence presented in FIG. 9 and Table III: “Res”: amino acid residue of SEQ ID NO: 32 or FIG. 8; “Position”: position of the corresponding residue within SEQ ID NO: 32 or FIG. 8; J: Alpha, Regions - Gamier-Robson; II: Alpha, Regions - Chou-Fasman; III: Beta, Regions - Gamier-Robson; IV: Beta, Regions - Chou-Fasman; V: Turn, Regions - Gamier-Robson; VI: Turn, Regions - Chou-Fasman; VII: Coil, Regions - Gamier-Robson; VIII: Hydrophilicity Plot - Kyte-Doolittle; IX: Hydrophobicity Plot - Hopp-Woods; X: Alpha, Amphipathic Regions - Eisenberg; XI: Beta, Amphipathic Regions - Eisenberg; XII: Flexible Regions - Karplus-Schulz; XIII: Antigenic Index - Jameson-Wolf; and XIV: Surface Probability Plot - Emini.

### DETAILED DESCRIPTION

Definitions

The following definitions are provided to facilitate understanding of certain terms used throughout this specification.

In the present invention, “isolated” refers to material removed from its original environment (e.g., the natural environment if it is naturally occurring), and thus is altered “by the hand of man” from its natural state. For example, an isolated polynucleotide could be part of a vector or a composition of matter, or could be contained within a cell, and still be “isolated” because that vector, composition of matter, or particular cell is not the original environment of the polynucleotide. However, a nucleic acid contained in a clone that is a member of a library (e.g., a genomic or cDNA library) that has not been isolated from other members of the library (e.g., in the form of a homogeneous solution containing the clone and other members of the library) or which is contained on a chromosome preparation (e.g., a chromosome spread), is not “isolated” for the purposes of this invention.

In the present invention, a “secreted” IL-21 or IL-22 protein refers to a protein capable of being directed to the ER, secretory vesicles, or the extracellular space as a result of a signal sequence, as well as an IL-21 or IL-22 protein released into the extracellular space without necessarily containing a signal sequence. If the IL-21 or IL-22 secreted protein is released into the extracellular space, the IL-21 or IL-22 secreted protein can undergo extracellular processing to produce a “mature” IL-21 or IL-22 protein. Release into the extracellular space can occur by many mechanisms, including exocytosis and proteolytic cleavage.

As used herein, an IL-21 or IL-22 “polynucleotide” refers to a molecule having a nucleic acid sequence contained in SEQ ID NO: 1 or in SEQ ID NO: 3, respectively, or the cDNA contained within the respective clones deposited with the ATCC. For example, the IL-21 or IL-22 polynucleotide can contain the nucleotide sequence of the full-length cDNA sequence, including the 5′ and 3′ untranslated sequences, the coding region, with or without the signal sequence, the secreted protein coding region, as well as fragments, epitopes, domains, and variants of the nucleic acid sequence. Moreover, as used herein, an IL-21 or IL-22 “polypeptide” refers to a molecule having the translated amino acid sequence generated from the polynucleotide as broadly defined.

As used herein, an IL-21 “polynucleotide” refers to a molecule having a nucleic acid sequence contained in SEQ ID NO: 1 or in SEQ ID NO: 28, or the cDNA contained within the respective clones deposited with the ATCC. For example, the IL-21 polynucleotide can contain the nucleotide sequence of the full-length cDNA sequence, including the 5′ and 3′ untranslated sequences, the coding region, with or without the signal sequence, the secreted protein coding region, as well as fragments, epitopes, domains, and variants of the nucleic acid sequence. Moreover, as used herein, an IL-21 “polypeptide” refers to a molecule having the translated amino acid sequence generated from the polynucleotide as broadly defined.

As used herein , an IL-22 “polynucleotide” refers to a molecule having a nucleic acid sequence contained in SEQ ID NO: 3 or in SEQ ID NO: 31, or the cDNA contained within the respective clones deposited with the ATCC. For example, the IL-22 polynucleotide can contain the nucleotide sequence of the full-length cDNA sequence, including the 5′ and 3′ untranslated sequences, the coding region, with or without the signal sequence, the secreted protein coding region, as well as fragments, epitopes, domains, and variants of the nucleic acid sequence. Moreover, as used herein, an IL-22 “polypeptide” refers to a molecule having the translated amino acid sequence generated from the polynucleotide as broadly defined.

A representative clone containing all or most of the sequence for SEQ ID NO: 1 (designated HTGED19) was deposited with the American Type Culture Collection (“ATCC”) on Mar. 5, 1998, and was given the ATCC Deposit Number 209666. In addition, a representative clone containing all or most of the sequence for SEQ ID NO: 3 (designated HFPBX96) was also deposited with the ATCC on Mar. 5, 1998, and was given the ATCC Deposit Number 209665. The ATCC is located at 10801 University Blvd., Manassas, Va. 20110-2209, USA. The ATCC deposit was made pursuant to the terms of the Budapest Treaty on the international recognition of the deposit of microorganisms for purposes of patent procedure.

An IL-21 “polynucleotide” also includes those polynucleotides capable of hybridizing, under stringent hybridization conditions, to sequences contained in SEQ ID NO: 1 or SEQ ID NO: 28, the complements thereof, or the cDNA within the deposited clone. Further, an IL-22 “polynucleotide” also includes those polynucleotides capable of hybridizing, under stringent hybridization conditions, to sequences contained in SEQ ID NO: 3 or SEQ ID NO: 31, the complements thereof, or the cDNA within the deposited clone. “Stringent hybridization conditions” refers to an overnight incubation at 42 C in a solution comprising 50% formamide, 5x SSC (750 mM NaCl, 75 mM sodium citrate), 50 mM sodium phosphate (pH 7.6), 5x Denhardt's solution, 10% dextran sulfate, and 20 μg/ml denatured, sheared salmon sperm DNA, followed by washing the filters in 0.1x SSC at about 65 C.

Also contemplated are nucleic acid molecules that hybridize to the IL-21 and the IL-22 polynucleotides at lower stringency hybridization conditions. Changes in the stringency of hybridization and signal detection are primarily accomplished through the manipulation of formamide concentration (lower percentages of formamide result in lowered stringency); salt conditions, or temperature. For example, lower stringency conditions include an overnight incubation at 37 C in a solution comprising 6X SSPE (20X SSPE=3M NaCl; 0.2M NaH₂PO₄; 0.02M EDTA, pH 7.4), 0.5% SDS, 30% formamide, 100 μg/ml salmon sperm blocking DNA; followed by washes at 50 C with 1XSSPE, 0.1% SDS. In addition, to achieve even lower stringency, washes performed following stringent hybridization can be done at higher salt concentrations (e.g. 5X SSC).

Note that variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

Of course, a polynucleotide which hybridizes only to polyA+ sequences (such as any 3′ terminal polyA+ tract of a cDNA shown in the sequence listing), or to a complementary stretch of T (or U) residues, would not be included in the definition of “polynucleotide,” since such a polynucleotide would hybridize to any nucleic acid molecule containing a polyA+ stretch or the complement thereof (e.g., practically any double-stranded cDNA clone).

The IL-21 and IL-22 polynucleotides can be composed of any polyribonucleotide or polydeoxyribonucleotide, which may be unmodified RNA or DNA or modified RNA or DNA. For example, the IL-21 and IL-22 polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, the IL-21 polynucleotides can be composed of triple-stranded regions comprising RNA or DNA or both RNA and DNA. IL-21 polynucleotides may also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

IL-21 and IL-22 polypeptides can be composed of amino acids joined to each other by peptide bonds or modified peptide bonds, i.e., peptide isosteres, and may contain amino acids other than the 20 gene-encoded amino acids. The IL-21 and IL-22 polypeptides may be modified by either natural processes, such as posttranslational processing, or by chemical modification techniques which are well known in the art. Such modifications are well described in basic texts and in more detailed monographs, as well as in a voluminous research literature. Modifications can occur anywhere in the IL-21 and IL-22 polypeptides, including the peptide backbone, the amino acid side-chains and the amino or carboxyl termini. It will be appreciated that the same type of modification may be present in the same or varying degrees at several sites in a given IL-21 or IL-22 polypeptide. Also, a given IL-21 or IL-22 polypeptide may contain many types of modifications. IL-21 or IL-22 polypeptides may be branched, for example, as a result of ubiquitination, and they may be cyclic, with or without branching. Cyclic, branched, and branched cyclic IL-21 and IL-22 polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, PROTEINS - STRUCTURE AND MOLECULAR PROPERTIES, 2nd Ed., T. E. Creighton, W. H. Freeman and Company, New York (1993); POSTTRANSLATIONAL COVALENT MODIFICATION OF PROTEINS, B. C. Johnson, Ed., Academic Press, New York, pp. 1-12 (1983); Seifter, et al., Meth. Enzymol. 182:626-646 (1990); Rattan, et al., Ann. NY Acad. Sci. 663:48-62 (1992)).

“SEQ ID NO: 1” and “SEQ ID NO: 28” refer to an IL-21 polynucleotide sequence while “SEQ ID NO: 2” and SEQ ID NO: 29 refer to an IL-21 polypeptide sequence. Likewise, “SEQ ID NO: 3” and SEQ ID NO: 31 refer to an IL-22 polynucleotide sequence while “SEQ ID NO: 4” and SEQ ID NO: 32 refer to an IL-22 polypeptide sequence.

An IL-21 polypeptide “having biological activity” refers to polypeptides exhibiting activity similar, but not necessarily identical to, an activity of an IL-21 polypeptide, including mature forms, as measured in a particular biological assay, with or without dose-dependency. In addition, an IL-22 polypeptide “having biological activity” refers to polypeptides exhibiting activity similar, but not necessarily identical to, an activity of an IL-22 polypeptide, including mature forms, as measured in a particular biological assay, with or without dose-dependency. In the case where dose-dependency does exist, it need not be identical to that of the IL-21 or IL-22 polypeptide, but rather substantially similar to the dose-dependence in a given activity as compared to the IL-21 or IL-22 polypeptides (i.e., the candidate polypeptide will exhibit greater activity or not more than about 25-fold less and, preferably, not more than about tenfold less activity, and most preferably, not more than about three-fold less activity relative to the IL-21 polypeptide).

IL-21 and IL-22 Polynucleotides and Polypeptides

Clone HTGED19, encoding IL-21, was isolated from a cDNA library derived from apoptotic T-cells. This clone contains the entire coding region identified as SEQ ID NO: 2. The deposited clone contains a cDNA having a total of 705 nucleotides, which encodes a partial predicted open reading frame of 87 amino acid residues (see FIG. 1). The partial open reading frame begins at a point in the complete IL-21 ORF such that the “G” in position 1 of SEQ ID NO: 1 is actually in position 3 of a coding triplet. As such, the partial predicted IL-21 polypeptide sequence is shown beginning in-frame with an alanine residue at position 1 of SEQ ID NO: 2. The alanine residue at position 1 of SEQ ID NO: 2 is encoded by nucleotides 2-4 of the nucleotide sequence shown as SEQ ID NO: 1. The ORF shown as SEQ ID NO: 2 ends at a stop codon at nucleotide position 263-265 of the nucleotide sequence shown as SEQ ID NO: 1. The predicted molecular weight of the partial IL-21 protein should be about 9,558 Daltons.

An initial BLAST analysis of the expression of the IL-21 cDNA sequence against the HGS EST database has also revealed a highly specific expression of this cDNA clone. In such an analysis, the HTGED19 cDNA sequence appears to be found only in apoptotic T-cells. Thus, IL-21 appears to be expressed in a highly restricted pattern limited to apoptotic T-cells, and, for example, other subpopulatons of lymphocytes or other cells in a state of activation or quiescence.

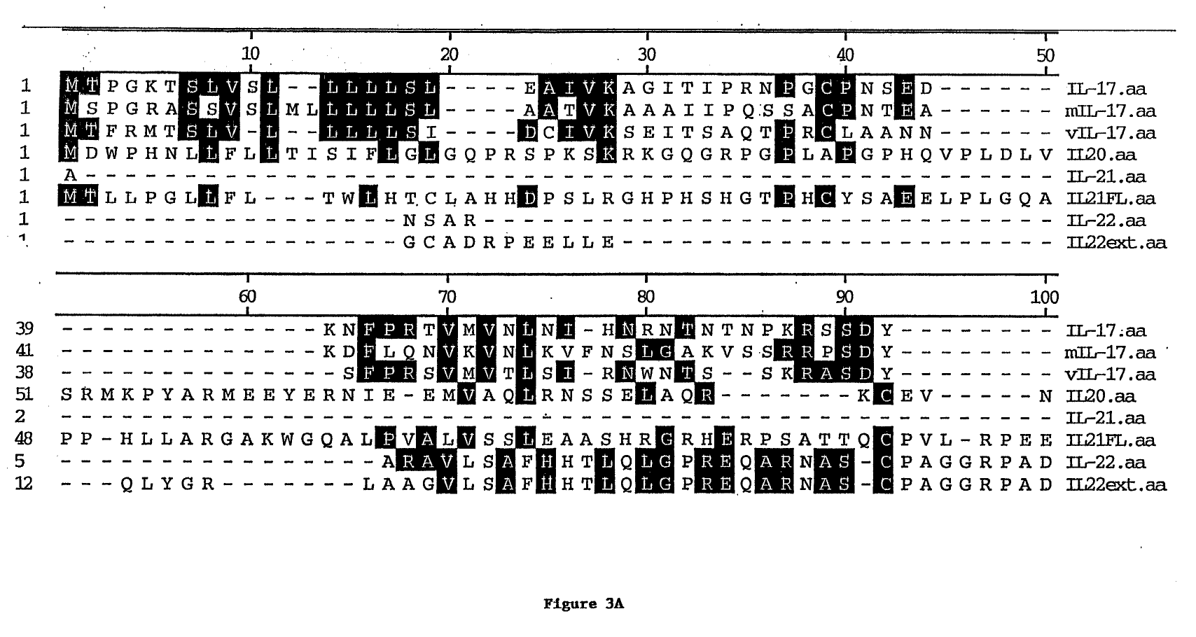

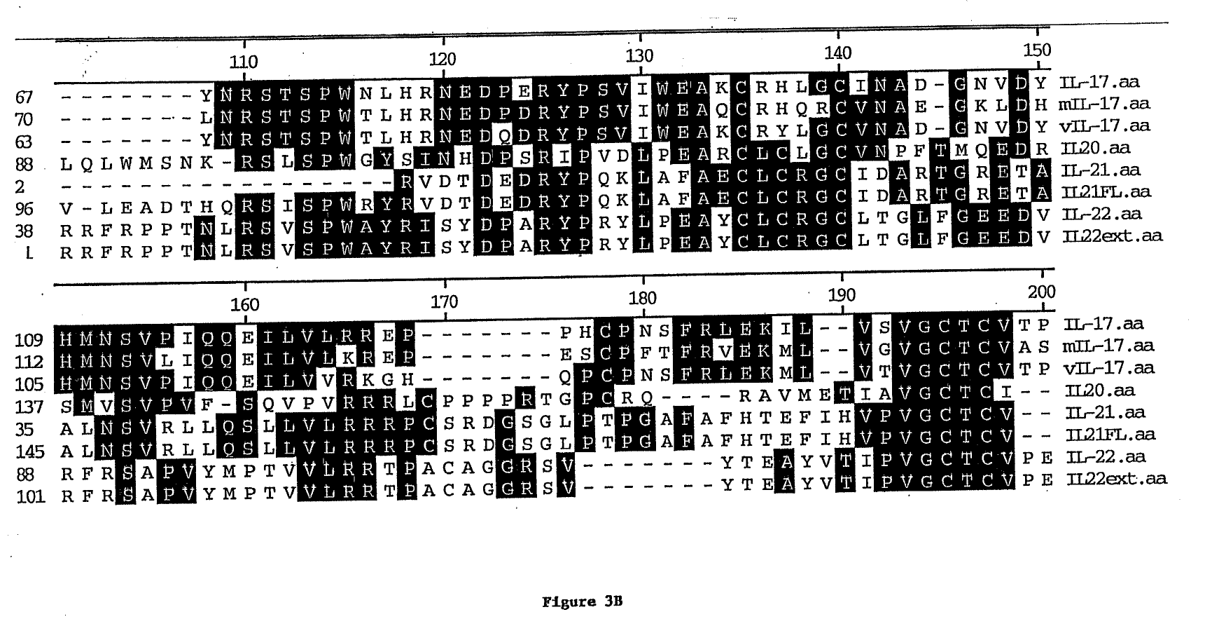

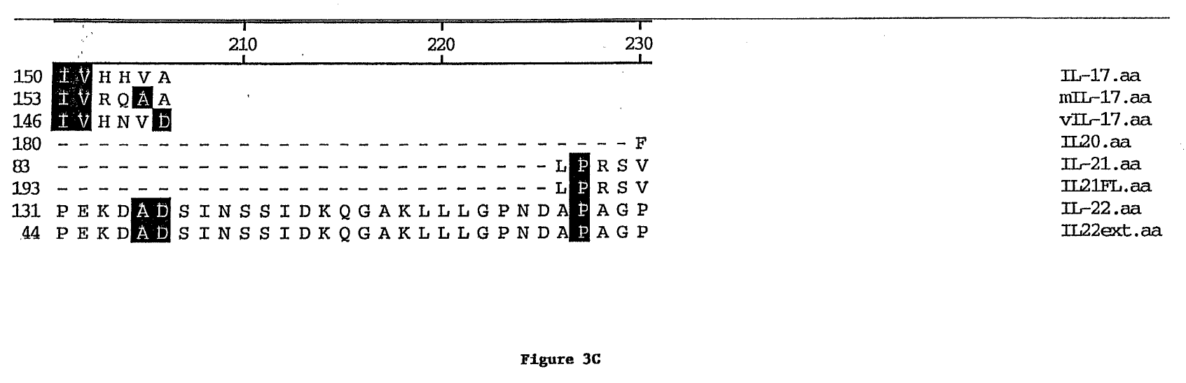

Clone HTGED19, encoding IL-21, was used to screen a panel of bacterial artificial chromosomes containing various segments of human genomic DNA (Research Genetics, Inc.). A positive clone was sequenced to identify potential splice donor and acceptor sites. Analysis of several sites revealed an upstream partial ORF that, when placed immediately 5′ and in frame with the existing IL-21 DNA sequence, generated a complete ORF which encodes a polypeptide with additional sequence identity to the IL-17 family (See FIGS. 3A, 3B, and 3C). A clone of the full-length IL-21 ORF has been constructed by combination of the IL-21 exons PCR-amplified from the HTGED19 genomic clone. The clone has been deposited with the ATCC as ATCC Deposit No. PTA-69 on May 14, 1999. The nucleotide sequence of the full-length IL-21 clone contains the entire coding region identified as SEQ ID NO: 29. The resultant clone contains an insert having a total of 1067 nucleotides, which encodes a predicted open reading frame of 197 amino acid residues (see FIGS. 6A and 6B). The open reading frame begins at nucleotide position 34 in the complete IL-21 polynucleotide shown as SEQ ID NO: 28 (FIGS. 6A and 6B). The ORF ends at a stop codon at nucleotide position 625-627 of the nucleotide sequence shown as SEQ ID NO: 28 (FIGS. 6A and 6B). The predicted molecular weight of the IL-21 polypeptide shown in FIGS. 6A and 6B and as SEQ ID NO: 29 should be about 21,764 Daltons.

Further BLAST analysis of the expression of the full-length IL-21 cDNA sequence against the HGS EST database has also revealed a highly specific expression of this cDNA clone. In such an analysis, the full-length HTGED19 cDNA sequence appears to be found only in apoptotic T-cells. Thus, IL-21 appears to be expressed in a highly restricted pattern limited to apoptotic T-cells, and, for example, other subpopulatons of lymphocytes or other cells in a state of activation or quiescence.

A PCR product comprising exons 1 and 2 (based on the genomic organization predicted above) has been amplified using a 12 week old early stage cDNA library as template DNA. This PCR product confirms that at least exons 1 and 2 of the genomic organization predicted above exists as messenger RNA in at least 12 week old early stage human embyo.

Clone HFPBX96, encoding IL-22, was isolated from a cDNA library derived from epileptic frontal cortex. This clone contains the entire coding region identified as SEQ ID NO: 4. The deposited clone contains a cDNA having a total of 1,642 nucleotides, which encodes a partial predicted open reading frame of 160 amino acid residues (see FIGS. 2A and 2B). The partial open reading frame begins at a point in the complete IL-22 ORF such that the “G” in position 1 of SEQ ID NO: 3 is actually in position two of a coding triplet. As such, the partial predicted IL-22 polypeptide sequence is shown beginning in-frame with an asparagine residue at position 1 of SEQ ID NO: 4. The asparagine residue at position 1 of SEQ ID NO: 4 is encoded by nucleotides 3-5 of the nucleotide sequence shown as SEQ ID NO: 3. The ORF shown as SEQ ID NO: 4 ends at a stop codon at nucleotide position 483-485 of the nucleotide sequence shown as SEQ ID NO: 3. The predicted molecular weight of the partial IL-22 protein should be about 17,436 Daltons.

Clone HFPBX96, encoding IL-22, was used to screen a human fetal brain cDNA library containing approximately one million cDNA clones (Genome Systems, Inc.). A positive clone was sequenced to identify 59 nucleotides of additional 5′ sequence. The cDNA clone has been deposited with the ATCC as ATCC Deposit No. PTA-70 on May 14, 1999. Analysis of the extended IL-22 ORF reveals a polypeptide with additional sequence identity to the IL-17 family (see FIGS. 3A, 3B, and 3C). The nucleotide sequence of the extended, but still apparently partial-length IL-22 clone contains the entire coding region identified as SEQ ID NO: 31. The resultant clone contains an insert having a total of 522 nucleotides, which encodes a predicted open reading frame of 174 amino acid residues (see FIG. 8). The open reading frame begins at nucleotide position 1 in the complete IL-22 polynucleotide shown as SEQ ID NO: 31 (FIG. 8). The ORF ends at a stop codon at nucleotide position 520-522 of the nucleotide sequence shown as SEQ ID NO: 31 (FIG. 8). The predicted molecular weight of the IL-22 polypeptide shown in FIG. 8 and as SEQ ID NO: 31 is about 19,636 Daltons.

Using BLAST and MegAlign analyses, SEQ ID NO: 2, SEQ ID NO: 4, SEQ ID NO: 29, and SEQ ID NO: 32 were each found to be highly homologous to several members of the Interleukin family. Particularly, SEQ ID NO: 2, SEQ ID NO: 4, SEQ ID NO: 29, and SEQ ID NO: 32 contain at least four domains homologous to the translation products of the human mRNA for Interleukin (IL)-20 (copending U.S. Provisional application Ser. No. 60/060,140; filed Sep. 26, 1997; SEQ ID NO: 8), IL-17 (GenBank Accession No. U32659; SEQ ID NO: 5; see also FIGS. 3A, 3B, and 3C), the murine mRNA for Interleukin (IL)-17 (GenBank Accession No. U43088; SEQ ID NO: 6; see also FIGS. 3A, 3B, and 3C), and the human viral mRNA for Interleukin (IL)-17 (GenBank Accession No. X64346; SEQ ID NO: 7; see also FIGS. 3A, 3B, and 3C).

Specifically, the molecules of the present invention, in particular, SEQ ID NO: 2, SEQ ID NO: 4, SEQ ID NO: 29, and SEQ ID NO: 32 share a high degree of sequence identity with IL-20, IL-17, mIL-17, and vIL-17 in the following conserved domains: (a) a predicted NXDPXRYP domain (where X represents any amino acid) located at about amino acids valine-3 to proline-11 of SEQ ID NO: 2, serine-57 to proline-64 of SEQ ID NO: 4, valine-113 to proline-121 of SEQ ID NO: 29, serine-70 to proline-77 of SEQ ID NO: 32, and asparagine-79 to proline-86 of the human IL-17 amino acid sequence (SEQ ID NO: 5); (b) a predicted CLCXGC domain (where X represents any amino acid) located at about amino acids cysteine-19 to cysteine-24 of SEQ ID NO: 2, cysteine-72 to cysteine-77 of SEQ ID NO: 4, cysteine-129 to cysteine-134 of SEQ ID NO: 29, cysteine-85 to cysteine-90 of SEQ ID NO: 32, and cysteine-94 to cysteine-99 of the human IL-17 amino acid sequence (SEQ ID NO: 5); (c) a predicted LVLRRXP domain (where X represents any amino acid) located at about amino acids leucine-46 to proline-52 of SEQ ID NO: 2, valine-99 to proline-105 of SEQ ID NO: 4, leucine-156 to proline-162 of SEQ ID NO: 29, valine-112 to proline-118 of SEQ ID NO: 32, and leucine-120 to proline-126 of the human IL- 17 amino acid sequence (SEQ ID NO: 5); and (d) a predicted VXVGCTCV domain (where X represents any amino acid) located at about amino acids valine-75 to valine-82 of SEQ ID NO: 2, isoleucine-121 to valine-128 of SEQ ID NO: 4, valine-187 to valine-192 of SEQ ID NO: 29, isoleucine-134 to valine-141 of SEQ ID NO: 32, and valine-140 to valine-147 of the human IL-17 amino acid sequence (SEQ ID NO: 5).

In addition, the full-length IL-21 molecule shown in FIGS. 6A and 6B (SEQ ID NO: 29) and the IL-22 molecule shown in FIG. 8 (SEQ ID NO: 32) exhibit several additional conserved domains when compared with IL-20 and the other members of the IL-17 family as shown in FIGS. 3A, 3B, and 3C). These conserved Domains are underlined in FIGS. 6A and 6B and in FIG. 8 and are labeled as conserved Domains V, VI, and VII. Specifically, the molecules of the present invention, in particular, SEQ ID NO: 29 and SEQ ID NO: 32, share a high degree of sequence identity with IL-20, IL-17, mIL- 17, and vIL- 17 in the following conserved domains: (a) a predicted PXCXSAE domain (where X represents any amino acid) located at about amino acids proline-34 to glutamic acid-40 of SEQ ID NO: 29; (b) a predicted PXXLVS domain (where X represents any amino acid) located at about amino acids proline-63 to serine-68 of SEQ ID NO: 29 and at about amino acids alanine-18 to serine-23 of SEQ ID NO: 32; and (c) a predicted RSXSPW domain (where X represents any amino acid) located at about amino acids arginine-104 to tryptophan-109 of SEQ ID NO: 29 and at about amino acids arginine-60 to tryptophan-65 of SEQ ID NO: 32. These polypeptide fragments of IL-21 and IL-22 are specifically contemplated in the present invention. Because each of these IL-17 and IL-17-like molecules is thought to be important immunoregulatory molecules, the homology between these IL-17 and IL-17-like molecules and IL-21 and IL-22 suggests that IL-21 and IL-22 may also be important immunoregulatory molecules.

Moreover, based on their apparent sequence identities with IL-17 and IL-20 (see FIGS. 3A, 3B, and 3C), the full-length IL-21 and IL-22 polypeptides are each likely to have an amino terminal secretory signal peptide leader sequence. Since the present invention appears to be partial cDNA clones of the IL-21 (SEQ ID NOs: 1 and 2) and IL-22 (SEQ ID NOs: 3 and 4) molecules (in addition to the full-length IL-21 molecule shown as SEQ ID NOs: 28 and 29 and the IL-22 molecule shown as SEQ ID NOs: 31 and 32), it is also contemplated that the translation products of SEQ ID NOs: 2, 4, and 32 of the present invention will be caused to enter the cellular secretory pathway by virtue of being expressed as a fusion proteins comprising several different portions of the N-terminus of the IL-20 molecule of copending U.S. Provisional application Ser. No. 60/060,140 fused to the known coding sequence of the IL-21 or IL-22 molecules of the present invention. Such expression constructs will secrete hybrid IL-20/IL-21 or IL-20/IL-22 molecules from the host cell.

In one embodiment, the mature IL-21 protein used in these fusion proteins encompasses about amino acids 12-87 of SEQ ID NO: 2, while the IL-20/21 fusion protein encompasses about the 104 or 113 N-terminal amino acids of IL-20 encoded in frame with about amino acids 12-87 of the IL-21 of SEQ ID NO: 2. In other embodiments, an IL-20/21 fusion protein encompasses about the 104 or 113 N-terminal amino acids of IL-20 encoded in frame with about amino acids 3-87 of the IL-21 protein of SEQ ID NO: 2. These polypeptide fragments of IL-21 are specifically contemplated in the present invention.

In another embodiment, the mature IL-22 protein used to generate these fusion proteins encompasses about amino acids 1-160 of SEQ ID NO: 4, while the IL-20/22 fusion protein encompasses about the 95, 104 or 113 N-terminal amino acids of IL-20 encoded in frame with about amino acids 1-160 of the IL-22 of SEQ ID NO: 4. In other embodiments, the IL-22 protein used to generate these fusion proteins encompasses about amino acids 47-160 of SEQ ID NO: 4, while the IL-20/22 fusion protein encompasses about the 95, 104 or 113 N-terminal amino acids of IL-20 encoded in frame with about amino acids 1-160 of the IL-22 of SEQ ID NO: 4. In still other embodiments, the IL-22 protein used to generate these fusion proteins encompasses about amino acids 56-160 of SEQ ID NO: 4, while the IL-20/22 fusion protein encompasses about the 95, 104 or 113 N-terminal amino acids of IL-20 encoded in frame with about amino acids 1-160 of the IL-22 of SEQ ID NO: 4. In yet other embodiments, the IL-22 protein used to generate these fusion proteins encompasses about amino acids 65-160 of SEQ ID NO: 4, while the IL-20/22 fusion protein encompasses about the 95, 104 or 113 N-terminal amino acids of IL-20 encoded in frame with about amino acids 1-160 of the IL-22 of SEQ ID NO: 4. These polypeptide fragments of IL-22 are specifically contemplated in the present invention.

In yet another embodiment, the mature IL-22 protein used to generate these fusion proteins encompasses about amino acids 1-173 of SEQ ID NO: 32, while the IL-20/22 fusion protein encompasses about the 95, 104 or 113 N-terminal amino acids of IL-20 encoded in frame with about amino acids 1-173 of the IL-22 of SEQ ID NO: 32. These polypeptide fragments of IL-22 are specifically contemplated in the present invention.

The IL-21 and IL-22 nucleotide sequences identified as SEQ ID NO: 1 and SEQ ID NO: 3, respectively, were assembled from partially homologous (“overlapping”) sequences obtained from the deposited clones. The IL-21 nucleotide sequence identified as SEQ ID NO: 28 was assembled from partially homologous (“overlapping”) sequences obtained from the deposited clone and a genomic DNA clone. The IL-22 nucleotide sequence identified as SEQ ID NO: 32 was assembled from partially homologous (“overlapping”) sequences obtained from the deposited clones (ATCC Deposit No. 209665 and ATCC Deposit No. PTA-70). The overlapping sequences specific to the partial IL-21 and IL-22 molecules of the invention and the full-length IL-21 molecule of the invention were each assembled into single contiguous sequences of high redundancy (usually three to five overlapping sequences at each nucleotide position), resulting in four final sequences identified as SEQ ID NO: 1, SEQ ID NO: 3, SEQ ID NO: 28, and SEQ ID NO: 31.

Therefore, SEQ ID NO: 1 and the translated SEQ ID NO: 2; SEQ ID NO: 3 and the translated SEQ ID NO: 4; SEQ ID NO: 31 and the translated SEQ ID NO: 32; and SEQ ID NO: 28 and the translated SEQ ID NO: 29, are sufficiently accurate and otherwise suitable for a variety of uses well known in the art and described further below. For instance, SEQ ID NO: 1, SEQ ID NO: 3, SEQ ID NO: 28, and SEQ ID NO: 31 are useful for designing nucleic acid hybridization probes that will detect nucleic acid sequences contained in SEQ ID NO: 1, SEQ ID NO: 3, SEQ ID NO: 28, and SEQ ID NO: 31, or the cDNA contained in the respective deposited cDNA clones. These probes will also hybridize to nucleic acid molecules in biological samples, thereby enabling a variety of forensic and diagnostic methods of the invention. Similarly, polypeptides identified from SEQ ID NO: 2 and SEQ ID NO: 29 may be used to generate antibodies which bind specifically to IL-21 and polypeptides identified from SEQ ID NO: 4 and SEQ ID NO: 32 may be used to generate antibodies which bind specifically to IL-22.

Nevertheless, DNA sequences generated by sequencing reactions can contain sequencing errors. The errors exist as misidentified nucleotides, or as insertions or deletions of nucleotides in the generated DNA sequence. The erroneously inserted or deleted nucleotides cause frame shifts in the reading frames of the predicted amino acid sequence. In these cases, the predicted amino acid sequence diverges from the actual amino acid sequence, even though the generated DNA sequence may be greater than 99.9% identical to the actual DNA sequence (for example, one base insertion or deletion in an open reading frame of over 1000 bases).

Accordingly, for those applications requiring precision in the nucleotide sequence or the amino acid sequence, the present invention provides not only the generated nucleotide sequence identified as SEQ ID NO: 1 and the predicted translated amino acid sequence identified as SEQ ID NO: 2, and the generated nucleotide sequence identified as SEQ ID NO: 28 and the predicted translated amino acid sequence identified as SEQ ID NO: 29, but also a sample of plasmid DNA containing a human cDNA of IL-21 deposited with the ATCC. In addition, the present invention also provides not only the generated nucleotide sequence identified as SEQ ID NO: 3 and the predicted translated amino acid sequence identified as SEQ ID NO: 4, and the generated nucleotide sequence identified as SEQ ID NO: 3 and the predicted translated amino acid sequence identified as SEQ ID NO: 4, but also a sample of plasmid DNA containing a human cDNA of IL-22 deposited with the ATCC. Accordingly, the nucleotide sequence of the deposited IL-21 and IL-22 clones can be readily determined by sequencing the deposited clone in accordance with known methods. The predicted IL-21 and IL-22 amino acid sequences can then be verified from such deposits. Moreover, the amino acid sequence of the protein encoded by the deposited clone can also be directly determined by peptide sequencing or by expressing the protein in a suitable host cell containing the deposited human IL-21 or IL-22 cDNAs, collecting the protein, and determining its sequence.

The present invention also relates to the IL-21 gene corresponding to SEQ ID NO: 1, SEQ ID NO: 2, SEQ ID NO: 28, SEQ ID NO: 29 or the deposited clone which encodes a partial IL-21. The present invention further relates to the IL-22 gene corresponding to SEQ ID NO: 3, SEQ ID NO: 4, SEQ ID NO: 31, SEQ ID NO: 32 or the deposited clone which encodes IL-22. The IL-21 and IL-22 genes can be isolated in accordance with known methods using the sequence information disclosed herein. Such methods include preparing probes or primers from the disclosed sequences and identifying or amplifying the IL-21 and IL-22 genes from appropriate sources of genomic material.

Also provided in the present invention are species homologs of IL-21 and IL-22. Species homologs may be isolated and identified by making suitable probes or primers from the sequences provided herein and screening a suitable nucleic acid source for the desired homolog.

The IL-21 and IL-22 polypeptides can be prepared in any suitable manner. Such polypeptides include isolated naturally occurring polypeptides, recombinantly produced polypeptides, synthetically produced polypeptides, or polypeptides produced by a combination of these methods. Means for preparing such polypeptides are well understood in the art.

The IL-21 and IL-22 polypeptides may be in the form of the secreted protein, including the mature form, or may be a part of a larger protein, such as a fusion protein. It is often advantageous to include an additional amino acids which comprise secretory or leader sequences, pro-sequences, sequences which aid in purification, such as multiple histidine residues, or an additional sequence for stability during recombinant production.

IL-21 and IL-22 polypeptides are preferably provided in an isolated form, and preferably are substantially purified. A recombinantly produced version of an IL-21 or IL-22 polypeptide, including the secreted polypeptide, can be substantially purified by the one-step method described in the publication by Smith and Johnson (Gene 67:31-40 (1988)). IL-21 and IL-22 polypeptides also can be purified from natural or recombinant sources using antibodies of the invention raised against the IL-21 and IL-22 proteins, respectively, in methods which are well known in the art.

Polynucleotide and Polypeptide Variants

“Variant” refers to a polynucleotide or polypeptide differing from the IL-21 and IL-22 polynucleotides or polypeptides, but retaining essential properties thereof. Generally, variants are overall closely similar, and, in many regions, identical to the IL-21 and IL-22 polynucleotide or polypeptide.

By a polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence encoding the IL-21 or IL-22 polypeptides. In other words, to obtain a polynucleotide having a nucleotide sequence at least 95% identical to a reference nucleotide sequence, up to 5% of the nucleotides in the reference sequence may be inserted, deleted or substituted with another nucleotide. The query sequence may be an entire sequence shown of SEQ ID NO: 1, SEQ ID NO: 3, SEQ ID NO: 28, SEQ ID NO: 31, the ORF (open reading frame) of either IL-21 or IL-22, or any fragment specified as described herein.

As a practical matter, whether any particular nucleic acid molecule or polypeptide is at least 90%, 95%, 96%, 97%, 98% or 99% identical to (or 10%, 5%, 4%, 3%, 2% or 1% different from) a nucleotide sequence of the presence invention can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag and colleagues (Comp. App. Biosci. 6:237-245 (1990)). In a sequence alignment the query and subject sequences are both DNA sequences. An RNA sequence can be compared by converting (uridine residues (U) to thymidine residues (T). The result of said global sequence alignment is in percent identity. Preferred parameters used in a FASTDB alignment of DNA sequences to calculate percent identiy are: Matrix=Unitary, k-tuple=4, Mismatch Penalty=1, Joining Penalty=30, Randomization Group Length=0, Cutoff Score=1, Gap Penalty=5, Gap Size Penalty 0.05, Window Size=500 or the length of the subject nucleotide sequence, whichever is shorter.

If the subject sequence is shorter than the query sequence because of 5′ or 3′ deletions, but not because of internal deletions, a manual correction must be made to the results. This is because the FASTDB algorithm does not account for 5′ and 3′ truncations of the subject sequence when calculating percent identity. For subject sequences truncated at the 5′ or 3′ ends, relative to the the query sequence, the percent identity is corrected by calculating the number of bases of the query sequence that are 5′ and 3′ of the subject sequence, which are not matched/aligned, as a percent of the total bases of the query sequence. Whether a nucleotide is matched/aligned is determined by results of the FASTDB sequence alignment. This percentage is then subtracted from the percent identity, calculated by the above FASTDB program using the specified parameters, to arrive at a final percent identity score. This corrected score is what is used for the purposes of the present invention. Only bases outside the 5′ and 3′ bases of the subject sequence, as displayed by the FASTDB alignment, which are not matched/aligned with the query sequence, are calculated for the purposes of manually adjusting the percent identity score.

For example, a 90 base subject sequence is aligned to a 100 base query sequence to determine percent identity. The deletions occur at the 5′ end of the subject sequence and therefore, the FASTDB alignment does not show a matched/alignment of the first 10 bases at 5′ end. The 10 unpaired bases represent 10% of the sequence ((number of bases at the 5′ and 3′ ends not matched)/(total number of bases in the query sequence)), so 10% is subtracted from the percent identity score calculated by the FASTDB program. If the remaining 90 bases were perfectly matched the final percent identity would be 90%. In another example, a 90 base subject sequence is compared with a 100 base query sequence. This time the deletions are internal deletions so that there are no bases on the 5′ or 3′ of the subject sequence which are not matched/aligned with the query. In this case the percent identity calculated by FASTDB is not manually corrected. Once again, only bases 5′ and 3′ of the subject sequence which are not matched/aligned with the query sequence are manually corrected for. No other manual corrections are to made for the purposes of the present invention.

By a polypeptide having an amino acid sequence which is, at least, for example, 95% “identical” to (or 5% different from) a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, (insertions and deletions are collectively referred to as “indels” in the art) or substituted with another amino acid. These alterations of the reference sequence may occur at the amino- or carboxy-terminal positions of the reference amino acid sequence or anywhere between those terminal positions, interspersed either individually among residues in the reference sequence or in one or more contiguous groups within the reference sequence.

As a practical matter, whether any particular polypeptide is at least 90%, 95%, 96%, 97%, 98% or 99% identical to (or 10%, 5%, 4%, 3%, 2% or 1% different from), for instance, the amino acid sequences shown in SEQ ID NO: 2 or SEQ ID NO: 29, or that shown in SEQ ID NO: 4 or SEQ ID NO: 32, or to the amino acid sequence encoded by deposited cDNA clones, can be determined conventionally using known computer programs. A preferred method for determing the best overall match between a query sequence (a sequence of the present invention) and a subject sequence, also referred to as a global sequence alignment, can be determined using the FASTDB computer program based on the algorithm of Brutlag and colleagues (Comp. App. Biosci. 6:237-245 (1990)). In a sequence alignment the query and subject sequences are either both nucleotide sequences or both amino acid sequences. The result of said global sequence alignment is in percent identity. Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter.

If the subject sequence is shorter than the query sequence due to N- or C-terminal deletions, not because of internal deletions, a manual correction must be made to the results. This is becuase the FASTDB program does not account for N- and C-terminal truncations of the subject sequence when calculating global percent identity. For subject sequences truncated at the N- and C-termini, relative to the the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence.

For example, a 90 amino acid residue subject sequence is aligned with a 100 residue query sequence to determine percent identity. The deletion occurs at the N-terminus of the subject sequence and therefore, the FASTDB alignment does not show a matching/alignment of the first 10 residues at the N-terminus. The 10 unpaired residues represent 10% of the sequence (number of residues at the N- and C-termini not matched/total number of residues in the query sequence), so 10% is subtracted from the percent identity score calculated by the FASTDB program. If the remaining 90 residues were perfectly matched the final percent identity would be 90%. In another example, a 90 residue subject sequence is compared with a 100 residue query sequence. This time the deletions are internal deletions so there are no residues at the N- or C-termini of the subject sequence which are not matched/aligned with the query. In this case the percent identity calculated by FASTDB is not manually corrected. Once again, only residue positions outside the N- and C-terminal ends of the subject sequence, as displayed in the FASTDB alignment, which are not matched/aligned with the query sequence are manually corrected for. No other manual corrections are to made for the purposes of the present invention.

The IL-21 and IL-22 variants may contain alterations in the coding regions, non-coding regions, or both. Especially preferred are polynucleotide variants containing alterations which produce silent substitutions, additions, or deletions, but do not alter the properties or activities of the encoded polypeptide. Nucleotide variants produced by silent substitutions due to the degeneracy of the genetic code are preferred. Moreover, variants in which 5-10, 1-5, or 1-2 amino acids are substituted, deleted, or added in any combination are also preferred. IL-21 and IL-22 polynucleotide variants can be produced for a variety of reasons, e.g., to optimize codon expression for a particular host (change codons in the human mRNA to those preferred by a bacterial host such as E. coli).

Naturally occurring IL-21 and IL-22 variants are called “allelic variants,” and refer to one of several alternate forms of a gene occupying a given locus on a chromosome of an organism (Genes II, Lewin, B., ed., John Wiley & Sons, New York (1985)). These allelic variants can vary at either the polynucleotide and/or polypeptide level. Alternatively, non-naturally occurring variants may be produced by mutagenesis techniques or by direct synthesis.

Using known methods of protein engineering and recombinant DNA technology, variants may be generated to improve or alter the characteristics of the IL-21 and IL-22 polypeptides. For instance, one or more amino acids can be deleted from the N-terminus or C-terminus of the secreted protein without substantial loss of biological function. Ron and coworkers reported variant KGF proteins having heparin binding activity even after deleting 3, 8, or 27 amino-terminal amino acid residues (J. Biol. Chem. 268:2984-2988 (1993)). Similarly, Interferon gamma exhibited up to ten times higher activity after deleting 8-10 amino acid residues from the carboxy terminus of this protein (Dobeli, et al., J. Biotechnol. 7:199-216 (1988)).

In the present case, since the IL-21 and IL-22 proteins of the invention are highly related to the Interleukin-17-like polypeptide family, deletions of N-terminal amino acids up to the cysteine at position 19 of SEQ ID NO: 2 and up to the cysteine at position 29 of SEQ ID NO: 4 may retain some biological activity. Polypeptides having further N-terminal deletions including the cysteine-19 residue in SEQ ID NO: 2 and the cysteine-29 residue in SEQ ID NO: 4 would not be expected to retain such biological activities because it is likely that these residues are required for forming a disulfide bridge to provide structural stability which is needed for receptor binding and signal transduction.

However, even if deletion of one or more amino acids from the N-terminus of a protein results in modification or loss of one or more biological functions of the protein, other biological activities may still be retained. Thus, the ability of the shortened protein to induce and/or bind to antibodies which recognize the complete or mature IL-21 or IL-22 proteins generally will be retained when less than the majority of the residues of the complete or mature IL-21 or IL-22 proteins are removed from the N-termini of the respective proteins. Whether a particular polypeptide lacking N-terminal residues of a complete protein retains such immunologic activities can readily be determined by routine methods described herein and otherwise known in the art.

Accordingly, the present invention further provides polypeptides having one or more residues deleted from the amino terminus of the amino acid sequence of the IL-21 polypeptide shown in SEQ ID NO: 2, up to the cysteine residue at position number 19, and polynucleotides encoding such polypeptides. In addition, the present invention further provides polypeptides having one or more residues deleted from the amino terminus of the amino acid sequence of the IL-22 polypeptide shown in SEQ ID NO: 4, up to the cysteine residue at position number 29, and polynucleotides encoding such polypeptides. In particular, the present invention provides polypeptides comprising the amino acid sequence of residues n¹-87 of SEQ ID NO: 2, where n¹ is an integer in the range of 1 to 18, and 19 is the position of the first residue from the N-terminus of the complete IL-21 polypeptide (shown in SEQ ID NO: 2) believed to be required for the receptor binding activity of the IL-21 protein. Likewise, the present invention provides polypeptides comprising the amino acid sequence of residues n²-160 of SEQ ID NO: 4, where n² is an integer in the range of 1 to 28, and 29 is the position of the first residue from the N-terminus of the complete IL-22 polypeptide (shown in SEQ ID NO: 4) believed to be required for the receptor binding activity of the IL-22 protein.

More in particular, the invention provides polynucleotides encoding polypeptides comprising, or alternatively consisting of, the amino acid sequence of residues 1-87, 2-87, 3-87, 4-87, 5-87, 6-87, 7-87, 8-87, 9-87, 10-87, 11-87, 12-87, 13-87, 14-87, 15-87, 16-87, 17-87, 18-87, and 19-87 of SEQ ID NO: 2. These polypeptide fragments may retain the biological activity of IL-21 polypeptides of the invention and/or may be useful to generate or screen for antibodies, as described further below. Polypeptides encoded by these polynucleotides are also provided. The present application is also directed to nucleic acid molecules comprising, or alternatively, consisting of, a polynucleotide sequence at least 90%, 95%, 96%, 97%, 98% or 99% identical to the polynucleotide sequence encoding the IL-21 polypeptides described above. The present invention also encompasses the above polynucleotide sequences fused to a heterologous polynucleotide sequence.

The invention also provides polynucleotides encoding polypeptides comprising, or alternatively consisting of, the amino acid sequence of residues 1-160, 2-160, 3-160, 4-160, 5-160, 6-160, 7-160, 8-160, 9-160, 10-160, 11-160, 12-160, 13-160, 14-160, 15-160, 16-160, 17-160, 18-160, 19-160, 20-160, 21-160, 22-160, 23-160, 24-160, 25-160, 26-160, 27-160, 28-160, and 29-160 of SEQ ID NO: 4. These polypeptide fragments may retain the biological activity of IL-22 polypeptides of the invention and/or may be useful to generate or screen for antibodies, as described further below. Polypeptides encoded by these polynucleotides are also provided. The present application is also directed to nucleic acid molecules comprising, or alternatively, consisting of, a polynucleotide sequence at least 90%, 95%, 96%, 97%, 98% or 99% identical to the polynucleotide sequence encoding the IL-22 polypeptides described above. The present invention also encompasses the above polynucleotide sequences fused to a heterologous polynucleotide sequence.

In addition, since the IL-21 and IL-22 proteins of the invention are highly related to the IL-17-like polypeptide family, deletions of C-terminal amino acids up to the leucine at postion 83 of SEQ ID NO: 2 and up to the proline at position 129 of SEQ ID NO: 4 may retain some biological activity. Polypeptides having further C-terminal deletions including the leucine residue at position 83 of SEQ ID NO: 2 and the proline at position 129 of SEQ ID NO: 4 would not be expected to retain such biological activities since these residues are in the beginning of the conserved domain required for biological activities.

However, even if deletion of one or more amino acids from the C-terminus of a protein results in modification of loss of one or more biological functions of the protein, other biological activities may still be retained. Thus, the ability of the shortened protein to induce and/or bind to antibodies which recognize the complete or mature IL-21 and IL-22 proteins generally will be retained when less than the majority of the residues of the complete or mature IL-21 and IL-22 proteins are removed from the C-terminus. Whether a particular polypeptide lacking C-terminal residues of a complete protein retains such immunologic activities can readily be determined by routine methods described herein and otherwise known in the art.

Accordingly, the present invention further provides polypeptides having one or more residues removed from the carboxy terminus of the amino acid sequence of the IL-21 polypeptide shown in SEQ ID NO: 2, up to the leucine residue at position 83 of SEQ ID NO: 2, and polynucleotides encoding such polypeptides. In addition, the present invention further provides polypeptides having one or more residues removed from the carboxy terminus of the amino acid sequence of the IL-22 polypeptide shown in SEQ ID NO: 4, up to the proline residues at position 129 of SEQ ID NO: 4. In particular, the present invention provides polypeptides having the amino acid sequence of residues 1-m¹ of the amino acid sequence in SEQ ID NO: 2, where m¹ is any integer in the range of 83 to 87, and residue 82 is the position of the first residue from the C-terminus of the complete IL-21 polypeptide (shown in SEQ ID NO: 2) believed to be required for activity of the IL-21 protein. In addition, the present invention also provides polypeptides having the amino acid sequence of residues 1-m² of the amino acid sequence in SEQ ID NO: 4, where m² is any integer in the range of 129 to 160, and residue 128 is the position of the first residue from the C-terminus of the complete IL-22 polypeptide (shown in SEQ ID NO: 4) believed to be required for activity of the IL-22 protein.

More in particular, the invention provides polynucleotides encoding polypeptides comprising, or alternatively consisting of, the amino acid sequence of residues 1-83, 1-84, 1-85, 1-86, and 1-87 of SEQ ID NO: 2. Polypeptides encoded by these polynucleotides are also provided. The present application is also directed to nucleic acid molecules comprising, or alternatively, consisting of, a polynucleotide sequence at least 90%, 95%, 96%, 97%, 98% or 99% identical to the polynucleotide sequence encoding the IL-21 polypeptides described above. The present invention also encompasses the above polynucleotide sequences fused to a heterologous polynucleotide sequence.

The present invention also provides polynucleotides encoding polypeptides comprising, or alternatively consisting of, the amino acid sequence of residues 1-129, 1-130, 1-131, 1-132, 1-133, 1-134, 1-135, 1-136, 1-137, 1-138, 1-139, 1-140, 1-141, 1-142, 1-143, 1-144, 1-145, 1-146, 1-147, 1-148, 1-149, 1-150, 1-151, 1-152, 1-153, 1-154, 1-155, 1-156, 1-157, 1-158, 1-159, and 1-160 of SEQ ID NO: 4. Polypeptides encoded by these polynucleotides are also provided. These polypeptide fragments may retain the biological activity of IL-22 polypeptides of the invention and/or may be useful to generate or screen for antibodies, as described further below. The present application is also directed to nucleic acid molecules comprising, or alternatively, consisting of, a polynucleotide sequence at least 90%, 95%, 96%, 97%, 98% or 99% identical to the polynucleotide sequence encoding the IL-22 polypeptides described above. The present invention also encompasses the above polynucleotide sequences fused to a heterologous polynucleotide sequence.

The invention also provides polypeptides having one or more amino acids deleted from both the amino and the carboxyl termini of IL-21, which may be described generally as having residues n¹-m¹ of SEQ ID NO: 2, where n¹ and m¹ are integers as described above. Likewise, the invention also provides polypeptides having one or more amino acids deleted from both the amino and the carboxyl termini of IL-22, which may be described generally as having residues n²-m² of SEQ ID NO: 4, where n² and m² are integers as described above.

Moreover, ample evidence demonstrates that variants often retain a biological activity similar to that of the naturally occurring protein. For example, Gayle and coworkers conducted extensive mutational analysis of human cytokine IL-1 a (J. Biol. Chem. 268:22105-22111 (1993)). They used random mutagenesis to generate over 3,500 individual IL-1a mutants that averaged 2.5 amino acid changes per variant over the entire length of the molecule. Multiple mutations were examined at every possible amino acid position. The investigators found that “[m]ost of the molecule could be altered with little effect on either [binding or biological activity]” (see, Abstract). In fact, only 23 unique amino acid sequences, out of more than 3,500 nucleotide sequences examined, produced a protein that significantly differed in activity from wild-type.

Furthermore, even if deleting one or more amino acids from the N-terminus or C-terminus of a polypeptide results in modification or loss of one or more biological functions, other biological activities may still be retained. For example, the ability of a deletion variant to induce and/or to bind antibodies which recognize the secreted form will likely be retained when less than the majority of the residues of the secreted form are removed from the N-terminus or C-terminus. Whether a particular polypeptide lacking N- or C-terminal residues of a protein retains such immunogenic activities can readily be determined by routine methods described herein and otherwise known in the art.

As mentioned above, even if deletion of one or more amino acids from the N-terminus of a protein results in modification or loss of one or more biological functions of the protein, other biological activities may still be retained. Thus, the ability of the shortened protein to induce and/or bind to antibodies which recognize the complete or mature IL-21 or IL-22 proteins generally will be retained when less than the majority of the residues of the complete or mature IL-21 or IL-22 proteins are removed from the N-termini of the respective proteins. Whether a particular polypeptide lacking N-terminal residues of a complete protein retains such immunologic activities can readily be determined by routine methods described herein and otherwise known in the art.

Accordingly, the present invention further provides polypeptides having one or more residues deleted from the amino terminus of the amino acid sequence of the IL-21 polypeptide shown in SEQ ID NO: 2, up to the valine residue at position number 82, and polynucleotides encoding such polypeptides. In addition, the present invention further provides polypeptides having one or more residues deleted from the amino terminus of the amino acid sequence of the IL-22 polypeptide shown in SEQ ID NO: 4, up to the aspartic acid residue at position number 155, and polynucleotides encoding such polypeptides. In particular, the present invention provides polypeptides comprising the amino acid sequence of residues n³-87 of SEQ ID NO: 2, where n³ is an integer in the range of 1 to 82, and 83 is the position of the last residue deleted from the N-terminus of the complete IL-21 polypeptide (shown in SEQ ID NO: 2) believed to be required for immunogenic activity of the IL-21 polypeptide. Likewise, the present invention provides polypeptides comprising the amino acid sequence of residues n⁴-160 of SEQ ID NO: 4, where n⁴ is an integer in the range of 1 to 155, and 156 is the position of the last residue deleted from the N-terminus of the complete IL-22 polypeptide (shown in SEQ ID NO: 4) believed to be required for immunogenic activity of the IL-22 polypeptide.

More in particular, the invention provides polynucleotides encoding polypeptides comprising, or alternatively consisting of, the amino acid sequence of residues R-2 to V-87; V-3 to V-87; D-4 to V-87; T-5 to V-87; D-6 to V-87; E-7 to V-87; D-8 to V-87; R-9 to V-87; Y-10 to V-87; P-11 to V-87; Q-12 to V-87; K-13 to V-87; L-14 to V-87; A-15 to V-87; F-16 to V-87; A-17 to V-87; E-18 to V-87; C-19 to V-87; L-20 to V-87; C-21 to V-87; R-22 to V-87; G-23 to V-87; C-24 to V-87; I-25 to V-87; D-26 to V-87; A-27 to V-87; R-28 to V-87; T-29 to V-87; G-30 to V-87; R-31 to V-87; E-32 to V-87; T-33 to V-87; A-34 to V-87; A-35 to V-87; L-36 to V-87; N-37 to V-87; S-38 to V-87; V-39 to V-87; R-40 to V-87; L-41 to V-87; L-42 to V-87; Q-43 to V-87; S-44 to V-87; L-45 to V-87; L-46 to V-87; V-47 to V-87; L-48 to V-87; R-49 to V-87; R-50 to V-87; R-51 to V-87; P-52 to V-87; C-53 to V-87; S-54 to V-87; R-55 to V-87; D-56 to V-87; G-57 to V-87; S-58 to V-87; G-59 to V-87; L-60 to V-87; P-61 to V-87; T-62 to V-87; P-63 to V-87; G-64 to V-87; A-65 to V-87; F-66 to V-87; A-67 to V-87; F-68 to V-87; H-69 to V-87; T-70 to V-87; E-71 to V-87; F-72 to V-87; I-73 to V-87; H-74 to V-87; V-75 to V-87; P-76 to V-87; V-77 to V-87; G-78 to V-87; C-79 to V-87; T-80 to V-87; C-81 to V-87; and V-82 to V-87 of SEQ ID NO: 2. These polypeptide fragments may retain the biological activity of IL-21 polypeptides of the invention and/or may be useful to generate or screen for antibodies, as described further below. Polypeptides encoded by these polynucleotides are also provided. The present application is also directed to nucleic acid molecules comprising, or alternatively, consisting of, a polynucleotide sequence at least 90%, 95%, 96%, 97%, 98% or 99% identical to the polynucleotide sequence encoding the IL-21 polypeptides described above. The present invention also encompasses the above polynucleotide sequences fused to a heterologous polynucleotide sequence.

Further, the invention provides polynucleotides encoding polypeptides comprising, or alternatively consisting of, the amino acid sequence of residues S-2 to P-160; A-3 to P-160; R-4 to P-160; A-5 to P-160; R-6 to P-160; A-7 to P-160; V-8 to P-160; L-9 to P-160; S-10 to P-160; A-11 to P-160; F-12 to P-160; H-13 to P-160; H-14 to P-160; T-15 to P-160; L-16 to P-160; Q-17 to P-160; L-18 to P-160; G-19 to P-160; P-20 to P-160; R-21 to P-160; E-22 to P-160; Q-23 to P-160; A-24 to P-160; R-25 to P-160; N-26 to P-160; A-27 to P-160; S-28 to P-160; C-29 to P-160; P-30 to P-160; A-31 to P-160; G-32 to P-160; G-33 to P-160; R-34 to P-160; P-35 to P-160; A-36 to P-160; D-37 to P-160; R-38 to P-160; R-39 to P-160; F-40 to P-160; R-41 to P-160; P-42 to P-160; P-43 to P-160; T-44 to P-160; N-45 to P-160; L-46 to P-160; R-47 to P-160; S-48 to P-160; V-49 to P-160; S-50 to P-160; P-51 to P-160; W-52 to P-160; A-53 to P-160; Y-54 to P-160; R-55 to P-160; I-56 to P-160; S-57 to P-160; Y-58 to P-160; D-59 to P-160; P-60 to P-160; A-61 to P-160; R-62 to P-160; Y-63 to P-160; P-64 to P-160; R-65 to P-160; Y-66 to P-160; L-67 to P-160; P-68 to P-160; E-69 to P-160; A-70 to P-160; Y-71 to P-160; C-72 to P-160; L-73 to P-160; C-74 to P-160; R-75 to P-160; G-76 to P-160; C-77 to P-160; L-78 to P-160; T-79 to P-160; G-80 to P-160; L-81 to P-160; F-82 to P-160; G-83 to P-160; E-84 to P-160; E-85 to P-160; D-86 to P-160; V-87 to P-160; R-88 to P-160; F-89 to P-160; R-90 to P-160; S-91 to P-160; A-92 to P-160; P-93 to P-160; V-94 to P-160; Y-95 to P-160; M-96 to P-160; P-97 to P-160; T-98 to P-160; V-99 to P-160; V-100 to P-160; L-101 to P-160; R-102 to P-160; R-103 to P-160; T-104 to P-160; P-105 to P-160; A-106 to P-160; C-107 to P-160; A-108 to P-160; G-109 to P-160; G-110 to P-160; R-111 to P-160; S-112 to P-160; V-113 to P-160; Y-114 to P-160; T-115 to P-160; E-116 to P-160; A-117 to P-160; Y-118 to P-160; V-119 to P-160; T-120 to P-160; I-121 to P-160; P-122 to P-160; V-123 to P-160; G-124 to P-160; C-125 to P-160; T-126 to P-160; C-127 to P-160; V-128 to P-160; P-129 to P-160; E-130 to P-160; P-131 to P-160; E-132 to P-160; K-133 to P-160; D-134 to P-160; A-135 to P-160; D-136 to P-160; S-137 to P-160; I-138 to P-160; N-139 to P-160; S-140 to P-160; S-141 to P-160; I-142 to P-160; D-143 to P-160; K-144 to P-160; Q-145 to P-160; G-146 to P-160; A-147 to P-160; K-148 to P-160; L-149 to P-160; L-150 to P-160; L-151 to P-160; G-152 to P-160; P-153 to P-160; N-154 to P-160; and D-155 to P-160 of SEQ ID NO: 4. These polypeptide fragments may retain the biological activity of IL-22 polypeptides of the invention and/or may be useful to generate or screen for antibodies, as described further below. Polypeptides encoded by these polynucleotides are also provided. The present application is also directed to nucleic acid molecules comprising, or alternatively, consisting of, a polynucleotide sequence at least 90%, 95%, 96%, 97%, 98% or 99% identical to the polynucleotide sequence encoding the IL-22 polypeptides described above. The present invention also encompasses the above polynucleotide sequences fused to a heterologous polynucleotide sequence.

Also as mentioned above, even if deletion of one or more amino acids from the C-terminus of a protein results in modification of loss of one or more biological functions of the protein, other biological activities may still be retained. Thus, the ability of the shortened protein to induce and/or bind to antibodies which recognize the complete or mature IL-21 and IL-22 proteins generally will be retained when less than the majority of the residues of the complete or mature IL-21 and IL-22 proteins are removed from the C-terminus. Whether a particular polypeptide lacking C-terminal residues of a complete protein retains such immunologic activities can readily be determined by routine methods described herein and otherwise known in the art.

Accordingly, the present invention further provides polypeptides having one or more residues removed from the carboxy terminus of the amino acid sequence of the IL-21 polypeptide shown in SEQ ID NO: 2, up to the aspartic acid residue at position 6 of SEQ ID NO: 2, and polynucleotides encoding such polypeptides. In addition, the present invention further provides polypeptides having one or more residues removed from the carboxy terminus of the amino acid sequence of the IL-22 polypeptide shown in SEQ ID NO: 4, up to the arginine residues at position 6 of SEQ ID NO: 4. In particular, the present invention provides polypeptides having the amino acid sequence of residues 1-m³ of the amino acid sequence in SEQ ID NO: 2, where m³ is any integer in the range of 6 to 87, and residue 5 is the position of the last residue deleted from the C-terminus of the complete IL-21 polypeptide (shown in SEQ ID NO: 2) believed to be required for immunogenic activity of the IL-21 polypeptide. In addition, the present invention also provides polypeptides having the amino acid sequence of residues 1-m⁴ of the amino acid sequence in SEQ ID NO: 4, where m⁴is any integer in the range of 6 to 160, and residue 5 is the position of the last residue deleted from the C-terminus of the complete IL-22 polypeptide (shown in SEQ ID NO: 4) believed to be required for immunogenic activity of the IL-22 polypeptide.

More in particular, the invention provides polynucleotides encoding polypeptides comprising, or alternatively consisting of, the amino acid sequence of residues A-1 to S-86; A-1 to R-85; A-1 to P-84; A-1 to L-83; A-1 to V-82; A-1 to C-81; A-1 to T-80; A-1 to C-79; A-1 to G-78; A-1 to V-77; A-1 to P-76; A-1 to V-75; A-1 to H-74; A-1 to I-73; A-1 to F-72; A-1 to E-71; A-1 to T-70; A-1 to H-69; A-1 to F-68; A-1 to A-67; A-1 to F-66; A-1 to A-65; A-1 to G-64; A-1 to P-63; A-1 to T-62; A-1 to P-61; A-1 to L-60; A-1 to G-59; A-1 to S-58; A-1 to G-57; A-1 to D-56; A-1 to R-55; A-1 to S-54; A-1 to C-53; A-1 to P-52; A-1 to R-51; A-1 to R-50; A-1 to R-49; A-1 to L-48; A-1 to V-47; A-1 to L-46; A-1 to L-45; A-1 to S-44; A-1 to Q-43; A-1 to L-42; A-1 to L-41; A-1 to R-40; A-1 to V-39; A-1 to S-38; A-1 to N-37; A-1 to L-36; A-1 to A-35; A-1 to A-34; A-1 to T-33; A-1 to E-32; A-1 to R-31; A-1 to G-30; A-1 to T-29; A-1 to R-28; A-1 to A-27; A-1 to D-26; A-1 to I-25; A-1 to C-24; A-1 to G-23; A-1 to R-22; A-1 to C-21; A-1 to L-20; A-1 to C-19; A-1 to E-18; A-1 to A-17; A-1 to F-16; A-1 to A-15; A-1 to L-14; A-1 to K-13; A-1 to Q-12; A-1 to P-11; A-1 to Y-10; A-1 to R-9; A-1 to D-8; A-1 to E-7; and A-1 to D-6 of SEQ ID NO: 2. These polypeptide fragments may retain the biological activity of IL-21 polypeptides of the invention and/or may be useful to generate or screen for antibodies, as described further below. Polynucleotides encoding these polypeptides are also provided. The present application is also directed to nucleic acid molecules comprising, or alternatively, consisting of, a polynucleotide sequence at least 90%, 95%, 96%, 97%, 98% or 99% identical to the polynucleotide sequence encoding the IL-21 polypeptides described above. The present invention also encompasses the above polynucleotide sequences fused to a heterologous polynucleotide sequence.

Moreover, the invention also provides polynucleotides encoding polypeptides comprising, or alternatively consisting of, the amino acid sequence of residues N-1 to G-159; N-1 to A-158; N-1 to P-157; N-1 to A-156; N-1 to D-155; N-1 to N-154; N-1 to P-153; N-1 to G-152; N-1 to L-151; N-1 to L-150; N-1 to L-149; N-1 to K-148; N-1 to A-147; N-1 to G-146; N-1 to Q-145; N-1 to K-144; N-1 to D-143; N-1 to I-142; N-1 to S-141; N-1 to S-140; N-1 to N-139; N-1 to I-138; N-1 to S-137; N-1 to D-136; N-1 to A-135; N-1 to D-134; N-1 to K-133; N-1 to E-132; N-1 to P-131; N-1 to E-130; N-1 to P-129; N-1 to V-128; N-1 to C-127; N-1 to T-126; N-1 to C-125; N-1 to G-124; N-1 to V-123; N-1 to P-122; N-1 to I-121; N-1 to T-120; N-1 to V-119; N-1 to Y-118; N-1 to A-117; N-1 to E-116; N-1 to T-115; N-1 to Y-114; N-1 to V-113; N-1 to S-112; N-1 to R-111; N-1 to G-110; N-1 to G-109; N-1 to A-108; N-1 to C-107; N-1 to A-106; N-1 to P-105; N-1 to T-104; N-1 to R-103; N-1 to R-102; N-1 to L-101; N-1 to V-100; N-1 to V-99; N-1 to T-98; N-1 to P-97; N-1 to M-96; N-1 to Y-95; N-1 to V-94; N-1 to P-93; N-1 to A-92; N-1 to S-91; N-1 to R-90; N-1 to F-89; N-1 to R-88; N-1 to V-87; N-1 to D-86; N-1 to E-85; N-1 to E-84; N-1 to G-83; N-1 to F-82; N-1 to L-81; N-1 to G-80; N-1 to T-79; N-1 to L-78; N-1 to C-77; N-1 to G-76; N-1 to R-75; N-1 to C-74; N-1 to L-73; N-1 to C-72; N-1 to Y-71; N-1 to A-70; N-1 to E-69; N-1 to P-68; N-1 to L-67; N-1 to Y-66; N-1 to R-65; N-1 to P-64; N-1 to Y-63; N-1 to R-62; N-1 to A-61; N-1 to P-60; N-1 to D-59; N-1 to Y-58; N-1 to S-57; N-1 to I-56; N-1 to R-55; N-1 to Y-54; N-1 to A-53; N-1 to W-52; N-1 to P-51; N-1 to S-50; N-1 to V-49; N-1 to S-48; N-1 to R-47; N-1 to L-46; N-1 to N-45; N-1 to T-44; N-1 to P-43; N-1 to P-42; N-1 to R-41; N-1 to F-40; N-1 to R-39; N-1 to R-38; N-1 to D-37; N-1 to A-36; N-1 to P-35; N-1 to R-34; N-1 to G-33; N-1 to G-32; N-1 to A-31; N-1 to P-30; N-1 to C-29; N-1 to S-28; N-1 to A-27; N-1 to N-26; N-1 to R-25; N-1 to A-24; N-1 to Q-23; N-1 to E-22; N-1 to R-21; N-1 to P-20; N-1 to G-19; N-1 to L-18; N-1 to Q-17; N-1 to L-16; N-1 to T-15; N-1 to H-14; N-1 to H-13; N-1 to F-12; N-1 to A-11; N-1 to S-10; N-1 to L-9; N-1 to V-8; N-1 to A-7; and N-1 to R-6 of SEQ ID NO: 4. These polypeptide fragments may retain the biological activity of IL-22 polypeptides of the invention and/or may be useful to generate or screen for antibodies, as described further below. Polypeptides encoded by these polynucleotides are also provided. The present application is also directed to nucleic acid molecules comprising, or alternatively, consisting of, a polynucleotide sequence at least 90%, 95%, 96%, 97%, 98% or 99% identical to the polynucleotide sequence encoding the IL-22 polypeptides described above. The present invention also encompasses the above polynucleotide sequences fused to a heterologous polynucleotide sequence.

The invention also provides polypeptides having one or more amino acids deleted from both the amino and the carboxyl termini of IL-21, which may be described generally as having residues n³-m³ of SEQ ID NO: 2, where n³ and m³ are integers as described above. Likewise, the invention also provides polypeptides having one or more amino acids deleted from both the amino and the carboxyl termini of IL-22, which may be described generally as having residues n⁴-m⁴ of SEQ ID NO: 4, where n⁴ and m⁴ are integers as described above.

Moreover, any polypeptide having one or more amino acids deleted from both the amino and the carboxyl termini of IL-22, described specifically as having residues n⁴-m⁴ of SEQ ID NO: 4 (where n⁴ and m⁴ are integers as described above) may be excluded from the invention. In particular, any polypeptide having one or more amino acids deleted from both the amino and the carboxyl termini of IL-22 and which is defined by residues n⁴-m⁴ of SEQ ID NO: 4, where n⁴ is equal to 21, 22, 23, 24 or 25 and m⁴ is equal to 155, 156, 157, 158, 159 or 160 maybe excluded from the invention.

Also as mentioned above, even if deletion of one or more amino acids from the N-terminus of a protein results in modification or loss of one or more biological functions of the protein, other biological activities may still be retained. Thus, the ability of the shortened protein to induce and/or bind to antibodies which recognize the full-length or mature IL-21 polypeptides generally will be retained when less than the majority of the residues of the full-length or mature IL-21 polypeptides are removed from the N-terminus. Whether a particular polypeptide lacking N-terminal residues of a complete or full-length polypeptide retains such immunologic activities can readily be determined by routine methods described herein and otherwise known in the art.

Accordingly, the present invention further provides polypeptides having one or more residues deleted from the amino terminus of the amino acid sequence of the IL-21 polypeptide shown in SEQ ID NO: 29, up to the valine residue at position number 192, and polynucleotides encoding such polypeptides. In particular, the present invention provides polypeptides comprising the amino acid sequence of residues n⁵-197 of SEQ ID NO: 29, where n⁵ is an integer in the range of 1 to 192, and 193 is the position of the last residue deleted from the N-terminus of the full-length IL-21 polypeptide (shown in SEQ ID NO: 29) believed to be required for immunogenic activity of the IL-21 polypeptide.

More in particular, the invention provides polynucleotides encoding polypeptides comprising, or alternatively consisting of, the amino acid sequence of residues T-2 to V-197; L-3 to V-197; L-4 to V-197; P-5 to V-197; G-6 to V-197; L-7 to V-197; L-8 to V-197; F-9 to V-197; L-10 to V-197; T-11 to V-197; W-12 to V-197; L-13 to V-197; H-14 to V-197; T-15 to V-197; C-16 to V-197; L-17 to V-197; A-18 to V-197; H-19 to V-197; H-20 to V-197; D-21 to V-197; P-22 to V-197; S-23 to V-197; L-24 to V-197; R-25 to V-197; G-26 to V-197; H-27 to V-197; P-28 to V-197; H-29 to V-197; S-30 to V-197; H-31 to V-197; G-32 to V-197; T-33 to V-197; P-34 to V-197; H-35 to V-197; C-36 to V-197; Y-37 to V-197; S-38 to V-197; A-39 to V-197; E-40 to V-197; E-41 to V-197; L-42 to V-197; P-43 to V-197; L-44 to V-197; G-45 to V-197; Q-46 to V-197; A-47 to V-197; P-48 to V-197; P-49 to V-197; H-50 to V-197; L-51 to V-197; L-52 to V-197; A-53 to V-197; R-54 to V-197; G-55 to V-197; A-56 to V-197; K-57 to V-197; W-58 to V-197; G-59 to V-197; Q-60 to V-197; A-61 to V-197; L-62 to V-197; P-63 to V-197; V-64 to V-197; A-65 to V-197; L-66 to V-197; V-67 to V-197; S-68 to V-197; S-69 to V-197; L-70 to V-197; E-71 to V-197; A-72 to V-197; A-73 to V-197; S-74 to V-197; H-75 to V-197; R-76 to V-197; G-77 to V-197; R-78 to V-197; H-79 to V-197; E-80 to V-197; R-81 to V-197; P-82 to V-197; S-83 to V-197; A-84 to V-197; T-85 to V-197; T-86 to V-197; Q-87 to V-197; C-88 to V-197; P-89 to V-197; V-90 to V-197; L-91 to V-197; R-92 to V-197; P-93 to V-197; E-94 to V-197; E-95 to V-197; V-96 to V-197; L-97 to V-197; E-98 to V-197; A-99 to V-197; D-100 to V-197; T-101 to V-197; H-102 to V-197; Q-103 to V-197; R-104 to V-197; S-105 to V-197; I-106 to V-197; S-107 to V-197; P-108 to V-197; W-109 to V-197; R-110 to V-197; Y-111 to V-197; R-112 to V-197; V-113 to V-197; D-114 to V-197; T-115 to V-197; D-116 to V-197; E-117 to V-197; D-118 to V-197; R-119 to V-197; Y-120 to V-197; P-121 to V-197; Q-122 to V-197; K-123 to V-197; L-124 to V-197; A-125 to V-197; F-126 to V-197; A-127 to V-197; E-128 to V-197; C-129 to V-197; L-130 to V-197; C-131 to V-197; R-132 to V-197; G-133 to V-197; C-134 to V-197; I-135 to V-197; D-136 to V-197; A-137 to V-197; R-138 to V-197; T-139 to V-197; G-140 to V-197; R-141 to V-197; E-142 to V-197; T-143 to V-197; A-144 to V-197; A-145 to V-197; L-146 to V-197; N-147 to V-197; S-148 to V-197; V-149 to V-197; R-150 to V-197; L-151 to V-197; L-152 to V-197; Q-153 to V-197; S-154 to V-197; L-155 to V-197; L-156 to V-197; V-157 to V-197; L-158 to V-197; R-159 to V-197; R-160 to V-197; R-161 to V-197; P-162 to V-197; C-163 to V-197; S-164 to V-197; R-165 to V-197; D-166 to V-197; G-167 to V-197; S-168 to V-197; G-169 to V-197; L-170 to V-197; P-171 to V-197; T-172 to V-197; P-173 to V-197; G-174 to V-197; A-175 to V-197; F-176 to V-197; A-177 to V-197; F-178 to V-197; H-179 to V-197; T-180 to V-197; E-181 to V-197; F-182 to V-197; I-183 to V-197; H-184 to V-197; V-185 to V-197; P-186 to V-197; V-187 to V-197; G-188 to V-197; C-189 to V-197; T-190 to V-197; C-191 to V-197; and V-192 to V-197 of SEQ ID NO: 29. These polypeptide fragments may retain the biological activity of IL-21 polypeptides of the invention and/or may be useful to generate or screen for antibodies, as described further below. Polypeptides encoded by these polynucleotides also are provided. The present application is also directed to nucleic acid molecules comprising, or alternatively, consisting of, a polynucleotide sequence at least 90%, 95%, 96%, 97%, 98% or 99% identical to the polynucleotide sequence encoding the IL-21 polypeptides described above. The present invention also encompasses the above polynucleotide sequences fused to a heterologous polynucleotide sequence.

Also as mentioned above, even if deletion of one or more amino acids from the C-terminus of a protein results in modification of loss of one or more biological functions of the protein, other biological activities may still be retained. Thus, the ability of the shortened polypeptide to induce and/or bind to antibodies which recognize the full-length or mature IL-21polypeptide generally will be retained when less than the majority of the residues of the full-length or mature IL-21 polypeptides are removed from the C-terminus. Whether a particular polypeptide lacking C-terminal residues of a complete protein retains such immunologic activities can readily be determined by routine methods described herein and otherwise known in the art.

Accordingly, the present invention further provides polypeptides having one or more residues removed from the carboxy terminus of the amino acid sequence of the IL-21 polypeptide shown in SEQ ID NO: 29, up to the glycine residue at position 6 of SEQ ID NO: 29, and polynucleotides encoding such polypeptides. In particular, the present invention provides polypeptides having the amino acid sequence of residues 1-m⁵ of the amino acid sequence in SEQ ID NO: 29, where m⁵ is any integer in the range of 6 to 196, and residue 5 is the position of the last residue deleted from the C-terminus of the full-length IL-21 polypeptide (shown in SEQ ID NO: 29) believed to be required for immunogenic activity of the IL-21 polypeptide.

More in particular, the invention provides polynucleotides encoding polypeptides comprising, or alternatively consisting of, the amino acid sequence of residues M-1 to S-196; M-1 to R-195; M-1 to P-194; M-1 to L-193; M-1 to V-192; M-1 to C-191; M-1 to T-190; M-1 to C-189; M-1 to G-188; M-1 to V-187; M-1 to P-186; M-1 to V-185; M-1 to H-184; M-1 to I-183; M-1 to F-182; M-1 to E-181; M-1 to T-180; M-1 to H-179; M-1 to F-178; M-1 to A-177; M-1 to F-176; M-1 to A-175; M-1 to G-174; M-1 to P-173; M-1 to T-172; M-1 to P-171; M-1 to L-170; M-1 to G-169; M-1 to S-168; M-1 to G-167; M-1 to D-166; M-1 to R-165; M-1 to S-164; M-1 to C-163; M-1 to P-162; M-1 to R-161; M-1 to R-160; M-1 to R-159; M-1 to L-158; M-1 to V-157; M-1 to L-156; M-1 to L-155; M-1 to S-154; M-1 to Q-153; M-1 to L-152; M-1 to L-151; M-1 to R-150; M-1 to V-149; M-1 to S-148; M-1 to N-147; M-1 to L-146; M-1 to A-145; M-1 to A-144; M-1 to T-143; M-1 to E-142; M-1 to R-141; M-1 to G-140; M-1 to T-139; M-1 to R-138; M-1 to A-137; M-1 to D-136; M-1 to I-135; M-1 to C-134; M-1 to G-133; M-1 to R-132; M-1 to C-131; M-1 to L-130; M-1 to C-129; M-1 to E-128; M-1 to A-127; M-1 to F-126; M-1 to A-125; M-1 to L-124; M-1 to K-123; M-1 to Q-122; M-1 to P-121; M-1 to Y-120; M-1 to R-119; M-1 to D-118; M-1 to E-117; M-1 to D-116; M-1 to T-115; M-1 to D-114; M-1 to V-113; M-1 to R-112; M-1 to Y-111; M-1 to R-110; M-1 to W-109; M-1 to P-108; M-1 to S-107; M-1 to I-106; M-1 to S-105; M-1 to R-104; M-1 to Q-103; M-1 to H-102; M-1 to T-101; M-1 to D-100; M-1 to A-99; M-1 to E-98; M-1 to L-97; M-1 to V-96; M-1 to E-95; M-1 to E-94; M-1 to P-93; M-1 to R-92; M-1 to L-91; M-1 to V-90; M-1 to P-89; M-1 to C-88; M-1 to Q-87; M-1 to T-86; M-1 to T-85; M-1 to A-84; M-1 to S-83; M-1 to P-82; M-1 to R-81; M-1 to E-80; M-1 to H-79; M-1 to R-78; M-1 to G-77; M-1 to R-76; M-1 to H-75; M-1 to S-74; M-1 to A-73; M-1 to A-72; M-1 to E-71; M-1 to L-70; M-1 to S-69; M-1 to S-68; M-1 to V-67; M-1 to L-66; M-1 to A-65; M-1 to V-64; M-1 to P-63; M-1 to L-62; M-1 to A-61; M-1 to Q-60; M-1 to G-59; M-1 to W-58; M-1 to K-57; M-1 to A-56; M-1 to G-55; M-1 to R-54; M-1 to A-53; M-1 to L-52; M-1 to L-51; M-1 to H-50; M-1 to P-49; M-1 to P-48; M-1 to A-47; M-1 to Q-46; M-1 to G-45; M-1 to L-44; M-1 to P-43; M-1 to L-42; M-1 to E-41; M-1 to E-40; M-1 to A-39; M-1 to S-38; M-1 to Y-37; M-1 to C-36; M-1 to H-35; M-1 to P-34; M-1 to T-33; M-1 to G-32; M-1 to H-31; M-1 to S-30; M-1 to H-29; M-1 to P-28; M-1 to H-27; M-1 to G-26; M-1 to R-25; M-1 to L-24; M-1 to S-23; M-1 to P-22; M-1 to D-21; M-1 to H-20; M-1 to H-19; M-1 to A-18; M-1 to L-17; M-1 to C-16; M-1 to T-15; M-1 to H1-14; M-1 to L-13; M-1 to W-12; M-1 to T-11; M-1 to L-10; M-1 to F-9; M-1 to L-8; M-1 to L-7; and M-1 to G-6 of SEQ ID NO: 29. These polypeptide fragments may retain the biological activity of IL-22 polypeptides of the invention and/or may be useful to generate or screen for antibodies, as described further below. Polypeptides encoded by these polynucleotides also are provided. The present application is also directed to nucleic acid molecules comprising, or alternatively, consisting of, a polynucleotide sequence at least 90%, 95%, 96%, 97%, 98% or 99% identical to the polynucleotide sequence encoding the IL-22 polypeptides described above. The present invention also encompasses the above polynucleotide sequences fused to a heterologous polynucleotide sequence.

The invention also provides polypeptides having one or more amino acids deleted from both the amino and the carboxyl termini of IL-21, which may be described generally as having residues n⁵-m⁵ of SEQ ID NO: 29, where n⁵ and m⁵ are integers as described above. Polynucleotides encoding such polypeptides are also provided.

Moreover, any polypeptide having one or more amino acids deleted from both the amino and the carboxyl termini of IL-21, described specifically as having residues n⁵-m⁵ of SEQ ID NO: 29 (where n⁵ and m⁵ are integers as described above) may be excluded from the invention.

Also as mentioned above, even if deletion of one or more amino acids from the N-terminus of a protein results in modification or loss of one or more biological functions of the protein, other biological activities may still be retained. Thus, the ability of the shortened protein to induce and/or bind to antibodies which recognize the full-length, partial-length or mature IL-22 polypeptides generally will be retained when less than the majority of the residues of the full-length, partial-length or mature IL-22 polypeptides are removed from the N-terminus. Whether a particular polypeptide lacking N-terminal residues of a complete or full-length polypeptide retains such immunologic activities can readily be determined by routine methods described herein and otherwise known in the art.

Accordingly, the present invention further provides polypeptides having one or more residues deleted from the amino terminus of the amino acid sequence of the IL-22 polypeptide shown in SEQ ID NO: 32, up to the aspartic acid residue at position number 168, and polynucleotides encoding such polypeptides. In particular, the present invention provides polypeptides comprising the amino acid sequence of residues n⁶- 173 of SEQ ID NO: 32, where n⁶ is an integer in the range of 1 to 168, and 168 is the position of the last residue deleted from the N-terminus of the IL-22 polypeptide (shown in SEQ ID NO: 32) believed to be required for immunogenic activity of the IL-22 polypeptide.

More in particular, the invention provides polynucleotides encoding polypeptides comprising, or alternatively consisting of, the amino acid sequence of residues C-2 to P-173; A-3 to P-173; D-4 to P-173; R-5 to P-173; P-6 to P-173; E-7 to P-173; E-8 to P-173; L-9 to P-173; L-10 to P-173; E-11 to P-173; Q-12 to P-173; L-13 to P-173; Y-14 to P-173; G-15 to P-173; R-16 to P-173; L-17 to P-173; A-18 to P-173; A-19 to P-173; G-20 to P-173; V-21 to P-173; L-22 to P-173; S-23 to P-173; A-24 to P-173; F-25 to P-173; H-26 to P-173; H-27 to P-173; T-28 to P-173; L-29 to P-173; Q-30 to P-173; L-31 to P-173; G-32 to P-173; P-33 to P-173; R-34 to P-173; E-35 to P-173; Q-36 to P-173; A-37 to P-173; R-38 to P-173; N-39 to P-173; A-40 to P-173; S-41 to P-173; C-42 to P-173; P-43 to P-173; A-44 to P-173; G-45 to P-173; G-46 to P-173; R-47 to P-173; P-48 to P-173; A-49 to P-173; D-50 to P-173; R-51 to P-173; R-52 to P-173; F-53 to P-173; R-54 to P-173; P-55 to P-173; P-56 to P-173; T-57 to P-173; N-58 to P-173; L-59 to P-173; R-60 to P-173; S-61 to P-173; V-62 to P-173; S-63 to P-173; P-64 to P-173; W-65 to P-173; A-66 to P-173; Y-67 to P-173; R-68 to P-173; I-69 to P-173; S-70 to P-173; Y-71 to P-173; D-72 to P-173; P-73 to P-173; A-74 to P-173; R-75 to P-173; Y-76 to P-173; P-77 to P-173; R-78 to P-173; Y-79 to P-173; L-80 to P-173; P-81 to P-173; E-82 to P-173; A-83 to P-173; Y-84 to P-173; C-85 to P-173; L-86 to P-173; C-87 to P-173; R-88 to P-173; G-89 to P-173; C-90 to P-173; L-91 to P-173; T-92 to P-173; G-93 to P-173; L-94 to P-173; F-95 to P-173; G-96 to P-173; E-97 to P-173; E-98 to P-173; D-99 to P-173; V-100 to P-173; R-101 to P-173; F-102 to P-173; R-103 to P-173; S-104 to P-173; A-105 to P-173; P-106 to P-173; V-107 to P-173; Y-108 to P-173; M-109 to P-173; P-110 to P-173; T-111 to P-173; V-112 to P-173; V-113 to P-173; L-114 to P-173; R-115 to P-173; R-116 to P-173; T-117 to P-173; P-118 to P-173; A-119 to P-173; C-120 to P-173; A-121 to P-173; G-122 to P-173; G-123 to P-173; R-124 to P-173; S-125 to P-173; V-126 to P-173; Y-127 to P-173; T-128 to P-173; E-129 to P-173; A-130 to P-173; Y-131 to P-173; V-132 to P-173; T-133 to P-173; I-134 to P-173; P-135 to P-173; V-136 to P-173; G-137 to P-173; C-138 to P-173; T-139 to P-173; C-140 to P-173; V-141 to P-173; P-142 to P-148 E-143 to P-173; P-144 to P-173; E-145 to P-173; K-146 to P-173; D-147 to P-173; A-148 to P-173; D-149 to P-173; S-150 to P-173; I-151 to P-173; N-152 to P-173; S-153 to P-173; S-154 to P-173; I-155 to P-173; D-156 to P-173; K-157 to P-173; Q-158 to P-173; G-159 to P-173; A-160 to P-173; K-161 to P-173; L-162 to P-173; L-163 to P-173; L-164 to P-173; G-165 to P-173; P-166 to P-173; N-167 to P-173; and D-168 to P-173 of SEQ ID NO: 32. These polypeptide fragments may retain the biological activity of IL-21 polypeptides of the invention and/or may be useful to generate or screen for antibodies, as described further below. Polypeptides encoded by these polynucleotides are also provided. The present application is also directed to nucleic acid molecules comprising, or alternatively, consisting of, a polynucleotide sequence at least 90%, 95%, 96%, 97%, 98% or 99% identical to the polynucleotide sequence encoding the IL-21 polypeptides described above. The present invention also encompasses the above polynucleotide sequences fused to a heterologous polynucleotide sequence.

Also as mentioned above, even if deletion of one or more amino acids from the C-terminus of a protein results in modification of loss of one or more biological functions of the protein, other biological activities may still be retained. Thus, the ability of the shortened polypeptide to induce and/or bind to antibodies which recognize the full-length, partial-length or mature IL-22 polypeptide generally will be retained when less than the majority of the residues of the full-length, partial-length or mature IL-22 polypeptides are removed from the C-terminus. Whether a particular polypeptide lacking C-terminal residues of a complete protein retains such immunologic activities can readily be determined by routine methods described herein and otherwise known in the art.

Accordingly, the present invention further provides polypeptides having one or more residues removed from the carboxy terminus of the amino acid sequence of the IL-22 polypeptide shown in SEQ ID NO: 32, up to the proline residue at position 6 of SEQ ID NO: 32, and polynucleotides encoding such polypeptides. In particular, the present invention provides polypeptides having the amino acid sequence of residues 1-m⁶ of the amino acid sequence in SEQ ID NO: 32, where m⁶ is any integer in the range of 6 to 173, and residue 6 is the position of the last residue deleted from the C-terminus of the IL-22 polypeptide (shown in SEQ ID NO: 32) believed to be required for immunogenic activity of the IL-22 polypeptide.

More in particular, the invention provides polynucleotides encoding polypeptides comprising, or alternatively consisting of, the amino acid sequence of residues G-1 to G-172; G-1 to A-171; G-1 to P-170; G-1 to A-169; G-1 to D-168; G-1 to N-167; G-1 to P-166; G-1 to G-165; G-1 to L-164; G-1 to L-163; G-1 to L-162; G-1 to K-161; G-1 to A-160; G-1 to G-159; G-1 to Q-158; G-1 to K-157; G-1 to D-156; G-1 to I-155; G-1 to S-154; G-1 to S-153; G-1 to N-152; G-1 to I-151; G-1 to S-150; G-1 to D-149; G-1 to A-148; G-1 to D-147; G-1 to K-146; G-1 to E-145; G-1 to P-144; G-1 to E-143; G-1 to P-142; G-1 to V-141; G-1 to C-140; G-1 to T-139; G-1 to C-138; G-1 to G-137; G-1 to V-136; G-1 to P-135; G-1 to I-134; G-1 to T-133; G-1 to V-132; G-1 to Y-131; G-1 to A-130; G-1 to E-129; G-1 to T-128; G-1 to Y-127; G-1 to V-126; G-1 to S-125; G-1 to R-124; G-1 to G-123; G-1 to G-122; G-1 to A-121; G-1 to C-120; G-1 to A-119; G-1 to P-118; G-1 to T-117; G-1 to R-116; G-1 to R-115; G-1 to L-114; G-1 to V-113; G-1 to V-112; G-1 to T-111; G-1 to P-110; G-1 to M-109; G-1 to Y-108; G-1 to V-107; G-1 to P-106; G-1 to A-105; G-1 to S-104; G-1 to R-103; G-1 to F-102; G-1 to R-101; G-1 to V-100; G-1 to D-99; G-1 to E-98; G-1 to E-97; G-1 to G-96; G-1 to F-95; G-1 to L-94; G-1 to G-93; G-1 to T-92; G-1 to L-91; G-1 to C-90; G-1 to G-89; G-1 to R-88; G-1 to C-87; G-1 to L-86; G-1 to C-85; G-1 to Y-84; G-1 to A-83; G-1 to E-82; G-1 to P-81; G-1 to L-80; G-1 to Y-79; G-1 to R-78; G-1 to P-77; G-1 to Y-76; G-1 to R-75; G-1 to A-74; G-1 to P-73; G-1 to D-72; G-1 to Y-71; G-1 to S-70; G-1 to I-69; G-1 to R-68; G-1 to Y-67; G-1 to A-66; G-1 to W-65; G-1 to P-64; G-1 to S-63; G-1 to V-62; G-1 to S-61; G-1 to R-60; G-1 to L-59; G-1 to N-58; G-1 to T-57; G-1 to P-56; G-1 to P-55; G-1 to R-54; G-1 to F-53; G-1 to R-52; G-1 to R-51; G-1 to D-50; G-1 to A-49; G-1 to P-48; G-1 to R-47; G-1 to G-46; G-1 to G-45; G-1 to A-44; G-1 to P-43; G-1 to C-42; G-1 to S-41; G-1 to A-40; G-1 to N-39; G-1 to R-38; G-1 to A-37; G-1 to Q-36; G-1 to E-35; G-1 to R-34; G-1 to P-33; G-1 to G-32; G-1 to L-31; G-1 to Q-30; G-1 to L-29; G-1 to T-28; G-1 to H-27; G-1 to H-26; G-1 to F-25; G-1 to A-24; G-1 to S-23; G-1 to L-22; G-1 to V-21; G-1 to G-20; G-1 to A-19; G-1 to A-18; G-1 to L-17; G-1 to R-16; G-1 to G-15; G-1 to Y-14; G-1 to L-13; G-1 to Q-12; G-1 to E-11; G-1 to L-10; G-1 to L-9; G-1 to E-8; G-1 to E-7; and G-1 to P-6 of SEQ ID NO: 32. These polypeptide fragments may retain the biological activity of IL-22 polypeptides of the invention and/or may be useful to generate or screen for antibodies, as described further below. Polypeptides encoded by these polynucleotides are also provided. The present application is also directed to nucleic acid molecules comprising, or alternatively, consisting of, a polynucleotide sequence at least 90%, 95%, 96%, 97%, 98% or 99% identical to the polynucleotide sequence encoding the IL-22 polypeptides described above. The present invention also encompasses the above polynucleotide sequences fused to a heterologous polynucleotide sequence.

The invention also provides polypeptides having one or more amino acids deleted from both the amino and the carboxyl termini of IL-22, which may be described generally as having residues n⁶-m⁶ of SEQ ID NO: 32, where n⁶ and m⁶ are integers as described above. Polynucleotides encoding these polypeptides are alos provided.

Moreover, any polypeptide having one or more amino acids deleted from both the amino and the carboxyl termini of IL-22, described specifically as having residues n⁶-m⁶ of SEQ ID NO: 32 (where n⁶ and m⁶ are integers as described above) may be excluded from the invention, as may polynucleotides encoding such polypeptides.

Additional preferred polypeptide fragments of IL-21 comprise, or alternatively consist of, the amino acid sequence of residues: M-1 to T-15; T-2 to C-16; L-3 to L-17; L-4 to A-18; P-5 to H-19; G-6 to H-20; L-7 to D-21; L-8 to P-22; F-9 to S-23; L-10 to L-24; T-11 to R-25; W-12 to G-26; L-13 to H-27; H-14 to P-28; T-15 to H-29; C-16 to S-30; L-17 to H-31; A-18 to G-32; H-19 to T-33; H-20 to P-34; D-21 to H-35; P-22 to C-36; S-23 to Y-37; L-24 to S-38; R-25 to A-39; G-26 to E-40; H-27 to E-41; P-28 to L-42; H-29 to P-43; S-30 to L-44; H-31 to G-45; G-32 to Q-46; T-33 to A-47; P-34 to P-48; H-35 to P-49; C-36 to H-50; Y-37 to L-51; S-38 to L-52; A-39 to A-53; E-40 to R-54; E-41 to G-55; L-42 to A-56; P-43 to K-57; L-44 to W-58; G-45 to G-59; Q-46 to Q-60; A-47 to A-61; P-48 to L-62; P-49 to P-63; H-50 to V-64; L-51 to A-65; L-52 to L-66; A-53 to V-67; R-54 to S-68; G-55 to S-69; A-56 to L-70; K-57 to E-71; W-58 to A-72; G-59 to A-73; Q-60 to S-74; A-61 to H-75; L-62 to R-76; P-63 to G-77; V-64 to R-78; A-65 to H-79; L-66 to E-80; V-67 to R-81; S-68 to P-82; S-69 to S-83; L-70 to A-84; E-71 to T-85; A-72 to T-86; A-73 to Q-87; S-74 to C-88; H-75 to P-89; R-76 to V-90; G-77 to L-91; R-78 to R-92; H-79 to P-93; E-80 to E-94; R-81 to E-95; P-82 to V-96; S-83 to L-97; A-84 to E-98; T-85 to A-99; T-86 to D-100; Q-87 to T-101; C-88 to H-102; P-89to Q-103; V-90 to R-104; L-91 to S-105; R-92 to I-106; P-93 to S-107; E-94 to P-108; E-95 to W-109; V-96 to R-110; L-97 to Y-111; E-98 to R-112; A-99 to V-113; D-100 to D-114; T-101 to T-115; H-102 to D-116; Q-103 to E-117; R-104 to D-118; S-105 to R-119;I-106 to Y-120; S-107 to P-121; P-108 to Q-122; W-109 to K-123; R-110 to L-124; Y-111 to A-125; R-112 to F-126; V-113 to A-127; D-114 to E-128; T-115 to C-129; D-116 to L-130; E-117 to C-131; D-118 to R-132; R-119 to G-133; Y-120 to C-134; P-121 to I-135; Q-122 to D-136; K-123 to A-137; L-124 to R-138; A-125 to T-139; F-126 to G-140; A-127 to R-141; E-128 to E-142; C-129 to T-143; L-130 to A-144; C-131 to A-145; R-132 to L-146; G-133 to N-147; C-134 to S-148; I-135 to V-149; D-136 to R-150; A-137 to L-151; R-138 to L-152; T-139 to Q-153; G-140 to S-154; R-141 to L-155; E-142 to L-156; T-143 to V-157; A-144 to L-158; A-145 to R-159; L-146 to R-160; N-147 to R-161; S-148 to P-162; V-149 to C-163; R-150 to S-164; L-151 to R-165; L-152 to D-166; Q-153 to G-167; S-154 to S-168; L-155 to G-169; L-156 to L-170; V-157 to P-171; L-158 to T-172; R-159 to P-173; R-160 to G-174; R-161 to A-175; P-162 to F-176; C-163 to A-177; S-164 to F-178; R-165 to H-179; D-166 to T-180; G-167 to E-181; S-168 to F-182; G-169 to I-183; L-170 to H-184; P-171 to V-185; T-172 to P-186; P-173 to V-187; G-174 to G-188; A-175 to C-189; F-176 to T-190; A-177 to C-191; F-178 to V-192; H-179 to L-193; T-180 to P-194; E-181 to R-195; F-182 to S-196;I-183 to V-197 of SEQ ID NO: 29. These polypeptide fragments may retain the biological activity of IL-21 polypeptides of the invention and/or may be useful to generate or screen for antibodies, as described further below. Polynucleotides encoding these polypeptide fragments are also encompassed by the invention. The present application is also directed to nucleic acid molecules comprising, or alternatively, consisting of, a polynucleotide sequence at least 90%, 95%, 96%, 97%, 98% or 99% identical to the polynucleotide sequence encoding the IL-21 polypeptides described above. The present invention also encompasses the above polynucleotide sequences fused to a heterologous polynucleotide sequence.

Additional preferred polypeptide fragments of IL-22 comprise, or alternatively consist of, the amino acid sequence of residues: G-1 to G-15; C-2 to R-16; A-3 to L-17; D-4 to A-18; R-5 to A-19; P-6 to G-20; E-7 to V-21; E-8 to L-22; L-9 to S-23; L-10 to A-24; E-11 to F-25; Q-12 to H-26; L-13 to H-27; Y-14 to T-28; G-15 to L-29; R-16 to Q-30; L-17 to L-31; A-18 to G-32; A-19 to P-33; G-20 to R-34; V-21 to E-35; L-22 to Q-36; S-23 to A-37; A-24 to R-38; F-25 to N-39; H-26 to A-40; H-27 to S-41; T-28 to C-42; L-29 to P-43; Q-30 to A-44; L-31 to G-45; G-32 to G-46; P-33 to R-47; R-34 to P-48; E-35 to A-49; Q-36 to D-50; A-37 to R-51; R-38 to R-52; N-39 to F-53; A-40 to R-54; S-41 to P-55; C-42 to P-56; P-43 to T-57; A-44 to N-58; G-45 to L-59; G-46 to R-60; R-47 to S-61; P-48 to V-62; A-49 to S-63; D-50 to P-64; R-51 to W-65; R-52 to A-66; F-53 to Y-67; R-54 to R-68; P-55 to I-69; P-56 to S-70; T-57 to Y-71; N-58 to D-72; L-59 to P-73; R-60 to A-74; S-61 to R-75; V-62 to Y-76; S-63 to P-77; P-64 to R-78; W-65 to Y-79; A-66 to L-80; Y-67 to P-81; R-68 to E-82; I-69 to A-83; S-70 to Y-84; Y-71 to C-85; D-72 to L-86; P-73 to C-87; A-74 to R-88; R-75 to G-89; Y-76 to C-90; P-77 to L-91; R-78 to T-92; Y-79 to G-93; L-80 to L-94; P-81 to F-95; E-82 to G-96; A-83 to E-97; Y-84 to E-98; C-85 to D-99; L-86 to V-100; C-87 to R-101; R-88 to F-102; G-89 to R-103; C-90 to S-104; L-91 to A-105; T-92 to P-106; G-93 to V-107; L-94 to Y-108; F-95 to M-109; G-96 to P-110; E-97 to T-111; E-98 to V-112; D-99 to V-113; V-100 to L-114; R-101 to R-115; F-102 to R-116; R-103 to T-117; S-104 to P-118; A-105 to A-119; P-106 to C-120; V-107 to A-121; Y-108 to G-122; M-109 to G-123; P-110 to R-124; T-111 to S-125; V-112 to V-126; V-113 to Y-127; L-114 to T-128; R-115 to E-129; R-116 to A-130; T-117 to Y-131; P-118 to V-132; A-119 to T-133; C-120 to I-134; A-121 to P-135; G-122 to V-136; G-123 to G-137; R-124 to C-138; S-125 to T-139; V-126 to C-140; Y-127 to V-141; T-128 to P-142; E-129 to E-143; A-130 to P-144; Y-131 to E-145; V-132 to K-146; T-133 to D-147; I-134 to A-148; P-135 to D-149; V-136 to S-150; G-137 to I-151; C-138 to N-152; T-139 to S-153; C-140 to S-154; V-141 to I-155; P-142 to D-156; E-143 to K-157; P-144 to Q-158; E-145 to G-159; K-146 to A-160; D-147 to K-161; A-148 to L-162; D-149 to L-163; S-150 to L-164; I-151 to G-165; N-152 to P-166; S-153 to N-167; S-154 to D-168; I-155 to A-169; D-156 to P-170; K-157 to A-171; Q-158 to G-172; G-159 to P-173 of SEQ ID NO: 32. These polypeptide fragments may retain the biological activity of IL-22 polypeptides of the invention and/or may be useful to generate or screen for antibodies, as described further below. Polynucleotides encoding these polypeptide fragments are also encompassed by the invention. The present application is also directed to nucleic acid molecules comprising, or alternatively, consisting of, a polynucleotide sequence at least 90%, 95%, 96%, 97%, 98% or 99% identical to the polynucleotide sequence encoding the IL-22 polypeptides described above. The present invention also encompasses the above polynucleotide sequences fused to a heterologous polynucleotide sequence.

The invention further includes IL-21 and IL-22 polypeptide variants which show substantial biological activity. Such variants include deletions, insertions, inversions, repeats, and substitutions selected according to general rules known in the art so as have little effect on activity. For example, guidance concerning how to make phenotypically silent amino acid substitutions is provided by Bowie and colleagues (Science 247:1306-1310 (1990)), wherein the authors indicate that there are two main strategies for studying the tolerance of an amino acid sequence to change.

The first strategy exploits the tolerance of amino acid substitutions by natural selection during the process of evolution. By comparing amino acid sequences in different species, conserved amino acids can be identified. These conserved amino acids are likely important for protein function. In contrast, the amino acid positions where substitutions have been tolerated by natural selection indicates that these positions are not critical for protein function. Thus, positions tolerating amino acid substitution could be modified while still maintaining biological activity of the protein.

The second strategy uses genetic engineering to introduce amino acid changes at specific positions of a cloned gene to identify regions critical for protein function. For example, site directed mutagenesis or alanine-scanning mutagenesis (introduction of single alanine mutations at every residue in the molecule) can be used (Cunningham and Wells, Science 244:1081-1085 (1989)). The resulting mutant molecules can then be tested for biological activity.

As the authors state, these two strategies have revealed that proteins are surprisingly tolerant of amino acid substitutions. The authors further indicate which amino acid changes are likely to be permissive at certain amino acid positions in the protein. For example, most buried (within the tertiary structure of the protein) amino acid residues require nonpolar side chains, whereas few features of surface side chains are generally conserved. Moreover, tolerated conservative amino acid substitutions involve replacement of an aliphatic or hydrophobic amino acid with another aliphatic or hydrophobic amino acid such as Ala, Val, Leu or Ile; replacement of a hydroxyl residue with another hydroxyl residue such as Ser or Thr; replacement of an acidic residue with another acidic residue such as Asp or Glu; replacement of an amide residue with another amide residue such as Asn or Gln, replacement of a basic residue with another basic residue such as Lys, Arg, or His; replacement of an aromatic residue with another aromatic residue such as Phe, Tyr, or Trp, and replacement of a small-sized amino acid with another small-sized residue such as Ala, Ser, Thr, Met, or Gly.

Besides conservative amino acid substitution, variants of IL-21 and IL-22 include (i) substitutions with one or more of the non-conserved amino acid residues, where the substituted amino acid residues may or may not be one encoded by the genetic code, or (ii) substitution with one or more of amino acid residues having a substituent group, or (iii) fusion of the mature polypeptide with another compound, such as a compound to increase the stability and/or solubility of the polypeptide (for example, polyethylene glycol), or (iv) fusion of the polypeptide with additional amino acids, such as an IgG Fc fusion region peptide, or leader or secretory sequence, or a sequence facilitating purification. Such variant polypeptides are deemed to be within the scope of those skilled in the art from the teachings herein.

For example, IL-21 and IL-22 polypeptide variants containing amino acid substitutions of charged amino acids with other charged or neutral amino acids may produce proteins with improved characteristics, such as less aggregation. Aggregation of pharmaceutical formulations both reduces activity and increases clearance due to the aggregates immunogenic activity (Pinckard, et al., Clin. Exp. Immunol. 2:331-340 (1967); Robbins, et al., Diabetes 36:838-845 (1987); Cleland, et al, Crit. Rev. Ther. Drug Carrier Systems 10:307-377 (1993)).

Polynucleotide and Polypeptide Fragments

The invention provides nucleic acid molecules having nucleotide sequences related to extensive portions of SEQ ID NO: 3 and SEQ ID NO: 31 which have been determined from the following related cDNA clones: HE2CD08R (SEQ ID NO: 24); HAGBX04R (SEQ ID NO: 25); HCEBA24FB (SEQ ID NO: 26); and HCELE59R (SEQ ID NO: 27). Furthermore, the invention provides nucleic acid molecules having nucleotide sequences related to extensive portions of SEQ ID NO: 28 which has been determined from a related cDNA clone designated HTGED19RB (SEQ ID NO: 30). Such polynucleotides (i.e., SEQ ID NOs: 24, 25, 26, and 30) may preferably be excluded from the present invention.

In the present invention, a “polynucleotide fragment” refers to a short polynucleotide having a nucleic acid sequence contained in the deposited clones or shown in SEQ ID NO: 1, SEQ ID NO: 3, SEQ ID NO: 28 or SEQ ID NO: 31. The short nucleotide fragments are preferably at least about 15 nt, and more preferably at least about 20 nt, still more preferably at least about 30 nt, and even more preferably, at least about 40 nt in length. A fragment “at least 20 nt in length,” for example, is intended to include 20 or more contiguous bases from the cDNA sequence contained in the deposited clones or the nucleotide sequences shown in SEQ ID NO: 1, SEQ ID NO: 3, SEQ ID NO: 28 or SEQ ID NO: 31. These nucleotide fragments are useful as diagnostic probes and primers as discussed herein. Of course, larger fragments (e.g., 50, 150, 500, 600, 2000 nucleotides) are preferred.

Moreover, representative examples of IL-21 polynucleotide fragments include, for example, fragments having a sequence from about nucleotide number 1-50, 51-100, 101-150, 151-200, 201-250, 251-300, 301-350, 351-400, 401-450, 451-500, 501-550, 551-600, 651-700, or 701 to the end of SEQ ID NO: I or the cDNA contained in the deposited clone. In addition, representative examples of IL-22 polynucleotide fragments include, for example, fragments having a sequence from about nucleotide number 1-50, 51-100, 101-150, 151-200, 201-250, 251-300, 301-350, 351-400, 401-450, 451-500, 501-550, 551-600, 601-650, 651-700, 701-750, 751-800, 801-850, 851-900, 901-950, 951-1000, 1001-1050, 1051-1100, 1101-1150, 1151-1200, 1201-1250, 1251-1300, 1301-1350, 1351-1400, 1401-1450, 1451-1500, 1551-1600, or 1601 to the end of SEQ ID NO: 3 or the cDNA contained in the deposited clone. Moreover, representative examples of the full-length IL-21 polynucleotide fragments include, for example, fragments having a sequence from about nucleotide number 1-1025, 50-1025, 100-1025, 150-1025, 200-1025, 250-1025, 300-1025, 350-1025, 400-1025, 450-1025, 500-1025, 550-1025, 600-1025, 650-1025, 700-1025, 750-1025, 800-1025, 850-1025, 900-1025, 950-1025, 1000-1025, 1-1000, 50-1000, 100-1000, 150-1000, 200-1000, 250-1000, 300-1000, 350-1000, 400-1000, 450-1000, 500-1000, 550-1000, 600-1000, 650-1000, 700-1000, 750-1000, 800-1000, 850-1000, 900-1000, 950-1000, 1-950, 50-950, 100-950, 150-950, 200-950, 250-950, 300-950, 350-950, 400-950, 450-950, 500-950, 550-950, 600-950, 650-950, 700-950, 750-950, 800-950, 850-950, 900-950, 1-900, 50-900, 100-900, 150-900, 200-900, 250-900, 300-900, 350-900, 400-900, 450-900, 500-900, 550-900, 600-900, 650-900, 700-900, 750-900, 800-900, 850-900, 1-850, 50-850, 100-850, 150-850, 200-850, 250-850, 300-850, 350-850, 400-850, 450-850, 500-850, 550-850, 600-850, 650-850, 700-850, 750-850, 800-850, 1-800, 50-800, 100-800, 150-800, 200-800, 250-800, 300-800, 350-800, 400-800, 450-800, 500-800, 550-800, 600-800, 650-800, 700-800, 750-800, 1-750, 50-750, 100-750, 150-750, 200-750, 250-750, 300-750, 350-750, 400-750, 450-750, 500-750, 550-750, 600-750, 650-750, 700-750, 1-700, 50-700, 100-700, 150-700, 200-700, 250-700, 300-700, 350-700, 400-700, 450-700, 500-700, 550-700, 600-700, 650-700, 1-650, 50-650, 100-650, 150-650, 200-650, 250-650, 300-650, 350-650, 400-650, 450-650, 500-650, 550-650, 600-650, 1-600, 50-600, 100-600, 150-600, 200-600, 250-600, 300-600, 350-600, 400-600, 450-600, 500-600, 550-600, 1-550, 50-550, 100-550, 150-550, 200-550, 250-550, 300-550, 350-550, 400-550, 450-550, 500-550, 1-500, 50-500, 100-500, 150-500, 200-500, 250-500, 300-500, 350-500, 400-500, 450-500, 1-450, 50-450, 100-450, 150-450, 200-450, 250-450, 300-450, 350-450, 400-450, 1-400, 50-400, 100-400, 150-400, 200-400, 250-400, 300-400, 350-400, 1-350, 50-350, 100-350, 150-350, 200-350, 250-350, 300-350, 1-300, 50-300, 100-300, 150-300, 200-300, 250-300, 1-250, 50-250, 100-250, 150-250, 200-250, 1-200, 50-200, 100-200, 150-200, 1-150, 50-150, 100-150, 1-100, 50-100, and 1-50 of SEQ ID NO: 28. In this context about includes the particularly recited ranges, larger or smaller by several (5, 4, 3, 2, or 1) nucleotides, at either terminus or at both termini. Preferably, these fragments encode a polypeptide which has biological activity.

Further, the invention includes a polynucleotide comprising any portion of at least about 30 nucleotides, preferably at least about 50 nucleotides, of SEQ ID NO: 1 from residue 1 to 650, 25 to 650, 50 to 650, 75 to 650, 100 to 650, 125 to 650, 150 to 650, 175 to 650, 200 to 650, 225 to 650, 250 to 650, 275 to 650, 300 to 650, 325 to 650, 350 to 650, 375 to 650, 400 to 650, 425 to 650, 500 to 650, 525 to 650, 550 to 650, 575 to 650, 600 to 650, 625 to 650, 1 to 600, 25 to 600, 50 to 600, 75 to 600, 100 to 600, 125 to 600, 150 to 600, 175 to 600, 200 to 600, 225 to 600, 250 to 600, 275 to 600, 300 to 600, 325 to 600, 350 to 600, 375 to 600, 400 to 600, 425 to 600, 500 to 600, 525 to 600, 550 to 600, 575 to 600 , 1 to 550, 25 to 550, 50 to 550, 75 to 550, 100 to 550, 125 to 550, 150 to 550, 175 to 550, 200 to 550, 225 to 550, 250 to 550, 275 to 550, 300 to 550, 325 to 550, 350 to 550, 375 to 550, 400 to 550, 425 to 550, 300 to 550, 525 to 550, 1 to 500, 25 to 500, 50 to 500, 75 to 500, 100 to 500, 125 to 500, 150 to 500, 175 to 500, 200 to 500, 225 to 500, 250 to 500, 275 to 500, 300 to 500, 325 to 500, 350 to 500, 375 to 500, 400 to 500, 425 to 500, 450 to 500, 475 to 500, 1 to 450, 25 to 450, 50 to 450, 75 to 450, 100 to 450, 125 to 450, 150 to 450, 175 to 450, 200 to 450, 225 to 450, 250 to 450, 275 to 450, 300 to 450, 325 to 450, 350 to 450, 375 to 450, 400 to 450, 425 to 450, 1 to 400, 25 to 400, 50 to 400, 75 to 400, 100 to 400, 125 to 400, 150 to 400, 175 to 400, 200 to 400, 225 to 400, 250 to 400, 275 to 400, 300 to 400, 325 to 400, 350 to 400, 375 to 400, 1 to 350, 25 to 350, 50 to 350, 75 to 350, 100 to 350, 125 to 350, 150 to 350, 175 to 350, 200 to 350, 225 to 350, 250 to 350, 275 to 350, 300 to 350, 325 to 350, 1 to 300, 25 to 300, 50 to 300, 75 to 300, 100 to 300, 125 to 300, 150 to 300, 175 to 300, 200 to 300, 225 to 300, 250 to 300, 275 to 300, 1 to 250, 25 to 250, 50 to 250, 75 to 250, 100 to 250, 125 to 250, 150 to 250, 175 to 250, 200 to 250, 225 to 250, 1 to 200, 25 to 200, 50 to 200, 75 to 200, 100 to 200, 125 to 200, 150 to 200, 175 to 200, 1 to 150, 25 to 150, 50 to 150, 75 to 150, 100 to 150, 125 to 150, 1 to 100, 25 to 100, 50 to 100, 75 to 100, 1 to50,and 25 to 50.

Moreover, the invention includes a polynucleotide comprising any portion of at least about 30 nucleotides, preferably at least about 50 nucleotides, of SEQ ID NO: 3 from residue 300 to 850. More preferably, the invention includes a polynucleotide comprising nucleotide residues 50 to 850, 75 to 850, 100 to 850, 125 to 850, 150 to 850, 175 to 850, 200 to 850, 225 to 850, 250 to 850, 275 to 850, 300 to 850, 325 to 850, 350 to 850, 375 to 850, 400 to 850, 425 to 850, 450 to 850, 475 to 850, 500 to 850, 525 to 850, 550 to 850, 575 to 850, 600 to 850, 625 to 850, 650 to 850, 675 to 850, 700 to 850, 750 to 850, 775 to 850, 800 to 850, 50 to 800, 75 to 800, 100 to 800, 125 to 800, 150 to 800, 175 to 800, 200 to 800, 225 to 800, 250 to 800, 275 to 800, 300 to 800, 325 to 800, 350 to 800, 375 to 800, 400 to 800, 425 to 800, 450 to 800, 475 to 800, 500 to 800, 525 to 800, 550 to 800, 575 to 800, 600 to 800, 625 to 800, 650 to 800, 675 to 800, 700 to 800, 750 to 800, 50 to 750, 75 to 750, 100 to 750, 125 to 750, 150 to 750, 175 to 750, 200 to 750, 225 to 750, 250 to 750, 275 to 750, 300 to 750, 325 to 750, 350 to 750, 375 to 750, 400 to 750, 425 to 750, 450 to 750, 475 to 750, 500 to 750, 525 to 750, 550to 750, 575 to 750, 600 to 750, 625 to 750, 650 to 750, 675 to 750, 700 to 750, 50 to 700, 75 to 700, 100 to 700, 125 to 700, 150 to 700, 175 to 700, 200 to 700, 225 to 700, 250 to 700, 275 to 700, 300 to 700, 325 to 700, 350 to 700, 375 to 700, 400 to 700, 425 to 700, 450 to 700, 475 to 700, 500 to 700, 525 to 700, 550 to 700, 575 to 700, 600 to 700, 625 to 700, 650 to 700, 50 to 650, 75 to 650, 100 to 650, 125 to 650, 150 to 650, 175 to 650, 200 to 650, 225 to 650, 250 to 650, 275 to 650, 300 to 650, 325 to 650, 350 to 650, 375 to 650, 400 to 650, 425 to 650, 450 to 650, 475 to 650, 500 to 650, 525 to 650, 550 to 650, 575 to 650, 600 to 650, 50 to 600, 75 to 600, 100 to 600, 125 to 600, 150 to 600, 175 to 600, 200 to 600, 225 to 600, 250 to 600, 275 to 600, 300 to 600, 325 to 600, 350 to 600, 375 to 600, 400 to 600, 425 to 600, 450 to 600, 475 to 600, 500 to 600, 525 to 600, 550 to 600, 50 to 550, 75 to 550, 100 to 550, 125 to 550, 150 to 550, 175 to 550, 200 to 550, 225 to 550, 250 to 550, 275 to 550, 300 to 550, 325 to 550, 350 to 550, 375 to 550, 400 to 550, 425 to 550, 450 to 550, 475 to 550, 500 to 550, 50 to 500, 75 to 500, 100 to 500, 125 to 500, 150 to 500, 175 to 500, 200 to 500, 225 to 500, 250 to 500, 275 to 500, 300 to 500, 325 to 500, 350 to 500, 375 to 500, 400 to 500, 425 to 500, 450 to 500, 50 to 450, 75 to 450, 100 to 450, 125 to 450, 150 to 450, 175 to 450, 200 to 450, 225 to 450, 250 to 450, 275 to 450, 300 to 450, 325 to 450, 350 to 450, 375 to 450, 400 to 450, 50 to 400, 75 to 400, 100 to 400, 125 to 400, 150 to 400, 175 to 400, 200 to 400, 225 to 400, 250 to 400, 275 to 400, 300 to 400, 325 to 400, 350 to 400, 50 to 350, 75 to 350, 100 to 350, 125 to 350, 150 to 350, 175 to 350, 200 to 350, 225 to 350, 250 to 350, 275 to 350, 300 to 350, 50 to 300, 75 to 300, 100 to 300, 125 to 300, 150 to 300, 175 to 300, 200 to 300, 225 to 300, and 250 to 300.

In the present invention, a “polypeptide fragment” refers to a short amino acid sequence contained in SEQ ID NO: 2, SEQ ID NO: 4, SEQ ID NO: 29, SEQ ID NO: 32 or encoded by the cDNAs contained in the deposited clones. Protein fragments may be “free-standing,” or comprised within a larger polypeptide of which the fragment forms a part or region, most preferably as a single continuous region. Representative examples of polypeptide fragments of the partial IL-21 invention, include, for example, fragments from about amino acid number 1-20, 21-40, 41-60, 61-83 or to the end of the coding region. Moreover, polypeptide fragments of IL-21 can be about 10, 20, 30, 40, 50, 60, 70, or 80 amino acids in length. Representative examples of polypeptide fragments of the IL-22 invention, include, for example, fragments from about amino acid number 1-20, 21-40, 41-60, 61-80, 81-100, 100-120, 120-140, 140-160, or to the end of the coding region. Moreover, polypeptide fragments of IL-22 can be about 10, 20, 30, 40, 50, 60, 70, 80, 100, 120, 140, or 150 amino acids in length. Representative examples of polypeptide fragments of the full-length IL-21 of the invention, include, for example, fragments from about amino acid number 1-20, 21-40, 41-60, 61-80, 81-100, 100-120, 120-140, 140-160, 160-180, 180-200 or 180-to the end of the coding region. Moreover, polypeptide fragments of the full-length IL-21 can be about 10, 20, 30, 40, 50, 60, 70, 80, 100, 120, 140, 150, 160, 170, 180 or 190 amino acids in length. In this context “about” includes the particularly recited ranges, larger or smaller by several (5, 4, 3, 2, or 1) amino acids, at either extreme or at both extremes.

A further embodiment of the invention relates to a peptide or polypeptide which comprises the amino acid sequence of an IL-21 or IL-22 polypeptide having an amino acid sequence which contains at least one conservative amino acid substitution, but not more than 50 conservative amino acid substitutions, even more preferably, not more than 40 conservative amino acid substitutions, still more preferably, not more than 30 conservative amino acid substitutions, and still even more preferably, not more than 20 conservative amino acid substitutions. Of course, in order of ever-increasing preference, it is highly preferable for a peptide or polypeptide to have an amino acid sequence which comprises the amino acid sequence of an IL-21 or IL-22 polypeptide, which contains at least one, but not more than 10, 9, 8, 7, 6, 5, 4, 3, 2 or 1 conservative amino acid substitutions.

Preferred polypeptide fragments include the secreted IL-21 and IL-22 proteins as well as the mature forms. Further preferred polypeptide fragments include the secreted IL-21 and IL-22 proteins or the mature forms having a continuous series of deleted residues from the amino or the carboxy terminus, or both. For example, any number of amino acids, ranging from 1-60, can be deleted from the amino terminus of either the secreted or the mature form of the IL-21 and IL-22 polypeptides. Similarly, any number of amino acids, ranging from 1-30, can be deleted from the carboxy terminus of the secreted or the mature form of the IL-21 and IL-22 polypeptides. Furthermore, any combination of the above amino and carboxy terminus deletions are preferred. Similarly, polynucleotide fragments encoding these IL-21 and IL-22 polypeptide fragments are also preferred.

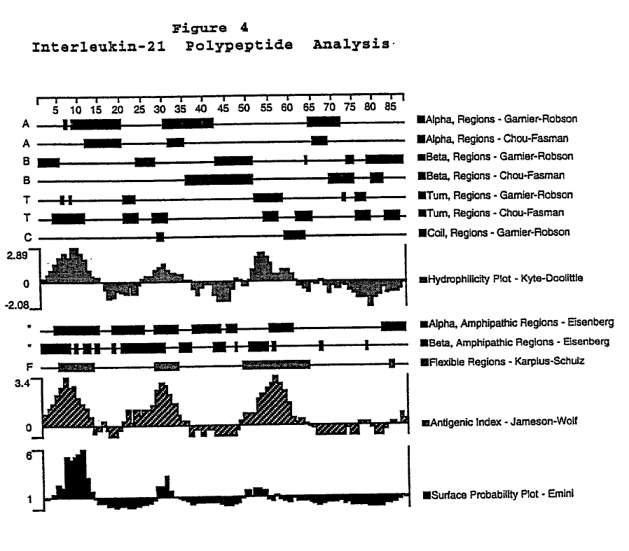

Also preferred are IL-21 and IL-22 polypeptide and polynucleotide fragments characterized by structural or functional domains, such as fragments that comprise alpha-helix and alpha-helix forming regions, beta-sheet and beta-sheet-forming regions, turn and turn-forming regions, coil and coil-forming regions, hydrophilic regions, hydrophobic regions, alpha amphipathic regions, beta amphipathic regions, flexible regions, surface-forming regions, substrate binding region, and high antigenic index regions. Polypeptide fragments of SEQ ID NO: 2, SEQ ID NO: 4, SEQ ID NO: 29 or SEQ ID NO: 32 falling within conserved domains are specifically contemplated by the present invention (FIGS. 4, 5, 7, and 9). Moreover, polynucleotide fragments encoding these domains are also contemplated.

In additional embodiments, the polynucleotides of the invention encode functional attributes of IL-21 or IL-22. Preferred embodiments of the invention in this regard include fragments that comprise alpha-helix and alpha-helix forming regions (“alpha-regions”), beta-sheet and beta-sheet forming regions (“beta-regions”), turn and turn-forming regions (“turn-regions”), coil and coil-forming regions (“coil-regions”), hydrophilic regions, hydrophobic regions, alpha amphipathic regions, beta amphipathic regions, flexible regions, surface-forming regions and high antigenic index regions of IL-21 or IL-22.

The data representing the structural or functional attributes of IL-21 set forth in FIG. 7 and/or Table I, as described above, was generated using the various modules and algorithms of the DNA*STAR set on default parameters. The data representing the structural or functional attributes of IL-22 set forth in FIG. 5 and/or Table II, in FIG. 9 and/or Table III, as described above, was generated using the various modules and algorithms of the DNA*STAR set on default parameters. In a preferred embodiment, the data presented in columns VIII, IX, XIII, and XIV of Table I can be used to determine regions of IL-21 which exhibit a high degree of potential for antigenicity. In an additional preferred embodiment, the data presented in columns VIII, IX, XIII, and XIV of Tables II and/or III can be used to determine regions of IL-22 which exhibit a high degree of potential for antigenicity. Regions of high antigenicity are determined from the data presented in columns VIII, IX, XIII, and/or XIV by choosing values which represent regions of the polypeptide which are likely to be exposed on the surface of the polypeptide in an environment in which antigen recognition may occur in the process of initiation of an immune response.

Certain preferred regions in these regards are set out in FIG. 7, but may, as shown in Tables I, be represented or identified by using tabular representations of the data presented in FIG. 7. The DNA*STAR computer algorithm used to generate FIG. 7 (set on the original default parameters) was used to present the data in FIG. 7 in a tabular format (See Table I). The tabular format of the data in FIG. 7 may be used to easily determine specific boundaries of a preferred region.

Certain preferred regions in these regards are set out in FIGS. 5 and 8, but may, as shown in Tables II and III, respectively, be represented or identified by using tabular representations of the data presented in FIGS. 5 and 8, respectively. The DNA*STAR computer algorithm used to generate FIGS. 5 and 8 (set on the original default parameters) was used to present the data in FIGS. 5 and 8 in a tabular format (See Tables II and III, respectively). The tabular format of the data in FIGS. 5 and 8 may be used to easily determine specific boundaries of a preferred region.

The above-mentioned preferred regions set out in FIGS. 5, 7, and 9, and in Tables II, I, and III, respectively, include, but are not limited to, regions of the aforementioned types identified by analysis of the amino acid sequence set out in FIGS. 2A-B, 6A-B, and 8, respectively. As set out in FIG. 7 and in Table I, and in FIG. 5 and Table II, and in FIG. 8 and Table III, such preferred regions include Garnier-Robson alpha-regions, beta-regions, turn-regions, and coil-regions, Chou-Fasman alpha-regions, beta-regions, and turn-regions, Kyte-Doolittle hydrophilic regions and hydrophobic regions, Eisenberg alpha- and beta-amphipathic regions, Karplus-Schulz flexible regions, Emini surface-forming regions and Jameson-Wolf regions of high antigenic index.

TABLE I

| Res | Position | I | II | III | IV | V | VI | VII | VIII | IX | X | XI | XII | XIII | XIV |
|---|---|---|---|---|---|---|---|---|---|---|---|---|---|---|---|
| Met | 1 | A | — | — | — | — | — | — | −0.80 | 0.76 | — | — | — | −0.40 | 0.36 |
| Thr | 2 | — | — | B | — | — | — | — | −0.76 | 0.76 | — | — | — | −0.40 | 0.44 |
| Leu | 3 | A | — | — | — | — | — | — | −1.18 | 0.76 | — | — | — | −0.40 | 0.34 |
| Leu | 4 | A | — | — | — | — | T | — | −1.60 | 1.01 | — | — | — | −0.20 | 0.28 |
| Pro | S | A | — | — | — | — | T | — | −1.91 | 1.09 | — | — | F | −0.05 | 0.16 |
| Gly | 6 | A | — | — | — | — | T | — | −2.12 | 1.39 | — | — | — | −0.20 | 0.17 |
| Leu | 7 | A | — | — | — | — | T | — | −2.12 | 1.39 | — | — | — | −0.20 | 0.17 |
| Leu | 8 | A | A | — | — | — | — | — | −1.60 | 1.19 | — | — | — | −0.60 | 0.16 |
| Phe | 9 | A | A | — | — | — | — | — | −1.60 | 1.67 | — | — | — | −0.60 | 0.17 |
| Leu | 10 | A | A | — | — | — | — | — | −1.42 | 1.93 | — | — | — | −0.60 | 0.17 |
| Thr | 11 | A | A | — | — | — | — | — | −1.39 | 1.74 | — | — | — | −0.60 | 0.28 |
| Trp | 12 | A | A | — | — | — | — | — | −1.24 | 1.54 | — | — | — | −0.60 | 0.46 |
| Leu | 13 | A | A | — | — | — | — | — | −1.24 | 1.33 | — | — | — | −0.60 | 0.30 |
| His | 14 | A | A | — | — | — | — | — | −1.13 | 1.33 | * | — | — | −0.60 | 0.17 |
| Thr | 15 | A | A | — | — | — | — | — | −0.36 | 1.34 | — | — | — | −0.60 | 0.17 |
| Cys | 16 | A | A | — | — | — | — | — | −0.08 | 0.93 | — | — | — | −0.60 | 0.27 |
| Leu | 17 | A | A | — | — | — | — | — | 0.21 | 0.74 | — | — | — | −0.60 | 0.27 |
| Ala | 18 | — | A | — | — | T | — | — | 0.81 | 0.24 | — | — | — | 0.10 | 0.32 |
| His | 19 | — | A | — | — | T | — | — | 0.54 | 0.19 | — | — | — | 0.44 | 0.91 |
| His | 20 | — | A | — | — | — | — | C | 0.04 | −0.00 | — | * | — | 1.33 | 1.48 |
| Asp | 21 | — | — | — | — | — | T | C | 0.82 | −0.00 | — | * | F | 2.22 | 1.21 |
| Pro | 22 | — | — | — | — | T | T | — | 1.29 | −0.50 | — | * | F | 2.76 | 1.74 |
| Ser | 23 | — | — | — | — | T | T | — | 1.84 | −0.57 | — | * | F | 3.40 | 1.27 |
| Leu | 24 | — | — | — | — | T | T | — | 1.67 | −0.57 | — | * | F | 3.06 | 1.03 |
| Arg | 25 | — | — | — | — | T | — | — | 1.67 | −0.14 | * | * | F | 2.35 | 1.03 |
| Gly | 26 | — | — | — | — | T | — | — | 1.37 | −0.07 | * | * | F | 2.14 | 1.05 |
| His | 27 | — | — | — | — | — | T | C | 1.54 | −0.07 | — | * | — | 1.78 | 1.70 |
| Pro | 28 | — | — | — | — | — | T | C | 1.50 | −0.26 | — | * | — | 1.57 | 1.18 |
| His | 29 | — | — | — | — | T | T | — | 2.00 | 0.17 | — | * | — | 1.30 | 1.18 |
| Ser | 30 | — | — | — | — | T | T | — | 1.68 | 0.23 | — | * | — | 1.17 | 1.26 |
| His | 31 | — | — | — | — | T | — | — | 1.99 | 0.16 | — | — | — | 0.84 | 1.26 |
| Gly | 32 | — | — | — | — | T | — | — | 1.36 | 0.23 | — | — | F | 0.86 | 1.26 |
| Thr | 33 | — | — | — | — | — | T | C | 1.32 | 0.30 | — | — | F | 0.58 | 0.50 |
| Pro | 34 | — | — | — | — | — | T | C | 1.06 | 0.67 | — | — | F | 0.15 | 0.58 |
| His | 35 | — | — | — | — | T | T | — | 0.77 | 0.56 | — | — | — | 0.20 | 0.78 |
| Cys | 36 | — | — | — | — | T | T | — | 0.80 | 0.63 | — | — | — | 0.20 | 0.55 |
| Tyr | 37 | — | A | — | — | T | — | C | 1.14 | 0.14 | — | — | — | 0.10 | 0.61 |
| Ser | 38 | — | A | — | — | — | — | C | 0.64 | −0.29 | — | — | — | 0.50 | 0.78 |
| Ala | 39 | A | A | — | — | — | — | — | 0.64 | −0.10 | — | — | — | 0.45 | 1.20 |
| Glu | 40 | A | A | — | — | — | — | — | −0.13 | −0.24 | — | — | F | 0.60 | 1.19 |
| Glu | 41 | A | A | — | — | — | — | — | 0.19 | −0.31 | — | — | — | 0.30 | 0.73 |
| Leu | 42 | A | — | — | — | — | T | — | 0.43 | −0.27 | — | — | — | 0.70 | 0.72 |
| Pro | 43 | A | — | — | — | — | T | — | 0.14 | −0.37 | — | — | — | 0.70 | 0.72 |
| Leu | 44 | — | — | — | — | T | T | — | 0.52 | 0.13 | — | — | — | 0.50 | 0.42 |
| Gly | 45 | — | — | — | — | T | T | — | 0.31 | 0.56 | — | — | F | 0.35 | 0.78 |
| Gln | 46 | A | — | — | — | — | — | — | 0.28 | 0.30 | — | — | F | 0.05 | 0.78 |
| Ala | 47 | — | — | — | — | — | — | C | 0.28 | 0.37 | * | — | F | 0.40 | 1.29 |
| Pro | 48 | — | — | — | — | — | T | C | −0.32 | 0.37 | * | — | F | 0.60 | 1.08 |
| Pro | 49 | A | — | — | — | — | T | — | −0.10 | 0.63 | * | * | F | −0.05 | 0.51 |
| His | 50 | A | — | — | — | — | T | — | 0.36 | 0.73 | * | * | — | −0.20 | 0.51 |
| Leu | 51 | A | — | — | — | — | T | — | 0.01 | 0.23 | * | * | — | 0.10 | 0.65 |
| Leu | 52 | A | A | — | — | — | — | — | 0.01 | 0.23 | * | * | — | −0.30 | 0.42 |
| Ala | 53 | A | A | — | — | — | — | — | 0.27 | 0.30 | * | — | — | −0.30 | 0.31 |
| Arg | 54 | A | A | — | — | — | — | — | 0.19 | −0.20 | * | — | — | 0.30 | 0.75 |
| Gly | 55 | A | A | — | — | — | — | — | −0.12 | 0.03 | * | — | F | −0.15 | 0.95 |
| Ala | 56 | A | A | — | — | — | — | — | 0.69 | −0.23 | * | — | F | 0.45 | 0.93 |
| Lys | 57 | — | A | — | — | T | — | — | 0.91 | −0.33 | * | — | F | 0.85 | 0.83 |
| Trp | 58 | — | A | — | — | T | — | — | 0.69 | 0.17 | * | — | F | 0.25 | 0.84 |
| Gly | 59 | — | A | — | — | — | — | C | 0.37 | 0.43 | * | — | F | −0.25 | 0.69 |
| Gln | 60 | A | A | — | — | — | — | — | −0.14 | 0.36 | * | — | — | −0.30 | 0.53 |
| Ala | 61 | — | A | — | — | — | — | C | −0.14 | 1.00 | * | — | — | −0.40 | 0.38 |
| Leu | 62 | — | A | B | — | — | — | — | −1.00 | 0.59 | * | — | — | −0.60 | 0.38 |
| Pro | 63 | — | A | B | — | — | — | — | −1.57 | 0.84 | — | — | — | −0.60 | 0.18 |
| Val | 64 | A | A | — | — | — | — | — | −1.52 | 1.09 | — | — | — | −0.60 | 0.13 |
| Ala | 65 | A | A | — | — | — | — | — | −1.82 | 0.97 | — | — | — | −0.60 | 0.22 |
| Leu | 66 | A | A | — | — | — | — | — | −2.04 | 0.67 | — | — | — | −0.60 | 0.19 |
| Val | 67 | A | A | — | — | — | — | — | −1.23 | 0.93 | — | — | — | −0.60 | 0.21 |
| Ser | 68 | A | A | — | — | — | — | — | −1.61 | 0.29 | — | — | — | −0.30 | 0.36 |
| Ser | 69 | A | A | — | — | — | — | — | −1.34 | 0.29 | — | — | — | −0.30 | 0.44 |
| Leu | 70 | A | A | — | — | — | — | — | −1.06 | 0.10 | * | — | — | −0.30 | 0.60 |
| Glu | 71 | A | A | — | — | — | — | — | −0.28 | −0.16 | * | — | — | 0.30 | 0.60 |
| Ala | 72 | A | A | — | — | — | — | — | 0.69 | −0.04 | * | — | — | 0.30 | 0.61 |
| Ala | 73 | A | A | — | — | — | — | — | 0.64 | −0.43 | * | * | — | 0.79 | 1.45 |
| Ser | 74 | A | — | — | — | — | — | — | 1.06 | −0.69 | * | * | — | 1.48 | 0.83 |
| His | 75 | A | — | — | — | — | T | — | 1.83 | −0.69 | * | * | — | 2.17 | 1.60 |
| Arg | 76 | A | — | — | — | — | T | — | 1.83 | −0.69 | — | * | F | 2.66 | 2.16 |
| Gly | 77 | — | — | — | — | T | T | — | 2.53 | −1.19 | — | * | F | 3.40 | 2.79 |
| Arg | 78 | — | — | — | — | T | T | — | 2.91 | −1.57 | — | * | F | 3.06 | 4.02 |
| His | 79 | — | — | — | — | — | — | C | 2.91 | −1.64 | — | * | F | 2.66 | 3.17 |
| Glu | 60 | — | — | — | — | — | — | C | 2.36 | −1.26 | — | * | F | 2.66 | 4.29 |
| Arg | 81 | — | — | — | — | — | T | C | 1.93 | −1.19 | — | * | F | 2.86 | 2.21 |
| Pro | 82 | — | — | — | — | T | T | — | 1.97 | −0.70 | — | — | F | 3.06 | 2.35 |
| Ser | 83 | — | — | — | — | T | T | — | 1.86 | −0.71 | — | * | F | 3.40 | 1.96 |
| Ala | 84 | — | — | — | — | T | T | — | 1.22 | −0.31 | — | * | F | 2.76 | 1.73 |
| Thr | 85 | — | — | — | B | T | — | — | 1.01 | 0.26 | — | * | F | 1.27 | 0.60 |
| Thr | 86 | — | — | — | B | T | — | — | 0.04 | 0.26 | — | * | F | 0.93 | 0.69 |
| Gln | 87 | — | — | — | B | T | — | — | −0.56 | 0.51 | — | — | F | 0.29 | 0.51 |
| Cys | 88 | — | — | B | B | — | — | — | −0.14 | 0.70 | * | — | — | −0.60 | 0.29 |
| Pro | 89 | — | — | B | B | — | — | — | 0.23 | 0.21 | — | * | — | −0.30 | 0.39 |
| Val | 90 | — | — | — | B | — | — | C | 0.54 | 0.16 | — | — | — | −0.10 | 0.35 |
| Leu | 91 | — | A | — | — | — | — | C | 0.86 | −0.24 | — | — | — | 0.65 | 1.14 |
| Arg | 92 | — | A | — | — | — | — | C | 0.00 | −0.81 | * | — | F | 1.10 | 1.27 |
| Pro | 93 | A | A | — | — | — | — | — | −0.14 | −0.60 | * | * | F | 0.90 | 1.27 |
| Glu | 94 | A | A | — | — | — | — | — | 0.07 | −0.56 | * | * | F | 0.90 | 1.27 |
| Glu | 95 | A | A | — | — | — | — | — | 0.33 | −1.24 | * | * | F | 0.90 | 1.13 |
| Val | 96 | A | A | — | — | — | — | — | 1.14 | −0.74 | — | * | — | 0.60 | 0.74 |
| Leu | 97 | A | A | — | — | — | — | — | 0.72 | −1.17 | * | * | — | 0.60 | 0.71 |
| Glu | 98 | A | A | — | — | — | — | — | 0.90 | −0.69 | — | — | — | 0.60 | 0.59 |
| Ala | 99 | A | A | — | — | — | — | — | 0.90 | −0.19 | — | * | F | 0.60 | 1.08 |
| Asp | 100 | A | — | — | — | — | T | — | 1.01 | −0.43 | — | * | F | 1.00 | 2.28 |
| Thr | 101 | A | — | — | — | — | T | — | 1.57 | −1.11 | * | * | F | 1.30 | 2.58 |
| His | 102 | A | — | — | — | — | T | — | 1.49 | −0.73 | * | * | F | 1.30 | 3.42 |
| Gln | 103 | — | — | — | — | T | T | — | 1.19 | −0.54 | * | — | F | 1.91 | 1.43 |
| Arg | 104 | — | — | — | B | T | — | — | 1.57 | −0.16 | * | — | F | 1.42 | 1.33 |
| Ser | 105 | — | — | — | B | T | — | — | 1.28 | −0.21 | * | * | F | 1.63 | 1.51 |
| Ile | 106 | — | — | — | B | — | — | C | 1.70 | 0.20 | * | * | F | 0.89 | 0.92 |
| Ser | 107 | — | — | — | — | — | T | C | 1.49 | −0.20 | * | * | F | 2.10 | 0.92 |
| Pro | 108 | — | — | — | — | T | T | — | 1.60 | 0.56 | * | * | F | 1.34 | 1.07 |
| Trp | 109 | — | — | — | — | T | T | — | 0.63 | 0.17 | * | * | — | 1.28 | 3.00 |
| Arg | 110 | — | — | — | — | — | T | C | 0.93 | 0.13 | — | * | — | 0.87 | 1.66 |
| Tyr | 111 | — | — | — | B | T | — | — | 1.51 | −0.26 | — | * | — | 1.40 | 1.80 |
| Arg | 112 | — | — | — | B | T | — | — | 1.81 | −0.20 | — | * | — | 1.53 | 2.46 |
| Val | 113 | — | — | — | B | — | — | C | 2.02 | −1.11 | — | * | — | 1.97 | 2.10 |
| Asp | 114 | — | — | — | — | T | T | — | 2.31 | −1.11 | — | * | F | 3.06 | 2.32 |
| Thr | 115 | — | — | — | — | T | T | — | 2.31 | −1.87 | — | * | F | 3.40 | 1.98 |
| Asp | 116 | — | — | — | — | T | T | — | 2.31 | −1.87 | * | * | F | 3.06 | 5.23 |
| Glu | 117 | — | — | — | — | T | T | — | 1.99 | −1.76 | * | * | F | 2.72 | 4.90 |
| Asp | 118 | — | — | — | — | T | T | — | 2.84 | −1.33 | * | — | F | 2.38 | 5.25 |
| Arg | 119 | A | — | — | — | — | T | — | 2.89 | −1.41 | * | * | F | 1.64 | 5.45 |
| Tyr | 120 | A | — | — | — | — | T | — | 2.39 | −1.41 | * | — | F | 1.30 | 6.29 |
| Pro | 121 | A | — | — | — | — | T | — | 1.80 | −0.73 | * | * | F | 1.30 | 3.11 |
| Gln | 122 | A | A | — | — | — | — | — | 1.10 | −0.23 | * | * | F | 0.60 | 1.60 |
| Lys | 123 | A | A | — | — | — | — | — | 0.51 | 0.56 | * | * | F | −0.45 | 0.89 |
| Leu | 124 | A | A | — | — | — | — | — | 0.40 | 0.30 | * | * | — | −0.30 | 0.58 |
| Ala | 125 | A | A | — | — | — | — | — | −0.02 | −0.13 | — | — | — | 0.30 | 0.58 |
| Phe | 126 | A | A | — | — | — | — | — | −0.62 | 0.04 | — | — | — | −0.30 | 0.16 |
| Ala | 127 | A | A | — | — | — | — | — | −1.29 | 0.73 | * | — | — | −0.60 | 0.16 |
| Glu | 128 | A | A | — | — | — | — | — | −1.22 | 0.61 | * | * | — | −0.60 | 0.08 |
| Cys | 129 | A | A | — | — | — | — | — | −0.76 | 0.11 | * | — | — | −0.30 | 0.19 |
| Leu | 130 | A | A | — | — | — | — | — | −0.83 | −0.24 | * | * | — | 0.30 | 0.18 |
| Cys | 131 | — | — | — | — | T | T | — | −1.02 | −0.17 | * | * | — | 1.10 | 0.06 |
| Arg | 132 | — | — | — | — | T | T | — | −0.43 | 0.51 | * | * | — | 0.20 | 0.07 |
| Gly | 133 | — | — | — | — | T | T | — | −1.02 | −0.06 | * | * | — | 1.10 | 0.15 |
| Cys | 134 | — | — | — | — | T | T | — | −0.24 | −0.24 | * | * | — | 1.40 | 0.28 |
| Ile | 135 | A | — | — | — | — | — | — | 0.26 | −0.81 | * | * | — | 1.40 | 0.28 |
| Asp | 136 | — | — | — | — | T | — | — | 0.58 | −0.33 | — | * | — | 1.80 | 0.41 |
| Ala | 137 | — | — | — | — | T | — | — | 0.58 | −0.33 | — | * | — | 2.10 | 0.76 |
| Arg | 138 | — | — | — | — | — | T | C | 0.92 | −0.90 | * | * | F | 3.00 | 2.12 |
| Thr | 139 | — | — | — | — | — | T | C | 1.28 | −1.59 | * | * | F | 2.70 | 2.20 |
| Gly | 140 | — | — | — | — | — | T | C | 1.58 | −1.10 | * | * | F | 2.40 | 3.14 |
| Arg | 141 | A | — | — | — | — | T | — | 0.99 | −1.10 | * | — | F | 1.90 | 1.62 |
| Glu | 142 | A | A | — | — | — | — | — | 0.77 | −0.60 | * | * | F | 1.20 | 1.13 |
| Thr | 143 | A | A | — | — | — | — | — | 0.66 | −0.40 | * | — | F | 0.45 | 0.94 |
| Ala | 144 | A | A | — | — | — | — | — | 0.67 | −0.43 | — | * | — | 0.30 | 0.78 |
| Ala | 145 | A | A | — | — | — | — | — | 0.16 | −0.04 | — | * | — | 0.30 | 0.60 |
| Leu | 146 | A | — | — | B | — | — | — | 0.16 | 0.60 | — | * | — | −0.60 | 0.31 |
| Asn | 147 | A | — | — | B | — | — | — | −0.66 | 0.11 | * | — | — | −0.30 | 0.60 |
| Ser | 148 | A | — | — | B | — | — | — | −1.16 | 0.30 | * | — | — | −0.30 | 0.49 |
| Val | 149 | A | — | — | B | — | — | — | −0.57 | 0.49 | * | — | — | −0.60 | 0.49 |
| Arg | 150 | A | — | — | B | — | — | — | −0.28 | 0.20 | * | — | — | −0.30 | 0.53 |
| Leu | 151 | A | — | — | B | — | — | — | −0.28 | 0.19 | * | — | — | −0.30 | 0.53 |
| Leu | 152 | A | — | — | B | — | — | — | −1.09 | 0.49 | * | * | — | −0.60 | 0.58 |
| Gln | 153 | A | — | — | B | — | — | — | −1.64 | 0.53 | * | * | — | −0.60 | 0.25 |
| Ser | 154 | A | — | — | B | — | — | — | −1.60 | 1.17 | — | * | — | −0.60 | 0.22 |
| Leu | 155 | — | — | B | B | — | — | — | −1.60 | 1.17 | * | — | — | −0.60 | 0.22 |
| Leu | 156 | — | — | B | B | — | — | — | −0.68 | 0.49 | * | * | — | −0.60 | 0.25 |
| Val | 157 | — | — | B | B | — | — | — | 0.24 | 0.09 | * | * | — | −0.30 | 0.37 |
| Leu | 158 | — | — | B | B | — | — | — | 0.03 | −0.30 | * | — | — | 0.30 | 0.87 |
| Arg | 159 | — | — | — | B | T | — | — | −0.33 | −0.56 | — | — | F | 1.30 | 1.63 |
| Arg | 160 | — | — | — | B | T | — | — | 0.18 | −0.67 | — | * | F | 1.30 | 1.18 |
| Arg | 161 | — | — | — | B | — | — | C | 1.10 | −0.93 | — | * | F | 1.10 | 1.91 |
| Pro | 162 | — | — | — | — | T | — | — | 1.96 | −1.61 | — | * | F | 1.84 | 1.91 |
| Cys | 163 | — | — | — | — | T | — | — | 2.42 | −1.61 | — | * | F | 2.18 | 1.63 |
| Ser | 164 | — | — | — | — | T | T | — | 2.01 | −1.19 | — | * | F | 2.57 | 0.82 |
| Arg | 165 | — | — | — | — | T | T | — | 1.56 | −0.80 | * | — | F | 2.91 | 0.71 |
| Asp | 166 | — | — | — | — | T | T | — | 0.63 | −0.80 | * | * | F | 3.40 | 1.32 |
| Gly | 167 | — | — | — | — | T | T | — | 0.63 | −0.69 | * | — | F | 2.91 | 0.81 |
| Ser | 168 | — | — | — | — | T | — | — | 0.99 | −0.64 | * | — | F | 2.37 | 0.64 |
| Gly | 169 | — | — | — | — | — | — | C | 1.08 | −0.16 | * | — | F | 1.53 | 0.55 |
| Leu | 170 | — | — | — | — | — | — | C | 0.62 | 0.27 | * | — | F | 0.59 | 0.87 |
| Pro | 171 | — | — | — | — | — | — | C | 0.03 | 0.27 | — | — | F | 0.25 | 0.64 |
| Thr | 172 | — | — | — | — | — | T | C | −0.32 | 0.39 | — | — | F | 0.45 | 0.65 |
| Pro | 173 | — | — | — | — | — | T | C | −0.61 | 0.74 | — | — | F | 0.15 | 0.68 |
| Gly | 174 | — | — | — | — | — | T | C | −0.97 | 0.56 | — | — | F | 0.15 | 0.45 |
| Ala | 175 | A | — | — | — | — | T | — | −0.19 | 0.91 | — | — | — | −0.20 | 0.27 |
| Phe | 176 | A | A | — | — | — | — | — | −0.29 | 0.93 | — | — | — | −0.60 | 0.24 |
| Ala | 177 | A | A | — | — | — | — | — | 0.02 | 0.99 | — | * | — | −0.60 | 0.34 |
| Phe | 178 | A | A | — | — | — | — | — | −0.47 | 0.56 | — | — | — | −0.60 | 0.59 |
| His | 179 | A | A | — | — | — | — | — | −1.01 | 0.84 | — | — | — | −0.60 | 0.59 |
| Thr | 180 | A | — | — | B | — | — | — | −0.46 | 0.74 | — | — | — | −0.60 | 0.41 |
| Glu | 181 | A | — | — | B | — | — | — | −0.61 | 0.74 | — | — | — | −0.60 | 0.64 |
| Phe | 182 | A | — | — | B | — | — | — | −0.23 | 0.60 | — | — | — | −0.60 | 0.35 |
| Ile | 183 | — | — | — | B | T | — | — | −0.39 | 0.53 | — | — | — | −0.20 | 0.38 |
| His | 184 | — | — | — | B | T | — | — | −0.70 | 0.69 | — | — | — | −0.20 | 0.16 |
| Val | 185 | — | — | — | B | — | — | C | −1.06 | 1.11 | — | — | — | −0.40 | 0.18 |
| Pro | 186 | — | — | — | — | T | T | — | −1.37 | 0.90 | — | — | — | 0.20 | 0.14 |
| Val | 187 | — | — | — | — | T | T | — | −1.33 | 0.70 | — | — | — | 0.20 | 0.15 |
| Gly | 188 | — | — | — | — | T | T | — | −1.30 | 0.77 | — | * | — | 0.20 | 0.11 |
| Cys | 189 | — | — | — | — | T | T | — | −2.08 | 0.77 | — | — | — | 0.20 | 0.05 |
| Thr | 190 | — | — | B | B | — | — | — | −1.43 | 1.03 | — | * | — | −0.60 | 0.06 |
| Cys | 191 | — | — | B | B | — | — | — | −1.11 | 0.81 | — | — | — | −0.60 | 0.09 |
| Val | 192 | — | — | B | B | — | — | — | −0.56 | 0.39 | * | — | — | −0.30 | 0.33 |
| Leu | 193 | — | — | B | — | — | T | — | −1.07 | 0.20 | * | — | — | 0.28 | 0.31 |
| Pro | 194 | — | — | B | — | — | T | | −0.79 | 0.36 | * | — | F | 0.61 | 0.42 |
| Arg | 195 | — | — | — | — | T | T | — | −0.87 | 0.21 | * | — | — | 1.04 | 0.73 |
| Ser | 196 | — | — | — | — | T | T | — | −0.59 | −0.00 | * | — | — | 1.97 | 1.13 |
| Val | 197 | — | — | — | — | T | — | — | −0.12 | −0.26 | * | — | — | 1.80 | 0.93 |

TABLE II

| Res | Position | I | II | III | IV | V | VI | VII | VIII | IX | X | XI | XII | XIII |
|---|---|---|---|---|---|---|---|---|---|---|---|---|---|---|
| Asn | 1 | — | — | — | — | — | — | C | 0.58 | — | * | — | 0.85 | 1.60 |
| Ser | 2 | — | A | — | — | — | — | C | 1.08 | — | * | — | 0.65 | 1.26 |
| Ala | 3 | — | A | B | — | — | — | — | 0.88 | — | * | — | 0.75 | 1.93 |
| Arg | 4 | — | A | B | — | — | — | — | 0.41 | — | * | — | 0.75 | 1.22 |
| Ala | 5 | — | A | B | — | — | — | — | −0.01 | — | * | — | 0.30 | 0.67 |
| Arg | 6 | — | A | B | — | — | — | — | −0.31 | — | * | — | 0.30 | 0.55 |
| Ala | 7 | — | A | B | — | — | — | — | −0.60 | — | * | — | 0.30 | 0.38 |
| Val | 8 | — | A | B | — | — | — | — | −0.71 | — | * | — | −0.30 | 0.38 |
| Leu | 9 | — | A | B | — | — | — | — | −0.86 | * | * | — | −0.60 | 0.17 |
| Ser | 10 | — | A | B | — | — | — | — | −0.30 | * | * | — | −0.60 | 0.22 |
| Ala | 11 | — | A | B | — | — | — | — | −0.72 | * | — | — | −0.60 | 0.41 |
| Phe | 12 | — | A | B | — | — | — | — | −0.94 | * | — | — | −0.60 | 0.72 |
| His | 13 | — | A | B | — | — | — | — | −0.09 | * | — | — | −0.60 | 0.44 |
| His | 14 | — | A | B | — | — | — | — | −0.09 | * | — | — | −0.60 | 0.76 |
| Thr | 15 | — | A | B | — | — | — | — | −0.13 | * | — | — | −0.60 | 0.72 |
| Leu | 16 | — | A | — | — | — | — | C | 0.24 | * | * | — | −0.10 | 0.52 |
| Gln | 17 | — | A | — | — | T | — | — | 1.06 | * | * | — | 0.40 | 0.60 |
| Leu | 18 | — | A | — | — | — | — | C | 1.09 | — | * | — | 0.80 | 0.81 |
| Gly | 19 | — | — | — | — | — | T | C | 1.12 | — | * | F | 2.40 | 1.70 |
| Pro | 20 | — | — | — | — | — | T | C | 0.84 | * | * | F | 3.00 | 1.70 |
| Arg | 21 | — | — | — | — | — | T | C | 1.77 | * | * | F | 2.70 | 2.08 |
| Glu | 22 | — | — | B | — | — | T | — | 1.77 | * | * | F | 2.20 | 4.12 |
| Gln | 23 | — | — | B | — | — | — | — | 1.99 | * | * | F | 1.70 | 4.28 |
| Ala | 24 | — | — | — | — | T | — | — | 2.03 | * | * | F | 1.80 | 2.21 |
| Arg | 25 | — | — | — | — | T | — | — | 1.58 | * | * | F | 1.50 | 1.71 |
| Asn | 26 | — | — | — | — | T | — | — | 1.26 | * | * | F | 1.05 | 0.53 |
| Ala | 27 | — | — | — | — | T | — | — | 0.67 | * | — | — | 0.90 | 0.81 |
| Ser | 28 | — | — | B | — | — | — | — | 0.32 | — | — | — | 0.78 | 0.42 |
| Cys | 29 | — | — | B | — | — | T | — | 0.57 | — | * | — | 0.66 | 0.26 |
| Pro | 30 | — | — | — | — | T | T | — | 0.57 | — | * | — | 1.34 | 0.25 |
| Ala | 31 | — | — | — | — | T | T | — | 0.36 | — | * | F | 2.37 | 0.37 |
| Gly | 32 | — | — | — | — | T | T | — | 0.36 | — | — | F | 2.80 | 1.06 |
| Gly | 33 | — | — | — | — | — | — | C | 0.66 | * | * | F | 1.97 | 0.69 |
| Arg | 34 | — | — | B | — | — | — | — | 1.43 | * | — | F | 1.94 | 1.15 |
| Pro | 35 | — | — | B | — | — | T | — | 1.76 | * | — | F | 1.86 | 2.27 |
| Ala | 36 | — | — | B | — | — | T | — | 1.64 | * | * | F | 1.58 | 4.49 |
| Asp | 37 | — | — | B | — | — | T | — | 2.10 | * | * | F | 1.30 | 1.99 |
| Arg | 38 | — | — | B | — | — | T | — | 2.23 | * | * | F | 1.30 | 2.52 |
| Arg | 39 | — | — | B | — | — | — | — | 1.91 | * | * | F | 1.10 | 3.85 |
| Phe | 40 | — | — | B | — | — | — | — | 1.81 | * | * | F | 1.44 | 3.57 |
| Arg | 41 | — | — | B | — | — | — | — | 2.40 | * | * | F | 1.78 | 2.63 |
| Pro | 42 | — | — | — | — | — | T | C | 1.59 | — | * | F | 2.22 | 2.16 |
| Pro | 43 | — | — | — | — | T | T | — | 1.59 | — | * | F | 2.16 | 2.05 |
| Thr | 44 | — | — | — | — | T | T | — | 1.18 | — | * | F | 3.40 | 2.05 |
| Asn | 45 | — | — | — | — | — | T | C | 1.02 | * | * | F | 2.56 | 1.78 |
| Leu | 46 | — | — | B | B | — | — | — | 0.61 | * | * | F | 0.87 | 0.85 |
| Arg | 47 | — | — | B | B | — | — | — | 0.61 | * | — | F | 1.13 | 0.79 |
| Ser | 48 | — | — | B | B | — | — | — | 0.53 | * | — | F | 0.79 | 0.76 |
| Val | 49 | — | — | B | B | — | — | — | 0.26 | * | — | F | −0.45 | 0.97 |
| Ser | 50 | — | — | B | — | — | T | — | 0.01 | * | * | F | 0.25 | 0.50 |
| Pro | 51 | — | — | B | — | — | T | — | 0.93 | * | * | — | −0.20 | 0.59 |
| Trp | 52 | — | — | B | — | — | T | — | −0.07 | * | * | — | −0.05 | 1.55 |
| Ala | 53 | — | — | B | — | — | T | — | −0.07 | * | * | — | −0.20 | 0.81 |
| Tyr | 54 | — | — | B | B | — | — | — | 0.54 | * | * | — | −0.60 | 0.70 |
| Arg | 55 | — | — | B | B | — | — | — | 0.84 | — | * | — | −0.45 | 1.05 |
| Ile | 56 | — | — | B | B | — | — | — | 0.84 | * | * | — | 0.13 | 1.73 |
| Ser | 57 | — | — | B | — | — | — | — | 0.54 | * | * | — | 0.61 | 1.71 |
| Tyr | 58 | — | — | — | — | T | — | — | 1.24 | * | * | — | 1.74 | 0.88 |
| Asp | 59 | — | — | — | — | — | T | C | 1.24 | * | * | F | 2.32 | 2.46 |
| Pro | 60 | — | — | — | — | T | T | — | 0.92 | * | — | F | 2.80 | 2.88 |
| Ala | 61 | — | — | — | — | T | T | — | 1.92 | * | — | F | 2.52 | 2.84 |
| Arg | 62 | — | — | B | — | — | T | — | 1.98 | * | — | F | 2.14 | 3.33 |
| Tyr | 63 | — | — | B | — | — | T | — | 1.41 | * | — | — | 1.41 | 3.37 |
| Pro | 64 | — | — | B | — | — | T | — | 1.20 | * | — | — | 0.53 | 2.75 |
| Arg | 65 | — | — | — | — | T | T | — | 1.41 | * | — | — | 0.65 | 2.17 |
| Tyr | 66 | — | — | B | — | — | T | — | 1.41 | * | — | F | 0.40 | 2.40 |
| Leu | 67 | — | — | B | — | — | — | — | 1.06 | * | — | F | 0.80 | 1.57 |
| Pro | 68 | — | — | B | — | — | — | — | 0.63 | * | — | — | 0.05 | 1.26 |
| Glu | 69 | — | — | — | — | T | — | — | 0.03 | * | — | — | 0.00 | 0.43 |
| Ala | 70 | — | — | B | B | — | — | — | −0.74 | * | — | — | −0.60 | 0.43 |
| Tyr | 71 | — | — | B | B | — | — | — | −0.39 | * | — | — | −0.60 | 0.15 |
| Cys | 72 | — | — | B | B | — | — | — | 0.08 | * | — | — | −0.30 | 0.17 |
| Leu | 73 | — | — | B | B | — | — | — | −0.38 | — | * | — | −0.60 | 0.16 |
| Cys | 74 | — | — | B | — | — | T | — | −1.19 | — | * | — | −0.20 | 0.06 |
| Arg | 75 | — | — | B | — | — | T | — | −0.91 | * | * | — | −0.20 | 0.09 |
| Gly | 76 | — | — | B | — | — | T | — | −1.01 | * | — | — | −0.20 | 0.15 |
| Cys | 77 | — | — | B | — | — | T | — | −1.16 | — | * | — | 0.10 | 0.28 |
| Leu | 78 | — | — | B | B | — | — | — | −1.04 | — | — | — | −0.30 | 0.12 |
| Thr | 79 | — | — | B | B | — | — | — | −0.72 | — | * | — | −0.60 | 0.10 |
| Gly | 80 | — | — | — | B | — | — | C | −0.83 | — | * | — | −0.40 | 0.19 |
| Leu | 81 | — | — | — | B | — | — | C | −0.49 | — | — | — | −0.40 | 0.40 |
| Phe | 82 | — | — | B | B | — | — | — | 0.18 | — | — | F | 0.45 | 0.48 |
| Gly | 83 | — | — | — | B | — | — | C | 0.13 | — | * | F | 0.95 | 0.81 |
| Glu | 84 | — | A | B | — | — | — | — | 0.56 | — | * | F | 0.45 | 0.73 |
| Glu | 85 | — | A | B | — | — | — | — | 0.20 | — | * | F | 0.90 | 1.65 |
| Asp | 86 | — | A | B | B | — | — | — | 1.12 | — | * | F | 0.90 | 1.45 |
| Val | 87 | — | A | B | B | — | — | — | 1.52 | — | * | F | 0.90 | 1.63 |
| Arg | 88 | — | A | — | B | T | — | — | 1.28 | — | * | — | 1.15 | 1.26 |
| Phe | 89 | — | A | — | B | T | — | — | 1.07 | — | * | — | 1.00 | 0.77 |
| Arg | 90 | — | A | — | B | T | — | — | 0.21 | — | * | — | 0.85 | 1.59 |
| Ser | 91 | — | A | — | B | — | — | C | −0.03 | — | * | — | 0.50 | 0.60 |
| Ala | 92 | — | — | — | B | — | — | C | 0.22 | — | * | — | −0.25 | 1.09 |
| Pro | 93 | — | — | — | B | — | — | C | −0.10 | — | * | — | −0.10 | 0.55 |
| Val | 94 | — | — | — | B | T | — | — | 0.29 | * | — | — | −0.20 | 0.64 |
| Tyr | 95 | — | — | B | B | — | — | — | −0.68 | * | — | — | −0.60 | 0.91 |
| Met | 96 | — | — | B | B | — | — | — | −1.23 | — | — | — | −0.60 | 0.44 |
| Pro | 97 | — | — | B | B | — | — | — | −1.46 | — | * | — | −0.60 | 0.44 |
| Thr | 98 | — | — | B | B | — | — | — | −1.13 | * | — | — | −0.60 | 0.23 |
| Val | 99 | — | — | B | B | — | — | — | −0.17 | * | — | — | −0.60 | 0.46 |
| Val | 100 | — | — | B | B | — | — | — | −0.23 | — | — | — | 0.30 | 0.58 |
| Leu | 101 | — | — | B | B | — | — | — | 0.16 | — | — | — | 0.30 | 0.58 |
| Arg | 102 | — | — | B | B | — | — | — | −0.22 | — | — | F | 0.60 | 1.20 |
| Arg | 103 | — | — | B | B | — | — | — | −0.58 | — | — | F | 0.60 | 1.63 |
| Thr | 104 | — | — | B | B | — | — | — | −0.31 | — | — | F | 0.60 | 1.06 |
| Pro | 105 | — | — | B | B | — | — | — | 0.20 | * | — | F | 1.00 | 0.55 |
| Ala | 106 | — | — | B | — | — | — | — | 0.67 | — | * | — | 1.00 | 0.28 |
| Cys | 107 | — | — | B | — | — | T | — | 0.67 | — | — | — | 0.85 | 0.19 |
| Ala | 108 | — | — | — | — | T | T | — | 0.26 | * | * | — | 2.10 | 0.24 |
| Gly | 109 | — | — | — | — | T | T | — | −0.29 | * | — | F | 2.50 | 0.32 |
| Gly | 110 | — | — | — | — | T | T | — | −0.32 | * | — | F | 2.25 | 0.44 |
| Arg | 111 | — | — | B | B | — | — | — | −0.04 | * | — | F | 0.60 | 0.69 |
| Ser | 112 | — | — | B | B | — | — | — | 0.62 | * | — | F | 0.35 | 1.00 |
| Val | 113 | — | — | B | B | — | — | — | 0.62 | * | — | — | 0.70 | 1.75 |
| Tyr | 114 | — | — | B | — | — | — | — | 0.72 | — | — | — | 0.50 | 0.90 |
| Thr | 115 | — | — | B | — | — | — | — | 0.21 | — | — | — | −0.25 | 1.05 |
| Glu | 116 | — | — | B | B | — | — | — | −0.21 | — | * | — | −0.45 | 1.05 |
| Ala | 117 | — | — | B | B | — | — | — | −0.80 | — | * | — | −0.60 | 0.97 |
| Tyr | 118 | — | — | B | B | — | — | — | −0.16 | — | * | — | −0.60 | 0.47 |
| Val | 119 | — | — | B | B | — | — | — | −0.77 | — | — | — | −0.60 | 0.42 |
| Thr | 120 | — | — | B | B | — | — | — | −0.80 | — | * | — | −0.60 | 0.31 |
| Ile | 121 | — | — | B | B | — | — | — | −1.47 | — | * | — | −0.60 | 0.20 |
| Pro | 122 | — | — | B | — | — | T | — | −1.19 | — | * | — | −0.20 | 0.14 |
| Val | 123 | — | — | — | — | T | T | — | −1.61 | — | — | — | 0.20 | 0.14 |
| Gly | 124 | — | — | — | — | T | T | — | −1.61 | — | — | — | 0.20 | 0.11 |
| Cys | 125 | — | — | B | — | — | T | — | −1.51 | — | — | — | −0.20 | 0.05 |
| Thr | 126 | — | — | B | — | — | — | — | −0.62 | — | — | — | −0.40 | 0.11 |
| Cys | 127 | — | — | B | — | — | — | — | −0.62 | — | — | — | −0.10 | 0.19 |
| Val | 128 | — | — | B | — | — | T | — | 0.23 | — | — | — | 0.40 | 0.55 |
| Pro | 129 | — | — | B | — | — | T | — | 0.62 | — | — | F | 1.45 | 0.65 |
| Glu | 130 | — | — | B | — | — | T | — | 1.29 | * | — | F | 2.20 | 2.44 |
| Pro | 131 | — | — | B | — | — | T | — | 1.01 | * | — | F | 2.50 | 5.49 |
| Glu | 132 | — | — | — | — | T | — | — | 1.68 | * | — | F | 3.00 | 3.59 |
| Lys | 133 | A | — | — | — | — | — | — | 2.23 | * | — | F | 2.30 | 3.46 |
| Asp | 134 | A | — | — | — | — | T | — | 1.56 | * | — | F | 2.20 | 3.00 |
| Ala | 135 | A | — | — | — | — | T | — | 1.56 | * | — | F | 1.90 | 1.21 |
| Asp | 136 | A | — | — | — | — | T | — | 1.47 | * | — | F | 1.45 | 0.98 |
| Ser | 137 | — | — | B | — | — | T | — | 1.17 | * | — | F | 1.15 | 0.78 |
| Ile | 138 | — | — | B | — | — | — | — | 0.23 | * | — | F | 0.80 | 1.04 |
| Asn | 139 | — | — | B | — | — | T | — | 0.23 | * | — | F | 0.85 | 0.44 |
| Ser | 140 | — | — | B | — | — | T | — | 0.87 | * | — | F | 1.16 | 0.54 |
| Ser | 141 | — | — | B | — | — | T | — | 0.87 | * | — | F | 1.62 | 1.55 |
| Ile | 142 | — | — | B | — | — | T | — | 0.82 | — | * | F | 2.23 | 1.67 |
| Asp | 143 | — | — | B | — | — | T | — | 1.12 | * | * | F | 2.54 | 1.23 |
| Lys | 144 | — | — | — | — | V | T | — | 1.17 | * | — | F | 3.10 | 0.93 |
| Gln | 145 | — | — | B | — | — | T | — | 0.66 | * | — | F | 2.54 | 2.65 |
| Gly | 146 | — | — | B | — | — | T | — | 0.14 | * | — | F | 2.23 | 1.31 |
| Ala | 147 | — | A | B | — | — | — | — | 0.22 | * | — | F | 1.07 | 0.54 |
| Lys | 148 | — | A | B | — | — | — | — | −0.12 | — | — | F | 0.16 | 0.26 |
| Leu | 149 | — | A | B | — | — | — | — | −0.38 | * | — | — | −0.60 | 0.26 |
| Leu | 150 | — | A | B | — | — | — | — | −0.38 | — | — | — | −0.60 | 0.39 |
| Leu | 151 | — | A | B | — | — | — | — | −0.03 | — | — | — | −0.06 | 0.32 |
| Gly | 152 | — | — | B | — | — | T | — | −0.03 | — | — | F | 0.73 | 0.64 |
| Pro | 153 | — | — | — | — | — | T | C | −0.29 | — | — | F | 1.17 | 0.78 |
| Asn | 154 | — | — | — | — | T | T | — | −0.07 | — | — | F | 2.36 | 1.47 |
| Asp | 155 | — | — | — | — | — | T | C | 0.40 | — | — | F | 2.40 | 1.50 |
| Ala | 156 | — | — | — | — | — | — | C | 1.00 | — | — | F | 1.81 | 0.96 |
| Pro | 157 | — | — | — | — | — | T | C | 0.96 | — | — | F | 1.77 | 0.92 |
| Ala | 158 | — | — | — | — | — | T | C | 0.78 | — | — | — | 1.38 | 0.71 |
| Gly | 159 | — | — | — | — | — | T | C | 0.39 | — | — | — | 0.54 | 0.90 |
| Pro | 160 | — | — | B | — | — | T | — | 0.00 | — | — | — | 0.10 | 0.74 |

TABLE III

| Res | Position | I | II | III | IV | V | VI | VII | VIII | IX | X | XI | XII | XIII | XIV |
|---|---|---|---|---|---|---|---|---|---|---|---|---|---|---|---|
| Gly | 1 | — | A | — | — | T | — | — | 0.46 | −0.21 | | — | — | — | 0.70 |
| | 0.34 | | | | | | | | | | | | | | |
| Cys | 2 | — | A | — | — | T | — | — | 0.63 | −0.64 | | — | — | — | 1.00 |
| | 0.51 | | | | | | | | | | | | | | |
| Ala | 3 | — | A | — | — | — | — | C | 1.02 | −0.64 | | — | — | — | 0.80 |
| | 0.62 | | | | | | | | | | | | | | |
| Asp | 4 | — | A | — | — | — | — | C | 1.41 | −1.07 | | — | — | — | 0.95 |
| | 1.09 | | | | | | | | | | | | | | |
| Arg | 5 | A | A | — | — | — | — | — | 0.99 | −1.50 | | * | — | F | 0.90 |
| | 3.51 | | | | | | | | | | | | | | |
| Pro | 6 | A | A | — | — | — | — | — | 0.52 | −1.39 | | * | — | F | 0.90 |
| | 2.87 | | | | | | | | | | | | | | |
| Glu | 7 | A | A | — | — | — | — | — | 1.19 | −1.20 | | * | — | F | 0.90 |
| | 1.42 | | | | | | | | | | | | | | |
| Glu | 8 | A | A | — | — | — | — | — | 1.78 | −1.20 | | * | — | F | 0.90 |
| | 1.25 | | | | | | | | | | | | | | |
| Leu | 9 | A | A | — | — | — | — | — | 0.97 | −0.80 | | * | — | F | 0.90 |
| | 1.40 | | | | | | | | | | | | | | |
| Leu | 10 | A | A | — | — | — | — | — | 0.61 | −0.54 | | * | — | F | 0.75 |
| | 0.67 | | | | | | | | | | | | | | |
| Glu | 11 | A | A | — | — | — | — | — | 0.48 | 0.21 | * | * | — | −0.30 | 0.60 |
| Gln | 12 | A | A | — | B | — | — | — | 0.59 | 0.64 | * | * | — | −0.60 | 0.73 |
| Leu | 13 | A | A | — | B | — | — | — | −0.22 | −0.04 | | * | * | — | 0.45 |
| | 1.72 | | | | | | | | | | | | | | |
| Tyr | 14 | A | A | — | B | — | — | — | −0.00 | −0.04 | | * | * | — | 0.30 |
| | 0.82 | | | | | | | | | | | | | | |
| Gly | 15 | A | A | — | — | — | — | — | 0.22 | 0.46 | * | * | — | −0.60 | 0.48 |
| Arg | 16 | A | A | — | — | — | — | — | −0.12 | 0.56 | * | * | — | −0.60 | 0.59 |
| Leu | 17 | A | A | — | — | — | — | — | −0.98 | 0.30 | * | * | — | −0.30 | 0.37 |
| Ala | 18 | A | A | — | B | — | — | — | −0.98 | 0.19 | * | * | — | −0.30 | 0.28 |
| Ala | 19 | A | A | — | B | — | — | — | −1.03 | 0.44 | * | * | — | −0.60 | 0.12 |
| Gly | 20 | A | A | — | B | — | — | — | −1.28 | 0.83 | * | * | — | −0.60 | 0.19 |
| Val | 21 | A | A | — | B | — | — | — | −2.09 | 0.64 | * | — | — | −0.60 | 0.19 |
| Leu | 22 | A | A | — | B | — | — | — | −1.31 | 0.93 | — | — | — | −0.60 | 0.16 |
| Ser | 23 | A | A | — | — | — | — | — | −0.76 | 0.93 | — | — | — | −0.60 | 0.22 |
| Ala | 24 | A | A | — | — | — | — | — | −0.48 | 1.00 | — | — | — | −0.60 | 0.41 |
| Phe | 25 | A | A | — | B | — | — | — | −0.94 | 0.84 | * | — | — | −0.60 | 0.72 |
| His | 26 | A | A | — | B | — | — | — | −0.09 | 0.84 | * | * | — | −0.60 | 0.44 |
| His | 27 | — | A | B | B | — | — | — | −0.09 | 0.86 | * | — | — | −0.60 | 0.76 |
| Thr | 28 | — | A | B | B | — | — | — | −0.13 | 1.04 | — | — | — | −0.60 | 0.72 |
| Leu | 29 | — | A | — | B | — | — | C | 0.24 | 0.69 | * | * | — | −0.10 | 0.52 |
| Gln | 30 | — | A | — | B | T | — | — | 1.06 | 0.61 | * | * | — | 0.40 | 0.60 |
| Leu | 31 | — | A | — | B | — | — | C | 1.09 | 0.11 | — | * | — | 0.80 | 0.81 |
| Gly | 32 | — | — | — | — | — | T | C | 1.12 | −0.37 | — | | * | F | 2.40 |
| | 1.70 | | | | | | | | | | | | | | |
| Pro | 33 | — | — | — | — | — | T | C | 0.84 | −0.66 | | * | * | F | 3.00 |
| | 1.70 | | | | | | | | | | | | | | |
| Arg | 34 | — | — | — | — | — | T | C | 1.77 | −0.56 | | * | * | F | 2.70 |
| | 2.08 | | | | | | | | | | | | | | |
| Glu | 35 | A | — | — | — | — | T | — | 1.77 | −1.24 | | * | * | F | 2.20 |
| | 4.12 | | | | | | | | | | | | | | |
| Gln | 36 | A | — | — | — | — | — | — | 1.99 | −1.27 | | * | * | F | 1.70 |
| | 4.28 | | | | | | | | | | | | | | |
| Ala | 37 | — | — | — | — | T | — | — | 2.03 | −1.20 | | * | * | F | 1.80 |
| | 2.21 | | | | | | | | | | | | | | |
| Arg | 38 | — | — | — | — | T | — | — | 1.58 | −0.81 | | * | * | F | 1.50 |
| | 1.71 | | | | | | | | | | | | | | |
| Asn | 39 | — | — | — | — | T | — | — | 1.26 | −0.24 | | * | * | F | 1.05 |
| | 0.53 | | | | | | | | | | | | | | |
| Ala | 40 | — | — | — | — | T | — | — | 0.67 | −0.21 | | * | — | — | 0.90 |
| | 0.81 | | | | | | | | | | | | | | |
| Ser | 41 | — | — | B | — | — | — | — | 0.32 | −0.21 | | — | — | — | 0.78 |
| | 0.42 | | | | | | | | | | | | | | |
| Cys | 42 | — | — | B | — | — | T | — | 0.57 | 0.21 | — | * | — | 0.66 | 0.26 |
| Pro | 43 | — | — | — | — | T | T | — | 0.57 | 0.24 | — | * | — | 1.34 | 0.25 |
| Ala | 44 | — | — | — | — | T | T | — | 0.36 | −0.26 | | — | * | F | 2.37 |
| | 0.37 | | | | | | | | | | | | | | |
| Gly | 45 | — | — | — | — | T | T | — | 0.36 | −0.21 | | — | — | F | 2.80 |
| | 1.06 | | | | | | | | | | | | | | |
| Gly | 46 | — | — | — | — | — | — | C | 0.66 | −0.29 | | * | * | F | 1.97 |
| | 0.69 | | | | | | | | | | | | | | |
| Arg | 47 | — | — | B | — | — | — | — | 1.43 | −0.71 | | * | — | F | 1.94 |
| | 1.15 | | | | | | | | | | | | | | |
| Pro | 48 | — | — | B | — | — | T | — | 1.76 | −1.21 | | * | — | F | 1.86 |
| | 2.27 | | | | | | | | | | | | | | |
| Ala | 49 | — | — | B | — | — | T | — | 1.64 | −1.64 | | * | * | F | 1.58 |
| | 4.49 | | | | | | | | | | | | | | |
| Asp | 50 | — | — | B | — | — | T | — | 2.10 | −1.29 | | * | * | F | 1.30 |
| | 1.99 | | | | | | | | | | | | | | |
| Arg | 51 | — | — | B | — | — | T | — | 2.23 | −1.29 | | * | * | F | 1.30 |
| | 2.52 | | | | | | | | | | | | | | |
| Arg | 52 | — | — | B | — | — | — | — | 1.91 | −1.29 | | * | * | F | 1.10 |
| | 3.85 | | | | | | | | | | | | | | |
| Phe | 53 | — | — | B | — | — | — | — | 1.81 | −1.36 | | * | * | F | 1.44 |
| | 3.57 | | | | | | | | | | | | | | |
| Arg | 54 | — | — | B | — | — | — | — | 2.40 | −0.87 | | * | * | F | 1.78 |
| | 2.63 | | | | | | | | | | | | | | |
| Pro | 55 | — | — | — | — | — | T | C | 1.59 | −0.47 | | — | * | F | 2.22 |
| | 2.16 | | | | | | | | | | | | | | |
| Pro | 56 | — | — | — | — | T | T | — | 1.59 | 0.21 | — | * | F | 2.16 | 2.05 |
| Thr | 57 | — | — | — | — | T | T | — | 1.18 | −0.57 | | — | * | F | 3.40 |
| | 2.05 | | | | | | | | | | | | | | |
| Asn | 58 | — | — | — | — | — | T | C | 1.02 | −0.19 | | * | * | F | 2.56 |
| | 1.78 | | | | | | | | | | | | | | |
| Leu | 59 | — | — | B | B | — | — | — | 0.61 | 0.03 | * | * | F | 0.87 | 0.85 |
| Arg | 60 | — | — | B | B | — | — | — | 0.61 | −0.01 | | * | * | F | 1.13 |
| | 0.79 | | | | | | | | | | | | | | |
| Ser | 61 | — | — | B | B | — | — | — | 0.53 | −0.07 | | * | — | F | 0.79 |
| | 0.76 | | | | | | | | | | | | | | |
| Val | 62 | — | — | B | B | — | — | — | 0.26 | 0.44 | * | — | F | −0.45 | 0.97 |
| Ser | 63 | — | — | B | — | — | T | — | 0.01 | 0.26 | * | * | F | 0.25 | 0.50 |
| Pro | 64 | — | — | B | — | — | T | — | 0.93 | 1.01 | * | * | — | −0.20 | 0.59 |
| Trp | 65 | — | — | B | — | — | T | — | −0.07 | 0.63 | * | * | — | −0.05 | 1.55 |
| Ala | 66 | — | — | B | — | — | T | — | −0.07 | 0.67 | * | * | — | −0.20 | 0.81 |
| Tyr | 67 | — | — | B | B | — | — | — | 0.54 | 0.67 | * | * | — | −0.60 | 0.70 |
| Arg | 68 | — | — | B | B | — | — | — | 0.84 | 1.00 | — | * | — | −0.45 | 1.05 |
| Ile | 69 | — | — | B | B | — | — | — | 0.84 | 0.09 | * | * | — | 0.13 | 1.73 |
| Ser | 70 | — | — | B | — | — | — | — | 0.54 | 0.01 | * | * | — | 0.61 | 1.71 |
| Tyr | 71 | — | — | — | — | T | — | — | 1.24 | −0.24 | | * | * | — | 1.74 |
| | 0.88 | | | | | | | | | | | | | | |
| Asp | 72 | — | — | — | — | — | T | C | 1.24 | −0.24 | | * | * | F | 2.32 |
| | 2.46 | | | | | | | | | | | | | | |
| Pro | 73 | — | — | — | — | T | T | — | 0.92 | −0.17 | | * | * | F | 2.80 |
| | 2.88 | | | | | | | | | | | | | | |
| Ala | 74 | — | — | — | — | T | T | — | 1.92 | −0.13 | | * | — | F | 2.52 |
| | 2.84 | | | | | | | | | | | | | | |
| Arg | 75 | — | — | B | — | — | T | — | 1.98 | −0.89 | | * | — | F | 2.14 |
| | 3.33 | | | | | | | | | | | | | | |
| Tyr | 76 | — | — | B | — | — | T | — | 1.41 | −0.13 | | * | — | — | 1.41 |
| | 3.37 | | | | | | | | | | | | | | |
| Pro | 77 | — | — | B | — | — | T | — | 1.20 | 0.13 | * | — | — | 0.53 | 2.75 |
| Arg | 78 | — | — | — | — | T | T | — | 1.41 | 0.06 | * | — | — | 0.65 | 2.17 |
| Tyr | 79 | — | — | B | — | — | T | — | 1.41 | 0.06 | * | — | F | 0.40 | 2.40 |
| Leu | 80 | — | — | B | — | — | — | — | 1.06 | −0.20 | | * | — | F | 0.80 |
| | 1.57 | | | | | | | | | | | | | | |
| Pro | 81 | — | — | B | — | — | — | — | 0.63 | 0.13 | * | — | — | 0.05 | 1.26 |
| Glu | 82 | — | — | — | — | T | — | — | 0.03 | 0.70 | * | — | — | 0.00 | 0.43 |
| Ala | 83 | — | — | B | B | — | — | — | −0.74 | 0.63 | * | — | — | −0.60 | 0.43 |
| Tyr | 84 | — | — | B | B | — | — | — | −0.39 | 0.51 | — | — | — | −0.60 | 0.15 |
| Cys | 85 | — | — | B | B | — | — | — | 0.08 | 0.09 | * | — | — | −0.30 | 0.17 |
| Leu | 86 | — | — | B | B | — | — | — | −0.38 | 0.51 | — | * | — | −0.60 | 0.16 |
| Cys | 87 | — | — | B | — | — | T | — | −1.19 | 0.59 | — | * | — | −0.20 | 0.06 |
| Arg | 88 | — | — | B | — | — | T | — | −0.91 | 0.51 | * | * | — | −0.20 | 0.09 |
| Gly | 89 | — | — | B | — | — | T | — | −1.01 | 0.43 | * | — | — | −0.20 | 0.15 |
| Cys | 90 | — | — | B | — | — | T | — | −1.16 | 0.17 | — | * | — | 0.10 | 0.28 |
| Leu | 91 | — | — | B | B | — | — | — | −1.04 | 0.29 | — | — | — | −0.30 | 0.12 |
| Thr | 92 | — | — | B | B | — | — | — | −0.72 | 1.07 | — | * | — | −0.60 | 0.10 |
| Gly | 93 | — | — | — | B | — | — | C | −0.83 | 1.07 | — | * | — | −0.40 | 0.19 |
| Leu | 94 | — | — | — | B | — | — | C | −0.49 | 0.50 | — | — | — | −0.40 | 0.40 |
| Phe | 95 | — | — | B | B | — | — | — | 0.18 | −0.19 | | — | — | F | 0.45 |
| | 0.48 | | | | | | | | | | | | | | |
| Gly | 96 | A | — | — | B | — | — | — | 0.13 | −0.67 | | — | * | F | 0.75 |
| | 0.81 | | | | | | | | | | | | | | |
| Glu | 97 | A | A | — | — | — | — | — | 0.56 | −0.46 | | — | * | F | 0.45 |
| | 0.73 | | | | | | | | | | | | | | |
| Glu | 98 | A | A | — | — | — | — | — | 0.20 | −1.14 | | — | * | F | 0.90 |
| | 1.65 | | | | | | | | | | | | | | |
| Asp | 99 | A | A | — | B | — | — | — | 1.12 | −1.14 | | — | * | F | 0.90 |
| | 1.45 | | | | | | | | | | | | | | |
| Val | 100 | A | A | — | B | — | — | — | 1.52 | −1.57 | | — | * | F | 0.90 |
| | 1.63 | | | | | | | | | | | | | | |
| Arg | 101 | A | A | — | B | — | — | — | 1.28 | −1.19 | | — | * | — | 0.75 |
| | 1.26 | | | | | | | | | | | | | | |
| Phe | 102 | A | A | — | B | — | — | — | 1.07 | −0.69 | | — | * | — | 0.60 |
| | 0.77 | | | | | | | | | | | | | | |
| Arg | 103 | A | A | — | B | — | — | — | 0.21 | −0.26 | | — | * | — | 0.45 |
| | 1.59 | | | | | | | | | | | | | | |
| Ser | 104 | — | A | — | B | — | — | C | −0.03 | −0.26 | | — | * | — | 0.50 |
| | 0.60 | | | | | | | | | | | | | | |
| Ala | 105 | — | — | — | B | — | — | C | 0.22 | 0.50 | — | * | — | −0.25 | 1.09 |
| Pro | 106 | — | — | — | B | — | — | C | −0.10 | 0.33 | — | * | — | −0.10 | 0.55 |
| Val | 107 | — | — | — | B | T | — | — | 0.29 | 0.76 | * | — | — | −0.20 | 0.64 |
| Tyr | 108 | — | — | B | B | — | — | — | −0.68 | 0.86 | * | — | — | −0.60 | 0.91 |
| Met | 109 | — | — | B | B | — | — | — | −1.23 | 1.00 | — | — | — | −0.60 | 0.44 |
| Pro | 110 | — | — | B | B | — | — | — | −1.46 | 1.21 | — | * | — | −0.60 | 0.44 |
| Thr | 111 | — | — | B | B | — | — | — | −1.13 | 1.26 | * | — | — | −0.60 | 0.23 |
| Val | 112 | — | — | B | B | — | — | — | −0.17 | 0.50 | * | — | — | −0.60 | 0.46 |
| Val | 113 | — | — | B | B | — | — | — | −0.23 | −0.11 | | — | — | — | 0.30 |
| | 0.58 | | | | | | | | | | | | | | |
| Leu | 114 | — | — | B | B | — | — | — | 0.16 | −0.06 | | — | — | — | 0.30 |
| | 0.58 | | | | | | | | | | | | | | |
| Arg | 115 | — | — | B | B | — | — | — | −0.22 | −0.11 | | — | — | F | 0.60 |
| | 1.20 | | | | | | | | | | | | | | |
| Arg | 116 | — | — | B | B | — | — | — | −0.58 | −0.26 | | — | — | F | 0.60 |
| | 1.63 | | | | | | | | | | | | | | |
| Thr | 117 | — | — | B | B | — | — | — | −0.31 | −0.33 | | — | — | F | 0.60 |
| | 1.06 | | | | | | | | | | | | | | |
| Pro | 118 | — | — | B | B | — | — | — | 0.20 | −0.51 | | * | — | F | 1.00 |
| | 0.55 | | | | | | | | | | | | | | |
| Ala | 119 | — | — | B | — | — | — | — | 0.67 | −0.09 | | — | * | — | 1.00 |
| | 0.28 | | | | | | | | | | | | | | |
| Cys | 120 | — | — | B | — | — | T | — | 0.67 | 0.34 | — | * | — | 0.85 | 0.19 |
| Ala | 121 | — | — | — | — | T | T | — | 0.26 | −0.14 | | * | * | — | 2.10 |
| | 0.24 | | | | | | | | | | | | | | |
| Gly | 122 | — | — | — | — | T | T | — | −0.29 | −0.19 | | * | — | F | 2.50 |
| | 0.32 | | | | | | | | | | | | | | |
| Gly | 123 | — | — | — | — | T | T | — | −0.32 | −0.04 | | * | — | F | 2.25 |
| | 0.44 | | | | | | | | | | | | | | |
| Arg | 124 | — | — | B | B | — | — | — | −0.04 | 0.14 | — | — | F | 0.60 | 0.69 |
| Ser | 125 | — | — | B | B | — | — | — | 0.62 | 0.13 | — | — | F | 0.35 | 1.00 |
| Val | 126 | — | — | B | B | — | — | — | 0.62 | −0.30 | | — | — | — | 0.70 |
| | 1.75 | | | | | | | | | | | | | | |
| Tyr | 127 | — | — | B | — | — | — | — | 0.72 | −0.23 | | — | — | — | 0.50 |
| | 0.90 | | | | | | | | | | | | | | |
| Thr | 128 | — | — | B | — | — | — | — | 0.21 | 0.53 | — | — | — | −0.25 | 1.05 |
| Glu | 129 | — | — | B | B | — | — | — | −0.21 | 0.79 | — | * | — | −0.45 | 1.05 |
| Ala | 130 | — | — | B | B | — | — | — | −0.80 | 0.63 | — | * | — | −0.60 | 0.97 |
| Tyr | 131 | — | — | B | B | — | — | — | −0.16 | 0.56 | — | * | — | −0.60 | 0.47 |
| Val | 132 | — | — | B | B | — | — | — | −0.77 | 0.50 | — | * | — | −0.60 | 0.42 |
| Thr | 133 | — | — | B | B | — | — | — | −0.80 | 1.14 | — | * | — | −0.60 | 0.31 |
| Ile | 134 | — | — | B | B | — | — | — | −1.47 | 1.07 | — | * | — | −0.60 | 0.20 |
| Pro | 135 | — | — | B | — | — | T | — | −1.19 | 0.89 | — | * | — | −0.20 | 0.14 |
| Val | 136 | — | — | — | — | T | T | — | −1.61 | 0.73 | — | — | — | 0.20 | 0.14 |
| Gly | 137 | — | — | — | — | T | T | — | −1.61 | 0.81 | — | — | — | 0.20 | 0.11 |
| Cys | 138 | — | — | B | — | — | T | — | −1.51 | 0.77 | — | — | — | −0.20 | 0.05 |
| Thr | 139 | — | — | B | — | — | — | — | −0.62 | 0.77 | — | — | — | −0.40 | 0.11 |
| Cys | 140 | — | — | B | — | — | — | — | −0.62 | 0.13 | — | — | — | −0.10 | 0.19 |
| Val | 141 | — | — | B | — | — | T | — | 0.23 | 0.13 | — | — | — | 0.10 | 0.55 |
| Pro | 142 | — | — | B | — | — | T | — | 0.62 | −0.44 | | — | — | F | 0.85 |
| | 0.65 | | | | | | | | | | | | | | |
| Glu | 143 | — | — | B | — | — | T | — | 1.29 | −0.93 | | — | — | F | 1.30 |
| | 2.44 | | | | | | | | | | | | | | |
| Pro | 144 | A | — | — | — | — | T | — | 1.01 | −1.50 | | * | — | F | 1.30 |
| | 5.49 | | | | | | | | | | | | | | |
| Glu | 145 | A | — | — | — | — | — | — | 1.68 | −1.64 | | * | — | F | 1.10 |
| | 3.59 | | | | | | | | | | | | | | |
| Lys | 146 | A | — | — | — | — | — | — | 2.23 | −2.07 | | * | — | F | 1.10 |
| | 3.46 | | | | | | | | | | | | | | |
| Asp | 147 | A | — | — | — | — | T | — | 1.56 | −1.69 | | — | — | F | 1.30 |
| | 3.00 | | | | | | | | | | | | | | |
| Ala | 148 | A | — | — | — | — | T | — | 1.56 | −1.43 | | * | — | F | 1.30 |
| | 1.21 | | | | | | | | | | | | | | |
| Asp | 149 | A | — | — | — | — | T | — | 1.47 | −1.03 | | * | — | F | 1.15 |
| | 0.98 | | | | | | | | | | | | | | |
| Ser | 150 | A | — | — | — | — | T | — | 1.17 | −0.64 | | * | — | F | 1.15 |
| | 0.78 | | | | | | | | | | | | | | |
| Ile | 151 | A | — | — | — | — | — | — | 0.23 | −0.26 | | * | * | F | 0.80 |
| | 1.04 | | | | | | | | | | | | | | |
| Asn | 152 | — | — | B | — | — | T | — | 0.23 | −0.07 | | * | — | F | 0.85 |
| | 0.44 | | | | | | | | | | | | | | |
| Ser | 153 | — | — | B | — | — | T | — | 0.87 | −0.07 | | * | — | F | 0.85 |
| | 0.54 | | | | | | | | | | | | | | |
| Ser | 154 | — | — | B | — | — | T | — | 0.87 | −0.46 | | * | * | F | 1.00 |
| | 1.55 | | | | | | | | | | | | | | |
| Ile | 155 | A | — | — | — | — | T | — | 0.82 | −0.74 | | — | * | F | 1.30 |
| | 1.67 | | | | | | | | | | | | | | |
| Asp | 156 | A | — | — | — | — | T | — | 1.12 | −0.71 | | * | * | F | 1.30 |
| | 1.23 | | | | | | | | | | | | | | |
| Lys | 157 | A | — | — | — | — | T | — | 1.17 | −0.60 | | * | — | F | 1.15 |
| | 0.93 | | | | | | | | | | | | | | |
| Gln | 158 | A | — | — | — | — | T | — | 0.66 | −0.99 | | * | — | F | 1.30 |
| | 2.65 | | | | | | | | | | | | | | |
| Gly | 159 | — | — | B | — | — | T | — | 0.14 | −0.99 | | * | — | F | 1.30 |
| | 1.31 | | | | | | | | | | | | | | |
| Ala | 160 | — | A | B | — | — | — | — | 0.22 | −0.30 | | * | — | F | 0.45 |
| | 0.54 | | | | | | | | | | | | | | |
| Lys | 161 | — | A | B | — | — | — | — | −0.12 | 0.39 | — | — | F | −0.15 | 0.26 |
| Leu | 162 | — | A | B | — | — | — | — | −0.38 | 0.41 | — | — | — | −0.60 | 0.26 |
| Leu | 163 | — | A | B | — | — | — | — | −0.38 | 0.41 | — | — | — | −0.60 | 0.39 |
| Leu | 164 | — | A | B | — | — | — | — | −0.03 | 0.31 | — | — | — | −0.06 | 0.32 |
| Gly | 165 | — | — | B | — | — | T | — | −0.03 | 0.31 | — | — | F | 0.73 | 0.64 |
| Pro | 166 | — | — | — | — | — | T | C | −0.29 | 0.13 | — | — | F | 1.17 | 0.78 |
| Asn | 167 | — | — | — | — | T | T | — | −0.07 | −0.13 | | — | — | F | 2.36 |
| | 1.47 | | | | | | | | | | | | | | |
| Asp | 169 | — | — | — | — | — | T | C | 0.40 | −0.31 | | — | — | F | 2.40 |
| | 1.50 | | | | | | | | | | | | | | |
| Ala | 169 | — | — | — | — | — | — | C | 1.00 | −0.31 | | — | — | F | 1.81 |
| | 0.96 | | | | | | | | | | | | | | |
| Pro | 170 | — | — | — | — | — | T | C | 0.96 | −0.31 | | — | — | F | 1.77 |
| | 0.92 | | | | | | | | | | | | | | |
| Ala | 171 | — | — | — | — | — | T | C | 0.78 | −0.29 | | — | — | — | 1.38 |
| | 0.71 | | | | | | | | | | | | | | |
| Gly | 172 | — | — | — | — | — | T | C | 0.39 | 0.14 | — | — | — | 0.54 | 0.90 |
| Pro | 173 | — | — | P | — | — | T | — | 0.00 | 0.07 | — | — | — | 0.10 | 0.74 |

Among highly preferred fragments in this regard are those that comprise reigons of IL-21 or IL-22 that combine several structural features, such as several of the features set out above.

Other preferred fragments are biologically active IL-21 and IL-22 fragments. Biologically active fragments are those exhibiting activity similar, but not necessarily identical, to an activity of the IL-21 and IL-22 polypeptides. The biological activity of the fragments may include an improved desired activity, or a decreased undesirable activity.

Transgenics and “knock-outs”

The polypeptides of the invention can also be expressed in transgenic animals. Animals of any species, including, but not limited to, mice, rats, rabbits, hamsters, guinea pigs, pigs, micro-pigs, goats, sheep, cows and non-human primates, e.g., baboons, monkeys, and chimpanzees may be used to generate transgenic animals. In a specific embodiment, techniques described herein or otherwise known in the art, are used to express polypeptides of the invention in humans, as part of a gene therapy protocol.

Any technique known in the art may be used to introduce the transgene (i.e., polynucleotides of the invention) into animals to produce the founder lines of transgenic animals. Such techniques include, but are not limited to, pronuclear microinjection (Paterson et al., Appl. Microbiol. Biotechnol. 40:691-698 (1994); Carver et al., Biotechnology (NY) 11:1263-1270 (1993); Wright et al., Biotechnology (NY) 9:830-834 (1991); and Hoppe et al., U.S. Pat. No. 4,873,191 (1989)); retrovirus mediated gene transfer into germ lines (Van der Putten et al., Proc. Natl. Acad. Sci., USA 82:6148-6152 (1985)), blastocysts or embryos; gene targeting in embryonic stem cells (Thompson et al., Cell 56:313-321 (1989)); electroporation of cells or embryos (Lo, 1983, Mol Cell. Biol. 3:1803-1814 (1983)); introduction of the polynucleotides of the invention using a gene gun (see, e.g., Ulmer et al., Science 259:1745 (1993); introducing nucleic acid constructs into embryonic pleuripotent stem cells and transferring the stem cells back into the blastocyst; and sperm-mediated gene transfer (Lavitrano et al., Cell 57:717-723 (1989); etc. For a review of such techniques, see Gordon, “Transgenic Animals,” Intl. Rev. Cytol. 115:171-229 (1989), which is incorporated by reference herein in its entirety.

Any technique known in the art may be used to produce transgenic clones containing polynucleotides of the invention, for example, nuclear transfer into enucleated oocytes of nuclei from cultured embryonic, fetal, or adult cells induced to quiescence (Campell et al., Nature 380:64-66 (1996); Wilmut et al., Nature 385:810-813 (1997)).

The present invention provides for transgenic animals that carry the transgene in all their cells, as well as animals which carry the transgene in some, but not all their cells, i.e., mosaic animals or chimeric. The transgene may be integrated as a single transgene or as multiple copies such as in concatamers, e.g., head-to-head tandems or head-to-tail tandems. The transgene may also be selectively introduced into and activated in a particular cell type by following, for example, the teaching of Lasko et al. (Lasko et al., Proc. Natl. Acad. Sci. USA 89:6232-6236 (1992)). The regulatory sequences required for such a cell-type specific activation will depend upon the particular cell type of interest, and will be apparent to those of skill in the art. When it is desired that the polynucleotide transgene be integrated into the chromosomal site of the endogenous gene, gene targeting is preferred. Briefly, when such a technique is to be utilized, vectors containing some nucleotide sequences homologous to the endogenous gene are designed for the purpose of integrating, via homologous recombination with chromosomal sequences, into and disrupting the function of the nucleotide sequence of the endogenous gene. The transgene may also be selectively introduced into a particular cell type, thus inactivating the endogenous gene in only that cell type, by following, for example, the teaching of Gu et al. (Gu et al., Science 265:103-106 (1994)). The regulatory sequences required for such a cell-type specific inactivation will depend upon the particular cell type of interest, and will be apparent to those of skill in the art.

In specific preferred embodiments, IL-21 or IL-22 polynucleotides of the invention may be expressed under the direction of a murine transferrin receptor promoter construct thereby restricting expression to the liver of transgenic animals. In other specific preferred embodiments, IL-21 or IL-22 polynucleotides of the invention are expressed under the direction of a murine beta-actin promoter construct thereby effecting ubiquitous expression of the IL-21 or IL-22 polynucleotide.

Once transgenic animals have been generated, the expression of the recombinant gene may be assayed utilizing standard techniques. Initial screening may be accomplished by Southern blot analysis or PCR techniques to analyze animal tissues to verify that integration of the transgene has taken place. The level of mRNA expression of the transgene in the tissues of the transgenic animals may also be assessed using techniques which include, but are not limited to, Northern blot analysis of tissue samples obtained from the animal, in situ hybridization analysis, and reverse transcriptase-PCR (rt-PCR) and “TaqMAN” real time PCR. Samples of transgenic gene-expressing tissue may also be evaluated immunocytochemically or immunohistochemically using antibodies specific for the transgene product.

Once the founder animals are produced, they may be bred, inbred, outbred, or crossbred to produce colonies of the particular animal. Examples of such breeding strategies include, but are not limited to: outbreeding of founder animals with more than one integration site in order to establish separate lines; inbreeding of separate lines in order to produce compound transgenics that express the transgene at higher levels because of the effects of additive expression of each transgene; crossing of heterozygous transgenic animals to produce animals homozygous for a given integration site in order to both augment expression and eliminate the need for screening of animals by DNA analysis; crossing of separate homozygous lines to produce compound heterozygous or homozygous lines; and breeding to place the transgene on a distinct background that is appropriate for an experimental model of interest.

Transgenic and “knock-out” animals of the invention have uses which include, but are not limited to, animal model systems useful in elaborating the biological function of IL-21 and/or IL-22 polypeptides, studying conditions and/or disorders associated with aberrant IL-2 1 and/or IL-22 expression, and in screening for compounds effective in ameliorating such conditions and/or disorders.

In further embodiments of the invention, cells that are genetically engineered to express the polypeptides of the invention, or alternatively, that are genetically engineered not to express the polypeptides of the invention (e.g., knockouts) are administered to a patient in vivo. Such cells may be obtained from the patient (i.e., animal, including human) or an MHC compatible donor and can include, but are not limited to fibroblasts, bone marrow cells, blood cells (e.g., lymphocytes), adipocytes, muscle cells, endothelial cells etc. The cells are genetically engineered in vitro using recombinant DNA techniques to introduce the coding sequence of polypeptides of the invention into the cells, or alternatively, to disrupt the coding sequence and/or endogenous regulatory sequence associated with the polypeptides of the invention, e.g., by transduction (using viral vectors, and preferably vectors that integrate the transgene into the cell genome) or transfection procedures, including, but not limited to, the use of plasmids, cosmids, YACs, naked DNA, electroporation, liposomes, etc. The coding sequence of the polypeptides of the invention can be placed under the control of a strong constitutive or inducible promoter or promoter/enhancer to achieve expression, and preferably secretion, of the polypeptides of the invention. The engineered cells which express and preferably secrete the polypeptides of the invention can be introduced into the patient systemically, e.g., in the circulation, or intraperitoneally.

Alternatively, the cells can be incorporated into a matrix and implanted in the body, e.g., genetically engineered fibroblasts can be implanted as part of a skin graft; genetically engineered endothelial cells can be implanted as part of a lymphatic or vascular graft. (See, for example, Anderson, et al. U.S. Pat. No. 5,399,349; and Mulligan & Wilson, U.S. Pat. No. 5,460,959 each of which is incorporated by reference herein in its entirety).

When the cells to be administered are non-autologous or non-MHC compatible cells, they can be administered using well known techniques which prevent the development of a host immune response against the introduced cells. For example, the cells may be introduced in an encapsulated form which, while allowing for an exchange of components with the immediate extracellular environment, does not allow the introduced cells to be recognized by the host immune system.

Epitopes & Antibodies

The present invention encompasses polypeptides comprising, or alternatively consisting of, an epitope of the polypeptide having an amino acid sequence of SEQ ID NO: 2, SEQ ID NO: 4, SEQ ID NO: 29 or SEQ ID NO: 32, or an epitope of the polypeptide sequence encoded by a polynucleotide sequence contained in ATCC Deposit Nos: 209666, 209665, PTA-69 or PTA-70 or encoded by a polynucleotide that hybridizes to the complement of the sequence of SEQ ID NO: 1, SEQ ID NO: 3, SEQ ID NO: 28, or SEQ ID NO: 31 or contained in ATCC Deposit Nos: 209666, 209665, PTA-69 or PTA-70 under stringent hybridization conditions or lower stringency hybridization conditions as defined supra. The present invention further encompasses polynucleotide sequences encoding an epitope of a polypeptide sequence of the invention (such as, for example, the sequence disclosed in SEQ ID NO: 1, SEQ ID NO: 3, SEQ ID NO: 28, or SEQ ID NO: 31), polynucleotide sequences of the complementary strand of a polynucleotide sequence encoding an epitope of the invention, and polynucleotide sequences which hybridize to the complementary strand under stringent hybridization conditions or lower stringency hybridization conditions defined supra.

The term “epitopes,” as used herein, refers to portions of a polypeptide having antigenic or immunogenic activity in an animal, preferably a mammal, and most preferably in a human. In a preferred embodiment, the present invention encompasses a polypeptide comprising an epitope, as well as the polynucleotide encoding this polypeptide. An “immunogenic epitope,” as used herein, is defined as a portion of a protein that elicits an antibody response in an animal, as determined by any method known in the art, for example, by the methods for generating antibodies described infra. (See, for example, Geysen et al., Proc. Natl. Acad. Sci. USA 81:3998-4002 (1983)). The term “antigenic epitope,” as used herein, is defined as a portion of a protein to which an antibody can immunospecifically bind its antigen as determined by any method well known in the art, for example, by the immunoassays described herein. Immunospecific binding excludes non-specific binding but does not necessarily exclude cross-reactivity with other antigens. Antigenic epitopes need not necessarily be immunogenic.

Fragments which function as epitopes may be produced by any conventional means. (See, e.g., Houghten, Proc. Natl. Acad. Sci. USA 82:5131-5135 (1985), further described in U.S. Pat. No. 4,631,211).

In the present invention, antigenic epitopes preferably contain a sequence of at least 4, at least 5, at least 6, at least 7, more preferably at least 8, at least 9, at least 10, at least 11, at least 12, at least 13, at least 14, at least 15, at least 20, at least 25, at least 30, at least 40, at least 50, and, most preferably, between about 15 to about 30 amino acids. Preferred polypeptides comprising immunogenic or antigenic epitopes are at least 10, 15, 20, 25, 30, 35, 40, 45, 50, 55, 60, 65, 70, 75, 80, 85, 90, 95, or 100 amino acid residues in length. Additional non-exclusive preferred antigenic epitopes include the antigenic epitopes disclosed herein, as well as portions thereof. Antigenic epitopes are useful, for example, to raise antibodies, including monoclonal antibodies, that specifically bind the epitope. Preferred antigenic epitopes include the antigenic epitopes disclosed herein, as well as any combination of two, three, four, five or more of these antigenic epitopes. Antigenic epitopes can be used as the target molecules in immunoassays. (See, for instance, Wilson et al., Cell 37:767-778 (1984); Sutcliffe et al., Science 219:660-666 (1983)).

Similarly, immunogenic epitopes can be used, for example, to induce antibodies according to methods well known in the art. (See, for instance, Sutcliffe et al., supra; Wilson et al., supra; Chow et al., Proc. Natl. Acad. Sci. USA 82:910-914; and Bittle et al., J. Gen. Virol. 66:2347-2354 (1985). Preferred immunogenic epitopes include the immunogenic epitopes disclosed herein, as well as any combination of two, three, four, five or more of these immunogenic epitopes. The polypeptides comprising one or more immunogenic epitopes may be presented for eliciting an antibody response together with a carrier protein, such as an albumin, to an animal system (such as rabbit or mouse), or, if the polypeptide is of sufficient length (at least about 25 amino acids), the polypeptide may be presented without a carrier. However, immunogenic epitopes comprising as few as 8 to 10 amino acids have been shown to be sufficient to raise antibodies capable of binding to, at the very least, linear epitopes in a denatured polypeptide (e.g., in Western blotting).

Epitope-bearing polypeptides of the present invention may be used to induce antibodies according to methods well known in the art including, but not limited to, in vivo immunization, in vitro immunization, and phage display methods. See, e.g., Sutcliffe et al., supra; Wilson et al., supra, and Bittle et al., J. Gen. Virol., 66:2347-2354 (1985). If in vivo immunization is used, animals may be immunized with free peptide; however, anti-peptide antibody titer may be boosted by coupling the peptide to a macromolecular carrier, such as keyhole limpet hemacyanin (KLH) or tetanus toxoid. For instance, peptides containing cysteine residues may be coupled to a carrier using a linker such as maleimidobenzoyl- N-hydroxysuccinimide ester (MBS), while other peptides may be coupled to carriers using a more general linking agent such as glutaraldehyde. Animals such as rabbits, rats and mice are immunized with either free or carrier-coupled peptides, for instance, by intraperitoneal and/or intradermal injection of emulsions containing about 100 μg of peptide or carrier protein and Freund's adjuvant or any other adjuvant known for stimulating an immune response. Several booster injections may be needed, for instance, at intervals of about two weeks, to provide a useful titer of anti-peptide antibody which can be detected, for example, by ELISA assay using free peptide adsorbed to a solid surface. The titer of anti-peptide antibodies in serum from an immunized animal may be increased by selection of anti-peptide antibodies, for instance, by adsorption to the peptide on a solid support and elution of the selected antibodies according to methods well known in the art.

As one of skill in the art will appreciate, and as discussed above, the polypeptides of the present invention comprising an immunogenic or antigenic epitope can be fused to other polypeptide sequences. For example, the polypeptides of the present invention may be fused with the constant domain of immunoglobulins (IgA, IgE, IgG, IgM), or portions thereof (CH1, CH2, CH3, or any combination thereof and portions thereof) resulting in chimeric polypeptides. Such fusion proteins may facilitate purification and may increase half-life in vivo. This has been shown for chimeric proteins consisting of the first two domains of the human CD4-polypeptide and various domains of the constant regions of the heavy or light chains of mammalian immunoglobulins. See, e.g., EP 394,827; Traunecker et al., Nature, 331:84-86 (1988). Enhanced delivery of an antigen across the epithelial barrier to the immune system has been demonstrated for antigens (e.g., insulin) conjugated to an FcRn binding partner such as IgG or Fc fragments (see, e.g., PCT Publications WO 96/22024 and WO 99/04813). IgG Fusion proteins that have a disulfide-linked dimeric structure due to the IgG portion desulfide bonds have also been found to be more efficient in binding and neutralizing other molecules than monomeric polypeptides or fragments thereof alone. See, e.g., Fountoulakis et al., J. Biochem., 270:3958-3964 (1995). Nucleic acids encoding the above epitopes can also be recombined with a gene of interest as an epitope tag (e.g., the hemagglutinin (“HA”) tag or flag tag) to aid in detection and purification of the expressed polypeptide. For example, a system described by Janknecht et al. allows for the ready purification of non-denatured fusion proteins expressed in human cell lines (Janknecht et al., 1991, Proc. Natl. Acad. Sci. USA 88:8972-897). In this system, the gene of interest is subcloned into a vaccinia recombination plasmid such that the open reading frame of the gene is translationally fused to an amino-terminal tag consisting of six histidine residues. The tag serves as a matrix binding domain for the fusion protein. Extracts from cells infected with the recombinant vaccinia virus are loaded onto Ni2+ nitriloacetic acid-agarose column and histidine-tagged proteins can be selectively eluted with imidazole-containing buffers.

Additional fusion proteins of the invention may be generated through the techniques of gene-shuffling, motif-shuffling, exon-shuffling, and/or codon-shuffling (collectively referred to as “DNA shuffling”). DNA shuffling may be employed to modulate the activities of polypeptides of the invention, such methods can be used to generate polypeptides with altered activity, as well as agonists and antagonists of the polypeptides. See, generally, U.S. Pat. Nos. 5,605,793; 5,811,238; 5,830,721; 5,834,252; and 5,837,458, and Patten et al., Curr. Opinion Biotechnol. 8:724-33 (1997); Harayama, Trends Biotechnol. 16(2):76-82 (1998); Hansson, et al., J. Mol. Biol. 287:265-76 (1999); and Lorenzo and Blasco, Biotechniques 24(2):308-13 (1998) (each of these patents and publications are hereby incorporated by reference in its entirety). In one embodiment, alteration of polynucleotides corresponding to SEQ ID NO: 1 or SEQ ID NO: 28 and the polypeptides encoded by these polynucleotides may be achieved by DNA shuffling. In a further embodiment, alteration of polynucleotides corresponding to SEQ ID NO: 3 or SEQ ID NO: 3 1 and the polypeptides encoded by these polynucleotides may be achieved by DNA shuffling. DNA shuffling involves the assembly of two or more DNA segments by homologous or site-specific recombination to generate variation in the polynucleotide sequence. In another embodiment, polynucleotides of the invention, or the encoded polypeptides, may be altered by being subjected to random mutagenesis by error-prone PCR, random nucleotide insertion or other methods prior to recombination. In another embodiment, one or more components, motifs, sections, parts, domains, fragments, etc., of a polynucleotide encoding a polypeptide of the invention may be recombined with one or more components, motifs, sections, parts, domains, fragments, etc. of one or more heterologous molecules.

Antibodies

Further polypeptides of the invention relate to antibodies and T-cell antigen receptors (TCR) which immuno specifically bind a polypeptide, polypeptide fragment, or variant of SEQ ID NO: 2, SEQ ID NO: 4, SEQ ID NO: 29 or SEQ ID NO: 32, and/or an epitope, of the present invention (as determined by immunoassays well known in the art for assaying specific antibody-antigen binding). Antibodies of the invention include, but are not limited to, polyclonal, monoclonal, multispecific, human, humanized or chimeric antibodies, single chain antibodies, Fab fragments, F(ab′) fragments, fragments produced by a Fab expression library, anti-idiotypic (anti-Id) antibodies (including, e.g., anti-Id antibodies to antibodies of the invention), and epitope-binding fragments of any of the above. The term “antibody,” as used herein, refers to immunoglobulin molecules and immunologically active portions of immunoglobulin molecules, i.e., molecules that contain an antigen binding site that immunospecifically binds an antigen. The immunoglobulin molecules of the invention can be of any type (e.g., IgG, IgE, IgM, IgD, IgA and IgY), class (e.g., IgG1, IgG2, IgG3, IgG4, IgA1 and IgA2) or subclass of immunoglobulin molecule.

Most preferably the antibodies are human antigen-binding antibody fragments of the present invention and include, but are not limited to, Fab, Fab′ and F(ab′)2, Fd, single-chain Fvs (scFv), single-chain antibodies, disulfide-linked Fvs (sdFv) and fragments comprising either a VL or VH domain. Antigen-binding antibody fragments, including single-chain antibodies, may comprise the variable region(s) alone or in combination with the entirety or a portion of the following: hinge region, CH1, CH2, and CH3 domains. Also included in the invention are antigen-binding fragments also comprising any combination of variable region(s) with a hinge region, CH1, CH2, and CH3 domains. The antibodies of the invention may be from any animal origin including birds and mammals. Preferably, the antibodies are human, murine (e.g., mouse and rat), donkey, ship rabbit, goat, guinea pig, camel, horse, or chicken. As used herein, “human” antibodies include antibodies having the amino acid sequence of a human immunoglobulin and include antibodies isolated from human immunoglobulin libraries or from animals transgenic for one or more human immunoglobulin and that do not express endogenous immunoglobulins, as described infra and, for example in, U.S. Pat. No. 5,939,598 by Kucherlapati et al.

The antibodies of the present invention may be monospecific, bispecific, trispecific or of greater multispecificity. Multispecific antibodies may be specific for different epitopes of a polypeptide of the present invention or may be specific for both a polypeptide of the present invention as well as for a heterologous epitope, such as a heterologous polypeptide or solid support material. See, e.g., PCT publications WO 93/17715; WO 92/08802; WO 91/00360; WO 92/05793; Tutt, et al., J. Immunol. 147:60-69 (1991); U.S. Pat. Nos. 4,474,893; 4,714,681; 4,925,648; 5,573,920; 5,601,819; Kostelny et al., J. Immunol. 148:1547-1553 (1992).

Antibodies of the present invention may be described or specified in terms of the epitope(s) or portion(s) of a polypeptide of the present invention which they recognize or specifically bind. The epitope(s) or polypeptide portion(s) may be specified as described herein, e.g., by N-terminal and C-terminal positions, by size in contiguous amino acid residues, or listed in the Tables and Figures. Preferred epitopes of the invention include: from about Arg-2 to about Pro-11, from about Cys-24 to about Glu-32, and from about Arg-51 to about Gly-59 of SEQ ID NO: 2, as well as polynucleotides that encode these epitopes. Additional preferred epitopes of the invention include: from about Gly-19 to about Ala-27, from about Pro-30 to about Arg-38, from about Phe-40 to about Ser-48, from about Tyr-58 to about Leu-67, from about Pro-105 to about Val-113, from about Pro-129 to about Ser-137, from about Asn-139 to about Ala-147, and from about Leu-151 to about Gly-159 of SEQ ID NO: 4, as well as polynucleotides that encode these epitopes. Antibodies which specifically bind any epitope or polypeptide of the present invention may also be excluded. Therefore, the present invention includes antibodies that specifically bind polypeptides of the present invention, and allows for the exclusion of the same.

Antibodies of the present invention may also be described or specified in terms of their cross-reactivity. Antibodies that do not bind any other analog, ortholog, or homolog of a polypeptide of the present invention are included. Antibodies that bind polypeptides with at least 95%, at least 90%, at least 85%, at least 80%, at least 75%, at least 70%, at least 65%, at least 60%, at least 55%, and at least 50% identity (as calculated using methods known in the art and described herein) to a polypeptide of the present invention are also included in the present invention. In specific embodiments, antibodies of the present invention cross-react with murine, rat and/or rabbit homologs of human proteins and the corresponding epitopes thereof. Antibodies that do not bind polypeptides with less than 95%, less than 90%, less than 85%, less than 80%, less than 75%, less than 70%, less than 65%, less than 60%, less than 55%, and less than 50% identity (as calculated using methods known in the art and described herein) to a polypeptide of the present invention are also included in the present invention. In a specific embodiment, the above-described cross-reactivity is with respect to any single specific antigenic or immunogenic polypeptide, or combination(s) of 2, 3, 4, 5, or more of the specific antigenic and/or immunogenic polypeptides disclosed herein. Further included in the present invention are antibodies which bind polypeptides encoded by polynucleotides which hybridize to a polynucleotide of the present invention under stringent hybridization conditions (as described herein). Antibodies of the present invention may also be described or specified in terms of their binding affinity to a polypeptide of the invention. Preferred binding affinities include those with a dissociation constant or Kd less than 5×10⁻²M, 10⁻²M, 5×10⁻³M, 10⁻³M, 5×10⁻⁴M, 10⁻⁴M, 5×10⁻⁵M, 10⁻⁵M, 5×10⁻⁶M, 10⁻⁶M, 5×10⁻⁷M, 10⁷M, 5×10⁻⁸M, 10⁻⁸M, 5×10⁻⁹M, 10⁻⁹M, 5×10⁻¹⁰M, 10⁻¹⁰M, 5×10⁻¹¹M, 10⁻¹¹M, 5×10⁻¹²M, 10⁻¹²M, 5×10⁻¹³M, 10⁻¹³M, 5×10⁻¹⁴M, 10⁻¹⁴M, 5×10⁻¹⁵M, or 10⁻¹⁵M.

The invention also provides antibodies that competitively inhibit binding of an antibody to an epitope of the invention as determined by any method known in the art for determining competitive binding, for example, the immunoassays described herein. In preferred embodiments, the antibody competitively inhibits binding to the epitope by at least 95%, at least 90%, at least 85 %, at least 80%, at least 75%, at least 70%, at least 60%, or at least 50%.

Antibodies of the present invention may act as agonists or antagonists of the polypeptides of the present invention. For example, the present invention includes antibodies which disrupt the receptor/ligand interactions with the polypeptides of the invention either partially or fully. Preferrably, antibodies of the present invention bind an antigenic epitope disclosed herein, or a portion thereof. The invention features both receptor-specific antibodies and ligand-specific antibodies. The invention also features receptor-specific antibodies which do not prevent ligand binding but prevent receptor activation. Receptor activation (i.e., signaling) may be determined by techniques described herein or otherwise known in the art. For example, receptor activation can be determined by detecting the phosphorylation (e.g., tyrosine or serine/threonine) of the receptor or its substrate by immunoprecipitation followed by western blot analysis (for example, as described supra). In specific embodiments, antibodies are provided that inhibit ligand activity or receptor activity by at least 95%, at least 90%, at least 85%, at least 80%, at least 75%, at least 70%, at least 60%, or at least 50% of the activity in absence of the antibody.

The invention also features receptor-specific antibodies which both prevent ligand binding and receptor activation as well as antibodies that recognize the receptor-ligand complex, and, preferably, do not specifically recognize the unbound receptor or the unbound ligand. Likewise, included in the invention are neutralizing antibodies which bind the ligand and prevent binding of the ligand to the receptor, as well as antibodies which bind the ligand, thereby preventing receptor activation, but do not prevent the ligand from binding the receptor. Further included in the invention are antibodies which activate the receptor. These antibodies may act as receptor agonists, i.e., potentiate or activate either all or a subset of the biological activities of the ligand-mediated receptor activation, for example, by inducing dimerization of the receptor. The antibodies may be specified as agonists, antagonists or inverse agonists for biological activities comprising the specific biological activities of the peptides of the invention disclosed herein. The above antibody agonists can be made using methods known in the art. See, e.g., PCT publication WO 96/40281; U.S. Pat. No. 5,811,097; Deng et al., Blood 92(6):1981-1988 (1998); Chen et al., Cancer Res. 58(16):3668-3678 (1998); Harrop et al., J. Immunol. 161(4):1786-1794 (1998); Zhu et al., Cancer Res. 58(15):3209-3214 (1998); Yoon et al., J. Immunol. 160(7):3170-3179 (1998); Prat et al., J. Cell. Sci. 111(Pt2):237-247 (1998); Pitard et al., J. Immunol. Methods 205(2):177-190 (1997); Liautard et al., Cytokine 9(4):233-241 (1997); Carlson et al., J. Biol. Chem. 272(17):11295-11301 (1997); Taryman et al., Neuron 14(4):755-762 (1995); Muller et al., Structure 6(9):1153-1167 (1998); Bartunek et al., Cytokine 8(1):14-20 (1996) (which are all incorporated by reference herein in their entireties).

Antibodies of the present invention may be used, for example, but not limited to, to purify, detect, and target the polypeptides of the present invention, including both in vitro and in vivo diagnostic and therapeutic methods. For example, the antibodies have use in immunoassays for qualitatively and quantitatively measuring levels of the polypeptides of the present invention in biological samples. See, e.g., Harlow et al., Antibodies: A Laboratory Manual, (Cold Spring Harbor Laboratory Press, 2nd ed. 1988) (incorporated by reference herein in its entirety).

As discussed in more detail below, the antibodies of the present invention may be used either alone or in combination with other compositions. The antibodies may further be recombinantly fused to a heterologous polypeptide at the N- or C-terminus or chemically conjugated (including covalently and non-covalently conjugations) to polypeptides or other compositions. For example, antibodies of the present invention may be recombinantly fused or conjugated to molecules useful as labels in detection assays and effector molecules such as heterologous polypeptides, drugs, radionuclides, or toxins. See, e.g., PCT publications WO 92/08495; WO 91/14438; WO 89/12624; U.S. Pat. No. 5,314,995; and EP 396,387.

The antibodies of the invention include derivatives that are modified, i.e, by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

The antibodies of the present invention may be generated by any suitable method known in the art. Polyclonal antibodies to an antigen-of-interest can be produced by various procedures well known in the art. For example, a polypeptide of the invention can be administered to various host animals including, but not limited to, rabbits, mice, rats, etc. to induce the production of sera containing polyclonal antibodies specific for the antigen. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art and are discussed in detail in the Examples. In a non-limiting example, mice can be immunized with a polypeptide of the invention or a cell expressing such peptide. Once an immune response is detected, e.g., antibodies specific for the antigen are detected in the mouse serum, the mouse spleen is harvested and splenocytes isolated. The splenocytes are then fused by well known techniques to any suitable myeloma cells, for example cells from cell line SP20 available from the ATCC. Hybridomas are selected and cloned by limited dilution. The hybridoma clones are then assayed by methods known in the art for cells that secrete antibodies capable of binding a polypeptide of the invention. Ascites fluid, which generally contains high levels of antibodies, can be generated by immunizing mice with positive hybridoma clones.

Accordingly, the present invention provides methods of generating monoclonal antibodies as well as antibodies produced by the method comprising culturing a hybridoma cell secreting an antibody of the invention wherein, preferably, the hybridoma is generated by fusing splenocytes isolated from a mouse immunized with an antigen of the invention with myeloma cells and then screening the hybridomas resulting from the fusion for hybridoma clones that secrete an antibody able to bind a polypeptide of the invention.

Antibody fragments which recognize specific epitopes may be generated by known techniques. For example, Fab and F(ab′)2 fragments of the invention may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1 domain of the heavy chain.

For example, the antibodies of the present invention can also be generated using various phage display methods known in the art. In phage display methods, functional antibody domains are displayed on the surface of phage particles which carry the polynucleotide sequences encoding them. In a particular embodiment, such phage can be utilized to display antigen binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein. Examples of phage display methods that can be used to make the antibodies of the present invention include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187:9-18 (1997); Burton et al., Advances in Immunology 57:191-280 (1994); PCT application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described in detail below. For example, techniques to recombinantly produce Fab, Fab′ and F(ab′)2 fragments can also be employed using methods known in the art such as those disclosed in PCT publication WO 92/22324; Mullinax et al., BioTechniques 12(6):864-869 (1992); and Sawai et al., AJRI 34:26-34 (1995); and Better et al., Science 240:1041-1043 (1988) (said references incorporated by reference in their entireties).

Examples of techniques which can be used to produce single-chain Fvs and antibodies include those described in U.S. Pat. Nos. 4,946,778 and 5,258,498; Huston et al., Methods in Enzymology 203:46-88 (1991); Shu et al., PNAS 90:7995-7999 (1993); and Skerra et al., Science 240:1038-1040 (1988). For some uses, including in vivo use of antibodies in humans and in vitro detection assays, it may be preferable to use chimeric, humanized, or human antibodies. A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. See e.g., Morrison, Science 229:1202 (1985); Oi et al., BioTechniques 4:214 (1986); Gillies et al., (1989) J. Immunol. Methods 125:191-202; U.S. Pat. Nos. 5,807,715; 4,816,567; and 4,816397, which are incorporated herein by reference in their entirety. Humanized antibodies are antibody molecules from non-human species antibody that binds the desired antigen having one or more complementarity determining regions (CDRs) from the non-human species and a framework regions from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., Nature 332:323 (1988), which are incorporated herein by reference in their entireties.) Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology 28(⅘):489-498 (1991); Studnicka et al., Protein Engineering 7(6):805-814 (1994); Roguska. et al., PNAS 91:969-973 (1994)), and chain shuffling (U.S. Pat. No. 5,565,332).

Completely human antibodies are particularly desirable for therapeutic treatment of human patients. Human antibodies can be made by a variety of methods known in the art including phage display methods described above using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated herein by reference in its entirety.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar, Int. Rev. Immunol. 13:65-93 (1995). For a detailed discussion of this technology for producing human antibodies and human monoclonal antibodies and protocols for producing such antibodies, see, e.g., PCT publications WO 98/24893; WO 92/01047; WO 96/34096; WO 96/33735; European Patent No. 0 598 877; U.S. Pat. Nos. 5,413,923; 5,625,126; 5,633,425; 5,569,825; 5,661,016; 5,545,806; 5,814,318; 5,885,793; 5,916,771; and 5,939,598, which are incorporated by reference herein in their entirety. In addition, companies such as Abgenix, Inc. (Freemont, Calif.) and Genpharm (San Jose, Calif.) can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al., Bio/technology 12:899-903 (1988)).

Further, antibodies to the polypeptides of the invention can, in turn, be utilized to generate anti-idiotype antibodies that “mimic” polypeptides of the invention using techniques well known to those skilled in the art. (See, e.g., Greenspan & Bona, FASEB J. 7(5):437-444; (1989) and Nissinoff, J. Immunol. 147(8):2429-2438 (1991)). For example, antibodies which bind to and competitively inhibit polypeptide multimerization and/or binding of a polypeptide of the invention to a ligand can be used to generate anti-idiotypes that “mimic” the polypeptide multimerization and/or binding domain and, as a consequence, bind to and neutralize polypeptide and/or its ligand. Such neutralizing anti-idiotypes or Fab fragments of such anti-idiotypes can be used in therapeutic regimens to neutralize polypeptide ligand. For example, such anti-idiotypic antibodies can be used to bind a polypeptide of the invention and/or to bind its ligands/receptors, and thereby block its biological activity.

Polynucleotides Encoding Antibodies

The invention further provides polynucleotides comprising a nucleotide sequence encoding an antibody of the invention and fragments thereof. The invention also encompasses polynucleotides that hybridize under stringent or lower stringency hybridization conditions, e.g., as defined supra, to polynucleotides that encode an antibody, preferably, that specifically binds to a polypeptide of the invention, preferably, an antibody that binds to a polypeptide having the amino acid sequence of SEQ ID NO: 2, SEQ ID NO: 4, SEQ ID NO: 29, or SEQ ID NO: 32.

The polynucleotides may be obtained, and the nucleotide sequence of the polynucleotides determined, by any method known in the art. For example, if the nucleotide sequence of the antibody is known, a polynucleotide encoding the antibody may be assembled from chemically synthesized oligonucleotides (e.g., as described in Kutmeier et al., BioTechniques 17:242 (1994)), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an antibody may be generated from nucleic acid from a suitable source. If a clone containing a nucleic acid encoding a particular antibody is not available, but the sequence of the antibody molecule is known, a nucleic acid encoding the immunoglobulin may be chemically synthesized or obtained from a suitable source (e.g., an antibody cDNA library, or a cDNA library generated from, or nucleic acid, preferably poly A+RNA, isolated from, any tissue or cells expressing the antibody, such as hybridoma cells selected to express an antibody of the invention) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR may then be cloned into replicable cloning vectors using any method well known in the art.

Once the nucleotide sequence and corresponding amino acid sequence of the antibody is determined, the nucleotide sequence of the antibody may be manipulated using methods well known in the art for the manipulation of nucleotide sequences, e.g., recombinant DNA techniques, site directed mutagenesis, PCR, etc. (see, for example, the techniques described in Sambrook et al., 1990, Molecular Cloning, A Laboratory Manual, 2d Ed., Cold Spring Harbor Laboratory, Cold Spring Harbor, N.Y. and Ausubel et al., eds., 1998, Current Protocols in Molecular Biology, John Wiley & Sons, NY, which are both incorporated by reference herein in their entireties), to generate antibodies having a different amino acid sequence, for example to create amino acid substitutions, deletions, and/or insertions.

In a specific embodiment, the amino acid sequence of the heavy and/or light chain variable domains may be inspected to identify the sequences of the complementarity determining regions (CDRs) by methods that are well know in the art, e.g., by comparison to known amino acid sequences of other heavy and light chain variable regions to determine the regions of sequence hypervariability. Using routine recombinant DNA techniques, one or more of the CDRs may be inserted within framework regions, e.g., into human framework regions to humanize a non-human antibody, as described supra. The framework regions maybe naturally occurring or consensus framework regions, and preferably human framework regions (see, e.g., Chothia et al., J. Mol. Biol. 278:457-479 (1998) for a listing of human framework regions). Preferably, the polynucleotide generated by the combination of the framework regions and CDRs encodes an antibody that specifically binds a polypeptide of the invention. Preferably, as discussed supra, one or more amino acid substitutions may be made within the framework regions, and, preferably, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the present invention and within the skill of the art.

In addition, techniques developed for the production of “chimeric antibodies” (Morrison et al., Proc. Natl. Acad. Sci. 81:851-855 (1984); Neuberger et al., Nature 312:604-608 (1984); Takeda et al., Nature 314:452-454 (1985)) by splicing genes from a mouse antibody molecule of appropriate antigen specificity together with genes from a human antibody molecule of appropriate biological activity can be used. As described supra, a chimeric antibody is a molecule in which different portions are derived from different animal species, such as those having a variable region derived from a murine mAb and a human immunoglobulin constant region, e.g., humanized antibodies.

Alternatively, techniques described for the production of single chain antibodies (U.S. Pat. No. 4,946,778; Bird, Science 242:423- 42 (1988); Huston et al., Proc. Natl. Acad. Sci. USA 85:5879-5883 (1988); and Ward et al., Nature 334:544-54 (1989)) can be adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli may also be used (Skerra et al., Science 242:1038- 1041 (1988)).

Methods of Producing Antibodies

The antibodies of the invention can be produced by any method known in the art for the synthesis of antibodies, in particular, by chemical synthesis or preferably, by recombinant expression techniques.

Recombinant expression of an antibody of the invention, or fragment, derivative or analog thereof, (e.g., a heavy or light chain of an antibody of the invention or a single chain antibody of the invention), requires construction of an expression vector containing a polynucleotide that encodes the antibody. Once a polynucleotide encoding an antibody molecule or a heavy or light chain of an antibody, or portion thereof (preferably containing the heavy or light chain variable domain), of the invention has been obtained, the vector for the production of the antibody molecule may be produced by recombinant DNA technology using techniques well known in the art. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, or a heavy or light chain thereof, or a heavy or light chain variable domain, operably linked to a promoter. Such vectors may include the nucleotide sequence encoding the constant region of the antibody molecule (see, e.g., PCT Publication WO 86/05807; PCT Publication WO 89/01036; and U.S. Pat. No. 5,122,464) and the variable domain of the antibody may be cloned into such a vector for expression of the entire heavy or light chain.

The expression vector is transferred to a host cell by conventional techniques and the transfected cells are then cultured by conventional techniques to produce an antibody of the invention. Thus, the invention includes host cells containing a polynucleotide encoding an antibody of the invention, or a heavy or light chain thereof, or a single chain antibody of the invention, operably linked to a heterologous promoter. In preferred embodiments for the expression of double-chained antibodies, vectors encoding both the heavy and light chains may be co-expressed in the host cell for expression of the entire immunoglobulin molecule, as detailed below.

A variety of host-expression vector systems may be utilized to express the antibody molecules of the invention. Such host-expression systems represent vehicles by which the coding sequences of interest may be produced and subsequently purified, but also represent cells which may, when transformed or transfected with the appropriate nucleotide coding sequences, express an antibody molecule of the invention in situ. These include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant antibody molecule, are used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., Gene 45:101 (1986); Cockett et al., Bio/Technology 8:2 (1990)).

In bacterial systems, a number of expression vectors may be advantageously selected depending upon the use intended for the antibody molecule being expressed. For example, when a large quantity of such a protein is to be produced, for the generation of pharmaceutical compositions of an antibody molecule, vectors which direct the expression of high levels of fusion protein products that are readily purified may be desirable. Such vectors include, but are not limited, to the E. coli expression vector pUR278 (Ruther et al., EMBO J. 2:1791 (1983)), in which the antibody coding sequence may be ligated individually into the vector in frame with the lac Z coding region so that a fusion protein is produced; pIN vectors (Inouye & Inouye, Nucleic Acids Res. 13:3101-3109 (1985); Van Heeke & Schuster, J. Biol. Chem. 24:5503-5509 (1989)); and the like. pGEX vectors may also be used to express foreign polypeptides as fusion proteins with glutathione S-transferase (GST). In general, such fusion proteins are soluble and can easily be purified from lysed cells by adsorption and binding to matrix glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non- essential region of the viral genome (e.g., region E1 or E3) will result in a recombinant virus that is viable and capable of expressing the antibody molecule in infected hosts. (e.g., see Logan & Shenk, Proc. Natl. Acad. Sci. USA 81:355-359 (1984)). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., Methods in Enzymol. 153:51-544 (1987)).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, Hela, COS, MDCK, 293, 3T3, WI38, and in particular, breast cancer cell lines such as, for example, BT483, Hs578T, HTB2, BT20 and T47D, and normal mammary gland cell line such as, for example, CRL7030 and Hs578Bst.

For long-term, high-yield production of recombinant proteins, stable expression is preferred. For example, cell lines which stably express the antibody molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the antibody molecule. Such engineered cell lines may be particularly useful in screening and evaluation of compounds that interact directly or indirectly with the antibody molecule.

A number of selection systems may be used, including but not limited to the herpes simplex virus thymidine kinase (Wigler et al., Cell 11:223 (1977)), hypoxanthine-guanine phosphoribosyltransferase (Szybalska & Szybalski, Proc. Natl. Acad. Sci. USA 48:202 (1992)), and adenine phosphoribosyltransferase (Lowy et al., Cell 22:817 (1980)) genes can be employed in tk-, hgprt- or aprt- cells, respectively. Also, antimetabolite resistance can be used as the basis of selection for the following genes: dhfr, which confers resistance to methotrexate (Wigler et al., Natl. Acad. Sci. USA 77:357 (1980); O'Hare et al., Proc. Natl. Acad. Sci. USA 78:1527 (1981)); gpt, which confers resistance to mycophenolic acid (Mulligan & Berg, Proc. Natl. Acad. Sci. USA 78:2072 (1981)); neo, which confers resistance to the aminoglycoside G-418 Clinical Pharmacy 12:488-505; Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); May, 1993, TIB TECH 11(5):155-215); and hygro, which confers resistance to hygromycin (Santerre et al., Gene 30:147 (1984)). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, N.Y. (1990); and in Chapters 12 and 13, Dracopoli et al. (eds), Current Protocols in Human Genetics, John Wiley & Sons, N.Y. (1994); Colberre-Garapin et al., J. Mol. Biol. 150:1 (1981), which are incorporated by reference herein in their entireties.

The expression levels of an antibody molecule can be increased by vector amplification (for a review, see Bebbington and Hentschel, The use of vectors based on gene amplification for the expression of cloned genes in mammalian cells in DNA cloning, Vol.3. (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., Mol. Cell. Biol. 3:257 (1983)).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain polypeptides. In such situations, the light chain should be placed before the heavy chain to avoid an excess of toxic free heavy chain (Proudfoot, Nature 322:52 (1986); Kohler, Proc. Natl. Acad. Sci. USA 77:2197 (1980)). The coding sequences for the heavy and light chains may comprise cDNA or genomic DNA.

Once an antibody molecule of the invention has been produced by an animal, chemically synthesized, or recombinantly expressed, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. In addition, the antibodies of the present invention or fragments thereof can be fused to heterologous polypeptide sequences described herein or otherwise known in the art, to facilitate purification.

The present invention encompasses antibodies recombinantly fused or chemically conjugated (including both covalently and non-covalently conjugations) to a polypeptide (or portion thereof, preferably at least 10, 20, 30, 40, 50, 60, 70, 80, 90 or 100 amino acids of the polypeptide) of the present invention to generate fusion proteins. The fusion does not necessarily need to be direct, but may occur through linker sequences. The antibodies may be specific for antigens other than polypeptides (or portion thereof, preferably at least 10, 20, 30, 40, 50, 60, 70, 80, 90 or 100 amino acids of the polypeptide) of the present invention. For example, antibodies may be used to target the polypeptides of the present invention to particular cell types, either in vitro or in vivo, by fusing or conjugating the polypeptides of the present invention to antibodies specific for particular cell surface receptors. Antibodies fused or conjugated to the polypeptides of the present invention may also be used in in vitro immunoassays and purification methods using methods known in the art. See e.g., Harbor et al., supra, and PCT publication WO 93/21232; EP 439,095; Naramura et al., Immunol. Lett. 39:91-99 (1994); U.S. Pat. No. 5,474,981; Gillies et al., PNAS 89:1428-1432 (1992); Fell et al., J. Immunol. 146:2446-2452(1991), which are incorporated by reference in their entireties.

The present invention further includes compositions comprising the polypeptides of the present invention fused or conjugated to antibody domains other than the variable regions. For example, the polypeptides of the present invention may be fused or conjugated to an antibody Fc region, or portion thereof. The antibody portion fused to a polypeptide of the present invention may comprise the constant region, hinge region, CH1 domain, CH2 domain, and CH3 domain or any combination of whole domains or portions thereof. The polypeptides may also be fused or conjugated to the above antibody portions to form multimers. For example, Fe portions fused to the polypeptides of the present invention can form dimers through disulfide bonding between the Fe portions. Higher multimeric forms can be made by fusing the polypeptides to portions of IgA and IgM. Methods for fusing or conjugating the polypeptides of the present invention to antibody portions are known in the art. See, e.g., U.S. Pat. Nos. 5,336,603; 5,622,929; 5,359,046; 5,349,053; 5,447,851; 5,112,946; EP 307,434; EP 367,166; PCT publications WO 96/04388; WO 91/06570; Ashkenazi et al., Proc. Natl. Acad. Sci. USA 88:10535-10539 (1991); Zheng et al., J. Immunol. 154:5590-5600 (1995); and Vil et al., Proc. Natl. Acad. Sci. USA 89:11337-11341(1992) (said references incorporated by reference in their entireties).

As discussed, supra, the polypeptides corresponding to a polypeptide, polypeptide fragment, or a variant of SEQ ID NO: 2, SEQ ID NO: 4, SEQ ID NO: 29, or SEQ ID NO: 32 may be fused or conjugated to the above antibody portions to increase the in vivo half life of the polypeptides or for use in immunoassays using methods known in the art. Further, the polypeptides corresponding to SEQ ID NO: 2, SEQ ID NO: 4, SEQ ID NO: 29, or SEQ ID NO: 32 may be fused or conjugated to the above antibody portions to facilitate purification. One reported example describes chimeric proteins consisting of the first two domains of the human CD4-polypeptide and various domains of the constant regions of the heavy or light chains of mammalian immunoglobulins. (EP 394,827; Traunecker et al., Nature 331:84-86 (1988). The polypeptides of the present invention fused or conjugated to an antibody having disulfide-linked dimeric structures (due to the IgG) may also be more efficient in binding and neutralizing other molecules, than the monomeric secreted protein or protein fragment alone. (Fountoulakis et al., J. Biochem. 270:3958-3964 (1995)). In many cases, the Fc part in a fusion protein is beneficial in therapy and diagnosis, and thus can result in, for example, improved pharmacokinetic properties. (EP A 232,262). Alternatively, deleting the Fc part after the fusion protein has been expressed, detected, and purified, would be desired. For example, the Fc portion may hinder therapy and diagnosis if the fusion protein is used as an antigen for immunizations. In drug discovery, for example, human proteins, such as hIL-5, have been fused with Fc portions for the purpose of high-throughput screening assays to identify antagonists of hIL-5. (See, Bennett et al., J. Molecular Recognition 8:52-58 (1995); Johanson et al., J. Biol. Chem. 270:9459-9471 (1995).

Moreover, the antibodies or fragments thereof of the present invention can be fused to marker sequences, such as a peptide to facilitate purification. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., Proc. Natl. Acad. Sci. USA 86:821-824 (1989), for instance, hexa-histidine provides for convenient purification of the fusion protein. Other peptide tags useful for purification include, but are not limited to, the “HA” tag, which corresponds to an epitope derived from the influenza hemagglutinin protein (Wilson et al., Cell 37:767 (1984)) and the “flag” tag.

The present invention further encompasses antibodies or fragments thereof conjugated to a diagnostic or therapeutic agent. The antibodies can be used diagnostically to, for example, monitor the development or progression of a tumor as part of a clinical testing procedure to, e.g., determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, radioactive materials, positron emitting metals using various positron emission tomographies, and nonradioactive paramagnetic metal ions. The detectable substance may be coupled or conjugated either directly to the antibody (or fragment thereof) or indirectly, through an intermediate (such as, for example, a linker known in the art) using techniques known in the art. See, for example, U.S. Pat. No. 4,741,900 for metal ions which can be conjugated to antibodies for use as diagnostics according to the present invention. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include 125I, 131I, 111In or 99Tc.

Further, an antibody or fragment thereof may be conjugated to a therapeutic moiety such as a cytotoxin, e.g., a cytostatic or cytocidal agent, a therapeutic agent or a radioactive metal ion, e.g., alpha-emitters such as, for example, 213Bi. A cytotoxin or cytotoxic agent includes any agent that is detrimental to cells. Examples include paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the therapeutic agent or drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, a-interferon, B-interferon, nerve growth factor, platelet derived growth factor, tissue plasminogen activator, an apoptotic agent, e.g., TNF-alpha, TNF-beta, AIM I (See, International Publication No. WO 97/33899), AIM II (See, International Publication No. WO 97/34911), Fas Ligand (Takahashi et al., Int. Immunol., 6:1567-1574 (1994)), VEGI (See, International Publication No. WO 99/23105), a thrombotic agent or an anti- angiogenic agent, e.g., angiostatin or endostatin; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

Antibodies may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev. 62:119-58 (1982).

Alternatively, an antibody can be conjugated to a second antibody to form an antibody heteroconjugate as described by Segal in U.S. Pat. No. 4,676,980, which is incorporated herein by reference in its entirety.

An antibody, with or without a therapeutic moiety conjugated to it, administered alone or in combination with cytotoxic factor(s) and/or cytokine(s) can be used as a therapeutic.

Immunophenotyping

The antibodies of the invention may be utilized for immunophenotyping of cell lines and biological samples. The translation product of the gene of the present invention may be useful as a cell specific marker, or more specifically as a cellular marker that is differentially expressed at various stages of differentiation and/or maturation of particular cell types. Monoclonal antibodies directed against a specific epitope, or combination of epitopes, will allow for the screening of cellular populations expressing the marker. Various techniques can be utilized using monoclonal antibodies to screen for cellular populations expressing the marker(s), and include magnetic separation using antibody-coated magnetic beads, “panning” with antibody attached to a solid matrix (i.e., plate), and flow cytometry (See, e.g., U.S. Pat. No. 5,985,660; and Morrison et al., Cell, 96:737-49 (1999)).

These techniques allow for the screening of particular populations of cells, such as might be found with hematological malignancies (i.e. minimal residual disease (MRD) in acute leukemic patients) and “non-self” cells in transplantations to prevent Graft-versus-Host Disease (GVHD). Alternatively, these techniques allow for the screening of hematopoietic stem and progenitor cells capable of undergoing proliferation and/or differentiation, as might be found in human umbilical cord blood.

Assays For Antibody Binding

The antibodies of the invention may be assayed for immunospecific binding by any method known in the art. The immunoassays which can be used include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al, eds, 1994, Current Protocols in Molecular Biology, Vol. 1, John Wiley & Sons, Inc., New York, which is incorporated by reference herein in its entirety). Exemplary immunoassays are described briefly below (but are not intended by way of limitation).

Immunoprecipitation protocols generally comprise lysing a population of cells in a lysis buffer such as RIPA buffer (1% NP-40 or Triton X- 100, 1% sodium deoxycholate, 0.1% SDS, 0.15M NaCl, 0.01M sodium phosphate at pH 7.2, 1% Trasylol) supplemented with protein phosphatase and/or protease inhibitors (e.g., EDTA, PMSF, aprotinin, sodium vanadate), adding the antibody of interest to the cell lysate, incubating for a period of time (e.g., 1-4 hours) at 4° C., adding protein A and/or protein G sepharose beads to the cell lysate, incubating for about an hour or more at 4° C., washing the beads in lysis buffer and resuspending the beads in SDS/sample buffer. The ability of the antibody of interest to immunoprecipitate a particular antigen can be assessed by, e.g., western blot analysis. One of skill in the art would be knowledgeable as to the parameters that can be modified to increase the binding of the antibody to an antigen and decrease the background (e.g., pre-clearing the cell lysate with sepharose beads). For further discussion regarding immunoprecipitation protocols see, e.g., Ausubel et al, eds, 1994, Current Protocols in Molecular Biology, Vol. 1, John Wiley & Sons, Inc., New York at 10.16.1.

Western blot analysis generally comprises preparing protein samples, electrophoresis of the protein samples in a polyacrylamide gel (e.g., 8%- 20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with primary antibody (the antibody of interest) diluted in blocking buffer, washing the membrane in washing buffer, blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., 32P or 125I) diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols see, e.g., Ausubel et al, eds, 1994, Current Protocols in Molecular Biology, Vol. 1, John Wiley & Sons, Inc., New York at 10.8.1.

ELISAs comprise preparing antigen, coating the well of a 96 well microtiter plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art. For further discussion regarding ELISAs see, e.g., Ausubel et al, eds, 1994, Current Protocols in Molecular Biology, Vol. 1, John Wiley & Sons, Inc., New York at 11.2.1.

The binding affinity of an antibody to an antigen and the off-rate of an antibody-antigen interaction can be determined by competitive binding assays. One example of a competitive binding assay is a radioimmunoassay comprising the incubation of labeled antigen (e.g., 3H or 125I) with the antibody of interest in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest conjugated to a labeled compound (e.g., 3H or 125I) in the presence of increasing amounts of an unlabeled second antibody.

Therapeutic Uses

The present invention is further directed to antibody-based therapies which involve administering antibodies of the invention to an animal, preferably a mammal, and most preferably a human, patient for treating, preventing, and/or diagnosing one or more of the disclosed diseases, disorders, or conditions. Therapeutic compounds of the invention include, but are not limited to, antibodies of the invention (including fragments, analogs and derivatives thereof as described herein) and nucleic acids encoding antibodies of the invention (including fragments, analogs and derivatives thereof and anti-idiotypic antibodies as described herein). The antibodies of the invention can be used to treat, inhibit or prevent diseases, disorders or conditions associated with aberrant expression and/or activity of a polypeptide of the invention, including, but not limited to, any one or more of the diseases, disorders, or conditions described herein. The treatment, prevention, and/or diagnosis of diseases, disorders, or conditions associated with aberrant expression and/or activity of a polypeptide of the invention includes, but is not limited to, alleviating symptoms associated with those diseases, disorders or conditions. Antibodies of the invention may be provided in pharmaceutically acceptable compositions as known in the art or as described herein.

A summary of the ways in which the antibodies of the present invention may be used therapeutically includes binding polynucleotides or polypeptides of the present invention locally or systemically in the body or by direct cytotoxicity of the antibody, e.g. as mediated by complement (CDC) or by effector cells (ADCC). Some of these approaches are described in more detail below. Armed with the teachings provided herein, one of ordinary skill in the art will know how to use the antibodies of the present invention for diagnostic, monitoring or therapeutic purposes without undue experimentation.

The antibodies of this invention may be advantageously utilized in combination with other monoclonal or chimeric antibodies, or with lymphokines or hematopoietic growth factors (such as, e.g., IL-2, IL-3 and IL-7), for example, which serve to increase the number or activity of effector cells which interact with the antibodies.

The antibodies of the invention may be administered alone or in combination with other types of treatments (e.g., radiation therapy, chemotherapy, hormonal therapy, immunotherapy and anti-tumor agents). Generally, administration of products of a species origin or species reactivity (in the case of antibodies) that is the same species as that of the patient is preferred. Thus, in a preferred embodiment, human antibodies, fragments derivatives, analogs, or nucleic acids, are administered to a human patient for therapy or prophylaxis.

It is preferred to use high affinity and/or potent in vivo inhibiting and/or neutralizing antibodies against polypeptides or polynucleotides of the present invention, fragments or regions thereof, for both immunoassays directed to and therapy of disorders related to polynucleotides or polypeptides, including fragments thereof, of the present invention. Such antibodies, fragments, or regions, will preferably have an affinity for polynucleotides or polypeptides of the invention, including fragments thereof. Preferred binding affinities include those with a dissociation constant or Kd less than 5×10⁻²M, 10⁻²M, 5×10⁻³M, 10⁻³M, 5×10⁻⁴M, 10⁻⁴M, 5×10⁻⁵M, 10⁻⁵M, 5×10⁻⁶M, 10⁻⁶M, 5×10⁻⁷M, 10⁻⁷M, 5×10⁻⁸M, 10⁻⁸M, 5×10⁻⁹M, 10⁻⁹M, 5×10⁻¹⁰M, 10⁻¹⁰M, 5×10⁻¹¹M, 10⁻¹¹M, 5×10⁻¹²M, 10⁻¹²M, 5×10⁻¹³M, 10⁻¹³M, 5×10⁻¹⁴M, 10⁻¹⁴M, 5×10⁻¹⁵M, and 10⁻¹⁵M.

Vectors, Host Cells, and Protein Production

The present invention also relates to vectors containing the IL-2 1 and IL-22 polynucleotides, host cells, and the production of polypeptides by recombinant techniques. The vector may be, for example, a phage, plasmid, viral, or retroviral vector. Retroviral vectors may be replication competent or replication defective. In the latter case, viral propagation generally will occur only in complementing host cells.

IL-21 and IL-22 polynucleotides may be joined to a vector containing a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells.

The IL-21 and IL-22 polynucleotide inserts should be operatively linked to an appropriate promoter, such as the phage lambda PL promoter, the E. coli lac, trp, phoA and tac promoters, the SV40 early and late promoters and promoters of retroviral LTRs, to name a few. Other suitable promoters will be known to the skilled artisan. The expression constructs will further contain sites for transcription initiation, termination, and, in the transcribed region, a ribosome binding site for translation. The coding portion of the transcripts expressed by the constructs will preferably include a translation initiating codon at the beginning and a termination codon (UAA, UGA or UAG) appropriately positioned at the end of the polypeptide to be translated.

As indicated, the expression vectors will preferably include at least one selectable marker. Such markers include dihydrofolate reductase, G418 or neomycin resistance for eukaryotic cell culture and tetracycline, kanamycin or ampicillin resistance genes for culturing in E. coli and other bacteria. Representative examples of appropriate hosts include, but are not limited to, bacterial cells, such as E. coli, Streptomyces, and Salmonella typhimurium cells; fungal cells, such as yeast cells (e.g., Saccharomyces cerevisiae or Pichia pastoris (ATCC Accession No. 201178)); insect cells such as Drosophila S2 and Spodoptera Sf9 cells; animal cells such as CHO, COS, 293, and Bowes melanoma cells; and plant cells. Appropriate culture mediums and conditions for the above-described host cells are known in the art.

Among vectors preferred for use in bacteria include pQE70, pQE60 and pQE-9, available from QIAGEN, Inc.; pBluescript vectors, Phagescript vectors, pNH8A, pNH16a, pNH18A, pNH46A, available from Stratagene Cloning Systems, Inc.; and ptrc99a, pKK223-3, pKK233-3, pDR540, pRIT5 available from Pharmacia Biotech, Inc. Preferred expression vectors for use in yeast systems include, but are not limited to, pYES2, pYD1, pTEF1/Zeo, pYES2/GS, pPICZ, pGAPZ, pGAPZalpha, pPIC9, pPIC3.5, pHIL-D2, pHIL-S1, pPIC3.5K, pPIC9K, and PAO815 (all available from Invitrogen, Carlsbad, Calif.). Among preferred eukaryotic vectors are pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Introduction of the construct into the host cell can be effected by calcium phosphate transfection, DEAE-dextran mediated transfection, cationic lipid-mediated transfection, electroporation, transduction, infection, or other methods. Such methods are described in many standard laboratory manuals, such as Davis et al., Basic Methods In Molecular Biology (1986). It is specifically contemplated that IL-21 and IL-22 polypeptides may in fact be expressed by a host cell lacking a recombinant vector.

In one embodiment, the yeast “Pichia pastoris” is used to express IL-21 or IL-22 protein in a eukaryotic system. Pichia pastoris is a methylotrophic yeast which can metabolize methanol as its sole carbon source. A main step in the methanol metabolization pathway is the oxidation of methanol to formaldehyde using O₂. This reaction is catalyzed by the enzyme alcohol oxidase. In order to metabolize methanol as its sole carbon source, Pichia pastoris must generate high levels of alcohol oxidase due, in part, to the relatively low affinity of alcohol oxidase for O₂. Consequently, in a growth medium depending on methanol as a main carbon source, the promoter region of one of the two alcohol oxidase genes (AOX1) is highly active. In the presence of methanol, alcohol oxidase produced from the AOX1 gene comprises up to approximately 30% of the total soluble protein in Pichia pastoris. See, Ellis, S. B., et al., Mol. Cell. Biol. 5:1111-21 (1985); Koutz, P. J, et al., Yeast 5:167-77 (1989); Tschopp, J. F., et al., Nucl. Acids Res. 15:3859-76 (1987). Thus, a heterologous coding sequence, such as, for example, a IL-21 or IL-22 polynucleotide of the present invention, under the transcriptional regulation of all or part of the AOX1 regulatory sequence is expressed at exceptionally high levels in Pichia yeast grown in the presence of methanol.

In one example, the plasmid vector pPIC9K is used to express DNA encoding a IL-21 or IL-22 polypeptide of the invention, as set forth herein, in a Pichia yeast system essentially as described in “Pichia Protocols: Methods in Molecular Biology,” D. R. Higgins and J. Cregg, eds. The Humana Press, Totowa, N.J., 1998. This expression vector allows expression and secretion of a IL-21 or IL-22 protein of the invention by virtue of the strong AOX1 promoter linked to the Pichia pastoris alkaline phosphatase (PHO) secretory signal peptide (i.e., leader) located upstream of a multiple cloning site.

Many other yeast vectors could be used in place of pPIC9K, such as, pYES2, pYD1, pTEF1/Zeo, pYES2/GS, pPICZ, pGAPZ, pGAPZalpha, pPIC9, pPIC3.5, pHIL-D2, pHIL-S1, pPIC3.5K, and PAO815, as one skilled in the art would readily appreciate, as long as the proposed expression construct provides appropriately located signals for transcription, translation, secretion (if desired), and the like, including an in-frame AUG as required.

In another embodiment, high-level expression of a heterologous coding sequence, such as, for example, an IL-21 or IL-22 polynucleotide of the present invention, may be achieved by cloning the heterologous polynucleotide of the invention into an expression vector such as, for example, pGAPZ or pGAPZalpha, and growing the yeast culture in the absence of methanol.

Transcription of the DNA encoding the polypeptides of the present invention by higher eukaryotes is increased by inserting an enhancer sequence into the vector. Enhancers are cis-acting elements of DNA, usually about from 10 to 300 bp that act on a promoter to increase its transcription. Examples including the SV40 enhancer on the late side of the replication origin bp 100 to 270, a cytomegalovirus early promoter enhancer, the polyoma enhancer on the late side of the replication origin, and adenovirus enhancers.

Various mammalian cell culture systems can also be employed to express recombinant protein. Examples of mammalian expression systems include the COS-7 lines of monkey kidney fibroblasts, described by Gluzman (Cell 23:175 (1981)), and other cell lines capable of expressing a compatible vector, for example, the C127, 3T3, CHO, HeLa and BHK cell lines. Mammalian expression vectors will comprise an origin of replication, a suitable promoter and enhancer, and also any necessary ribosome binding sites, polyadenylation site, splice donor and acceptor sites, transcriptional termination sequences, and 5′ flanking nontranscribed sequences. DNA sequences derived from the SV40 splice, and polyadenylation sites may be used to provide the required nontranscribed genetic elements.

In addition to encompassing host cells containing the vector constructs discussed herein, the invention also encompasses primary, secondary, and immortalized host cells of vertebrate origin, particularly mammalian origin, that have been engineered to delete or replace endogenous genetic material (e.g., IL-21 or IL-22 coding sequence), and/or to include genetic material (e.g., heterologous polynucleotide sequences) that is operably associated with IL-21 or IL-22 polynucleotides of the invention, and which activates, alters, and/or amplifies endogenous IL-21 or IL-22 polynucleotides. For example, techniques known in the art may be used to operably associate heterologous control regions (e.g., promoter and/or enhancer) and endogenous IL-21 or IL-22 polynucleotide sequences via homologous recombination (see, e.g., U.S. Pat. No. 5,641,670, issued Jun. 24, 1997; International Publication No. WO 96/29411, published Sep. 26, 1996; International Publication No. WO 94/12650, published Aug. 4, 1994; Koller et al., Proc. Natl. Acad. Sci. USA 86:8932-8935 (1989); and Zijlstra et al., Nature 342:435-438 (1989), the disclosures of each of which are incorporated by reference in their entireties).

The host cells described infra can be used in a conventional manner to produce the gene product encoded by the recombinant sequence. Alternatively, cell-free translation systems can also be employed to produce the polypeptides of the invention using RNAs derived from the DNA constructs of the present invention.

The polypeptide of the invention may be expressed or synthesized in a modified form, such as a fusion protein (comprising the polypeptide joined via a peptide bond to a heterologous protein sequence (of a different protein)), and may include not only secretion signals, but also additional heterologous functional regions. Such a fusion protein can be made by ligating polynucleotides of the invention and the desired nucleic acid sequence encoding the desired amino acid sequence to each other, by methods known in the art, in the proper reading frame, and expressing the fusion protein product by methods known in the art. Alternatively, such a fusion protein can be made by protein synthetic techniques, e.g., by use of a peptide synthesizer. Thus, for instance, a region of additional amino acids, particularly charged amino acids, may be added to the N-terminus of the polypeptide to improve stability and persistence in the host cell, during purification, or during subsequent handling and storage. Also, peptide moieties may be added to the polypeptide to facilitate purification. Such regions may be removed prior to final preparation of the polypeptide. The addition of peptide moieties to polypeptides to engender secretion or excretion, to improve stability and to facilitate purification, among others, are familiar and routine techniques in the art.

In one embodiment, polynucleotides encoding IL-21 or IL-22 polypeptides of the invention may be fused to the pelB pectate lyase signal sequence to increase the efficiency to expression and purification of such polypeptides in Gram-negative bacteria. See, U.S. Pat. Nos. 5,576,195 and 5,846,818, the contents of which are herein incorporated by reference in their entireties.

A preferred fusion protein comprises a heterologous region from immunoglobulin that is useful to stabilize and purify proteins. For example, EP-A-O 464 533 (Canadian counterpart 2045869) discloses fusion proteins comprising various portions of constant region of immunoglobulin molecules together with another human protein or part thereof. In many cases, the Fc part in a fusion protein is thoroughly advantageous for use in therapy and diagnosis and thus results, for example, in improved pharmacokinetic properties (EP-A 0232 262). On the other hand, for some uses it would be desirable to be able to delete the Fc part after the fusion protein has been expressed, detected and purified in the advantageous manner described. This is the case when Fc portion proves to be a hindrance to use in therapy and diagnosis, for example when the fusion protein is to be used as antigen for immunizations. In drug discovery, for example, human proteins, such as hIL-5 has been fused with Fc portions for the purpose of high-throughput screening assays to identify antagonists of hIL-5. See, D. Bennett et al., J. Molecular Recognition 8:52-58 (1995) and K. Johanson et al., J. Biol. Chem. 270:9459-9471 (1995).

Polypeptides of the present invention include naturally purified products, products of chemical synthetic procedures, and products produced by recombinant techniques from a prokaryotic or eukaryotic host, including, for example, bacterial, yeast, higher plant, insect and mammalian cells. Depending upon the host employed in a recombinant production procedure, the polypeptides of the present invention may be glycosylated or may be non-glycosylated. In addition, polypeptides of the invention may also include an initial modified methionine residue, in some cases as a result of host-mediated processes.

Polypeptides of the invention can be chemically synthesized using techniques known in the art (e.g., see Creighton, 1983, Proteins: Structures and Molecular Principles, W. H. Freeman & Co., N.Y., and Hunkapiller, M., et al., 1984, Nature 310:105-111). For example, a peptide corresponding to a fragment of the complete IL-21 or IL-22 polypeptides of the invention can be synthesized by use of a peptide synthesizer. Furthermore, if desired, nonclassical amino acids or chemical amino acid analogs can be introduced as a substitution or addition into the IL-21 or IL-22 polynucleotide sequence. Non-classical amino acids include, but are not limited to, to the D-isomers of the common amino acids, 2,4-diaminobutyric acid, a-amino isobutyric acid, 4-aminobutyric acid, Abu, 2-amino butyric acid, g-Abu, e-Ahx, 6-amino hexanoic acid, Aib, 2-amino isobutyric acid, 3-amino propionic acid, ornithine, norleucine, norvaline, hydroxyproline, sarcosine, citrulline, homocitralline, cysteic acid, t-butylglycine, t-butylalanine, phenylglycine, cyclohexylalanine, b-alanine, fluoro-amino acids, designer amino acids such as b-methyl amino acids, Ca-methyl amino acids, Na-methyl amino acids, and amino acid analogs in general. Furthermore, the amino acid can be D (dextrorotary) or L (levorotary).

The invention encompasses IL-21 or IL-22 polypeptides which are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited, to specific chemical cleavage by cyanogen bromide, trypsin, chymotrypsin, papain, V8 protease, NaBH₄, acetylation, formylation, oxidation, reduction, metabolic synthesis in the presence of tunicamycin, etc.

Additional post-translational modifications encompassed by the invention include, for example, e.g., N-linked or O-linked carbohydrate chains, processing of N-terminal or C-terminal ends), attachment of chemical moieties to the amino acid backbone, chemical modifications of N-linked or O-linked carbohydrate chains, and addition or deletion of an N-terminal methionine residue as a result of procaryotic host cell expression. The polypeptides may also be modified with a detectable label, such as an enzymatic, fluorescent, isotopic or affinity label to allow for detection and isolation of the protein. In addition, polypeptides of the invention may be modified by iodination.

In one embodiment, IL-21 or IL-22 polypeptides of the invention may also be labeled with biotin. In other related embodiments, biotinylated IL-21 or IL-22 polypeptides of the invention may be used, for example, as an imaging agent or as a means of identifying one or more IL-21 or IL-22 receptor(s) or other coreceptor or coligand molecules.

Also provided by the invention are chemically modified derivatives of IL-21 or IL-22 which may provide additional advantages such as increased solubility, stability and circulating time of the polypeptide, or decreased immunogenicity (see U.S. Pat. No. 4,179,337). The chemical moieties for derivitization may be selected from water soluble polymers such as polyethylene glycol, ethylene glycol/propylene glycol copolymers, carboxymethylcellulose, dextran, polyvinyl alcohol and the like. The polypeptides may be modified at random positions within the molecule, or at predetermined positions within the molecule and may include one, two, three or more attached chemical moieties.

The polymer may be of any molecular weight, and may be branched or unbranched. For polyethylene glycol, the preferred molecular weight is between about 1 kDa and about 100 kDa (the term about indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog).

The polyethylene glycol molecules (or other chemical moieties) should be attached to the protein with consideration of effects on functional or antigenic domains of the protein. There are a number of attachment methods available to those skilled in the art, e.g., EP 0 401 384, herein incorporated by reference (coupling PEG to G-CSF), see also Malik et al., Exp. Hematol. 20:1028-1035 (1992) (reporting pegylation of GM-CSF using tresyl chloride). For example, polyethylene glycol may be covalently bound through amino acid residues via a reactive group, such as, a free amino or carboxyl group. Reactive groups are those to which an activated polyethylene glycol molecule may be bound. The amino acid residues having a free amino group may include lysine residues and the N-terminal amino acid residues; those having a free carboxyl group may include aspartic acid residues, glutamic acid residues, and the C-terminal amino acid residue. Sulfhydryl groups may also be used as a reactive group for attaching the polyethylene glycol molecules. Preferred for therapeutic purposes is attachment at an amino group, such as attachment at the N-terminus or lysine group.

One may specifically desire proteins chemically modified at the N-terminus. Using polyethylene glycol as an illustration, one may select from a variety of polyethylene glycol molecules (by molecular weight, branching, etc.), the proportion of polyethylene glycol molecules to protein (or peptide) molecules in the reaction mix, the type of pegylation reaction to be performed, and the method of obtaining the selected N-terminally pegylated protein. The method of obtaining the N-terminally pegylated preparation (i.e., separating this moiety from other monopegylated moieties if necessary) may be by purification of the N-terminally pegylated material from a population of pegylated protein molecules. Selective proteins chemically modified at the N-terminus modification may be accomplished by reductive alkylation which exploits differential reactivity of different types of primary amino groups (lysine versus the N-terminal) available for derivatization in a particular protein. Under the appropriate reaction conditions, substantially selective derivatization of the protein at the N-terminus with a carbonyl group containing polymer is achieved.

IL-21 and IL-22 polypeptides can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography (“HPLC”) is employed for purification.

IL-21 and IL-22 polypeptides, and preferably the secreted forms thereof, can also be recovered from: products purified from natural sources, including bodily fluids, tissues and cells, whether directly isolated or cultured; products of chemical synthetic procedures; and products produced by recombinant techniques from a prokaryotic or eukaryotic host, including, for example, bacterial, yeast, higher plant, insect, and mammalian cells. Depending upon the host employed in a recombinant production procedure, the IL-21 and IL-22 polypeptides may be glycosylated or may be non-glycosylated. In addition, IL-21 and IL-22 polypeptides may also include an initial modified methionine residue, in some cases as a result of host-mediated processes. Thus, it is well known in the art that the N-terminal methionine encoded by the translation initiation codon generally is removed with high efficiency from any protein after translation in all eukaryotic cells. While the N-terminal methionine on most proteins also is efficiently removed in most prokaryotes, for some proteins, this prokaryotic removal process is inefficient, depending on the nature of the amino acid to which the N-terminal methionine is covalently linked.

Uses of the IL-21 and IL-22 Polynucleotides

The IL-21 and IL-22 polynucleotides identified herein can be used in numerous ways as reagents. The following description should be considered exemplary and utilizes known techniques.

There exists an ongoing need to identify new chromosome markers, since few chromosome marking reagents, based on actual sequence data (repeat polymorphisms), are presently available. Clone HTGED19 and clone HFPBX96 can each be mapped to a specific chromosome. Thus, IL-21 and IL-22 polynucleotides can then be used in linkage analysis as a marker for those specific chromosome.

Briefly, sequences can be mapped to chromosomes by preparing PCR primers (preferably 15-25 bp) from the sequences shown in SEQ ID NO: 1, SEQ ID NO: 3, SEQ ID NO: 28, and SEQ ID NO: 3 1. Primers can be selected using computer analysis so that primers do not span more than one predicted exon in the genomic DNA. These primers are then used for PCR screening of somatic cell hybrids containing individual human chromosomes. Only those hybrids containing the human IL-21 or IL-22 genes corresponding to SEQ ID NO: 1, SEQ ID NO: 3, SEQ ID NO: 28 or SEQ ID NO: 31, respectively, will yield an amplified fragment.

Similarly, somatic hybrids provide a rapid method of PCR mapping the polynucleotides to particular chromosomes. Three or more clones can be assigned per day using a single thermal cycler. Moreover, sublocalization of the IL-21 and IL-22 polynucleotides can be achieved with panels of specific chromosome fragments. Other gene mapping strategies that can be used include in situ hybridization, prescreening with labeled flow-sorted chromosomes, and preselection by hybridization to construct chromosome specific-cDNA libraries.

Precise chromosomal location of the IL-21 and IL-22 polynucleotides can also be achieved using fluorescence in situ hybridization (FISH) of a metaphase chromosomal spread. This technique uses polynucleotides as short as 500 or 600 bases; however, polynucleotides 2,000-4,000 bp are preferred (For review, see Verma, et al., “Human Chromosomes: a Manual of Basic Techniques,” Pergamon Press, New York (1988)).

For chromosome mapping, the IL-21 and IL-22 polynucleotides can be used individually (to mark a single chromosome or a single site on that chromosome) or in panels (for marking multiple sites and/or multiple chromosomes). Preferred polynucleotides correspond to the noncoding regions of the cDNAs because the coding sequences are more likely conserved within gene families, thus increasing the chance of cross hybridization during chromosomal mapping.

In a preferred embodiment, the gene encoding IL-22 of the present invention has been mapped using FISH technology to a location on human chromosome 13 at position 13q11. In addition, the gene encoding IL-21 of the present invention has mapped to a location on human chromosome 7. See also, Example 4 infra.

Once a polynucleotide has been mapped to a precise chromosomal location, the physical position of the polynucleotide can be used in linkage analysis. Linkage analysis establishes coinheritance between a chromosomal location and presentation of a particular disease (disease mapping data are found, for example, in McKusick, V., Mendelian Inheritance in Man (available on line through Johns Hopkins University Welch Medical Library)). Assuming 1 megabase mapping resolution and one gene per 20 kb, a cDNA precisely localized to a chromosomal region associated with the disease could be one of 50-500 potential causative genes.

Thus, once coinheritance is established, differences in the IL-21 and IL-22 polynucleotides and the corresponding genes between affected and unaffected individuals can be examined. First, visible structural alterations in the chromosomes, such as deletions or translocations, are examined in chromosome spreads or by PCR. If no structural alterations exist, the presence of point mutations are ascertained. Mutations observed in some or all affected individuals, but not in normal individuals, indicates that the mutation may cause the disease. However, complete sequencing of the IL-21 and IL-22 polypeptides and the corresponding genes from several normal individuals is required to distinguish the mutation from a polymorphism. If a new polymorphism is identified, this polymorphic polypeptide can be used for further linkage analysis.

Furthermore, increased or decreased expression of the gene in affected individuals as compared to unaffected individuals can be assessed using IL-21 and IL-22 polynucleotides. Any of these alterations (altered expression, chromosomal rearrangement, or mutation) can be used as a diagnostic or prognostic marker.

In addition to the foregoing, an IL-21 or IL-22 polynucleotide can be used to control gene expression through triple helix formation or antisense DNA or RNA. Both methods rely on binding of the polynucleotide to DNA or RNA. For these techniques, preferred polynucleotides are usually 20 to 40 bases in length and complementary to either the region of the gene involved in transcription (triple helix—see Lee, et al., Nucl. Acids Res. 6:3073 (1979); Cooney, et al., Science 241:456 (1988); and Dervan, et al., Science 251:1360 (1991)) or to the mRNA itself (antisense—Okano, J. Neurochem. 56:560 (1991); Oligodeoxy-nucleotides as Antisense Inhibitors of Gene Expression, CRC Press, Boca Raton, Fla. (1988)). Triple helix formation optimally results in a shut-off of RNA transcription from DNA, while antisense RNA hybridization blocks translation of an mRNA molecule into polypeptide. Both techniques are effective in model systems, and the information disclosed herein can be used to design antisense or triple helix polynucleotides in an effort to treat, prevent, and/or diagnose disease.

IL-21 and IL-22 polynucleotides are also useful in gene therapy. One goal of gene therapy is to insert a normal gene into an organism having a defective gene, in an effort to correct the genetic defect. IL-21 and IL-22 offer means of targeting such genetic defects in a highly accurate manner. Another goal is to insert a new gene that was not present in the host genome, thereby producing a new trait in the host cell.

The IL-21 and IL-22 polynucleotides are also useful for identifying individuals from minute biological samples. The United States military, for example, is considering the use of restriction fragment length polymorphism (RFLP) for identification of its personnel. In this technique, an individuals genomic DNA is digested with one or more restriction enzymes, and probed on a Southern blot to yield unique bands for identifying personnel. This method does not suffer from the current limitations of “Dog Tags” which can be lost, switched, or stolen, making positive identification difficult. The IL-21 and IL-22 polynucleotides can be used as additional DNA markers for RFLP.

The IL-21 and IL-22 polynucleotides can also be used as an alternative to RFLP, by determining the actual base-by-base DNA sequence of selected portions of an individuals genome. These sequences can be used to prepare PCR primers for amplifying and isolating such selected DNA, which can then be sequenced. Using this technique, individuals can be identified because each individual will have a unique set of DNA sequences. Once an unique ID database is established for an individual, positive identification of that individual, living or dead, can be made from extremely small tissue samples.

Forensic biology also benefits from using DNA-based identification techniques as disclosed herein. DNA sequences taken from very small biological samples such as tissues, e.g., hair or skin, or body fluids, e.g., blood, saliva, semen, etc., can be amplified using PCR. In one prior art technique, gene sequences amplified from polymorphic loci, such as DQa class II HLA gene, are used in forensic biology to identify individuals (Erlich, H., PCR Technology, Freeman and Co. (1992)). Once these specific polymorphic loci are amplified, they are digested with one or more restriction enzymes, yielding an identifying set of bands on a Southern blot probed with DNA corresponding to the DQa class II HLA gene. Similarly, IL-21 and IL-22 polynucleotides can be used as polymorphic markers for forensic purposes.

There is also a need for reagents capable of identifying the source of a particular tissue. Such need arises, for example, in forensics when presented with tissue of unknown origin. Appropriate reagents can comprise, for example, DNA probes or primers specific to particular tissue prepared from IL-21 and IL-22 sequences. Panels of such reagents can identify tissue by species and/or by organ type. In a similar fashion, these reagents can be used to screen tissue cultures for contamination.

Because IL-21 is found expressed almost exclusively in apoptotic T-cells, IL-21 polynucleotides are useful as hybridization probes for differential identification of the tissue(s) or cell type(s) present in a biological sample. Similarly, polypeptides and antibodies directed to IL-21 polypeptides are useful to provide immunological probes for differential identification of the tissue(s) or cell type(s). In addition, for a number of disorders of the above tissues or cells, particularly of the Immune system, significantly higher or lower levels of IL-21 gene expression may be detected in certain tissues (e.g., cancerous and wounded tissues) or bodily fluids (e.g., serum, plasma, urine, synovial fluid or spinal fluid) taken from an individual having such a disorder, relative to a “standard” IL-21 gene expression level, i.e., the IL-21 expression level in healthy tissue from an individual not having the Immune system disorder.

Likewise, since IL-22 is found expressed in bone marrow, skeletal muscle, and brain, IL-22 polynucleotides are useful as hybridization probes for differential identification of the tissue(s) or cell type(s) present in a biological sample. Similarly, polypeptides and antibodies directed to IL-22 polypeptides are useful to provide immunological probes for differential identification of the tissue(s) or cell type(s). In addition, for a number of disorders of the above tissues or cells, particularly of the Immune system, significantly higher or lower levels of IL-22 gene expression may be detected in certain tissues (e.g., cancerous and wounded tissues) or bodily fluids (e.g., serum, plasma, urine, synovial fluid or spinal fluid) taken from an individual having such a disorder, relative to a “standard” IL-22 gene expression level, i.e., the IL-22 expression level in healthy tissue from an individual not having the Immune system disorder.

Thus, the invention provides a diagnostic method of a disorder, which involves: (a) assaying IL-21 or IL-22 gene expression level in cells or body fluid of an individual; (b) comparing the IL-21 or IL-22 gene expression level with a standard IL-21 or IL-22 gene expression level, respectively, whereby an increase or decrease in the assayed IL-21 or IL-22 gene expression level compared to the standard expression level is indicative of disorder in the Immune system.

In the very least, the IL-21 and IL-22 polynucleotides can be used as molecular weight markers on Southern gels, as diagnostic probes for the presence of a specific mRNA in a particular cell type, as a probe to “subtract-out” known sequences in the process of discovering novel polynucleotides, for selecting and making oligomers for attachment to a “gene chip” or other support, to raise anti-DNA antibodies using DNA immunization techniques, and as an antigen to elicit an immune response.

Gene Therapy

In a specific embodiment, nucleic acids comprising sequences encoding antibodies or functional derivatives thereof, are administered to treat, inhibit or prevent a disease or disorder associated with aberrant expression and/or activity of a polypeptide of the invention, by way of gene therapy. Gene therapy refers to therapy performed by the administration to a subject of an expressed or expressible nucleic acid. In this embodiment of the invention, the nucleic acids produce their encoded protein that mediates a therapeutic effect.

Any of the methods for gene therapy available in the art can be used according to the present invention. Exemplary methods are described below.

For general reviews of the methods of gene therapy, see Goldspiel et al., Clinical Pharmacy 12:488-505 (1993); Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); May, TIBTECH 11(5):155-215 (1993). Methods commonly known in the art of recombinant DNA technology which can be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

In a preferred aspect, the compound comprises nucleic acid sequences encoding an antibody, said nucleic acid sequences being part of expression vectors that express the antibody or fragments or chimeric proteins or heavy or light chains thereof in a suitable host. In particular, such nucleic acid sequences have promoters operably linked to the antibody coding region, said promoter being inducible or constitutive, and, optionally, tissue- specific. In another particular embodiment, nucleic acid molecules are used in which the antibody coding sequences and any other desired sequences are flanked by regions that promote homologous recombination at a desired site in the genome, thus providing for intrachromosomal expression of the antibody encoding nucleic acids (Koller and Smithies, Proc. Natl. Acad. Sci. USA 86:8932-8935 (1989); Zijlstra et al., Nature 342:435-438 (1989). In specific embodiments, the expressed antibody molecule is a single chain antibody; alternatively, the nucleic acid sequences include sequences encoding both the heavy and light chains, or fragments thereof, of the antibody.

Delivery of the nucleic acids into a patient may be either direct, in which case the patient is directly exposed to the nucleic acid or nucleic acid- carrying vectors, or indirect, in which case, cells are first transformed with the nucleic acids in vitro, then transplanted into the patient. These two approaches are known, respectively, as in vivo or ex vivo gene therapy.

In a specific embodiment, the nucleic acid sequences are directly administered in vivo, where it is expressed to produce the encoded product. This can be accomplished by any of numerous methods known in the art, e.g., by constructing them as part of an appropriate nucleic acid expression vector and administering it so that they become intracellular, e.g., by infection using defective or attenuated retrovirals or other viral vectors (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu, J. Biol. Chem. 262:4429-4432 (1987)) (which can be used to target cell types specifically expressing the receptors), etc. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180; WO 92/22635; WO92/20316; WO93/14188, WO 93/20221). Alternatively, the nucleic acid can be introduced intracellularly and incorporated within host cell DNA for expression, by homologous recombination (Koller and Smithies, Proc. Natl. Acad. Sci. USA 86:8932-8935 (1989); Zijlstra et al., Nature 342:435-438 (1989)).

In a specific embodiment, viral vectors that contains nucleic acid sequences encoding an antibody of the invention are used. For example, a retroviral vector can be used (see Miller et al., Meth. Enzymol. 217:581-599 (1993)). These retroviral vectors contain the components necessary for the correct packaging of the viral genome and integration into the host cell DNA. The nucleic acid sequences encoding the antibody to be used in gene therapy are cloned into one or more vectors, which facilitates delivery of the gene into a patient. More detail about retroviral vectors can be found in Boesen et al., Biotherapy 6:291-302 (1994), which describes the use of a retroviral vector to deliver the mdr1 gene to hematopoietic stem cells in order to make the stem cells more resistant to chemotherapy. Other references illustrating the use of retroviral vectors in gene therapy are: Clowes et al., J. Clin. Invest. 93:644-651 (1994); Kiem et al., Blood 83:1467-1473 (1994); Salmons and Gunzberg, Human Gene Therapy 4:129-141 (1993); and Grossman and Wilson, Curr. Opin. in Genetics and Devel. 3:110-114 (1993).

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, Current Opinion in Genetics and Development 3:499-503 (1993) present a review of adenovirus-based gene therapy. Bout et al., Human Gene Therapy 5:3-10 (1994) demonstrated the use of adenovirus vectors to transfer genes to the respiratory epithelia of rhesus monkeys. Other instances of the use of adenoviruses in gene therapy can be found in Rosenfeld et al., Science 252:431-434 (1991); Rosenfeld et al., Cell 68:143-155 (1992); Mastrangeli et al., J. Clin. Invest. 91:225-234 (1993); PCT Publication WO94/12649; and Wang, et al., Gene Therapy 2:775-783 (1995). In a preferred embodiment, adenovirus vectors are used.

Adeno-associated virus (AAV) has also been proposed for use in gene therapy (Walsh et al., Proc. Soc. Exp. Biol. Med. 204:289-300 (1993); U.S. Pat. No. 5,436,146).

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see, e.g., Loeffler and Behr, Meth. Enzymol. 217:599-618 (1993); Cohen et al., Meth. Enzymol. 217:618-644 (1993); Cline, Pharmac. Ther. 29:69-92m (1985) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a patient by various methods known in the art. Recombinant blood cells (e.g., hematopoietic stem or progenitor cells) are preferably administered intravenously. The amount of cells envisioned for use depends on the desired effect, patient state, etc., and can be determined by one skilled in the art.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; blood cells such as Tlymphocytes, Blymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc.

In a preferred embodiment, the cell used for gene therapy is autologous to the patient.

In an embodiment in which recombinant cells are used in gene therapy, nucleic acid sequences encoding an antibody are introduced into the cells such that they are expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the present invention (see e.g. PCT Publication WO 94/08598; Stemple and Anderson, Cell 71:973-985 (1992); Rheinwald, Meth. Cell Bio. 21A:229 (1980); and Pittelkow and Scott, Mayo Clinic Proc. 61:771 (1986)).

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription. Demonstration of Therapeutic or Prophylactic Activity

The compounds or pharmaceutical compositions of the invention are preferably tested in vitro, and then in vivo for the desired therapeutic or prophylactic activity, prior to use in humans. For example, in vitro assays to demonstrate the therapeutic or prophylactic utility of a compound or pharmaceutical composition include, the effect of a compound on a cell line or a patient tissue sample. The effect of the compound or composition on the cell line and/or tissue sample can be determined utilizing techniques known to those of skill in the art including, but not limited to, rosette formation assays and cell lysis assays. In accordance with the invention, in vitro assays which can be used to determine whether administration of a specific compound is indicated, include in vitro cell culture assays in which a patient tissue sample is grown in culture, and exposed to or otherwise administered a compound, and the effect of such compound upon the tissue sample is observed.

Fusion Proteins

Any IL-21 or IL-22 polypeptide can be used to generate fusion proteins. For example, the IL-21 or IL-22 polypeptides, when fused to a second protein, can be used as an antigenic tag. Antibodies raised against the IL-21 or IL-22 polypeptides can be used to indirectly detect a second protein by binding to IL-21 or IL-22, respectively. Moreover, because secreted proteins target cellular locations based on trafficking signals, the IL-21 and IL-22 polypeptides can be used as targeting molecules once fused to other proteins.

Examples of domains that can be fused to the IL-21 and IL-22 polypeptides include not only heterologous signal sequences, but also other heterologous functional regions. The fusion does not necessarily need to be direct, but may occur through linker sequences.

In certain preferred embodiments, IL-21 and IL-22 proteins of the invention comprise fusion proteins wherein the IL-21 and IL-22 polypeptides are those described above as n¹-m¹, n²-m², n³-m³, n⁴-m⁴, n⁵-m⁵, or n⁶-m⁶. In preferred embodiments, the application is directed to nucleic acid molecules at least 90%, 95%, 96%, 97%, 98% or 99% identical to the nucleic acid sequences encoding polypeptides having the amino acid sequence of the specific N- and C-terminal deletions recited herein. Polynucleotides encoding these polypeptides are also encompassed by the invention.

Moreover, fusion proteins may also be engineered to improve characteristics of the IL-21 and IL-22 polypeptides. For instance, a region of additional amino acids, particularly charged amino acids, may be added to the N-terminus of the IL-21 and IL-22 polypeptides to improve stability and persistence during purification from the host cell or during subsequent handling and storage. Also, peptide moieties may be added to the IL-21 and IL-22 polypeptides to facilitate purification. Such regions may be removed prior to final preparation of the IL-21 and IL-22 polypeptides. The addition of peptide moieties to facilitate handling of polypeptides are familiar and routine techniques in the art. As one of skill in the art will appreciate, polypeptides of the present invention and the epitope-bearing fragments thereof described above, can be combined with heterologous polypeptide sequences. For example, the polypeptides of the present invention may be fused with heterologous polypeptide sequences, for example, IL-21 and IL-22 polypeptides, including fragments, and specifically epitopes, can be combined with parts of the constant domain of immunoglobulins (IgA, IgE, IgG, IgM), or portions thereof (CH1, CH2, CH3, and any combination thereof, including both entire domains and portions thereof), resulting in chimeric polypeptides. These fusion proteins facilitate purification and show an increased half-life in vivo. One reported example describes chimeric proteins consisting of the first two domains of the human CD4-polypeptide and various domains of the constant regions of the heavy or light chains of mammalian immunoglobulins. (EP A 394,827; Traunecker et al., Nature 331:84-86 (1988).) Fusion proteins having disulfide-linked dimeric structures (due to the IgG) can also be more efficient in binding and neutralizing other molecules, than the monomeric secreted protein or protein fragment alone. (Fountoulakis et al., J. Biochem. 270:3958-3964 (1995).)

Similarly, EP-A-O 464 533 (Canadian counterpart 2045869) discloses fusion proteins comprising various portions of constant region of immunoglobulin molecules together with another human protein or part thereof. In many cases, the Fc part in a fusion protein is beneficial in therapy and diagnosis, and thus can result in, for example, improved pharmacokinetic properties. (EP-A 0232 262.) Alternatively, deleting the Fc part after the fusion protein has been expressed, detected, and purified, would be desired. For example, the Fc portion may hinder therapy and diagnosis if the fusion protein is used as an antigen for immunizations. In drug discovery, for example, human proteins, such as hIL-5, have been fused with Fc portions for the purpose of high-throughput screening assays to identify antagonists of hIL-5. (See, D. Bennett et al., J. Molecular Recognition 8:52-58 (1995); K. Johanson et al., J. Biol. Chem. 270:9459-9471 (1995).)

Moreover, the IL-21 and IL-22 polypeptides can be fused to marker sequences, such as a peptide which facilitates purification of IL-21 and IL-22, respectively. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described by Gentz and coworkers (Proc. Natl. Acad. Sci. USA 86:821-824 (1989)), for instance, hexa-histidine provides for convenient purification of the fusion protein. Another peptide tag useful for purification, the “HA” tag, corresponds to an epitope derived from the influenza hemagglutinin protein (Wilson, et al., Cell 37:767 (1984)).

In further preferred embodiments, IL-21 or IL-22 polynucleotides of the invention are fused to a polynucleotide encoding a “FLAG” polypeptide. Thus, an IL-21-FLAG or an IL-22-FLAG fusion protein is encompassed by the present invention. The FLAG antigenic polypeptide may be fused to an IL-21 or an IL-22 polypeptide of the invention at either or both the amino or the carboxy terminus. In preferred embodiments, an IL-21-FLAG or an IL-22-FLAG fusion protein is expressed from a pFLAG-CMV-5a or a pFLAG-CMV-1 expression vector (available from Sigma, St. Louis, Mo., USA). See, Andersson, S., et al., J. Biol. Chem. 264:8222-29 (1989); Thomsen, D. R., et al., Proc. Natl. Acad. Sci. USA, 81:659-63 (1984); and Kozak, M., Nature 308:241 (1984) (each of which is hereby incorporated by reference). In further preferred embodiments, an IL-21 -FLAG or an IL-22-FLAG fusion protein is detectable by anti-FLAG monoclonal antibodies (also available from Sigma).

Thus, any of the above fusion proteins can be engineered using IL-21 and/or IL-22 polynucleotides or the polypeptides of the invention.

Uses of IL-21 and IL-22 Polypeptides

IL-21 and IL-22 polypeptides can be used in numerous ways. The following description should be considered exemplary and utilizes known techniques.

IL-21 and IL-22 polypeptides can be used to assay protein levels in a biological sample using antibody-based techniques. For example, protein expression in tissues can be studied with classical immunohistological methods (Jalkanen, M., et al., J. Cell. Biol. 101:976-985 (1985); Jalkanen, M., et al., J. Cell. Biol. 105:3087-3096 (1987)). Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase, and radioisotopes, such as iodine (¹²⁵I, ¹²¹I), carbon (¹⁴C), sulfur (³⁵S), tritium (³H), indium (¹¹²In), and technetium (^{99m}Tc), and fluorescent labels, such as fluorescein and rhodamine, and biotin.

In addition to assaying secreted protein levels in a biological sample, proteins can also be detected in vivo by imaging. Antibody labels or markers for in vivo imaging of protein include those detectable by X-radiography, NMR or ESR. For X-radiography, suitable labels include radioisotopes such as barium or cesium, which emit detectable radiation but are not overtly harmful to the subject. Suitable markers for NMR and ESR include those with a detectable characteristic spin, such as deuterium, which may be incorporated into the antibody by labeling of nutrients for the relevant hybridoma.

A protein-specific antibody or antibody fragment which has been labeled with an appropriate detectable imaging moiety, such as a radioisotope (for example, ¹³¹I, ¹¹²In, ^{99m}Tc), a radio-opaque substance, or a material detectable by nuclear magnetic resonance, is introduced (for example, parenterally, subcutaneously, or intraperitoneally) into the mammal. It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of ^{99m}Tc. The labeled antibody or antibody fragment will then preferentially accumulate at the location of cells which contain the specific protein. In vivo tumor imaging is described by Burchiel and colleagues (“Immunopharmacokinetics of Radiolabeled Antibodies and Their Fragments.” (Chapter 13 in Tumor Imaging: The Radiochemical Detection of Cancer, S. W. Burchiel and B. A. Rhodes, eds., Masson Publishing Inc. (1982)).

Thus, the invention provides a diagnostic method of a disorder, which involves (a) assaying the expression of IL-21 or IL-22 polypeptides in cells or body fluid of an individual; (b) comparing the level of IL-21 or IL-22 gene expression with a standard gene expression level, whereby an increase or decrease in the assayed IL-21 or IL-22 polypeptide gene expression level compared to the standard expression level is indicative of a disorder.

Moreover, IL-21 and IL-22 polypeptides can be used to treat, prevent, and/or diagnose disease. For example, patients can be administered IL-21 and IL-22 polypeptides in an effort to replace absent or decreased levels of the IL-21 and IL-22 polypeptides, respectively, (e.g., insulin), to supplement absent or decreased levels of a different polypeptide (e.g., hemoglobin S for hemoglobin B), to inhibit the activity of a polypeptide (e.g., an oncogene), to activate the activity of a polypeptide (e.g., by binding to a receptor), to reduce the activity of a membrane bound receptor by competing with it for free ligand (e.g., soluble TNF receptors used in reducing inflammation), or to bring about a desired response (e.g., blood vessel growth).

Similarly, antibodies directed to IL-21 and IL-22 polypeptides can also be used to treat, prevent, and/or diagnose disease. For example, administration of an antibody directed to an IL-21 or IL-22 polypeptide can bind and reduce overproduction of the polypeptide. Similarly, administration of an antibody can activate the polypeptide, such as by binding to a polypeptide bound to a membrane (receptor).

At the very least, the IL-21 and IL-22 polypeptides can be used as molecular weight markers on SDS-PAGE gels or on molecular sieve gel filtration columns using methods well known to those of skill in the art. IL-21 and IL-22 polypeptides can also be used to raise antibodies, which, in turn, are used to measure protein expression from a recombinant cell, as a way of assessing transformation of the host cell. Moreover, IL-21 and IL-22 polypeptides can be used to test the following biological activities.

Biological Activities of IL-21 and IL-22

IL-21 and IL-22 polynucleotides and polypeptides can be used in assays to test for one or more biological activities. If IL-21 and IL-22 polynucleotides and polypeptides do exhibit activity in a particular assay, it is likely that IL-21 and IL-22 may be involved in the diseases associated with the biological activity. Therefore, IL-21 and IL-22 could be used to treat, prevent, and/or diagnose the associated disease.

The IL-21 and IL-22 proteins of the present invention modulate IL-6 secretion from NIH-3T3 cells. An in vitro ELISA assay which quantitates the amount of IL-6 secreted from cells in response to treatment with cytokines or the soluble extracellular domains of cytokine receptors has been described (Yao, Z., et al., Immunity 3:811-821 (1995)). Briefly, the assay involves plating the target cells at a density of approximately 5×10⁶ cells/mL in a volume of 500 μL in the wells of a 24 well flat-bottomed culture plate (Costar). The cultures are then treated with various concentrations of the cytokine or the soluble extracellular domain of cytokine receptor in question. The cells are then cultured for 24 hours at 37 C. At this time, 50 μL of supernatant is removed and assayed for the quantity of IL-6 essentially as described by the manufacturer (Genzyme, Boston, Mass.). IL-6 levels are then calculated by reference to a standard curve constructed with recombinant IL-17 cytokine. Such activity is useful for determining the level of L-21- or IL-22-mediated IL-6 secretion.

IL-21 and IL-22 protein modulates immune system cell proliferation and differentiation in a dose-dependent manner in the above-described assay. Thus, “a polypeptide having IL-21 or IL-22 protein activity” includes polypeptides that also exhibit any of the same stimulatory activities in the above-described assays in a dose-dependent manner. Although the degree of dose-dependent activity need not be identical to that of the IL-21 or IL-22 proteins, preferably, “a polypeptide having IL-21 or IL-22 protein activity” will exhibit substantially similar dose-dependence in a given activity as compared to the IL-21 or IL-22 protein (i.e., the candidate polypeptide will exhibit greater activity or not more than about 25-fold less and, preferably, not more than about tenfold less activity relative to the reference IL-21 or IL-22 protein).

Lymphocyte proliferation is another in vitro assay which may be performed to determine the activity of IL-21 and IL-22. For example, Yao and colleagues (Immunity 3:811-821 (1995)) have recently described an in vitro assay for determining the effects of various cytokines and soluble cytokine receptors on the proliferation of murine leukocytes. Briefly, lymphoid organs are harvested aseptically, lymphocytes are isolated from the harvested organs, and the resulting collection of lymphoid cells are suspended in standard culture medium as described by Fanslow and coworkers (J. Immunol. 147:535-5540 (1991)). The lymphoid cell suspensions may then be divided into several different subclasses of lymphoid cells including splenic T-cells, lymph node B-cells, CD4⁺ and CD8⁺ T-cells, and mature adult thymocytes. For splenic T-cells, spleen cell suspensions (200×10⁶ cells) are incubated with CD11b mAb and class II MHC mAb for 30 min at 4 C, loaded on a T-cell purification column (Pierce, Rockford, Ill.), and the T-cells eluted according to the manufacturer's instructions. Using this method, purity of the resulting T-cell populations should be >95% CD3⁺ and <1% sIgM⁺. For purification of lymph node subsets, B-cells are removed from by adherence to tissue culture dishes previously coated with goat anti-mouse IgG (10 μg/mL). Remaining cells were then incubated with anti-CD4 or anti-CD8 for 30 min at 4 C then washed and placed on tissue culture dishes previously coated with goat anti-rat IgG (20 μg/mL). After 45 min, nonadherent cells are removed and tested for purity by flow cytometry. CD4 and surface Ig-depleted cells should be >90% TCR-ab, CD8⁺, whereas CD8 and surface Ig-depleted cells should be >95% TCR-ab, CD4⁺. Finally, to enrich for mature adult thymocytes, cells are suspended at 10⁸/mL in 10% anti-HSA and 10% low tox rabbit complement (Cedarlane, Ontario, Canada), incubated for 45 min at 37 C, and remaining viable cells isolated over Ficoll-Hypaque (Pharmacia, Piscataway, N.J.). This procedure should yield between 90 and 95% CD3^{hi} cells that are either CD4⁺8⁻ or CD4⁻8⁺.

Immune Activity

IL-21 and IL-22 polypeptides or polynucleotides may be useful in treating, preventing, and/or diagnosing deficiencies or disorders of the immune system, by activating or inhibiting the proliferation, differentiation, or mobilization (chemotaxis) of immune cells. Immune cells develop through a process called hematopoiesis, producing myeloid (platelets, red blood cells, neutrophils, and macrophages) and lymphoid (B and T lymphocytes) cells from pluripotent stem cells. The etiology of these immune deficiencies or disorders may be genetic, somatic, such as cancer or some autoimmune disorders, acquired (e.g., by chemotherapy or toxins), or infectious. Moreover, IL-21 and IL-22 polynucleotides or polypeptides can be used as a marker or detector of a particular immune system disease or disorder.

IL-21 and IL-22 polynucleotides or polypeptides may be useful in treating, preventing, and/or diagnosing deficiencies or disorders of hematopoietic cells. IL-21 and IL-22 polypeptides or polynucleotides could be used to increase differentiation and proliferation of hematopoietic cells, including the pluripotent stem cells, in an effort to treat, prevent, and/or diagnose those disorders associated with a decrease in certain (or many) types hematopoietic cells. Examples of immunologic deficiency syndromes include, but are not limited to: blood protein disorders (e.g. agammaglobulinemia, dysgammaglobulinemia), ataxia telangiectasia, common variable immunodeficiency, Digeorge Syndrome, HIV infection, HTLV-BLV infection, leukocyte adhesion deficiency syndrome, lymphopenia, phagocyte bactericidal dysfunction, severe combined immunodeficiency (SCIDs), Wiskott-Aldrich Disorder, anemia, thrombocytopenia, or hemoglobinuna.

Moreover, IL-21 and IL-22 polypeptides or polynucleotides can also be used to modulate hemostatic (the stopping of bleeding) or thrombolytic activity (clot formation). For example, by increasing hemostatic or thrombolytic activity, IL-21 and IL-22 polynucleotides or polypeptides could be used to treat, prevent, and/or diagnose blood coagulation disorders (e.g., afibrinogenemia, factor deficiencies), blood platelet disorders (e.g. thrombocytopenia), or wounds resulting from trauma, surgery, or other causes. Alternatively, IL-21 and IL-22 polynucleotides or polypeptides that can decrease hemostatic or thrombolytic activity could be used to inhibit or dissolve clotting, important in the treatment and/or prevention of heart attacks (infarction), strokes, or scarring.

IL-21 and IL-22 polynucleotides or polypeptides may also be useful in treating, preventing, detecting, and/or diagnosing autoimmune disorders. Many autoimmune disorders result from inappropriate recognition of self as foreign material by immune cells. This inappropriate recognition results in an immune response leading to the destruction of the host tissue. Therefore, the administration of IL-21 and IL-22 polypeptides or polynucleotides that can inhibit an immune response, particularly the proliferation, differentiation, or chemotaxis of T-cells, may be an effective therapy in preventing autoimmune disorders.

Examples of autoimmune disorders that can be treated, prevented, diagnosed, and/or detected by IL-21 and IL-22 include, but are not limited to: Addison's Disease, hemolytic anemia, antiphospholipid syndrome, rheumatoid arthritis, dermatitis, allergic encephalomyelitis, glomerulonephritis, Goodpastures Syndrome, Grave's Disease, Multiple Sclerosis, Myasthenia Gravis, Neuritis, Ophthalmia, Bullous Pemphigoid, Pemphigns, Polyendocrinopathies, Purpura, Reiter's Disease, Stiff-Man Syndrome, Autoimmune Thyroiditis, Systemic Lupus Erythematosus, Autoimmune Pulmonary Inflammation, Guillain-Barre Syndrome, insulin dependent diabetes mellitis, and autoimmune inflammatory eye disease.

Similarly, allergic reactions and conditions, such as asthma (particularly allergic asthma) or other respiratory problems, may also be treated, prevented, and/or diagnosed by IL-21 and IL-22 polypeptides or polynucleotides. Moreover, IL-21 and IL-22 can be used to treat, prevent, and/or diagnose anaphylaxis, hypersensitivity to an antigenic molecule, or blood group incompatibility.

IL-21 and IL-22 polynucleotides or polypeptides may also be used to treat, prevent, and/or diagnose organ rejection or graft-versus-host disease (GVHD). Organ rejection occurs by host immune cell destruction of the transplanted tissue through an immune response. Similarly, an immune response is also involved in GVHD, but, in this case, the foreign transplanted immune cells destroy the host tissues. The administration of IL-21 and IL-22 polypeptides or polynucleotides that inhibits an immune response, particularly the proliferation, differentiation, or chemotaxis of T-cells, may be an effective therapy in preventing organ rejection or GVHD.

Similarly, IL-21 and IL-22 polypeptides or polynucleotides may also be used to modulate inflammation. For example, IL-21 and IL-22 polypeptides or polynucleotides may inhibit the proliferation and differentiation of cells involved in an inflammatory response. These molecules can be used to treat, prevent, and/or diagnose inflammatory conditions, both chronic and acute conditions, including inflammation associated with infection (e.g., septic shock, sepsis, or systemic inflammatory response syndrome (SIRS)), ischemia-reperfusion injury, endotoxin lethality, arthritis, complement-mediated hyperacute rejection, nephritis, cytokine or chemokine induced lung injury, inflammatory bowel disease, Crohn's disease, or resulting from over production of cytokines (e.g., TNF or IL-1.)

Hyperproliferative Disorders

IL-21 and IL-22 polypeptides or polynucleotides can be used to treat, prevent, detect, and/or diagnose hyperproliferative disorders, including neoplasms. IL-21 and IL-22 polypeptides or polynucleotides may inhibit the proliferation of the disorder through direct or indirect interactions. Alternatively, IL-21 and IL-22 polypeptides or polynucleotides may proliferate other cells which can inhibit the hyperproliferative disorder.

For example, by increasing an immune response, particularly increasing antigenic qualities of the hyperproliferative disorder or by proliferating, differentiating, or mobilizing T-cells, hyperproliferative disorders can be treated, prevented, and/or diagnosed. This immune response may be increased by either enhancing an existing immune response, or by initiating a new immune response. Alternatively, decreasing an immune response may also be a method of treating, preventing, and/or diagnosing hyperproliferative disorders, such as a chemotherapeutic agent.

Examples of hyperproliferative disorders that can be treated, prevented, and/or diagnosed or detected by IL-21 and IL-22 polynucleotides or polypeptides include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvic, skin, soft tissue, spleen, thoracic, and urogenital.

Similarly, other hyperproliferative disorders can also be treated, prevented, detected, and/or diagnosed by IL-21 and IL-22 polynucleotides or polypeptides. Examples of such hyperproliferative disorders include, but are not limited to: hypergammaglobulinemia, lymphoproliferative disorders, paraproteinemias, purpura, sarcoidosis, Sezary Syndrome, Waldenstron's Macroglobulinemia, Gaucher's Disease, histiocytosis, and any other hyperproliferative disease, besides neoplasia, located in an organ system listed above.

Infectious Disease

IL-21 and IL-22 polypeptides or polynucleotides can be used to treat, prevent, detect, and/or diagnose infectious agents. For example, by increasing the immune response, particularly increasing the proliferation and differentiation of B and/or T cells, infectious diseases may be treated, prevented, and/or diagnosed. The immune response may be increased by either enhancing an existing immune response, or by initiating a new immune response. Alternatively, IL-21 and IL-22 polypeptides or polynucleotides may also directly inhibit the infectious agent, without necessarily eliciting an immune response.

Viruses are one example of an infectious agent that can cause disease or symptoms that can be treated, prevented, detected, and/or diagnosed by IL-21 and IL-22 polynucleotides or polypeptides. Examples of viruses, include, but are not limited to the following DNA and RNA viral families: Arbovirus, Adenoviridae, Arenaviridae, Arterivirus, Bimaviridae, Bunyaviridae, Caliciviridae, Circoviridae, Coronaviridae, Flaviviridae, Hepadnaviridae (Hepatitis), Herpesviridae (such as, Cytomegalovirus, Herpes Simplex, Herpes Zoster), Mononegavirus (e.g., Paramyxoviridae, Morbillivirus, Rhabdoviridae), Orthomyxoviridae (e.g., Influenza), Papovaviridae, Parvoviridae, Picomaviridae, Poxviridae (such as Smallpox or Vaccinia), Reoviridae (e.g., Rotavirus), Retroviridae (HTLV-I, HTLV-II, Lentivirus), and Togaviridae (e.g., Rubivirus). Viruses falling within these families can cause a variety of diseases or symptoms, including, but not limited to: arthritis, bronchiollitis, encephalitis, eye infections (e.g., conjunctivitis, keratitis), chronic fatigue syndrome, hepatitis (A, B, C, E, Chronic Active, Delta), meningitis, opportunistic infections (e.g., AIDS), pneumonia, Burkitt's Lymphoma, chickenpox, hemorrhagic fever, Measles, Mumps, Parainfluenza, Rabies, the common cold, Polio, leukemia, Rubella, sexually transmitted diseases, skin diseases (e.g., Kaposis, warts), and viremia. IL-21 and IL-22 polypeptides or polynucleotides can be used to treat, prevent, detect, and/or diagnose any of these symptoms or diseases.

Similarly, bacterial or fungal agents that can cause disease or symptoms and that can be treated, prevented, detected, and/or diagnosed by IL-21 and IL-22 polynucleotides or polypeptides include, but not limited to, the following Gram-Negative and Gram-positive bacterial families and fungi: Actinomycetales (e.g., Corynebacterium, Mycobacterium, Norcardia), Aspergillosis, Bacillaceae (e.g., Anthrax, Clostridium), Bacteroidaceae, Blastomycosis, Bordetella, Borrelia, Brucellosis, Candidiasis, Campylobacter, Coccidioidomycosis, Cryptococcosis, Dermatocycoses, Enterobacteriaceae (Klebsiella, Salmonella, Serratia, Yersinia), Erysipelothrix, Helicobacter, Legionellosis, Leptospirosis, Listeria, Mycoplasmatales, Neisseriaceae (e.g., Acinetobacter, Gonorrhea, Menigococcal), Pasteurellacea Infections (e.g., Actinobacillus, Heamophilus, Pasteurella), Pseudomonas, Rickettsiaceae, Chlamydiaceae, Syphilis, and Staphylococcal. These bacterial or fungal families can cause the following diseases or symptoms, including, but not limited to: bacteremia, endocarditis, eye infections (conjunctivitis, tuberculosis, uveitis), gingivitis, opportunistic infections (e.g., AIDS related infections), paronychia, prosthesis-related infections, Reiter's Disease, respiratory tract infections, such as Whooping Cough or Empyema, sepsis, Lyme Disease, Cat-Scratch Disease, Dysentery, Paratyphoid Fever, food poisoning, Typhoid, pneumonia, Gonorrhea, meningitis, Chlamydia, Syphilis, Diphtheria, Leprosy, Paratuberculosis, Tuberculosis, Lupus, Botulism, gangrene, tetanus, impetigo, Rheumatic Fever, Scarlet Fever, sexually transmitted diseases, skin diseases (e.g., cellulitis, dermatocycoses), toxemia, urinary tract infections, wound infections. IL-21 and IL-22 polypeptides or polynucleotides can be used to treat, prevent, detect, and/or diagnose any of these symptoms or diseases.

Moreover, parasitic agents causing disease or symptoms that can be treated, prevented, detected, and/or diagnosed by IL-21 polynucleotides or polypeptides include, but not limited to, the following families: Amebiasis, Babesiosis, Coccidiosis, Cryptosporidiosis, Dientamoebiasis, Dourine, Ectoparasitic, Giardiasis, Helminthiasis, Leishmaniasis, Theileriasis, Toxoplasmosis, Trypanosomiasis, and Trichomonas. These parasites can cause a variety of diseases or symptoms, including, but not limited to: Scabies, Trombiculiasis, eye infections, intestinal disease (e.g., dysentery, giardiasis), liver disease, lung disease, opportunistic infections (e.g., AIDS related), Malaria, pregnancy complications, and toxoplasmosis. IL-21 and IL-22 polypeptides or polynucleotides can be used to treat, prevent, detect, and/or diagnose any of these symptoms or diseases.

Preferably, treatment, prevention, detection, and/or diagnosis using IL-21 and IL-22 polypeptides or polynucleotides could either be by administering an effective amount of IL-21 or IL-22 polypeptide to the patient, or by removing cells from the patient, supplying the cells with IL-21 and L-22 polynucleotide, and returning the engineered cells to the patient (ex vivo therapy). Moreover, the IL-21 and IL-22 polypeptide or polynucleotide can be used as an antigen in a vaccine to raise an immune response against infectious disease.

Regeneration

IL-21 and IL-22 polynucleotides or polypeptides can be used to differentiate, proliferate, and attract cells, leading to the regeneration of tissues (see, Science 276:59-87 (1997)). The regeneration of tissues could be used to repair, replace, or protect tissue damaged by congenital defects, trauma (wounds, burns, incisions, or ulcers), age, disease (e.g. osteoporosis, osteocarthritis, periodontal disease, liver failure), surgery, including cosmetic plastic surgery, fibrosis, reperfusion injury, or systemic cytokine damage.

Tissues that could be regenerated using the present invention include organs (e.g., pancreas, liver, intestine, kidney, skin, endothelium), muscle (smooth, skeletal or cardiac), vascular (including vascular endothelium), nervous, hematopoietic, and skeletal (bone, cartilage, tendon, and ligament) tissue. Preferably, regeneration occurs without or decreased scarring. Regeneration also may include angiogenesis.

Moreover, IL-21 and IL-22 polynucleotides or polypeptides may increase regeneration of tissues difficult to heal. For example, increased tendon/ligament regeneration would quicken recovery time after damage. IL-21 and IL-22 polynucleotides or polypeptides of the present invention could also be used prophylactically in an effort to avoid damage. Specific diseases that could be treated, prevented, detected, and/or diagnosed include of tendinitis, carpal tunnel syndrome, and other tendon or ligament defects. A further example of tissue regeneration of non-healing wounds includes pressure ulcers, ulcers associated with vascular insufficiency, surgical, and traumatic wounds.

Similarly, nerve and brain tissue could also be regenerated by using IL-21 and IL-22 polynucleotides or polypeptides to proliferate and differentiate nerve cells. Diseases that could be treated, prevented, detected, and/or diagnosed using this method include central and peripheral nervous system diseases, neuropathies, or mechanical and traumatic disorders (e.g., spinal cord disorders, head trauma, cerebrovascular disease, and stoke). Specifically, diseases associated with peripheral nerve injuries, peripheral neuropathy (e.g., resulting from chemotherapy or other medical therapies), localized neuropathies, and central nervous system diseases (e.g., Alzheimer's disease, Parkinson's disease, Huntington's disease, amyotrophic lateral sclerosis, and Shy-Drager syndrome), could all be treated, prevented, detected, and/or diagnosed using the IL-21 and IL-22 polynucleotides or polypeptides.

Chemotaxis

IL-21 and IL-22 polynucleotides or polypeptides may have chemotaxis activity. A chemotaxic molecule attracts or mobilizes cells (e.g., monocytes, fibroblasts, neutrophils, T-cells, mast cells, eosinophils, epithelial and/or endothelial cells) to a particular site in the body, such as inflammation, infection, or site of hyperproliferation. The mobilized cells can then fight off and/or heal the particular trauma or abnormality.

IL-21 and IL-22 polynucleotides or polypeptides may increase chemotaxic activity of particular cells. These chemotactic molecules can then be used to treat, prevent, detect, and/or diagnose inflammation, infection, hyperproliferative disorders, or any immune system disorder by increasing the number of cells targeted to a particular location in the body. For example, chemotaxic molecules can be used to treat, prevent, detect, and/or diagnose wounds and other trauma to tissues by attracting immune cells to the injured location. As a chemotactic molecule, IL-21 and IL-22 could also attract fibroblasts, which can be used to treat, prevent, detect, and/or diagnose wounds.

It is also contemplated that IL-21 and IL-22 polynucleotides or polypeptides may inhibit chemotactic activity. These molecules could also be used to treat, prevent, detect, and/or diagnose disorders. Thus, IL-21 and IL-22 polynucleotides or polypeptides could be used as an inhibitor of chemotaxis.

Binding Activity

IL-21 and IL-22 polypeptides may be used to screen for molecules that bind to IL-21 or IL-22 or for molecules to which IL-21 or IL-22 bind. The binding of IL-21 and IL-22 and the molecule may activate (agonist), increase, inhibit (antagonist), or decrease activity of the IL-21 and IL-22 or the molecule bound. Examples of such molecules include antibodies, oligonucleotides, proteins (e.g., receptors),or small molecules.

Preferably, the molecule is closely related to the natural ligand of IL-21 or IL-22, e.g., a fragment of the ligand, or a natural substrate, a ligand, a structural or functional mimetic (see, Coligan, et al., Current Protocols in Immunology 1(2):Chapter 5 (1991)). Similarly, the molecule can be closely related to the natural receptor to which IL-21 and IL-22 bind, or at least, a fragment of the receptor capable of being bound by IL-21 or IL-22 (e.g., active site). In either case, the molecule can be rationally designed using known techniques.

Preferably, the screening for these molecules involves producing appropriate cells which express IL-21 and IL-22, either as a secreted protein or on the cell membrane. Preferred cells include cells from mammals, yeast, Drosophila, or E. coli. Cells expressing IL-21 and IL-22 (or cell membrane containing the expressed polypeptide) are then preferably contacted with a test compound potentially containing the molecule to observe binding, stimulation, or inhibition of activity of either IL-21 and IL-22 or the molecule.

The assay may simply test binding of a candidate compound to IL-21 or IL-22, wherein binding is detected by a label, or in an assay involving competition with a labeled competitor. Further, the assay may test whether the candidate compound results in a signal generated by binding to IL-21 or IL-22.

Alternatively, the assay can be carried out using cell-free preparations, polypeptide/molecule affixed to a solid support, chemical libraries, or natural product mixtures. The assay may also simply comprise the steps of mixing a candidate compound with a solution containing IL-21 or IL-22, measuring IL-21/molecule or IL-22/molecule activity or binding, respectively, and comparing the IL-21/molecule or IL-22/molecule activity or binding to a standard.

Preferably, an ELISA assay can measure IL-21 and IL-22 levels or activities in a sample (e.g., biological sample) using a monoclonal or polyclonal antibody. The antibody can measure IL-21 and IL-22 levels or activities by either binding, directly or indirectly, to IL-21 or IL-22 or by competing with IL-21 or IL-22 for a substrate.

All of these above assays can be used as diagnostic or prognostic markers. The molecules discovered using these assays can be used to treat, prevent, detect, and/or diagnose disease or to bring about a particular result in a patient (e.g., blood vessel growth) by activating or inhibiting IL-21 or IL-22. Moreover, the assays can discover agents which may inhibit or enhance the production of IL-21 and IL-22 from suitably manipulated cells or tissues.

Therefore, the invention includes a method of identifying compounds which bind to IL-21 and IL-22 comprising the steps of: (a) incubating a candidate binding compound with IL-21 or IL-22; and (b) determining if binding has occurred. Moreover, the invention includes a method of identifying agonists/antagonists comprising the steps of: (a) incubating a candidate compound with IL-21 or IL-22, (b) assaying a biological activity, and (b) determining if a biological activity of IL-21 or IL-22, respectively, has been altered.

Other Activities

IL-21 and IL-22 polypeptides or polynucleotides may also increase or decrease the differentiation or proliferation of embryonic stem cells, besides, as discussed above, hematopoietic lineage.

IL-21 and IL-22 polypeptides or polynucleotides may also be used to modulate mammalian characteristics, such as body height, weight, hair color, eye color, skin, percentage of adipose tissue, pigmentation, size, and shape (e.g., cosmetic surgery). Similarly, IL-21 and IL-22 polypeptides or polynucleotides may be used to modulate mammalian metabolism affecting catabolism, anabolism, processing, utilization, and storage of energy.

IL-21 and IL-22 polypeptides or polynucleotides may be used to change a mammal's mental state or physical state by influencing biorhythms, caricadic rhythms, circadian rhythms, depression (including depressive disorders), tendency for violence, tolerance for pain (in preferred embodiments, analyzed by a rat hyperalgesic footpad pain assay), reproductive capabilities (preferably by Activin or Inhibin-like activity), hormonal or endocrine levels, appetite, libido, memory, stress, or other cognitive qualities.

IL-21 and IL-22 polypeptides or polynucleotides may also be used as a food additive or preservative, such as to increase or decrease storage capabilities, fat content, lipid, protein, carbohydrate, vitamins, minerals, cofactors or other nutritional components.

Having generally described the invention, the same will be more readily understood by reference to the following examples, which are provided by way of illustration and are not intended as limiting.

### EXAMPLES

In the case where the full-length IL-21 and the partial IL-22 are not specifically mentioned, specific details are provided in the following examples only for the partial-length IL-21 molecules of the present invention. However, the examples can also be easily performed for the full-length IL-21 and the full-length or partial-length IL-22 molecules of the present invention by using the details provided for the partial IL-21 and substituting appropriate nucleotides or amino acid residues of the full-length IL-21, the full-length or partial-length IL-22, and/or any deletion mutations or other variants of either IL-21 or IL-22, for example, in the design of suitable PCR primers, and the like. The use or applicability of another IL-21 or IL-22 in place of the IL-21 exemplified below is thus contemplated in each of the following examples. When provided with the nucleotide and amino acid sequences of IL-21 (SEQ ID NO: 1, SEQ ID NO: 2, SEQ ID NO: 28, and SEQ ID NO: 29) and IL-22 (SEQ ID NO: 3, SEQ ID NO: 4, SEQ ID NO: 30, and SEQ ID NO: 31) of the present invention, one of ordinary skill in the art could easily perform the following examples with the intent of isolating or further characterizing or manipulating another IL-21 or IL-22 in place of the IL-21 shown in the Examples below.

### Example 1: Isolation of the IL-21 and IL-22 cDNA Clones From the Deposited Samples.

The cDNAs encoding the partial IL-21 and IL-22 molecules are each inserted into the Eco RI and Xho I restriction sites of the multiple cloning site of pBluescript. pbluescript contains an ampicillin resistance gene and may be transformed into E. coli strain DH10B, available from Life Technologies (see, for instance, Gruber, C. E., et al., Focus 15:59 (1993)).

Two approaches can be used to isolate IL-21 from the deposited sample. First, a specific polynucleotide of SEQ ID NO: 1 with 30-40 nucleotides is synthesized using an Applied Biosystems DNA synthesizer according to the sequence reported. The oligonucleotide is labeled, for instance, with ³²P-gamma-ATP using T4 polynucleotide kinase and purified according to routine methods (e.g., Maniatis, et al., Molecular Cloning: A Laboratory Manual, Cold Spring Harbor Press, Cold Spring, N.Y. (1982)). The plasmid mixture is transformed into a suitable host (such as XL-1 Blue (Stratagene)) using techniques known to those of skill in the art, such as those provided by the vector supplier or in related publications or patents. The transformants are plated on 1.5% agar plates (containing the appropriate selection agent, e.g., ampicillin) to a density of about 150 transformants (colonies) per plate. These plates are screened using Nylon membranes according to routine methods for bacterial colony screening (e.g., Sambrook, et al., Molecular Cloning: A Laboratory Manual, 2nd Edit., (1989), Cold Spring Harbor Laboratory Press, pages 1.93 to 1.104), or other techniques known to those of skill in the art.

Alternatively, two primers of 17-20 nucleotides derived from both ends of the SEQ ID NO: 1 (i.e., within the region of SEQ ID NO: 1 bounded by the 5′ and 3′ nucleotides of the clone) are synthesized and used to amplify the IL-21 cDNA using the deposited cDNA plasmid as a template. The polymerase chain reaction is carried out under routine conditions, for instance, in 25 microliters of reaction mixture with 0.5 micrograms of the above cDNA template. A convenient reaction mixture is 1.5-5 mM MgCl₂, 0.01% (w/v) gelatin, 20 micromolar each of dATP, dCTP, dGTP, dTTP, 25 pmol of each primer and 0.25 Unit of Taq polymerase. Thirty five cycles of PCR (denaturation at 94 C for 1 min; annealing at 55 C for 1 min; elongation at 72 C for 1 min) are performed with a Perkin-Elmer Cetus automated thermal cycler. The amplified product is analyzed by agarose gel electrophoresis and the DNA band with expected molecular weight is excised and purified. The PCR product is verified to be the selected sequence by subdloning and sequencing the DNA product.

Several methods are available for the identification of the 5′ or 3′ non-coding portions of the IL-21 gene which may not be present in the deposited clone. These methods include, but are not limited to, filter probing, clone enrichment using specific probes, and protocols similar or identical to 5′ and 3′ RACE protocols which are well known in the art. For instance, a method similar to 5′ RACE is available for generating the missing 5′ end of a desired full-length transcript (Fromont-Racine, et al., Nucl. Acids Res. 21(7):1683-1684 (1993)).

Briefly, a specific RNA oligonucleotide is ligated to the 5′ ends of a population of RNA presumably containing full-length gene RNA transcripts. A primer set containing a primer specific to the ligated RNA oligonucleotide and a primer specific to a known sequence of the IL-21 gene of interest is used to PCR amplify the 5′ portion of the IL-21 full-length gene. This amplified product may then be sequenced and used to generate the full length gene.

This above method starts with total RNA isolated from the desired source, although poly-A+ RNA can be used. The RNA preparation can then be treated with a phosphatase, if necessary, to eliminate 5′ phosphate groups on degraded or damaged RNA which may interfere with the later RNA ligase step. The phosphatase should then be inactivated and the RNA treated with tobacco acid pyrophosphatase in order to remove the cap structure present at the 5′ ends of messenger RNA. This reaction leaves a 5′ phosphate group at the 5′ end of the cap cleaved RNA which can then be ligated to an RNA oligonucleotide using T4 RNA ligase.

This modified RNA preparation is used as a template for first strand cDNA synthesis using a gene specific oligonucleotide. The first strand synthesis reaction is used as a template for PCR amplification of the desired 5′ end using a primer specific to the ligated RNA oligonucleotide and a primer specific to the known sequence of the gene of interest. The resultant product is then sequenced and analyzed to confirm that the 5′ end sequence belongs to the IL-21 gene.

### Example 2: Isolation of IL-21 Genomic Clones.

A human genomic P1 library (Genomic Systems, Inc.) is screened by PCR using primers selected for the cDNA sequence corresponding to SEQ ID NO: 1., according to the method described in Example 1 (see also, Sambrook, et al., supra).

### Example 3: Tissue Distribution of IL-21.

Tissue distribution of mRNA expression of IL-21 is determined using protocols for Northern blot analysis, described by, among others, Sambrook and colleagues (supra). For example, an IL-21 probe produced by the method described in Example 1 is labeled with ³²P using the rediprime™ DNA labeling system (Amersham Life Science), according to manufacturer's instructions. After labeling, the probe is purified using a CHROMA SPIN-100™ column (Clontech Laboratories, Inc.), according to manufacturer's protocol number PT1200-1. The purified labeled probe is then used to examine various human tissues for mRNA expression.

Multiple Tissue Northern (MTN) blots containing various human tissues (H) or human immune system (IM) tissues (Clontech) are examined with the labeled probe using ExpressHyb™ hybridization solution (Clontech) according to manufacturers protocol number PT1190-1. Following hybridization and washing, the blots are mounted and exposed to film at −70 C overnight, and the films developed according to standard procedures.

Using essentially the above-prescribed protocol, Northern blot analyses were performed to determine the expression pattern of IL-21 and IL-22. In the case of IL-21, very light signals of 1.8 and 3.0 kb were detected in skeletal muscle, and signals of indeterminate sizes were detected in fetal lung and fetal kidney. In the case of IL-22, a major message of 2.4 kb was detected in conjunction with a minor band in all brain tissues examined, and was also detected to a lesser extent in skeletal muscle, heart, testis, spinal cord, bone marrow, small intestine, kidney, and lung. A minor band of 4.4 kb was also detected in skeletal muscle.

### Example 4: Chromosomal Mapping of IL-21.

An oligonucleotide primer set is designed according to the sequence at the 5′ end of SEQ ID NO: 1. This primer preferably spans about 100 nucleotides. This primer set is then used in a polymerase chain reaction under the following set of conditions : 30 seconds, 95 C; 1 minute, 56 C; 1 minute, 70 C. This cycle is repeated 32 times followed by one 5 minute cycle at 70 C. Human, mouse, and hamster DNA is used as template in addition to a somatic cell hybrid panel containing individual chromosomes or chromosome fragments (Bios, Inc). The reactions are analyzed on either 8% polyacrylamide gels or 3.5 % agarose gels. Chromosome mapping is determined by the presence of an approximately 100 bp PCR fragment in the particular somatic cell hybrid.

### Example 5: Bacterial Expression of IL-21.

An IL-21 polynucleotide encoding an IL-21 polypeptide of the invention is amplified using PCR oligonucleotide primers corresponding to the 5′ and 3′ ends of the DNA sequence, as outlined in Example 1, to synthesize insertion fragments. The primers used to amplify the cDNA insert should preferably contain restriction sites, such as Bam HI and Hin dIII, at the 5′ end of the primers in order to clone the amplified product into the expression vector. For example, Bam HI and Hin dIII correspond to the restriction enzyme sites on the bacterial expression vector pQE-9 (Qiagen Inc., Chatsworth, Calif.). This plasmid vector encodes antibiotic resistance (Amp^{R}), a bacterial origin of replication (ori), an IPTG-regulatable promoter/operator (P/O), a ribosome binding site (RBS), a 6-histidine tag (6-His), and restriction enzyme cloning sites.

Specifically, to clone the mature domain of the IL-21 protein in a bacterial vector, the 5′ primer has the sequence 5′-GAT CGC GGA TCC GAC ACG GAT GAG GAC CGC TAT CCA CAG AAG CTG-3′ (SEQ ID NO: 9) containing the underlined Bam HI restriction site followed several nucleotides of the amino terminal coding sequence of the mature IL-21 sequence in SEQ ID NO: 1. One of ordinary skill in the art would appreciate, of course, that the point in the protein coding sequence where the 5′ primer begins may be varied to amplify a DNA segment encoding any desired portion of the complete IL-21 protein shorter or longer than the mature form of the protein. The 3′ primer has the sequence 5′-CCC AAG CTT TCA CAC TGA ACG GGG CAG CAC GCA GGT GCA GC-3′ (SEQ ID NO: 10) containing the underlined Hin dIII restriction site followed by a number nucleotides complementary to the 3′ end of the coding sequence of the IL-21 DNA sequence of SEQ ID NO: 1.

The pQE-9 vector is digested with Bam HI and Hin dIII and the amplified fragment is ligated into the pQE-9 vector maintaining the reading frame initiated at the bacterial RBS. The ligation mixture is then used to transform the E. coli strain M15/rep4 (Qiagen, Inc.) which contains multiple copies of the plasmid pREP4, which expresses the lacI repressor and also confers kanamycin resistance (Kan^{R}). Transformants are identified by their ability to grow on LB plates and colonies are selected which are resistant to both ampicillin and kanamycin. Plasmid DNA is isolated and confirmed by restriction analysis.

Clones containing the desired constructs are grown overnight (O/N) in liquid culture in LB media supplemented with both Amp (100 μg/ml) and Kan (25 μg/ml). The O/N culture is used to inoculate a large culture at a ratio of 1:100 to 1:250. The cells are grown to an optical density 600 (O.D.₆₀₀) of between 0.4 and 0.6. IPTG (Isopropyl-B-D-thiogalacto pyranoside) is then added to a final concentration of 1 mM. IPTG induces by inactivating the lacd repressor, clearing the promoter/operator leading to increased gene expression.

Cells are grown for an additional 3 to 4 hours. Cells are then harvested by centrifugation (20 mins at 6000 X g). The cell pellet is solubilized in the chaotropic agent 6M Guanidine-HCl by stirring for 3-4 hours at 4 C. The cell debris is removed by centrifugation, and the supernatant containing the polypeptide is loaded onto a nickel-nitrilo-tri-acetic acid (“Ni-NTA”) affinity resin column (QIAGEN, Inc., supra). Proteins with a 6 x His tag bind to the Ni-NTA resin with high affinity and can be purified in a simple one-step procedure (for details see: The QIAexpressionist (1995) QIAGEN, Inc., supra).

Briefly, the supernatant is loaded onto the column in 6M guanidine-HCl, pH 8, the column is first washed with 10 volumes of 6M guanidine-HCl, pH 8, then washed with 10 volumes of 6M guanidine-HCl pH 6, and finally the polypeptide is eluted with 6M guanidine-HCl, pH 5.

The purified IL-21 protein is then renatured by dialyzing it against phosphate-buffered saline (PBS) or 50 mM Na-acetate, pH 6 buffer plus 200 mM NaCl. Alternatively, the IL-21 protein can be successfully refolded while immobilized on the Ni-NTA column. The recommended conditions are as follows: renature using a linear 6M-1M urea gradient in 500 mM NaCl, 20% glycerol, 20 mM Tris/HCl pH 7.4, containing protease inhibitors. The renaturation should be performed over a period of 1.5 hours or more. After renaturation the proteins are eluted by the addition of 250 mM immidazole. Immidazole is removed by a final dialyzing step against PBS or 50 mM sodium acetate pH 6 buffer plus 200 mM NaCl. The purified IL-21 protein is stored at 4° C. or frozen at −80 C.

In addition to the above expression vector, the present invention further includes an expression vector comprising phage operator and promoter elements operatively linked to an IL-21 polynucleotide, called pHE4a (ATCC Accession Number 209645, deposited Feb. 25, 1998). This vector contains: (1) a neomycin phosphotransferase gene as a selection marker, (2) an E. coli origin of replication, (3) a T5 phage promoter sequence, (4) two lac operator sequences, (5) a Shine-Delgamo sequence, and (6) the lactose operon repressor gene (lacIq). The origin of replication (oriC) is derived from pUC19 (LTI, Gaithersburg, Md.). The promoter sequence and operator sequences are made synthetically.

DNA can be inserted into the pHEa by restricting the vector with Nde I and Xba I, Bam HI, Xho I, or Asp 718, running the restricted product on a gel, and isolating the larger fragment (the stuffer fragment should be about 310 base pairs). The DNA insert is generated according to the PCR protocol described in Example 1, using PCR primers which encode restriction sites for Nde I (5′ primer) and Nde I and Xba I, Bam HI, Xho I, or Asp 718 (3′ primer). The PCR insert is gel purified and restricted with compatible enzymes. The insert and vector are ligated according to standard protocols.

The engineered vector could easily be substituted in the above protocol to express protein in a bacterial system.

### Example 6: Purification of IL-21 Polypeptide from an Inclusion Body.

The following alternative method can be used to purify IL-21 polypeptide expressed in E coli when it is present in the form of inclusion bodies. Unless otherwise specified, all of the following steps are conducted at 4-10 C.

Upon completion of the production phase of the E. coli fermentation, the cell culture is cooled to 4-10 C and the cells harvested by continuous centrifugation at 15,000 rpm (Heraeus Sepatech). On the basis of the expected yield of protein per unit weight of cell paste and the amount of purified protein required, an appropriate amount of cell paste, by weight, is suspended in a buffer solution containing 100 mM Tris, 50 mM EDTA, pH 7.4. The cells are dispersed to a homogeneous suspension using a high shear mixer.

The cells are then lysed by passing the solution through a microfluidizer (Microfuidics, Corp. or APV Gaulin, Inc.) twice at 4000-6000 psi. The homogenate is then mixed with NaCl solution to a final concentration of 0.5M NaCl, followed by centrifugation at 7000 xg for 15 min. The resultant pellet is washed again using 0.5M NaCl, 100 mM Tris, 50 mM EDTA, pH 7.4.

The resulting washed inclusion bodies are solubilized with 1.5M guanidine hydrochloride (GuHCl) for 2-4 hours. After 7000 x g centrifugation for 15 min., the pellet is discarded and the polypeptide containing supernatant is incubated at 4 C overnight to allow further GuHCl extraction.

Following high speed centrifugation (30,000 x g) to remove insoluble particles, the GuHCl solubilized protein is refolded by quickly mixing the GuHCl extract with 20 volumes of buffer containing 50 mM sodium, pH 4.5, 150 mM NaCl, 2 mM EDTA by vigorous stirring. The refolded diluted protein solution is kept at 4 C without mixing for 12 hours prior to further purification steps.

To clarify the refolded polypeptide solution, a previously prepared tangential filtration unit equipped with 0.16 micrometer membrane filter with appropriate surface area (e.g., Filtron), equilibrated with 40 mM sodium acetate, pH 6.0 is employed. The filtered sample is loaded onto a cation exchange resin (e.g., Poros HS-50, Perseptive Biosystems). The column is washed with 40 mM sodium acetate, pH 6.0 and eluted with 250 mM, 500 mM, 1000 mM, and 1500 mM NaCl in the same buffer, in a stepwise manner. The absorbance at 280 nm of the effluent is continuously monitored. Fractions are collected and further analyzed by SDS-PAGE.

Fractions containing the IL-21 polypeptide are then pooled and mixed with 4 volumes of water. The diluted sample is then loaded onto a previously prepared set of tandem columns of strong anion (Poros HQ-50, Perseptive Biosystems) and weak anion (Poros CM-20, Perseptive Biosystems) exchange resins. The columns are equilibrated with 40 mM sodium acetate, pH 6.0. Both columns are washed with 40 mM sodium acetate, pH 6.0, 200 mM NaCl. The CM-20 column is then eluted using a 10 column volume linear gradient ranging from 0.2M NaCl, 50 mM sodium acetate, pH 6.0 to 1.0M NaCl, 50 mM sodium acetate, pH 6.5. Fractions are collected under constant A₂₈₀ monitoring of the effluent. Fractions containing the polypeptide (determined, for instance, by 16% SDS-PAGE) are then pooled.

The resultant IL-21 polypeptide should exhibit greater than 95% purity after the above refolding and purification steps. No major contaminant bands should be observed from Commassie blue stained 16% SDS-PAGE gel when 5 micrograms of purified protein is loaded. The purified IL-21 protein can also be tested for endotoxin/LPS contamination, and typically the LPS content is less than 0.1 ng/ml according to LAL assays.

### Example 7: Cloning and Expression of IL-21 in a Baculovirus Expression System.

In this example, the plasmid shuttle vector pA2 is used to insert IL-21 polynucleotide into a baculovirus to express IL-21. This expression vector contains the strong polyhedrin promoter of the Autographa californica nuclear polyhedrosis virus (AcMNPV) followed by convenient restriction sites such as Bam HI, Xba I and Asp 718. The polyadenylation site of the simian virus 40 (“SV40”) is used for efficient polyadenylation. For easy selection of recombinant virus, the plasmid contains the beta-galactosidase gene from E. coli under control of a weak Drosophila promoter in the same orientation, followed by the polyadenylation signal of the polyhedrin gene. The inserted genes are flanked on both sides by viral sequences for cell-mediated homologous recombination with wild-type viral DNA to generate a viable virus that express the cloned IL-21 polynucleotide.

Many other baculovirus vectors can be used in place of the vector above, such as pAc373, pVL941, and pAcIM1, as one skilled in the art would readily appreciate, as long as the construct provides appropriately located signals for transcription, translation, secretion and the like, including a signal peptide and an in-frame AUG as required. Such vectors are described, for instance, by Luckow and colleagues (Virology 170:31-39 (1989)).

Specifically, the IL-21 cDNA sequence contained in the deposited clone, including the AUG initiation codon and any naturally associated leader sequence, is amplified using the PCR protocol described in Example 1. If the naturally occurring signal sequence is used to produce the secreted protein, the pA2 vector does not need a second signal peptide. However, since the predicted naturally occurring signal peptides of IL-21 and IL-22 are not known, the vector can be modified (now designated pA2GP) to include a baculovirus leader sequence, using the standard methods described by Summers and coworkers (“A Manual of Methods for Baculovirus Vectors and Insect Cell Culture Procedures,” Texas Agricultural Experimental Station Bulletin No. 1555 (1987)).

More specifically, the cDNA sequence encoding the full-length IL-21 protein in the deposited clone is amplified using PCR oligonucleotide primers corresponding to the 5′ and 3′ sequences of the gene. The 5′ primer has the sequence 5′-CGC CGC GGA TCC GCC ATC CGC ACG AGT GGA CAC GG-3′ (SEQ ID NO: 11) containing the Bam HI restriction enzyme site, an efficient signal for initiation of translation in eukaryotic cells (shown in the primer sequence in italics; Kozak, M., J. Mol. Biol. 196:947-950 (1987)), a “C” residue to preserve the reading frame, and 16 nucleotides of the sequence of the complete IL-21 protein shown in FIG. 1. The 3′ primer has the sequence 5′-CGC GGT ACC CAC TGA ACG GGG CAG CAC GC-3′ (SEQ ID NO: 12) containing the Asp 718 restriction site followed by 20 nucleotides complementary to the 3noncoding sequence in FIG. 1.

The amplified fragment is isolated from a 1% agarose gel using a commercially available kit (“Geneclean,” BIO 101 Inc., La Jolla, Calif.). The fragment then is digested with appropriate restriction enzymes and again purified on a 1% agarose gel.

The plasmid is digested with the corresponding restriction enzymes and optionally, can be dephosphorylated using calf intestinal phosphatase, using routine procedures known in the art. The DNA is then isolated from a 1% agarose gel using a commercially available kit (“Geneclean” BIO 101 Inc., La Jolla, Calif.).

The fragment and the dephosphorylated plasmid are ligated together with T4 DNA ligase. E. coli HB101 or other suitable E. coli hosts such as XL-1 Blue (Stratagene Cloning Systems, La Jolla, Calif.) cells are transformed with the ligation mixture and spread on culture plates. Bacteria containing the plasmid are identified by digesting DNA from individual colonies and analyzing the digestion product by gel electrophoresis. The sequence of the cloned fragment is confirmed by DNA sequencing.

Five micrograms of a plasmid containing the polynucleotide is co-transfected with 1.0 μg of a commercially available linearized baculovirus DNA (“BaculoGold™ baculovirus DNA”, Pharmingen, San Diego, Calif.), using the lipofection method described by Felgner and colleagues (Proc. Natl. Acad. Sci. USA 84:7413-7417 (1987)). One μg of BaculoGold™ virus DNA and 5 μg of the plasmid are mixed in a sterile well of a microtiter plate containing 50 μl of serum-free Graces medium (Life Technologies Inc., Gaithersburg, Md.). Afterwards, 10 μl Lipofectin plus 90 μl Grace's medium are added, mixed and incubated for 15 minutes at room temperature. Then the transfection mixture is added drop-wise to Sf9 insect cells (ATCC CRL 1711) seeded in a 35 mm tissue culture plate with 1 ml Graces medium without serum. The plate is then incubated for 5 hours at 27 C. The transfection solution is then removed from the plate and 1 ml of Graces insect medium supplemented with 10% fetal calf serum is added. Cultivation is then continued at 27 C for four days.

After four days the supernatant is collected and a plaque assay is performed, as described by Summers and Smith (supra). An agarose gel with “Blue Gal” (Life Technologies Inc., Gaithersburg) is used to allow easy identification and isolation of gal-expressing clones, which produce blue-stained plaques. (A detailed description of a “plaque assay” of this type can also be found in the users guide for insect cell culture and baculovirology distributed by Life Technologies Inc., Gaithersburg, page 9-10.) After appropriate incubation, blue stained plaques are picked with the tip of a micropipettor (e.g., Eppendorf). The agar containing the recombinant viruses is then resuspended in a microcentrifuge tube containing 200 μl of Graces medium and the suspension containing the recombinant baculovirus is used to infect Sf9 cells seeded in 35 mm dishes. Four days later the supernatants of these culture dishes are harvested and then they are stored at 4 C.

To verify the expression of the polypeptide, Sf9 cells are grown in Grace's medium supplemented with 10% heat-inactivated FBS. The cells are infected with the recombinant baculovirus containing the polynucleotide at a multiplicity of infection (“MOI”) of about 2. If radiolabeled proteins are desired, 6 hours later the medium is removed and is replaced with SF900 II medium minus methionine and cysteine (available from Life Technologies Inc., Rockville, Md.). After 42 hours, 5 μCi of ³⁵S-methionine and 5 μCi ³⁵S-cysteine (available from Amersham) are added. The cells are further incubated for 16 hours and then are harvested by centrifugation. The proteins in the supernatant as well as the intracellular proteins are analyzed by SDS-PAGE followed by autoradiography (if radiolabeled).

Microsequencing of the amino acid sequence of the amino terminus of purified protein may be used to determine the amino terminal sequence of the produced IL-21 protein.

### Example 8: Expression of IL-21 in Mammalian Cells.

IL-21 polypeptide can be expressed in a mammalian cell. A typical mammalian expression vector contains a promoter element, which mediates the initiation of transcription of mRNA, a protein coding sequence, and signals required for the termination of transcription and polyadenylation of the transcript. Additional elements include enhancers, Kozak sequences and intervening sequences flanked by donor and acceptor sites for RNA splicing. Highly efficient transcription is achieved with the early and late promoters from SV40, the long terminal repeats (LTRs) from Retroviruses, e.g., RSV, HTLV-I, HIV-1 and the early promoter of the cytomegalovirus (CMV). However, cellular elements can also be used (e.g., the human actin promoter).

Suitable expression vectors for use in practicing the present invention include, for example, vectors such as pSVL and pMSG (Pharmacia, Uppsala, Sweden), pRSVcat (ATCC 37152), pSV2dhfr (ATCC 37146), pBC12MI (ATCC 67109), pCMVSport 2.0, and pCMVSport 3.0. Mammalian host cells that could be used include, Hela, 293, H9 and Jurkat cells, mouse NIH3T3 and C127 cells, Cos 1, Cos 7 and CVI, quail QC1-3 cells, mouse L cells and Chinese hamster ovary (CHO) cells.

Alternatively, IL-21 polypeptide can be expressed in stable cell lines containing the IL-21 polynucleotide integrated into a chromosome. The co-transfection with a selectable marker such as dhfr, gpt, neomycin or hygromycin allows the identification and isolation of the transfected cells.

The transfected IL-21 gene can also be amplified to express large amounts of the encoded protein. The DHFR (dihydrofolate reductase) marker is useful in developing cell lines that carry several hundred or even several thousand copies of the gene of interes (see, e.g., Alt, F. W., et al, J. Biol. Chem. 253:1357-1370 (1978); Hamlin, J. L. and Ma, C., Biochem. et Biophys. Acta, 1097:107-143 (1990); Page, M. J. and Sydenham, M. A., Biotechnology 9:64-68 (1991)). Another useful selection marker is the enzyme glutamine synthase (GS; Murphy, et al., Biochem. J. 227:277-279 (1991); Bebbington, et al., Bio/Technology 10:169-175 (1992)). Using these markers, the mammalian cells are grown in selective medium and the cells with the highest resistance are selected. These cell lines contain the amplified gene(s) integrated into a chromosome. Chinese hamster ovary (CHO) and NSO cells are often used for the production of proteins.

Derivatives of the plasmid pSV2-dhfr (ATCC Accession No. 37146), the expression vectors pC4 (ATCC Accession No. 209646) and pC6 (ATCC Accession No.209647) contain the strong promoter (LTR) of the Rous Sarcoma Virus (Cullen, et al., Mol. Cell. Biol., 438-447 (March, 1985)) plus a fragment of the CMV-enhancer (Boshart, et al., Cell 41:521-530 (1985)). Multiple cloning sites, e.g., with the restriction enzyme cleavage sites Bam HI, Xba I and Asp 718, facilitate the cloning of IL-21. The vectors also contain the 3′ intron, the polyadenylation and termination signal of the rat preproinsulin gene, and the mouse DHFR gene under control of the SV40 early promoter.

Specifically, the plasmid pC6, for example, is digested with appropriate restriction enzymes and then dephosphorylated using calf intestinal phosphates by procedures known in the art. The vector is then isolated from a 1% agarose gel.

IL-21 polynucleotide is amplified according to the protocol outlined in Example 1. If the naturally occurring signal sequence is used to produce the secreted protein, the vector does not need a second signal peptide. Alternatively, if the naturally occurring signal sequence is not used, the vector can be modified to include a heterologous signal sequence (see, e.g., WO 96/34891).

The amplified fragment is isolated from a 1% agarose gel using a commercially available kit (“Geneclean”, BIO 101 Inc., La Jolla, Calif.). The fragment then is digested with appropriate restriction enzymes and again purified on a 1% agarose gel.

The amplified fragment is then digested with the same restriction enzyme and purified on a 1% agarose gel. The isolated fragment and the dephosphorylated vector are then ligated with T4 DNA ligase. E. coli HB 101 or XL-1 Blue cells are then transformed and bacteria are identified that contain the fragment inserted into plasmid pC6 using, for instance, restriction enzyme analysis.

Chinese hamster ovary cells lacking an active DHFR gene is used for transfection. Five μg of the expression plasmid pC6 is cotransfected with 0.5 μg of the plasmid pSVneo using lipofectin (Felgner, et al., supra). The plasmid pSV2-neo contains a dominant selectable marker, the neo gene from Tn5 encoding an enzyme that confers resistance to a group of antibiotics including G418. The cells are seeded in alpha minus MEM supplemented with 1 mg/ml G418. After 2 days, the cells are trypsinized and seeded in hybridoma cloning plates (Greiner, Germany) in alpha minus MEM supplemented with 10, 25, or 50 ng/ml of metothrexate plus 1 mg/ml G418. After about 10-14 days single clones are trypsinized and then seeded in 6-well petri dishes or 10 ml flasks using different concentrations of methotrexate (for example, 50 nM, 100 mM, 200 nM, 400 nM, 800 nM). Clones growing at the highest concentrations of methotrexate are then transferred to new 6-well plates containing even higher concentrations of methotrexate (1 μM, 2 μM, 5 μM, 10 mM, 20 mM). The same procedure is repeated until clones are obtained which grow at a concentration of 100-200 μM. Expression of IL-21 is analyzed, for instance, by SDS-PAGE and Western blot or by reverse phase HPLC analysis.

### Example 9: Protein Fusions of IL-21.

IL-21 polypeptides are preferably fused to other proteins. These fusion proteins can be used for a variety of applications. For example, fusion of IL-21 polypeptides to His-tag, HA-tag, protein A, IgG domains, and maltose binding protein facilitates purification (see Example 5; see also EP A 394,827; Traunecker, et al, Nature 331:84-86 (1988).) Similarly, fusion to IgG-1, IgG-3, and albumin increases the halflife time in vivo. Nuclear localization signals fused to IL-21 polypeptides can target the protein to a specific subcellular localization, while covalent heterodimer or homodimers can increase or decrease the activity of a fusion protein. Fusion proteins can also create chimeric molecules having more than one function. Finally, fusion proteins can increase solubility and/or stability of the fused protein compared to the non-fused protein. All of the types of fusion proteins described above can be made by modifying the following protocol, which outlines the fusion of a polypeptide to an IgG molecule, or the protocol described in Example 5.

Briefly, the human Fc portion of the IgG molecule can be PCR amplified, using primers that span the 5′ and 3′ ends of the sequence described below. These primers also should have convenient restriction enzyme sites that will facilitate cloning into an expression vector, preferably a mammalian expression vector.

For example, if pC4 (Accession No. 209646) is used, the human Fc portion can be ligated into the Bam HI cloning site. Note that the 3′ Bam HI site should be destroyed. Next, the vector containing the human Fc portion is again restricted with Bam HI, linearizing the vector, and IL-21 polynucleotide, isolated by the PCR protocol described in Example 1, is ligated into this Bam HI site. Note that the polynucleotide is cloned without a stop codon, otherwise a fusion protein will not be produced.

If the naturally occurring signal sequence is used to produce the secreted protein, pC4 does not need a second signal peptide. Alternatively, if the naturally occurring signal sequence is not used, the vector can be modified to include a heterologous signal sequence (see, e.g., WO 96/34891).

Human IgG Fc region (SEQ ID NO: 13):

GGGATCCGGAGCCCAAATCTTCTGACAAAACTCACACATGCCCACCGTGCCCAGCACCTGAATTCGAGGGTGC ACCGTCAGTCTTCCTCTTCCCCCCAAAACCCAAGGACACCCTCATGATCTCCCGGACTCCTGAGGTCACATGCGTGGTGG TGGACGTAAGCCACGAAGACCCTGAGGTCAAGTTCAACTGGTACGTGGACGGCGTGGAGGTGCATAATGCCAAGACAAAG CCGCGGGAGGAGCAGTACAACAGCACGTACCGTGTGGTCAGCGTCCTCACCGTCCTGCACCAGGACTGGCTGAATGGCAA GGAGTACAAGTGCAAGGTCTCCAACAAAGCCCTCCCAACCCCCATCGAGAAAACCATCTCCAAAGCCAAAGGGCAGCCCC GAGAACCACAGGTGTACACCCTGCCCCCATCCCGGGATGAGCTGACCAAGAACCAGGTCAGCCTGACCTGCCTGGTCAAA GGCTTCTATCCAAGCGACATCGCCGTGGAGTGGGAGAGCAATGGGCAGCCGGAGAACAACTACAAGACCACGCCTCCCGT GCTGGACTCCGACGGCTCCTTCTTCCTCTACAGCAAGCTCACCGTGGACAAGAGCAGGTGGCAGCAGGGGAACGTCTTCT CATGCTCCGTGATGCATGAGGCTCTGCACAACCACTACACGCAGAAGAGCCTCTCCCTGTCTCCGGGTAATGAGTGCGA CGGCCGCGACTCTAGAGGAT

### Example 10: Production of an Antibody.

a) Hybridoma Technology

The antibodies of the present invention can be prepared by a variety of methods. (See, Current Protocols, Chapter 2.) As one example of such methods, cells expressing polypeptide(s) of the invention are administered to an animal to induce the production of sera containing polyclonal antibodies. In a preferred method, a preparation of polypeptide(s) of the invention is prepared and purified to render it substantially free of natural contaminants. Such a preparation is then introduced into an animal in order to produce polyclonal antisera of greater specific activity.

Monoclonal antibodies specific for polypeptide(s) of the invention are prepared using hybridoma technology. (Kohler et al., Nature 256:495 (1975); Kohler et al., Eur. J. Immunol. 6:511 (1976); Kohler et al., Eur. J. Immunol. 6:292 (1976); Hammerling et al., in: Monoclonal Antibodies and T-Cell Hybridomas, Elsevier, N.Y., pp. 563-681 (1981)). In general, an animal (preferably a mouse) is immunized with polypeptide(s) of the invention or, more preferably, with a secreted polypeptide-expressing cell. Such polypeptide-expressing cells are cultured in any suitable tissue culture medium, preferably in Earle's modified Eagles medium supplemented with 10% fetal bovine serum (inactivated at about 56° C.), and supplemented with about 10 g/l of nonessential amino acids, about 1,000 U/ml of penicillin, and about 100 μg/ml of streptomycin.

The splenocytes of such mice are extracted and fused with a suitable myeloma cell line. Any suitable myeloma cell line may be employed in accordance with the present invention; however, it is preferable to employ the parent myeloma cell line (SP2O), available from the ATCC. After fusion, the resulting hybridoma cells are selectively maintained in HAT medium, and then cloned by limiting dilution as described by Wands et al. (Gastroenterology 80:225-232 (1981)). The hybridoma cells obtained through such a selection are then assayed to identify clones which secrete antibodies capable of binding the polypeptide(s) of the invention.

Alternatively, additional antibodies capable of binding to polypeptide(s) of the invention can be produced in a two-step procedure using anti-idiotypic antibodies. Such a method makes use of the fact that antibodies are themselves antigens, and therefore, it is possible to obtain an antibody which binds to a second antibody. In accordance with this method, protein specific antibodies are used to immunize an animal, preferably a mouse. The splenocytes of such an animal are then used to produce hybridoma cells, and the hybridoma cells are screened to identify clones which produce an antibody whose ability to bind to the protein-specific antibody can at be blocked by polypeptide(s) of the invention. Such antibodies comprise anti-idiotypic antibodies to the protein-specific antibody and are used to immunize an animal to induce formation of further protein-specific antibodies.

For in vivo use of antibodies in humans, an antibody is “humanized”. Such antibodies can be produced using genetic constructs derived from hybridoma cells producing the monoclonal antibodies described above. Methods for producing chimeric and humanized antibodies are known in the art and are discussed herein. (See, for review, Morrison, Science 229:1202 (1985); Oi et al., BioTechniques 4:214 (1986); Cabilly et al., U.S. Pat. No. 4,816,567; Taniguchi et al., EP 171496; Morrison et al., EP 173494; Neuberger et al., WO 8601533; Robinson et al., WO 8702671; Boulianne et al., Nature 312:643 (1984); Neuberger et al., Nature 314:268 (1985).)

b) Isolation Of Antibody Fragments Directed Against

Polypeptide(s) From A Library Of scFvs

Naturally occurring V-genes isolated from human PBLs are constructed into a library of antibody fragments which contain reactivities against polypeptide(s) of the invention to which the donor may or may not have been exposed (see e.g., U.S. Pat. No. 5,885,793 incorporated herein by reference in its entirety).

Rescue of the Library.

A library of scFvs is constructed from the RNA of human PBLs as described in PCT publication WO 92/01047. To rescue phage displaying antibody fragments, approximately 109 E. coli harboring the phagemid are used to inoculate 50 ml of 2xTY containing 1% glucose and 100 μg/ml of ampicillin (2xTY-AMP-GLU) and grown to an O.D. of 0.8 with shaking. Five ml of this culture is used to innoculate 50 ml of 2xTY-AMP-GLU, 2×108 TU of delta gene 3 helper (M13 delta gene III, see PCT publication WO 92/01047) are added and the culture incubated at 37° C. for 45 minutes without shaking and then at 37° C. for 45 minutes with shaking. The culture is centrifuged at 4000 r.p.m. for 10 min. and the pellet resuspended in 2 liters of 2xTY containing 100 μg/ml ampicillin and 50 ug/ml kanamycin and grown overnight. Phage are prepared as described in PCT publication WO 92/01047.

M13 delta gene III is prepared as follows: M13 delta gene III helper phage does not encode gene III protein, hence the phage(mid) displaying antibody fragments have a greater avidity of binding to antigen. Infectious M13 delta gene III particles are made by growing the helper phage in cells harboring a pUC19 derivative supplying the wild type gene III protein during phage morphogenesis. The culture is incubated for 1 hour at 37° C. without shaking and then for a further hour at 37° C. with shaking. Cells are spun down (IEC-Centra 8,400 r.p.m. for 10 min), resuspended in 300 ml 2xTY broth containing 100 μg ampicillin/ml and 25 μg kanamycin/ml (2xTY-AMP-KAN) and grown overnight, shaking at 37° C. Phage particles are purified and concentrated from the culture medium by two PEG-precipitations (Sambrook et al., 1990), resuspended in 2 ml PBS and passed through a 0.45 μm filter (Minisart NML; Sartorius) to give a final concentration of approximately 1013 transducing units/ml (ampicillin-resistant clones).

Panning of the Library.

Immunotubes (Nunc) are coated overnight in PBS with 4 ml of either 100 μg/ml or 10 μg/ml of a polypeptide of the present invention. Tubes are blocked with 2% Marvel-PBS for 2 hours at 37° C. and then washed 3 times in PBS. Approximately 1013 TU of phage is applied to the tube and incubated for 30 minutes at room temperature tumbling on an over and under turntable and then left to stand for another 1.5 hours. Tubes are washed 10 times with PBS 0.1% Tween-20 and 10 times with PBS. Phage are eluted by adding 1 ml of 100 mM triethylamine and rotating 15 minutes on an under and over turntable after which the solution is immediately neutralized with 0.5 ml of 1.0M Tris-HCl, pH 7.4. Phage are then used to infect 10 ml of mid-log E. coli TG1 by incubating eluted phage with bacteria for 30 minutes at 37° C. The E. coli are then plated on TYE plates containing 1% glucose and 100 μg/ml ampicillin. The resulting bacterial library is then rescued with delta gene 3 helper phage as described above to prepare phage for a subsequent round of selection. This process is then repeated for a total of 4 rounds of affinity purification with tube-washing increased to 20 times with PBS, 0.1% Tween-20 and 20 times with PBS for rounds 3 and 4.

Characterization of Binders.

Eluted phage from the 3rd and 4th rounds of selection are used to infect E. coli HB 2151 and soluble scFv is produced (Marks, et al., 1991) from single colonies for assay. ELISAs are performed with microtitre plates coated with either 10 pg/ml of the polypeptide of the present invention in 50 mM bicarbonate pH 9.6. Clones positive in ELISA are further characterized by PCR fingerprinting (see, e.g., PCT publication WO 92/01047) and then by sequencing. These ELISA positive clones may also be further characterized by techniques known in the art, such as, for example, epitope mapping, binding affinity, receptor signal transduction, ability to block or competitively inhibit antibody/antigen binding, and competitive agonistic or antagonistic activity.

### Example 11: Production Of IL-21 Protein For High-Throughput Screening Assays.

The following protocol produces a supernatant containing IL-21 polypeptide to be tested. This supernatant can then be used in the screening assays described subsequently in Examples 13-20.

First, dilute poly-D-lysine (644 587 Boehringer-Mannheim) stock solution (1 mg/ml in PBS) 1:20 in PBS (Phosphate Buffered Saline; w/o calcium or magnesium 17-516F Biowhittaker) for a working solution of 50 μg/ml. Add 200 μl of this solution to each well (24 well plates) and incubate at RT for 20 minutes. Be sure to distribute the solution over each well (note: a 12-channel pipetter may be used with tips on every other channel). Aspirate the poly-D-lysine solution and rinse with 1 ml PBS. The PBS should remain in the well until just prior to plating the cells and plates may be poly-lysine coated in advance for up to two weeks.

Plate 293T cells (do not carry cells past P+20) at 2×10⁵ cells/well in 0.5 ml DMEM (Dulbecco's Modified Eagle Medium) supplemented with 4.5 G/L glucose, L-glutamine (12-604F Biowhittaker)), 10% heat inactivated FBS (14-503F Biowhittaker), and 1x Penstrep (17-602E Biowhittaker). Let the cells grow overnight.

Following overnight incubation, mix together in a sterile solution basin: 300 μl Lipofectamine (18324-012 Gibco/BRL) and 5 ml Optimem I (31985070 Gibco/BRL) in each well of a 96-well plate. With a small volume multi-channel pipetter, aliquot approximately 2 μg of an expression vector containing a polynucleotide insert, produced by the methods described in Examples 8 or 9, into an appropriately labeled 96-well round bottom plate. With a multi-channel pipetter, add 50 μl of the Lipofectamine/Optimem I mixture to each well. Pipette up and down gently to mix. Incubate at RT for 15-45 minutes. After about 20 minutes, use a multi-channel pipetter to add 150 μl Optimem I to each well. As a control, one plate of vector DNA lacking an insert should be transfected with each set of transfections.

Preferably, the transfection should be performed by simultaneously performing the following tasks in a staggered fashion. Thus, hands-on time is cut in half, and the cells are not excessively incubated in PBS. First, person A aspirates the media from four 24-well plates of cells, and then person B rinses each well with 0.5-1 ml PBS. Person A then aspirates the PBS rinse, and person B, using a 12-channel pipetter with tips on every other channel, adds the 200 μl of DNA/Lipofectamine/Optimem I complex to the odd wells first, then to the even wells, to each row on the 24-well plates. Plates are then incubated at 37 C for 6 hours.

While cells are incubating, the appropriate media is prepared: either 1% BSA in DMEM with 1x penstrep, or HGS CHO-5 media (116.6 mg/L of CaCl₂ (anhyd); 0.00130 mg/L CuSO₄-5H₂O; 0.050 mg/L of Fe(NO₃)₃-9H₂O; 0.417 mg/L of FeSO₄-7H₂O; 311.80 mg/L of KCl; 28.64 mg/L of MgCl₂; 48.84 mg/L of MgSO₄; 6995.50 mg/L of NaCl; 2400.0 mg/L of NaHCO₃; 62.50 mg/L of NaH₂PO₄-H₂0; 71.02 mg/L of Na₂HPO₄; 4320 mg/L of ZnSO₄-7H₂O; 0.002 mg/L of Arachidonic Acid; 1.022 mg/L of Cholesterol; 0.070 mg/L of D-L-alpha-Tocopherol-Acetate; 0.0520 mg/L of Linoleic Acid; 0.010 mg/L of Linolenic Acid; 0.010 mg/L of Myristic Acid; 0.010 mg/L of Oleic Acid; 0.010 mg/L of Palmitric Acid; 0.010 mg/L of Palmitic Acid; 100 mg/L of Pluronic F-68; 0.010 mg/L of Stearic Acid; 2.20 mg/L of Tween 80; 4551 mg/L of D-Glucose; 130.85 mg/ml of L-Alanine; 147.50 mg/ml of L-Arginine-HCL; 7.50 mg/ml of L-Asparagine-H₂O; 6.65 mg/ml of L-Aspartic Acid; 29.56 mg/ml of L-Cystine-2HCL-H₂O; 31.29 mg/ml of L-Cystine-2HCl; 7.35 mg/ml of L-Glutamic Acid; 365.0 mg/ml of L-Glutamine; 18.75 mg/ml of Glycine; 52.48 mg/ml of L-Histidine-HCL-H₂O; 106.97 mg/ml of L-Isoleucine; 111.45 mg/ml of L-Leucine; 163.75 mg/ml of L-Lysine HCL; 32.34 mg/ml of L-Methionine; 68.48 mg/ml of L-Phenylalainine; 40.0 mg/ml of L-Proline; 26.25 mg/ml of L-Serine; 101.05 mg/ml of L-Threonine; 19.22 mg/ml of L-Tryptophan; 91.79 mg/ml of L-Tryrosine-2Na-2H₂O; and 99.65 mg/ml of L-Valine; 0.0035 mg/L of Biotin; 3.24 mg/L of D-Ca Pantothenate; 11.78 mg/L of Choline Chloride; 4.65 mg/L of Folic Acid; 15.60 mg/L of i-Inositol; 3.02 mg/L of Niacinamide; 3.00 mg/L of Pyridoxal HCL; 0.031 mg/L of Pyridoxine HCL; 0.319 mg/L of Riboflavin; 3.17 mg/L of Thiamine HCL; 0.365 mg/L of Thymidine; 0.680 mg/L of Vitamin B₁₂; 25 mM of HEPES Buffer; 2.39 mg/L of Na Hypoxanthine; 0.105 mg/L of Lipoic Acid; 0.081 mg/L of Sodium Putrescine-2HCL; 55.0 mg/L of Sodium Pyruvate; 0.0067 mg/L of Sodium Selenite; 20 μm of Ethanolamine; 0.122 mg/L of Ferric Citrate; 41.70 mg/L of Methyl-B-Cyclodextrin complexed with Linoleic Acid; 33.33 mg/L of Methyl-B-Cyclodextrin complexed with Oleic Acid; 10 mg/L of Methyl-B-Cyclodextrin complexed with Retinal Acetate. Adjust osmolarity to 327 mOsm) with 2 mm glutamine and 1x penstrep. (BSA (81-068-3 Bayer) 100 gm dissolved in 1L DMEM for a 10% BSA stock solution). Filter the media and collect 50 μl for endotoxin assay in 15 ml polystyrene conical.

The transfection reaction is terminated, again, preferably by two people, at the end of the incubation period. Person A aspirates the transfection media, while person B adds 1.5 ml of the appropriate media to each well. Incubate at 37 C for 45 or 72 hours, depending on the media used (1% BSA for 45 hours or CHO-5 for 72 hours).

On day four, using a 300 μl multichannel pipetter, aliquot 600 μl in one 1 ml deep well plate and the remaining supernatant into a 2 ml deep well. The supernatants from each well can then be used in the assays described in Examples 13-20.

It is specifically understood that when activity is obtained in any of the assays described below using a supernatant, the activity originates from either the IL-21 polypeptide directly (e.g., as a secreted protein) or by IL-21 inducing expression of other proteins, which are then secreted into the supernatant. Thus, the invention further provides a method of identifying the protein in the supernatant characterized by an activity in a particular assay.

### Example 12: Construction of GAS Reporter Construct.

One signal transduction pathway involved in the differentiation and proliferation of cells is called the Jaks-STATs pathway. Activated proteins in the Jaks-STATs pathway bind to gamma activation site (“GAS”) elements or interferon-sensitive responsive element (“ISRE”), located in the promoter of many genes. The binding of a protein to these elements alter the expression of the associated gene.

GAS and ISRE elements are recognized by a class of transcription factors called Signal Transducers and Activators of Transcription, or “STATs.” There are six members of the STATs family. Stat1 and Stat3 are present in many cell types, as is Stat2 (as response to IFN-alpha is widespread). Stat4 is more restricted and is not in many cell types though it has been found in T-helper class I, cells after treatment with IL-12. Stat5 was originally called mammary growth factor, but has been found at higher concentrations in other cells including myeloid cells. It can be activated in tissue culture cells by many cytokines.

The STATs are activated to translocate from the cytoplasm to the nucleus upon tyrosine phosphorylation by a set of kinases known as the Janus Kinase (“Jaks”) family. Jaks represent a distinct family of soluble tyrosine kinases and include Tyk2, Jak1, Jak2, and Jak3. These kinases display significant sequence similarity and are generally catalytically inactive in resting cells.

The Jaks are activated by a wide range of receptors summarized in the Table below (adapted from review by Schidler and Darnell, Ann. Rev. Biochem. 64:621-51 (1995))). A cytokine receptor family, capable of activating Jaks, is divided into two groups: (a) Class 1 includes receptors for IL-2, IL-3, IL-4, IL-6, IL-7, IL-9, IL-11, IL-12, IL-15, Epo, PRL, GH, G-CSF, GM-CSF, LIF, CNTF, and thrombopoietin; and (b) Class 2 includes IFN-alpha, IFN-gamma, and IL-10. The Class 1 receptors share a conserved cysteine motif (a set of four conserved cysteines and one tryptophan) and a WSXWS motif (a membrane proximal region encoding Trp-Ser-Xaa-Trp-Ser (where “Xaa” represents any amino acid; SEQ ID NO: 14)).

Thus, on binding of a ligand to a receptor, Jaks are activated, which in turn activate STATs, which then translocate and bind to GAS elements. This entire process is encompassed in the Jaks-STATs signal transduction pathway.

Therefore, activation of the Jaks-STATs pathway, reflected by the binding of the GAS or the ISRE element, can be used to indicate proteins involved in the proliferation and differentiation of cells. For example, growth factors and cytokines are known to activate the Jaks-STATs pathway (see Table below). Thus, by using GAS elements linked to reporter molecules, activators of the Jaks-STATs pathway can be identified.

| | JAKs | | | | | |
|---|---|---|---|---|---|---|
| Ligand | tyk2 | Jak1 | Jak2 | Jak3 | STATS | GAS(elements) or ISRE |
| IFN family | | | | | | |
| IFN-alpha/beta | | + | + | − | − | 1,2,3 ISRE |
| IFN-gamma | | + | + | − | 1 | GAS (IRF1 > Lys6 > IFP) |
| I1-10 | + | ? | ? | − | 1,3 | |
| gp130 family | | | | | | |
| IL-6 (Pleiotrohic) | + | + | + | ? | 1,3 | GAS (IRF1 > Lys6 > IFP) |
| I1-11 (Pleiotrohic) | ? | + | ? | ? | 1,3 | |
| OnM (Pleiotrohic) | ? | + | + | ? | 1,3 | |
| LIF (Pleiotrohic) | ? | + | + | ? | 1,3 | |
| CNTF (Pleiotrohic) | −/+ | + | + | ? | 1,3 | |
| G-CSF (Pleiotrohic) | ? | + | ? | ? | 1,3 | |
| IL-12 (Pleiotrohic) | + | − | + | + | 1,3 | |
| g-C family | | | | | | |
| IL-2 (lymphocytes) | − | + | − | + | 1,3,5 | GAS |
| IL-4 (lymph/myeloid) | − | + | − | + | 6 | GAS (IRF1 = IFP >> Ly6) (IgH) |
| IL-7 (lymphocytes) | − | + | − | + | 5 | GAS |
| IL-9 (lymphocytes) | − | + | − | + | 5 | GAS |
| IL-13 (lymphocyte) | − | + | ? | ? | 6 | GAS |
| IL-15 | ? | + | ? | + | 5 | GAS |
| gp140 family | | | | | | |
| IL-3 (myeloid) | − | − | + | − | 5 | GAS (IRF1 > IFP >> Ly6) |
| IL-5 (myeloid) | − | − | + | − | 5 | GAS |
| GM-CSF (myeloid) | − | − | + | − | 5 | GAS |

Growth hormone family

| | | | | | | |
|---|---|---|---|---|---|---|
| GH | ? | − | + | − | 5 | |
| PRL | ? | +/− | + | − | 1,3,5 | |
| EPO | ? | − | + | − | 5 | GAS (B-CAS > IRF1 = IFP>> Ly6) |

Receptor Tyrosine Kinases

| | | | | | | |
|---|---|---|---|---|---|---|
| EGF | ? | + | + | − | 1,3 | GAS (IRF1) |
| PDGF | ? | + | + | − | 1,3 | |
| CSF-1 | ? | + | + | − | 1,3 | GAS (not IRF1) |

To construct a synthetic GAS containing promoter element, which is used in the biological assays described in Examples 13-14, a PCR based strategy is employed to generate a GAS-SV40 promoter sequence. The 5′ primer contains four tandem copies of the GAS-binding site found in the IRF1 promoter and previously demonstrated to bind STATs upon induction with a range of cytokines (Rothman, et al., Immunity 1:457-468 (1994)), although other GAS or ISRE elements can be used instead. The 5′ primer also contains 18 bp of sequence complementary to the SV40 early promoter sequence and is flanked with an Xho I restriction site. The sequence of the 5′ primer is:

| | |
|---|---|
| 5′-GCG CCT | (SEQ ID NO:15). |
| CGA GAT TTC CCC GAA ATC TAG ATT TCC CCG AAA TGA TTT CCC CGA | |
| AAT GAT TTC CCC GAA ATA TCT GCC ATC TCA ATT AG-3′ | |

(SEQ ID NO: 15).

The downstream primer is complementary to the SV40 promoter and is flanked with a Hin dIII site:

5′-GCG GCA AGC TTT TTG CAA AGC CTA GGC-3′ (SEQ ID NO:16).

(SEQ ID NO: 16).

PCR amplification is performed using the SV40 promoter template present in the B-gal:promoter plasmid obtained from Clontech. The resulting PCR fragment is digested with Xho I and Hin dIII and subcloned into BLSK2- (Stratagene). Sequencing with forward and reverse primers confirms that the insert contains the following sequence:

| | |
|---|---|
| CTCGAGATTTCCCCGAAATCTAGATTTCCCCGAAATGATTTCCCCGAA | (SEQ ID NO:17). |
| ATGATTTCCCCGAAATATCTGCCATCTCAATTAGTCAGCAACCATAGTCCCGC | |
| CCCTAACTCCGCCCATCCCGCCCCTAACTCCGCCCAGTTCCGCCCATTCTCCG | |
| CCCCATGGCTGACTAATTTTTTTTATTTATGCAGAGGCCGAGGCCGCCTCGGC | |
| CTCTGAGCTATTCCAGAAGTAGTGAGGAGGCTTTTTTGGAGGCCTAGGCTTTT | |
| GCAAAAAGCTT | |

With this GAS promoter element linked to the SV40 promoter, a GAS: SEAP2 reporter construct is next engineered. Here, the reporter molecule is a secreted alkaline phosphatase, or “SEAP”. Clearly, however, any reporter molecule can be instead of SEAP, in this or in any of the other Examples. Well known reporter molecules that can be used instead of SEAP include chloramphenicol acetyltransferase (CAT), luciferase, alkaline phosphatase, B-galactosidase, green fluorescent protein (GFP), or any protein detectable by an antibody.

The above sequence confirmed synthetic GAS-SV40 promoter element is subcloned into the pSEAP-Promoter vector obtained from Clontech using Hin dIII and Xho I, effectively replacing the SV40 promoter with the amplified GAS:SV40 promoter element, to create the GAS-SEAP vector. However, this vector does not contain a neomycin resistance gene, and therefore, is not preferred for mammalian expression systems.

Thus, in order to generate mammalian stable cell lines expressing the GAS-SEAP reporter, the GAS-SEAP cassette is removed from the GAS-SEAP vector using Sal I and Not I, and inserted into a backbone vector containing the neomycin resistance gene, such as pGFP-1 (Clontech), using these restriction sites in the multiple cloning site, to create the GAS-SEAP/Neo vector. Once this vector is transfected into mammalian cells, this vector can then be used as a reporter molecule for GAS binding as described in Examples 13-14.

Other constructs can be made using the above description and replacing GAS with a different promoter sequence. For example, construction of reporter molecules containing NF-kappaB and EGR promoter sequences are described in Examples 15 and 16. However, many other promoters can be substituted using the protocols described in these Examples. For instance, SRE, IL-2, NFAT, or Osteocalcin promoters can be substituted, alone or in combination (e.g., GAS/NF-kappaB/EGR, GAS/NF-kappaB, Il-2/NFAT, or NF-kappaB/GAS). Similarly, other cell lines can be used to test reporter construct activity, such as HeLa (epithelial), HUVEC (endothelial), Reh (B-cell), Saos-2 (osteoblast), HUVAC (aortic), or Cardiomyocyte.

### Example 13: High-Throughput Screening Assay for T-cell Activity.

The following protocol is used to assess T-cell activity of IL-21 by determining whether IL-21 supernatant proliferates and/or differentiates T-cells. T-cell activity is assessed using the GAS/SEAP/Neo construct produced in Example 12. Thus, factors that increase SEAP activity indicate the ability to activate the Jaks-STATS signal transduction pathway. The T-cell used in this assay is Jurkat T-cells (ATCC Accession No. TIB-152), although Molt-3 cells (ATCC Accession No. CRL-1552) and Molt-4 cells (ATCC Accession No. CRL-1582) cells can also be used.

Jurkat T-cells are lymphoblastic CD4+ Th1 helper cells. In order to generate stable cell lines, approximately 2 million Jurkat cells are transfected with the GAS-SEAP/neo vector using DMRIE-C (Life Technologies; transfection procedure described below). The transfected cells are seeded to a density of approximately 20,000 cells per well and transfectants resistant to 1 mg/ml genticin selected. Resistant colonies are expanded and then tested for their response to increasing concentrations of interferon gamma. The dose response of a selected clone is demonstrated.

Specifically, the following protocol will yield sufficient cells for 75 wells containing 200 μl of cells. Thus, it is either scaled up, or performed in multiple to generate sufficient cells for multiple 96 well plates. Jurkat cells are maintained in RPMI+10% serum with 1% Pen-Strep. Combine 2.5 mls of OPTI-MEM (Life Technologies) with 10 μg of plasmid DNA in a T25 flask. Add 2.5 ml OPTI-MEM containing 50 μl of DMRIE-C and incubate at room temperature for 15-45 min.

During the incubation period, count cell concentration, spin down the required number of cells (10⁷ per transfection), and resuspend in OPTI-MEM to a final concentration of 10⁷ cells/ml. Then add 1 ml of 1×10⁷ cells in OPTI-MEM to T25 flask and incubate at 37° C. for 6 hrs. After the incubation, add 10 ml of RPMI+15% serum.

The Jurkat:GAS-SEAP stable reporter lines are maintained in RPMI+10% serum, 1 mg/ml Genticin, and 1% Pen-Strep. These cells are treated with supernatants containing IL-21 polypeptides or IL-21 induced polypeptides as produced by the protocol described in Example 11.

On the day of treatment with the supernatant, the cells should be washed and resuspended in fresh RPMI+10% serum to a density of 500,000 cells per ml. The exact number of cells required will depend on the number of supernatants being screened. For one 96 well plate, approximately 10 million cells (for 10 plates, 100 million cells) are required.

Transfer the cells to a triangular reservoir boat, in order to dispense the cells into a 96 well dish, using a 12 channel pipette. Using a 12 channel pipette, transfer 200 μl of cells into each well (therefore adding 100,000 cells per well).

After all the plates have been seeded, 50 μl of the supernatants are transferred directly from the 96 well plate containing the supernatants into each well using a 12 channel pipette. In addition, a dose of exogenous interferon gamma (0.1, 1.0, 10 ng) is added to wells H9, H10, and H11 to serve as additional positive controls for the assay.

The 96 well dishes containing Jurkat cells treated with supernatants are placed in an incubator for 48 hrs (note: this time is variable between 48-72 hrs). 35 μl samples from each well are then transferred to an opaque 96 well plate using a 12 channel pipette. The opaque plates should be covered (using sellophene covers) and stored at −20 C until SEAP assays are performed according to Example 17. The plates containing the remaining treated cells are placed at 4 C and serve as a source of material for repeating the assay on a specific well if desired.

As a positive control, 100 Unit/ml interferon gamma can be used which is known to activate Jurkat T cells. Over 30 fold induction is typically observed in the positive control wells.

### Example 14: High-Throughput Screening Assay Identifying Myeloid Activity.

The following protocol is used to assess myeloid activity of IL-21 by determining whether IL-21 proliferates and/or differentiates myeloid cells. Myeloid cell activity is assessed using the GAS/SEAP/Neo construct produced in Example 12. Thus, factors that increase SEAP activity indicate the ability to activate the Jaks-STATS signal transduction pathway. The myeloid cell used in this assay is U937, a pre-monocyte cell line, although TF-1, HL60, or KG1 can be used.

To transiently transfect U937 cells with the GAS/SEAP/Neo construct produced in Example 12, a DEAE-Dextran method (Kharbanda, et. al., Cell Growth & Differentiation, 5:259-265 (1994)) is used. First, harvest 2×10⁷ U937 cells and wash with PBS. The U937 cells are usually grown in RPMI 1640 medium containing 10% heat-inactivated fetal bovine serum (FBS) supplemented with 100 units/ml penicillin and 100 mg/ml streptomycin.

Next, suspend the cells in 1 ml of 20 mM Tris-HCl (pH 7.4) buffer containing 0.5 mg/ml DEAE-Dextran, 8 μg GAS-SEAP2 plasmid DNA, 140 mM NaCl, 5 mM KCl, 375 uM Na₂HPO₄7H₂O, 1 mM MgCl₂, and 675 uM CaCl₂. Incubate at 37 C for 45 min.

Wash the cells with RPMI 1640 medium containing 10% FBS and then resuspend in 10 ml complete medium and incubate at 37 C for 36 hr.

The GAS-SEAP/U937 stable cells are obtained by growing the cells in 400 μg/ml G418. The G418-free medium is used for routine growth but every one to two months, the cells should be re-grown in 400 ug/ml G418 for couple of passages.

These cells are tested by harvesting 1×10⁸ cells (this is enough for ten 96-well plates assay) and wash with PBS. Suspend the cells in 200 ml above described growth medium, with a final density of 5×10⁵ cells/ml. Plate 200 μl cells per well in the 96-well plate (or 1×10⁵ cells/well).

Add 50 μl of the supernatant prepared by the protocol described in Example 11. Incubate at 37 C for 48 to 72 hr. As a positive control, 100 U/ml interferon gamma can be used which is known to activate U937 cells. Over 30-fold induction is typically observed in the positive control wells. SEAP assay the supernatant according to the protocol described in Example 17.

### Example 15: High-Throughput Screening Assay Identifying Neuronal Activity.

When cells undergo differentiation and proliferation, a group of genes are activated through many different signal transduction pathways. One of these genes, EGR1 (early growth response gene 1), is induced in various tissues and cell types upon activation. The promoter of EGR1 is responsible for such induction. Using the EGR1 promoter linked to reporter molecules, activation of cells can be assessed by IL-21.

Particularly, the following protocol is used to assess neuronal activity in PC12 cell lines. PC12 cells (rat phenochromocytoma cells) are known to proliferate and/or differentiate by activation with a number of mitogens, such as TPA (tetradecanoyl phorbol acetate), NGF (nerve growth factor), and EGF (epidermal growth factor). The EGR1 gene expression is activated during this treatment. Thus, by stably transfecting PC12 cells with a construct containing an EGR promoter linked to SEAP reporter, activation of PC12 cells by IL-21 can be assessed.

The EGR/SEAP reporter construct can be assembled by the following protocol. The EGR-1 promoter sequence (nucleotides −633 to +1; Sakamoto, K., et al., Oncogene 6:867-871 (1991)) can be PCR amplified from human genomic DNA using the following primers: (A) 5′ Primer: 5′-GCG CTC GAG GGA TGA CAG CGA TAG AAC CCC GG-3′ (SEQ ID NO: 18) and (B) 3′ Primer: 5′-GCG AAG CTT CGC GAC TCC CCG GAT CCG CCT C-3′ (SEQ ID NO: 19).

Using the GAS:SEAP/Neo vector produced in Example 12, EGR1 amplified product can then be inserted into this vector. Linearize the GAS:SEAP/Neo vector using restriction enzymes Xho I and Hin dIII, removing the GAS/SV40 stuffer fragment. Digest the EGR1 amplified product with the same enzymes. Ligate the vector and the EGR1 promoter.

To prepare 96 well-plates for cell culture, 2 ml of a coating solution (1:30 dilution of collagen type I (Upstate Biotech Inc. Cat#08-115) in 30% ethanol (filter sterilized)) is added per one 10 cm plate or 50 ml per well of the 96-well plate, and allowed to air dry for 2 hr.

PC12 cells are routinely grown in RPMI-1640 medium (Bio Whittaker) containing 10% horse serum (JRH BIOSCIENCES, Cat. # 12449-78P), 5% heat-inactivated fetal bovine serum (FBS) supplemented with 100 units/ml penicillin and 100 μg/ml streptomycin on a precoated 10 cm tissue culture dish. A 1:4 split is done every three to four days. Cells are removed from the plates by scraping and resuspended with pipetting up and down for more than 15 times.

Transfect the EGR/SEAP/Neo construct into PC12 using the Lipofectamine protocol described in Example 11. EGR-SEAP/PC12 stable cells are obtained by growing the cells in 300 μg/ml G418. The G418-free medium is used for routine growth but every one to two months, the cells should be re-grown in 300 μg/ml G418 for several passages.

To assay for neuronal activity, a 10 cm plate with cells around 70 to 80% confluent is screened by removing the old medium. Wash the cells once with PBS. Then starve the cells in low serum medium (RPMI-1640 containing 1% horse serum and 0.5% FBS with antibiotics) overnight.

The next morning, remove the medium and wash the cells with PBS. Scrape off the cells from the plate, suspend the cells well in 2 ml low serum medium. Count the cell number and add more low serum medium to reach final cell density as 5×10⁵ cells/ml.

Add 200 μl of the cell suspension to each well of 96-well plate (equivalent to 1×10⁵ cells/well). Add 50 μl supernatant produced by Example 11, 37 C for 48 to 72 hr. As a positive control, a growth factor known to activate PC12 cells through EGR can be used, such as 50 ng/μl of Neuronal Growth Factor (NGF). Over fifty-fold induction of SEAP is typically seen in the positive control wells. SEAP assay the supernatant according to Example 17.

### Example 16: High-Throughput Screening Assay for T-cell Activity.

NF-kappaB (Nuclear Factor kappaB) is a transcription factor activated by a wide variety of agents including the inflammatory cytokines IL-1 and TNF, CD30 and CD40, lymphotoxin-a and lymphotoxin-b, by exposure to LPS or thrombin, and by expression of certain viral gene products. As a transcription factor, NF-kappaB regulates the expression of genes involved in immune cell activation, control of apoptosis (NF-kappaB appears to shield cells from apoptosis), B- and T-cell development, anti-viral and antimicrobial responses, and multiple stress responses.

In non-stimulated conditions, NF-kappaB is retained in the cytoplasm with I-kappaB (Inhibitor kappaB). However, upon stimulation, I- kappaB is phosphorylated and degraded, causing NF-kappaB to shuttle to the nucleus, thereby activating transcription of target genes. Target genes activated by NF-kappaB include IL-2, L-6, GM-CSF, ICAM-1 and class 1 MHC.

Due to its central role and ability to respond to a range of stimuli, reporter constructs utilizing the NF-kappaB promoter element are used to screen the supernatants produced in Example 11. Activators or inhibitors of NF-kappaB would be useful in treating diseases. For example, inhibitors of NF-kappaB could be used to treat those diseases related to the acute or chronic activation of NF-kappaB, such as rheumatoid arthritis.

To construct a vector containing the NF-kappaB promoter element, a PCR based strategy is employed. The upstream primer contains four tandem copies of the NF-kappaB binding site (5′-GGG GAC TTT CCC-3′; SEQ ID NO: 20), 18 bp of sequence complementary to the 5′ end of the SV40 early promoter sequence, and is flanked with an Xho I site: 5′-GCG GCC TCG AGG GGA CTT TCC CGG GGA CTT TCC GGG GAC TTT CCG GGA CTT TCC ATC CTG CCA TCT CAA TTA G-3 (SEQ ID NO: 21).

The downstream primer is complementary to the 3′ end of the SV40 promoter and is flanked with a Hin dIII site:

5′-GCG GCA AGC TTT TTG CAA AGC CTA GGC-3′ (SEQ ID NO:22).

(SEQ ID NO: 22).

PCR amplification is performed using the SV40 promoter template present in the pB-gal:promoter plasmid obtained from Clontech. The resulting PCR fragment is digested with Xho I and Hin dIII and subcloned into BLSK2- (Stratagene). Sequencing with the T7 and T3 primers confirms the insert contains the following sequence:

| | |
|---|---|
| 5′-CTCGAGGGGACTTTCCCGGGGACTTTCCGGGGACTTTCCGGGACTTT | (SEQ ID NO:23) |
| CCATCTGCCATCTCAATTAGTCAGCAACCATAGTCCCGCCCCTAACTCCGCCC | |
| ATCCCGCCCCTAACTCCGCCCAGTTCCGCCCATTCTCCGCCCCATGGCTGACT | |
| AATTTTTTTTATTTATGCAGAGGCCGAGGCCGCCTCGGCCTCTGAGCTATTCC | |
| AAAAAAAGCTT-3′ | |

(SEQ ID NO: 23)

Next, replace the SV40 minimal promoter element present in the pSEAP2-promoter plasmid (Clontech) with this NF-kappaB/SV40 fragment using Xho I and Hin dIII. However, this vector does not contain a neomycin resistance gene, and therefore, is not preferred for mammalian expression systems.

In order to generate stable mammalian cell lines, the NF-kappaB/SV40/SEAP cassette is removed from the above NF-kappaB/SEAP vector using restriction enzymes Sal I and Not I, and inserted into a vector containing neomycin resistance. Particularly, the NF-kappaB/SV40/SEAP cassette was inserted into pGFP-1 (Clontech), replacing the GFP gene, after restricting pGFP-1 with Sal I and Not I.

Once NF-kappaB/SV40/SEAP/Neo vector is created, stable Jurkat T-cells are created and maintained according to the protocol described in Example 13. Similarly, the method for assaying supernatants with these stable Jurkat T-cells is also described in Example 13. As a positive control, exogenous TNF-a (0.1,1, 10 ng) is added to wells H9, H10, and H11, with a 5-10 fold activation typically observed.

### Example 17: Assay for SEAP Activity.

As a reporter molecule for the assays described in Examples 13-16, SEAP activity is assayed using the Tropix Phospho-light Kit (Cat. BP-400) according to the following general procedure. The Tropix Phospho-light Kit supplies the Dilution, Assay, and Reaction Buffers used below.

Prime a dispenser with the 2.5x Dilution Buffer and dispense 15 μl of 2.5x dilution buffer into Optiplates containing 35 μl of a supernatant. Seal the plates with a plastic sealer and incubate at 65 C for 30 min. Separate the Optiplates to avoid uneven heating.

Cool the samples to room temperature for 15 minutes. Empty the dispenser and prime with the Assay Buffer. Add 50 μl Assay Buffer and incubate at room temperature 5 min. Empty the dispenser and prime with the Reaction Buffer (see the table below). Add 50 μl Reaction Buffer and incubate at room temperature for 20 minutes. Since the intensity of the chemiluminescent signal is time dependent, and it takes about 10 minutes to read 5 plates on luminometer, one should treat 5 plates at each time and start the second set 10 minutes later.

Read the relative light unit in the luminometer. Set H12 as blank, and print the results. An increase in chemiluminescence indicates reporter activity.

TABLE IV
Reaction Buffer Formulation:

| # of | Rxn buffer diluent | CSPD |
|---|---|---|
| plates | (ml) | (ml) |
| 10 | 60 | 3 |
| 11 | 65 | 3.25 |
| 12 | 70 | 3.5 |
| 13 | 75 | 3.75 |
| 14 | 80 | 4 |
| 15 | 85 | 4.25 |
| 16 | 90 | 4.5 |
| 17 | 95 | 4.75 |
| 18 | 100 | 5 |
| 19 | 105 | 5.25 |
| 20 | 110 | 5.5 |
| 21 | 115 | 5.75 |
| 22 | 120 | 6 |
| 23 | 125 | 6.25 |
| 24 | 130 | 6.5 |
| 25 | 135 | 6.75 |
| 26 | 140 | 7 |
| 27 | 145 | 7.25 |
| 28 | 150 | 7.5 |
| 29 | 155 | 7.75 |
| 30 | 160 | 8 |
| 31 | 165 | 8.25 |
| 32 | 170 | 8.5 |
| 33 | 175 | 8.75 |
| 34 | 180 | 9 |
| 35 | 185 | 9.25 |
| 36 | 190 | 9.5 |
| 37 | 195 | 9.75 |
| 38 | 200 | 10 |
| 39 | 205 | 10.25 |
| 40 | 210 | 10.5 |
| 41 | 215 | 10.75 |
| 42 | 220 | 11 |
| 43 | 225 | 11.25 |
| 44 | 230 | 11.5 |
| 45 | 235 | 11.75 |
| 46 | 240 | 12 |
| 47 | 245 | 12.25 |
| 48 | 250 | 12.5 |
| 49 | 255 | 12.75 |
| 50 | 260 | 13 |

### Example 18: High-Throughput Screening Assay Identifying Changes in Small Molecule Concentration and Membrane Permeability.

Binding of a ligand to a receptor is known to alter intracellular levels of small molecules, such as calcium, potassium, sodium, and pH, as well as alter membrane potential. These alterations can be measured in an assay to identify supernatants which bind to receptors of a particular cell. Although the following protocol describes an assay for calcium, this protocol can easily be modified to detect changes in potassium, sodium, pH, membrane potential, or any other small molecule which is detectable by a fluorescent probe.

The following assay uses Fluorometric Imaging Plate Reader (“FLIPR”) to measure changes in fluorescent molecules (Molecular Probes) that bind small molecules. Clearly, any fluorescent molecule detecting a small molecule can be used instead of the calcium fluorescent molecule, fluo-3, used here.

For adherent cells, seed the cells at 10,000-20,000 cells/well in a Co-star black 96-well plate with clear bottom. The plate is incubated in a CO₂ incubator for 20 hours. The adherent cells are washed two times in Biotek washer with 200 μl of HBSS (Hank's Balanced Salt Solution) leaving 100 ul of buffer after the final wash.

A stock solution of 1 mg/ml fluo-3 is made in 10% pluronic acid DMSO. To load the cells with fluo-3, 50 μl of 12 μg/ml fluo-3 is added to each well. The plate is incubated at 37 C in a CO₂ incubator for 60 min. The plate is washed four times in the Biotek washer with HBSS leaving 100 μl of buffer.

For non-adherent cells, the cells are spun down from culture media. Cells are re-suspended to 2-5×10⁶ cells/ml with HBSS in a 50-ml conical tube. Four μl of 1 mg/ml fluo-3 solution in 10% pluronic acid DMSO is added to each 1 ml of cell suspension. The tube is then placed in a 37 C water bath for 30-60 min. The cells are washed twice with HBSS, resuspended to 1×10⁶ cells/ml, and dispensed into a microplate, 100 μl/well. The plate is centrifuged at 1000 rpm for 5 min. The plate is then washed once in Denley CellWash with 200 μl, followed by an aspiration step to 100 μl final volume.

For a non-cell based assay, each well contains a fluorescent molecule, such as fluo-3. The supernatant is added to the well, and a change in fluorescence is detected.

To measure the fluorescence of intracellular calcium, the FLIPR is set for the following parameters: (1) System gain is 300-800 mW; (2) Exposure time is 0.4 second; (3) Camera F/stop is F/2; (4) Excitation is 488 nm; (5) Emission is 530 nm; and (6) Sample addition is 50 ul. Increased emission at 530 nm indicates an extracellular signaling event caused by the a molecule, either IL-21 or a molecule induced by IL-21, which has resulted in an increase in the intracellular Ca²⁺ concentration.

### Example 19: High-Throughput Screening Assay Identifying Tyrosine Kinase Activity.

The Protein Tyrosine Kinases (PTK) represent a diverse group of transmembrane and cytoplasmic kinases. Within the Receptor Protein Tyrosine Kinase RPTK) group are receptors for a range of mitogenic and metabolic growth factors including the PDGF, FGF, EGF, NGF, HGF and Insulin receptor subfamilies. In addition there are a large family of RPTKs for which the corresponding ligand is unknown. Ligands for RPTKs include mainly secreted small proteins, but also membrane-bound and extracellular matrix proteins.

Activation of RPTK by ligands involves ligand-mediated receptor dimerization, resulting in transphosphorylation of the receptor subunits and activation of the cytoplasmic tyrosine kinases. The cytoplasmic tyrosine kinases include receptor associated tyrosine kinases of the src-family (e.g., src, yes, lck, lyn, fyn) and non-receptor linked and cytosolic protein tyrosine kinases, such as the Jak family, members of which mediate signal transduction triggered by the cytokine superfamily of receptors (e.g., the Interleukins, Interferons, GM-CSF, and Leptin).

Because of the wide range of known factors capable of stimulating tyrosine kinase activity, identifying whether IL-21 or a molecule induced by IL-21 is capable of activating tyrosine kinase signal transduction pathways is of interest. Therefore, the following protocol is designed to identify such molecules capable of activating the tyrosine kinase signal transduction pathways.

Seed target cells (e.g., primary keratinocytes) at a density of approximately 25,000 cells per well in a 96 well Loprodyne Silent Screen Plates purchased from Nalge Nunc (Naperville, Ill.). The plates are sterilized with two 30 minute rinses with 100% ethanol, rinsed with water and dried overnight. Some plates are coated for 2 hr with 100 ml of cell culture grade type I collagen (50 mg/ml), gelatin (2%) or polylysine (50 mg/ml), all of which can be purchased from Sigma Chemicals (St. Louis, Mo.) or 10% Matrigel purchased from Becton Dickinson (Bedford, Mass.), or calf serum, rinsed with PBS and stored at 4 C. Cell growth on these plates is assayed by seeding 5,000 cells/well in growth medium and indirect quantitation of cell number through use of alamar Blue as described by the manufacturer Alamar Biosciences, Inc. (Sacramento, Calif.) after 48 hr. Falcon plate covers #3071 from Becton Dickinson (Bedford, Mass.) are used to cover the Loprodyne Silent Screen Plates. Falcon Microtest III cell culture plates can also be used in some proliferation experiments.

To prepare extracts, A431 cells are seeded onto the nylon membranes of Loprodyne plates (20,000/200ml/well) and cultured overnight in complete medium. Cells are quiesced by incubation in serum-free basal medium for 24 hr. After 5-20 minutes, treatment with EGF (60 ng/ml) or 50 μl of the supernatant produced in Example 11, the medium was removed and 100 ml of extraction buffer ((20 mM HEPES pH 7.5, 0.15M NaCl, 1% Triton X-100, 0.1% SDS, 2 mM Na3VO4, 2 mM Na4P2O7 and a cocktail of protease inhibitors (# 1836170) obtained from Boeheringer Mannheim (Indianapolis, Ind.) is added to each well and the plate is shaken on a rotating shaker for 5 minutes at 4 C. The plate is then placed in a vacuum transfer manifold and the extract filtered through the 0.45 mm membrane bottoms of each well using house vacuum. Extracts are collected in a 96-well catch/assay plate in the bottom of the vacuum manifold and immediately placed on ice. To obtain extracts clarified by centrifugation, the content of each well, after detergent solubilization for 5 minutes, is removed and centrifuged for 15 minutes at 4 C at 16,000 x g.

Test the filtered extracts for levels of tyrosine kinase activity. Although many methods of detecting tyrosine kinase activity are known, one method is described here.

Generally, the tyrosine kinase activity of a supernatant is evaluated by determining its ability to phosphorylate a tyrosine residue on a specific substrate (a biotinylated peptide). Biotinylated peptides that can be used for this purpose include PSK1 (corresponding to amino acids 6-20 of the cell division kinase cdc2-p34) and PSK2 (corresponding to amino acids 1- 17 of gastrin). Both peptides are substrates for a range of tyrosine kinases and are available from Boehringer Mannheim.

The tyrosine kinase reaction is set up by adding the following components in order. First, add 10 μl of 5 μM Biotinylated Peptide, then 10 μl ATP/Mg²⁺ (5 mM ATP/50 mM MgCl₂), then 10 μl of 5x Assay Buffer (40 mM imidazole hydrochloride, pH 7.3, 40 mM b-glycerophosphate, 1 mM EGTA, 100 mM MgCl₂, 5 mM MnCl₂, 0.5 mg/ml BSA), then 5 μl of Sodium Vanadate (1 mM), and then 5 μl of water. Mix the components gently and preincubate the reaction mix at 30 C for 2 min. Initial the reaction by adding 10 μl of the control enzyme or the filtered supernatant.

The tyrosine kinase assay reaction is then terminated by adding 10 μl of 120 mm EDTA and place the reactions on ice.

Tyrosine kinase activity is determined by transferring 50 μl aliquot of reaction mixture to a microtiter plate (MTP) module and incubating at 37 C for 20 min. This allows the streptavadin coated 96 well plate to associate with the biotinylated peptide. Wash the MTP module with 300 μl/well of PBS four times. Next add 75 μl of anti-phospotyrosine antibody conjugated to horse radish peroxidase(anti-P-Tyr-POD (0.5 μl/ml)) to each well and incubate at 37 C for one hour. Wash the well as above.

Next add 100 μl of peroxidase substrate solution (Boehringer Mannheim) and incubate at room temperature for at least 5 min (up to 30 min). Measure the absorbance of the sample at 405 nm by using ELISA reader. The level of bound peroxidase activity is quantitated using an ELISA reader and reflects the level of tyrosine kinase activity.

### Example 20: High-Throughput Screening Assay Identifying Phosphorylation Activity.

As a potential alternative and/or compliment to the assay of protein tyrosine kinase activity described in Example 19, an assay which detects activation (phosphorylation) of major intracellular signal transduction intermediates can also be used. For example, as described below one particular assay can detect tyrosine phosphorylation of the Erk-1 and Erk-2 kinases. However, phosphorylation of other molecules, such as Raf, JNK, p38 MAP, Map kinase kinase (MEK), MEK kinase, Src, Muscle specific kinase (MuSK), IRAK, Tec, and Janus, as well as any other phosphoserine, phosphotyrosine, or phosphothreonine molecule, can be detected by substituting these molecules for Erk-1 or Erk-2 in the following assay.

Specifically, assay plates are made by coating the wells of a 96-well ELISA plate with 0.1 ml of protein G (1 μg/ml) for 2 hr at room temp (RT). The plates are then rinsed with PBS and blocked with 3% BSA/PBS for 1 hr at RT. The protein G plates are then treated with 2 commercial monoclonal antibodies (100 ng/well) against Erk-1 and Erk-2 (1 hr at RT; available from Santa Cruz Biotechnology). To detect other molecules, this step can easily be modified by substituting a monoclonal antibody detecting any of the above described molecules. After 3-5 rinses with PBS, the plates are stored at 4 C until use.

A431 cells are seeded at 20,000/well in a 96-well Loprodyne filterplate and cultured overnight in growth medium. The cells are then starved for 48 hr in basal medium (DMEM) and then treated with EGF (6 ng/well) or 50 μl of the supernatants obtained in Example 11 for 5-20 minutes. The cells are then solubilized and extracts filtered directly into the assay plate.

After incubation with the extract for 1 hr at RT, the wells are again rinsed. As a positive control, a commercial preparation of MAP kinase (10 ng/well) is used in place of A431 extract. Plates are then treated with a commercial polyclonal (rabbit) antibody (1 μg/ml) which specifically recognizes the phosphorylated epitope of the Erk-1 and Erk-2 kinases (1 hr at RT). This antibody is biotinylated by standard procedures. The bound polyclonal antibody is then quantitated by successive incubations with Europium-streptavidin and Europium fluorescence enhancing reagent in the Wallac DELFIA instrument (time-resolved fluorescence). An increased fluorescent signal over background indicates a phosphorylation by IL-21 or a molecule induced by IL-21.

### Example 21: Method of Determining Alterations in the IL-21 Gene.

RNA isolated from entire families or individual patients presenting with a phenotype of interest (such as a disease) is be isolated. cDNA is then generated from these RNA samples using protocols known in the art (see, Sambrook, et al., supra) The cDNA is then used as a template for PCR, employing primers surrounding regions of interest in SEQ ID NO: 1. Suggested PCR conditions consist of 35 cycles at 95 C for 30 seconds; 60-120 seconds at 52-58 C; and 60-120 seconds at 70 C, using buffer solutions described by Sidransky and colleagues (Science 252:706 (1991)).

PCR products are then sequenced using primers labeled at the 5′ end with T4 polynucleotide kinase, employing SequiTherm Polymerase (Epicentre Technologies). The intron-exon borders of selected exons of IL-21 are also determined and genomic PCR products analyzed to confirm the results. PCR products harboring suspected mutations in IL-21 are then cloned and sequenced to validate the results of the direct sequencing.

PCR products of IL-21 are cloned into T-tailed vectors as described by Holton and Graham (Nucl. Acids Res. 19:1156 (1991)) and sequenced with T7 polymerase (United States Biochemical). Affected individuals are identified by mutations in IL-21 not present in unaffected individuals.

Genomic rearrangements are also observed as a method of determining alterations in the IL-21 gene. Genomic clones isolated according to Example 2 are nick-translated with digoxigenindeoxy-uridine 5′-triphosphate (Boehringer Manheim), and FISH performed as described by Johnson and coworkers (Methods Cell Biol. 35:73-99 (1991)). Hybridization with the labeled probe is carried out using a vast excess of human cot-1 DNA for specific hybridization to the IL-21 genomic locus.

Chromosomes are counterstained with 4,6-diamino-2-phenylidole and propidium iodide, producing a combination of C- and R-bands. Aligned images for precise mapping are obtained using a triple-band filter set (Chroma Technology, Brattleboro, Vt.) in combination with a cooled charge-coupled device camera (Photometrics, Tucson, Ariz.) and variable excitation wavelength filters (Johnson, C., et al., Genet. Anal. Tech. Appl. 8:75 (1991)). Image collection, analysis and chromosomal fractional length measurements are performed using the ISee Graphical Program System. (Inovision Corporation, Durham, N.C.). Chromosome alterations of the genomic region of IL-21 (hybridized by the probe) are identified as insertions, deletions, and translocations. These IL-21 alterations are used as a diagnostic marker for an associated disease.

### Example 22: Method of Detecting Abnormal Levels of IL-21 in a Biological Sample.

IL-2 1 polypeptides can be detected in a biological sample, and if an increased or decreased level of IL-21 is detected, this polypeptide is a marker for a particular phenotype. Methods of detection are numerous, and thus, it is understood that one skilled in the art can modify the following assay to fit their particular needs.

For example, antibody-sandwich ELISAs are used to detect IL-21 in a sample, preferably a biological sample. Wells of a microtiter plate are coated with specific antibodies to IL-21, at a final concentration of 0.2 to 10 μg/ml. The antibodies are either monoclonal or polyclonal and are produced by the method described in Example 10. The wells are blocked so that non-specific binding of IL-21 to the well is reduced.

The coated wells are then incubated for >2 hours at RT with a sample containing IL-21. Preferably, serial dilutions of the sample should be used to validate results. The plates are then washed three times with deionized or distilled water to remove unbounded IL-21.

Next, 50 μl of specific antibody-alkaline phosphatase conjugate, at a concentration of 25-400 ng, is added and incubated for 2 hours at room temperature. The plates are again washed three times with deionized or distilled water to remove unbounded conjugate.

Add 75 μl of 4-methylumbelliferyl phosphate (MUP) or p-nitrophenyl phosphate (NPP) substrate solution to each well and incubate 1 hour at room temperature. Measure the reaction by a microtiter plate reader. Prepare a standard curve, using serial dilutions of a control sample, and plot IL-21 polypeptide concentration on the X-axis (log scale) and fluorescence or absorbance of the Y-axis (linear scale). Interpolate the concentration of the IL-21 in the sample using the standard curve.

### Example 23: Formulating a Polypeptide.

The IL-21 composition will be formulated and dosed in a fashion consistent with good medical practice, taking into account the clinical condition of the individual patient (especially the side effects of treatment with the IL-21 polypeptide alone), the site of delivery, the method of administration, the scheduling of administration, and other factors known to practitioners. The “effective amount” for purposes herein is thus determined by such considerations.

As a general proposition, the total pharmaceutically effective amount of IL-21 administered parenterally per dose will be in the range of about 1 μg/kg/day to 10 mg/kg/day of patient body weight, although, as noted above, this will be subject to therapeutic discretion. More preferably, this dose is at least 0.01 mg/kg/day, and most preferably for humans between about 0.01 and 1 mg/kg/day for the hormone. If given continuously, IL-21 is typically administered at a dose rate of about 1 μg/kg/hour to about 50 μg/kg/hour, either by 1-4 injections per day or by continuous subcutaneous infusions, for example, using a mini-pump. An intravenous bag solution may also be employed. The length of treatment needed to observe changes and the interval following treatment for responses to occur appears to vary depending on the desired effect.

Using the equivalent surface area dosage conversion factors supplied by Freireich, E. J., et al. (Cancer Chemotherapy Reports 50(4):219-44 (1966)), one of ordinary skill in the art is able to conveniently convert data obtained from the use of IL-21 and/or IL-22 in a given experimental system into an accurate estimation of a pharmaceutically effective amount of IL-21 and/or IL-22 polypeptide to be administered per dose in another experimental system. Experimental data obtained through the administration of IL-21 and/or IL-22 in mice may converted through the conversion factors supplied by Freireich, et al., to accurate estimates of pharmaceutically effective doses of IL-21 and/or IL-22 in rat, monkey, dog, and human. The following conversion table (Table V) is a summary of the data provided by Freireich, et al. Table V gives approximate factors for converting doses expressed in terms of mg/kg from one species to an equivalent surface area dose expressed as mg/kg in another species tabulated.

Table V. Equivalent Surface Area Dosage Conversion Factors.

| | Mouse | Rat | Monkey | Dog | Human |
|---|---|---|---|---|---|
| FROM | (20 g) | (150 g) | (3.5 kg) | (8 kg) | (60 kg) |
| Mouse | 1 | ½ | ¼ | ⅙ | 1/12 |
| Rat | 2 | 1 | ½ | ¼ | 1/7 |
| Monkey | 4 | 2 | 1 | ⅗ | ⅓ |
| Dog | 6 | 4 | 5/3 | 1 | ½ |
| Human | 12 | 7 | 3 | 2 | 1 |

Thus, for example, using the conversion factors provided in Table III, a dose of 50 mg/kg in the mouse converts to an appropriate dose of 12.5 mg/kg in the monkey because (50 mg/kg)×(¼)=12.5 mg/kg. As an additional example, doses of 0.02, 0.08, 0.8, 2, and 8 mg/kg in the mouse equate to effect doses of 1.667 micrograms/kg, 6.67 micrograms/kg, 66.7 micrograms/kg, 166.7 micrograms/kg, and 0.667 mg/kg, respectively, in the human.

Pharmaceutical compositions containing IL-21 are administered orally, rectally, parenterally, intracistemally, intravaginally, intraperitoneally, topically (as by powders, ointments, gels, drops or transdermal patch), bucally, or as an oral or nasal spray. “Pharmaceutically acceptable carrier” refers to a non-toxic solid, semisolid or liquid filler, diluent, encapsulating material or formulation auxiliary of any type. The term “parenteral” as used herein refers to modes of administration which include intravenous, intramuscular, intraperitoneal, intrastemal, subcutaneous and intraarticular injection and infusion.

IL-21 is also suitably administered by sustained-release systems. Suitable examples of sustained-release compositions include semi-permeable polymer matrices in the form of shaped articles, e.g., films, or mirocapsules. Sustained-release matrices include polylactides (U.S. Pat. No. 3,773,919, EP 58,481), copolymers of L-glutamic acid and gamma-ethyl-L-glutamate (Sidman, U., et al., Biopolymers 22:547-556 (1983)), poly (2- hydroxyethyl methacrylate) (Langer, R., et al., J. Biomed. Mater. Res. 15:167-277 (1981); Langer, R. Chem. Tech. 12:98-105 (1982)), ethylene vinyl acetate (Langer, R., et al.) or poly-D- (−)-3-hydroxybutyric acid (EP 133,988). Sustained-release compositions also include liposomally entrapped IL-21 polypeptides. Liposomes containing the IL-21 are prepared by methods known per se (DE 3,218,121; Epstein, et al., Proc. Natl. Acad. Sci. USA 82:3688-3692 (1985); Hwang, et al., Proc. Natl. Acad. Sci. USA 77:4030-4034 (1980); EP 52,322; EP 36,676; EP 88,046; EP 143,949; EP 142,641; Japanese Pat. Appl. 83-118008; U.S. Pat. Nos. 4,485,045 and 4,544,545; and EP 102,324). Ordinarily, the liposomes are of the small (about 200-800 Angstroms) unilamellar type in which the lipid content is greater than about 30 mol. percent cholesterol, the selected proportion being adjusted for the optimal secreted polypeptide therapy.

For parenteral administration, in one embodiment, IL-21 is formulated generally by mixing it at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation. For example, the formulation preferably does not include oxidizing agents and other compounds that are known to be deleterious to polypeptides.

Generally, the formulations are prepared by contacting IL-21 uniformly and intimately with liquid carriers or finely divided solid carriers or both. Then, if necessary, the product is shaped into the desired formulation. Preferably the carrier is a parenteral carrier, more preferably a solution that is isotonic with the blood of the recipient. Examples of such carrier vehicles include water, saline, Ringers solution, and dextrose solution. Non-aqueous vehicles such as fixed oils and ethyl oleate are also useful herein, as well as liposomes.

The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, manose, or dextrins; chelating agents such as EDTA; sugar alcohols such as mannitol or sorbitol; counterions such as sodium; and/or nonionic surfactants such as polysorbates, poloxamers, or PEG.

IL-21 is typically formulated in such vehicles at a concentration of about 0.1 mg/ml to 100 mg/ml, preferably 1-10 mg/ml, at a pH of about 3 to 8. It will be understood that the use of certain of the foregoing excipients, carriers, or stabilizers will result in the formation of polypeptide salts.

IL-21 used for therapeutic administration can be sterile. Sterility is readily accomplished by filtration through sterile filtration membranes (e.g., 0.2 micron membranes). Therapeutic polypeptide compositions generally are placed into a container having a sterile access port, for example, an intravenous solution bag or vial having a stopper pierceable by a hypodermic injection needle.

IL-21 polypeptides ordinarily will be stored in unit or multi-dose containers, for example, sealed ampoules or vials, as an aqueous solution or as a lyophilized formulation for reconstitution. As an example of a lyophilized formulation, 10-ml vials are filled with 5 ml of sterile-filtered 1% (w/v) aqueous IL-21 polypeptide solution, and the resulting mixture is lyophilized. The infusion solution is prepared by reconstituting the lyophilized IL-21 polypeptide using bacteriostatic Water-For-Injection (WFI).

The invention also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration. In addition, IL-21 may be employed in conjunction with other therapeutic compounds.

### Example 24: Method of Treating Decreased Levels of IL-21.

The present invention relates to a method for treating an individual in need of a decreased level of IL-21 activity in the body comprising, administering to such an individual a composition comprising a therapeutically effective amount of IL-21 antagonist. Preferred antagonists for use in the present invention are IL-21-specific antibodies.

Moreover, it will be appreciated that conditions caused by a decrease in the standard or normal expression level of IL-21 in an individual can be treated by administering IL-21, preferably in the secreted form. Thus, the invention also provides a method of treatment of an individual in need of an increased level of IL-21 polypeptide comprising administering to such an individual a pharmaceutical composition comprising an amount of IL-21 to increase the activity level of IL-21 in such an individual.

For example, a patient with decreased levels of IL-21 polypeptide receives a daily dose 0.1-100 μg/kg of the polypeptide for six consecutive days. Preferably, the polypeptide is in the secreted form. The exact details of the dosing scheme, based on administration and formulation, are provided in Example 23.

### Example 25: Method of Treating Increased Levels of IL-21.

The present invention also relates to a method for treating an individual in need of an increased level of IL-21 activity in the body comprising administering to such an individual a composition comprising a therapeutically effective amount of IL-21 or an agonist thereof.

Antisense technology is used to inhibit production of IL-21. This technology is one example of a method of decreasing levels of IL-21 polypeptide, preferably a secreted form, due to a variety of etiologies, such as cancer.

For example, a patient diagnosed with abnormally increased levels of IL-21 is administered intravenously antisense polynucleotides at 0.5, 1.0, 1.5, 2.0 and 3.0 mg/kg day for 21 days. This treatment is repeated after a 7-day rest period if the treatment was well tolerated. The formulation of the antisense polynucleotide is provided in Example 23.

### Example 26: Method of Treatment Using Gene Therapy.

One method of gene therapy transplants fibroblasts, which are capable of expressing IL-21 polypeptides, onto a patient. Generally, fibroblasts are obtained from a subject by skin biopsy. The resulting tissue is placed in tissue-culture medium and separated into small pieces. Small chunks of the tissue are placed on a wet surface of a tissue culture flask, approximately ten pieces are placed in each flask. The flask is turned upside down, closed tight and left at room temperature over night. After 24 hours at room temperature, the flask is inverted and the chunks of tissue remain fixed to the bottom of the flask and fresh media (e.g., Ham's F12 media, with 10% FBS, penicillin and streptomycin) is added. The flasks are then incubated at 37 C for approximately one week.

At this time, fresh media is added and subsequently changed every several days. After an additional two weeks in culture, a monolayer of fibroblasts emerge. The monolayer is trypsinized and scaled into larger flasks.

pMV-7 (Kirschmeier, P. T., et al., DNA 7:219-25 (1988)), flanked by the long terminal repeats of the Moloney murine sarcoma virus, is digested with Eco RI and Hin dIII and subsequently treated with calf intestinal phosphatase. The linear vector is fractionated on agarose gel and purified, using glass beads.

The cDNA encoding IL-21 can be amplified using PCR primers which correspond to the 5′ and 3′ end sequences respectively as set forth in Example 1. Preferably, the 5′ primer contains an Eco RI site and the 3′ primer includes a Hin dIII site. Equal quantities of the Moloney murine sarcoma virus linear backbone and the amplified Eco RI and Hin dIII fragment are added together, in the presence of T4 DNA ligase. The resulting mixture is maintained under conditions appropriate for ligation of the two fragments. The ligation mixture is then used to transform bacteria HB101, which are then plated onto agar containing kanamycin for the purpose of confirming that the vector contains properly inserted IL-21.

The amphotropic pA317 or GP+am12 packaging cells are grown in tissue culture to confluent density in Dulbecco's Modified Eagles Medium (DMEM) with 10% calf serum (CS), penicillin and streptomycin. The MSV vector containing the IL-21 gene is then added to the media and the packaging cells transduced with the vector. The packaging cells now produce infectious viral particles containing the IL-21 gene (the packaging cells are now referred to as producer cells).

Fresh media is added to the transduced producer cells, and subsequently, the media is harvested from a 10 cm plate of confluent producer cells. The spent media, containing the infectious viral particles, is filtered through a millipore filter to remove detached producer cells and this media is then used to infect fibroblast cells. Media is removed from a sub-confluent plate of fibroblasts and quickly replaced with the media from the producer cells. This media is removed and replaced with fresh media. If the titer of virus is high, then virtually all fibroblasts will be infected and no selection is required. If the titer is very low, then it is necessary to use a retroviral vector that has a selectable marker, such as neo or his. Once the fibroblasts have been efficiently infected, the fibroblasts are analyzed to determine whether IL-21 protein is produced.

The engineered fibroblasts are then transplanted onto the host, either alone or after having been grown to confluence on cytodex 3 microcarrier beads.

It will be clear that the invention may be practiced otherwise than as particularly described in the foregoing description and examples. Numerous modifications and variations of the present invention are possible in light of the above teachings and, therefore, are within the scope of the appended claims.

The entire disclosure of each document cited (including patents, patent applications, journal articles, abstracts, laboratory manuals, books, or other disclosures) in the Background of the Invention, Detailed Description, and Examples is hereby incorporated herein by reference.

Further, the Sequence Listing submitted herewith, and each of the Sequence Listings submitted with U.S. application Ser. Nos. 60/169,837, filed on Dec. 9, 1999, 09/320,713, filed May 27, 1999, 60/087,340, filed on May 29, 1998, 60/099,805, filed on Sep. 10, 1998, and 60/131,965, filed on Apr. 30, 1999, and to International Application Serial No. U.S. 99/11644, filed May 27, 1999 (to each of which the present application claims benefit of the filing dates under 35 U.S.C. §§ 119(e) and/or 120), in both CRF and paper formats are hereby incorporated by reference in their entireties.

What is claimed is:

1. An isolated nucleic acid molecule comprising a polynucleotide having a nucleotide sequence at least 95% identical to a sequence selected from the group consisting of:
(a) a polynucleotide fragment of SEQ ID NO: 1 or a polynucleotide fragment of the cDNA sequence included in ATCC Deposit No: 209666;
(b) a polynucleotide encoding a polypeptide fragment of SEQ ID NO: 2 or the cDNA sequence included in ATCC Deposit No: 209666;
(c) a polynucleotide encoding conserved polypeptide domain I of SEQ ID NO: 2 or the cDNA sequence included in ATCC Deposit No: 209666;
(d) a polynucleotide encoding conserved polypeptide domain II of SEQ ID NO: 2 or the cDNA sequence included in ATCC Deposit No: 209666;
(e) a polynucleotide encoding conserved polypeptide domain III of SEQ ID NO: 2 or the cDNA sequence included in ATCC Deposit No: 209666;
(f) a polynucleotide encoding conserved polypeptide domain IV of SEQ ID NO: 2 or the cDNA sequence included in ATCC Deposit No: 209666;
(g) a polynucleotide encoding a polypeptide epitope of SEQ ID NO: 2 or the cDNA sequence included in ATCC Deposit No: 209666;
(h) a polynucleotide encoding a polypeptide of SEQ ID NO: 2 or the cDNA sequence included in ATCC Deposit No: 209666 having biological activity;
(i) a polynucleotide which is a variant of SEQ ID NO: 1;
(j) a polynucleotide which is an allelic variant of SEQ ID NO: 1;
(k) a polynucleotide which encodes a species homologue of the polypeptide whose amino acid sequence is shown in SEQ ID NO: 2;
(l) a polynucleotide capable of hybridizing under stringent conditions to any one of the polynucleotides specified in (a), (b), (c), (d), (e), (f), (g), (h), (i), (j) or (k), wherein said polynucleotide does not hybridize under stringent conditions to a nucleic acid molecule having a nucleotide sequence of only A residues or of only T residues; and
(m) a polynucleotide which is the complement of any one of the polynucleotides specificed in (a), (b), (c), (d), (e), (f), (g), (h), (i), (j), (k) or (m).

2. An isolated nucleic acid molecule comprising a polynucleotide having a nucleotide sequence at least 95% identical to a sequence selected from the group consisting of:
(a) a polynucleotide fragment of SEQ ID NO: 28;
(b) a polynucleotide encoding a polypeptide fragment of SEQ ID NO: 28;
(c) a polynucleotide encoding conserved polypeptide domain I of SEQ ID NO: 28;
(d) a polynucleotide encoding conserved polypeptide domain II of SEQ ID NO: 28;
(e) a polynucleotide encoding conserved polypeptide domain III of SEQ ID NO: 28;
(f) a polynucleotide encoding conserved polypeptide domain IV of SEQ ID NO: 28;
(g) a polynucleotide encoding conserved polypeptide domain V of SEQ ID NO: 28;
(h) a polynucleotide encoding conserved polypeptide domain VI of SEQ ID NO: 28;
(i) a polynucleotide encoding conserved polypeptide domain VII of SEQ ID NO: 28;
(j) a polynucleotide encoding a polypeptide epitope of SEQ ID NO: 28;
(k) a polynucleotide encoding a polypeptide of SEQ ID NO: 28 having biological activity;
(l) a polynucleotide which is a variant of SEQ ID NO: 28;
(m) a polynucleotide which is an allelic variant of SEQ ID NO: 28;
(n) a polynucleotide which encodes a species homologue of the polypeptide whose amino acid sequence is shown in SEQ ID NO: 28;
(o) a polynucleotide capable of hybridizing under stringent conditions to any one of the polynucleotides specified in (a), (b), (c), (d), (e), (f), (g), (h), (i), (j), (k), (l), (m) or (n), wherein said polynucleotide does not hybridize under stringent conditions to a nucleic acid molecule having a nucleotide sequence of only A residues or of only T residues; and
(p) a polynucleotide which is the complement of any one of the polynucleotides specificed in (a), (b), (c), (d), (e), (f), (g), (h), (i), (j), (k), (l), (m), (n) or (o).

3. An isolated nucleic acid molecule comprising a polynucleotide having a nucleotide sequence at least 95% identical to a sequence selected from the group consisting of:
(a) a polynucleotide fragment of SEQ ID NO: 3 or a polynucleotide fragment of the cDNA sequence included in ATCC Deposit No: 209665;
(b) a polynucleotide encoding a polypeptide fragment of SEQ ID NO: 4 or the cDNA sequence included in ATCC Deposit No: 209665;
(c) a polynucleotide encoding conserved polypeptide domain I of SEQ ID NO: 4 or the cDNA sequence included in ATCC Deposit No: 209665;
(d) a polynucleotide encoding conserved polypeptide domain II of SEQ ID NO: 4 or the cDNA sequence included in ATCC Deposit No: 209665;
(e) a polynucleotide encoding conserved polypeptide domain III of SEQ ID NO: 4 or the cDNA sequence included in ATCC Deposit No: 209665;
(f) a polynucleotide encoding conserved polypeptide domain IV of SEQ ID NO: 4 or the cDNA sequence included in ATCC Deposit No: 209665;
(g) a polynucleotide encoding a polypeptide epitope of SEQ ID NO: 4 or the cDNA sequence included in ATCC Deposit No: 209665;
(h) a polynucleotide encoding a polypeptide of SEQ ID NO: 4 or the cDNA sequence included in ATCC Deposit No: 209665 having biological activity;
(i) a polynucleotide which is a variant of SEQ ID NO: 3;
(j) a polynucleotide which is an allelic variant of SEQ ID NO: 3;
(k) a polynucleotide which encodes a species homologue of the polypeptide whose amino acid sequence is shown in SEQ ID NO: 4;
(l) a polynucleotide capable of hybridizing under stringent conditions to any one of the polynucleotides specified in (a), (b), (c), (d), (e), (f), (g), (h), (i), (j) or (k), wherein said polynucleotide does not hybridize under stringent conditions to a nucleic acid molecule having a nucleotide sequence of only A residues or of only T residues; and
(m) a polynucleotide which is the complement of any one of the polynucleotides specificed in (a), (b), (c), (d), (e), (f), (g), (h), (i), (j), (k) or (l).

4. The isolated nucleic acid molecule of claim 1, wherein the polynucleotide fragment comprises a nucleotide sequence encoding a mature form or a secreted protein.

5. The isolated nucleic acid molecule of claim 2, wherein the polynucleotide fragment comprises a nucleotide sequence encoding a mature form or a secreted protein.

6. The isolated nucleic acid molecule of claim 3, wherein the polynucleotide fragment comprises a nucleotide sequence encoding a mature form or a secreted protein.

7. The isolated nucleic acid molecule of claim 1, wherein the polynucleotide fragment comprises a nucleotide sequence encoding the sequence identified as SEQ ID NO: 2 or the coding sequence included in ATCC Deposit No: 209666.

8. The isolated nucleic acid molecule of claim 2, wherein the polynucleotide fragment comprises a nucleotide sequence encoding the sequence identified as SEQ ID NO: 28.

9. The isolated nucleic acid molecule of claim 3, wherein the polynucleotide fragment comprises a nucleotide sequence encoding the sequence identified as SEQ ID NO: 4 or the coding sequence included in ATCC Deposit No: 209665.

10. The isolated nucleic acid molecule of claim 1, wherein the polynucleotide fragment comprises the entire nucleotide sequence of SEQ ID NO: 1 or the cDNA sequence included in ATCC Deposit No: 209666.

11. The isolated nucleic acid molecule of claim 2, wherein the polynucleotide fragment comprises the entire nucleotide sequence of SEQ ID NO: 28.

12. The isolated nucleic acid molecule of claim 3, wherein the polynucleotide fragment comprises the entire nucleotide sequence of SEQ ID NO: 3 or the cDNA sequence included in ATCC Deposit No: 209665.

13. The isolated nucleic acid molecule of claim 5, wherein the nucleotide sequence comprises sequential nucleotide deletions from either the C-terminus or the N-terminus.

14. The isolated nucleic acid molecule of claim 8, wherein the nucleotide sequence comprises sequential nucleotide deletions from either the C-terminus or the N-terminus.

15. A recombinant vector comprising the isolated nucleic acid molecule of claim 2.

16. A method of making a recombinant host cell comprising the isolated nucleic acid molecule of claim 1.

17. A method of making a recombinant host cell comprising the isolated nucleic acid molecule of claim 2.

18. A method of making a recombinant host cell comprising the isolated nucleic acid molecule of claim 3.

19. A recombinant host cell produced by the method of claim 16.

20. A recombinant host cell produced by the method of claim 17.

21. A recombinant host cell produced by the method of claim 18.

22. The recombinant host cell of claim 19 comprising vector sequences.

23. The recombinant host cell of claim 20 comprising vector sequences.

24. The recombinant host cell of claim 21 comprising vector sequences.

25. An isolated polypeptide comprising an amino acid sequence at least 95% identical to a sequence selected from the group consisting of:
(a) a polypeptide fragment of SEQ ID NO: 2 or the encoded sequence included in ATCC Deposit No: 209666;
(b) a polypeptide fragment of SEQ ID NO: 2 or the encoded sequence included in ATCC Deposit No: 209666 having biological activity;
(c) a polypeptide domain of SEQ ID NO: 2 or the encoded sequence included in ATCC Deposit No: 209666;
(d) a polypeptide epitope of SEQ ID NO: 2 or the encoded sequence included in ATCC Deposit No: 209666;
(e) a mature form of a secreted protein;
(f) a full length secreted protein;
(g) a variant of SEQ ID NO: 2;
(h) an allelic variant of SEQ ID NO: 2; and
(i) a species homologue of the SEQ ID NO: 2.

26. An isolated polypeptide comprising an amino acid sequence at least 95% identical to a sequence selected from the group consisting of:
(a) a polypeptide fragment of SEQ ID NO: 29;
(b) a polypeptide fragment of SEQ ID NO: 29 having biological activity;
(c) a polypeptide domain of SEQ ID NO: 29;
(d) a polypeptide epitope of SEQ ID NO: 29;
(e) a mature form of a secreted protein of SEQ ID NO: 29;
(f) a full length secreted protein of SEQ ID NO: 29;
(g) a variant of SEQ ID NO: 29;
(h) an allelic variant of SEQ ID NO: 29; and
(i) a species homologue of the SEQ ID NO: 29.

27. An isolated polypeptide comprising an amino acid sequence at least 95% identical to a sequence selected from the group consisting of:
(a) a polypeptide fragment of SEQ ID NO: 4 or the encoded sequence included in ATCC Deposit No: 209665;
(b) a polypeptide fragment of SEQ ID NO: 4 or the encoded sequence included in ATCC Deposit No: 209665 having biological activity;
(c) a polypeptide domain of SEQ ID NO: 4 or the encoded sequence included in ATCC Deposit No: 209665;
(d) a polypeptide epitope of SEQ ID NO: 4 or the encoded sequence included in ATCC Deposit No: 209665;
(e) a mature form of a secreted protein;
(f) a full length secreted protein;
(g) a variant of SEQ ID NO: 4;
(h) an allelic variant of SEQ ID NO: 4; and
(i) a species homologue of the SEQ ID NO: 4.

28. The isolated polypeptide of claim 26, wherein the mature form or the full length secreted protein comprises sequential amino acid deletions from either the C-terminus or the N-terminus.

29. An isolated antibody that binds specifically to the isolated polypeptide of claim 26.

30. A recombinant host cell that expresses the isolated polypeptide of claim 25.

31. A recombinant host cell that expresses the isolated polypeptide of claim 26.

32. A recombinant host cell that expresses the isolated polypeptide of claim 27.

33. A method of making an isolated polypeptide comprising:
(a) culturing the recombinant host cell of claim 30 under conditions such that said polypeptide is expressed; and
(b) recovering said polypeptide.

34. A method of making an isolated polypeptide comprising:
(a) culturing the recombinant host cell of claim 31 under conditions such that said polypeptide is expressed; and
(b) recovering said polypeptide.

35. A method of making an isolated polypeptide comprising:
(a) culturing the recombinant host cell of claim 32 under conditions such that said polypeptide is expressed; and
(b) recovering said polypeptide.

36. The polypeptide produced by claim 34.

37. A method for preventing, treating, or ameliorating a medical condition which comprises administering to a mammalian subject a therapeutically effective amount of the polypeptide of claim 26.

38. A method of diagnosing a pathological condition or a susceptibility to a pathological condition in a subject related to expression or activity of a secreted protein comprising:
(a) determining the presence or absence of a mutation in the polynucleotide of claim 2;
(b) diagnosing a pathological condition or a susceptibility to a pathological condition based on the presence or absence of said mutation.

39. A method of diagnosing a pathological condition or a susceptibility to a pathological condition in a subject related to expression or activity of a secreted protein comprising:
(a) determining the presence or amount of expression of the polypeptide of claim 26 in a biological sample;
(b) diagnosing a pathological condition or a susceptibility to a pathological condition based on the presence or amount of expression of the polypeptide.

40. A method for identifying binding partner to the polypeptide of claim 26 comprising:
(a) contacting the polypeptide of claim 26 with a binding partner; and
(b) determining whether the binding partner effects an activity of the polypeptide.

41. The gene corresponding to the cDNA sequence of SEQ ID NO: 1.

42. The gene corresponding to the cDNA sequence of SEQ ID NO: 28.

43. The gene corresponding to the cDNA sequence of SEQ ID NO: 3.

44. A method of identifying an activity in a biological assay, wherein the method comprises:
(a) expressing SEQ ID NO: 1 in a cell;
(b) isolating the supernatant;
(c) detecting an activity in a biological assay; and
(d) identifying the protein in the supernatant having the activity.

45. A method of identifying an activity in a biological assay, wherein the method comprises:
(a) expressing SEQ ID NO: 28 in a cell;
(b) isolating the supernatant;
(c) detecting an activity in a biological assay; and
(d) identifying the protein in the supernatant having the activity.

46. A method of identifying an activity in a biological assay, wherein the method comprises:
(a) expressing SEQ ID NO: 3 in a cell;
(b) isolating the supernatant;
(c) detecting an activity in a biological assay; and
(d) identifying the protein in the supernatant having the activity.

47. The product produced by the method of claim 44.

48. The product produced by the method of claim 45.

49. The product produced by the method of claim 46.
